(19) United States
(12) Patent Application Publication
(10) Pub. No.: US 2026/0028348 A1
(43) Pub. Date: Jan. 29, 2026

(54) MODULATORS OF TNF-ALPHA ACTIVITY

(71) Applicant: Forward Therapeutics, Inc., Palm Beach Gardens, FL (US)

(72) Inventor: Toufike KANOUNI, Palm Beach Gardens, FL (US)

(21) Appl. No.: 19/144,287

(22) PCT Filed: Jan. 4, 2024
(86) PCT No.: PCT/US2024/010353

§ 371 Date: Jun. 27, 2025

Related U.S. Application Data
(60) Provisional application No. 63/478,643, filed on Jan. 5, 2023.

(51) Int. Cl. C07D 487/18; A61K 31/55; C07B 59/00; C07D 519/00

(52) U.S. Cl. CPC C07D 487/18; A61K 31/55; C07B 59/002; C07D 519/00

(57) ABSTRACT

Provided herein are inhibitors of TNF-α, pharmaceutical compositions comprising the inhibitory compounds, and methods for using the TNF-α inhibitory compounds for the treatment of diseases or disorders.

### CROSS-REFERENCE

This application claims the benefit of U.S. Provisional Application No. 63/478,643, filed Jan. 5, 2023, which is incorporated herein by reference in its entirety.

### BACKGROUND

Tumor necrosis factor alpha (TNF-α) is an inflammatory cytokine that is responsible for a wide range of signaling events within cells. Aberrant TNF-α signaling gives rise to inflammatory conditions and is thought to be an important component of inflammatory disease, such as rheumatoid arthritis.

### BRIEF SUMMARY OF THE INVENTION

Provided herein are inhibitors of TNF-α, pharmaceutical compositions comprising said inhibitory compounds, and methods for using said inhibitory compounds for the treatment of inflammatory or autoimmune disease or disorder.

One embodiment provides a compound of Formula (I), or pharmaceutically acceptable salt, solvate, or N-oxide thereof:

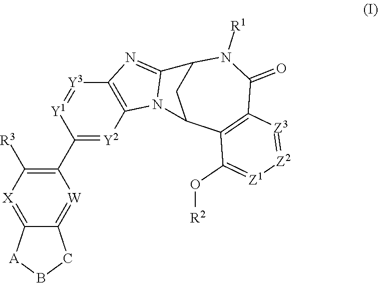

wherein,
W is N or C—R⁴;
X is N, C—F, or C—H;
Y¹ is N, C—F, or C—H;
Y² is N, C—F, or C—H;
Y³ is N, C—F, or C—H;
Z¹ is N or C—H;
Z² is N or C—H;
Z³ is N or C—H;
A is CR⁵R⁶, or N—R⁹;
B is CR⁷R⁸, or N—R¹⁰;
C is —CH₂—, —CH₂CH₂—, —NH—, —O— or —CH₂O—;
R¹ is selected from hydrogen, optionally substituted C1-C6 alkyl, optionally substituted C3-C6 cycloalkyl, or optionally substituted C4-C7 cycloalkylalkyl;
R² is hydrogen, or optionally substituted C1-C4 alkyl;
R³ is hydrogen, methyl, or halogen;
R⁴ is hydrogen, halogen, or optionally substituted C1 alkyl;
   R⁵ is —OH, —NH₂, or —NH(optionally substituted C1-C6 alkyl); or optionally, R⁵ and R⁶ are both —OH and join to form an oxo group;
R⁶ is hydrogen, —OH, or optionally substituted C1-C6 alkyl; or optionally, R⁶ and R⁸ are both optionally substituted C1-C6 alkyl and join to form a ring;
R⁷ is hydrogen, —OH, or optionally substituted C1-C6 alkyl; or optionally, R⁷ and R⁸ are both —OH and join to form an oxo group;
R⁸ is hydrogen, —OH, or optionally substituted C1-C6 alkyl;
   R⁹ is hydrogen, or optionally substituted C1-C6 alkyl; and
   R¹⁰ is hydrogen, or optionally substituted C1-C6 alkyl.

One embodiment provides a pharmaceutical composition comprising a compound of Formula (I), or pharmaceutically acceptable salt, solvate, or N-oxide thereof, and at least one pharmaceutically acceptable excipient.

One embodiment provides a method of treating a disease or disorder in a patient in need thereof comprising administering to the patient a compound of Formula (I), or pharmaceutically acceptable salt, solvate, or N-oxide thereof. Another embodiment provides the method wherein the disease or disorder is rheumatoid arthritis.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₅ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl). In certain embodiments, an optionally substituted alkyl is a haloalkyl. In other embodiments, an optionally substituted alkyl is a fluoroalkyl. In other embodiments, an optionally substituted alkyl is a —CF₃ group.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈ alkenylene). In other embodiments, an alkenylene comprises three to five carbon atoms (e.g., C₃-C₅ alkenylene).

Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from optionally substituted alkyl, optionally substituted alkenyl, optionally substituted alkynyl, halo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from optionally substituted alkyl, optionally substituted alkenyl, optionally substituted alkynyl, halo, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkynyl” refers to a radical of the formula —R^{c}-carbocyclyl where R^{c} is an alkynylene chain as defined above. The alkynylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic, or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from optionally substituted alkyl, optionally substituted alkenyl, optionally substituted alkynyl, halo, fluoroalkyl, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen, and sulfur. As used herein, the heteroaryl radical is a monocyclic, bicyclic, tricyclic, or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from optionally substituted alkyl, optionally substituted cycloalkylalkyl, optionally substituted heterocyclylalkyl, optionally substituted alkenyl, optionally substituted alkynyl, halo, optionally substituted fluoroalkyl, optionally substituted haloalkenyl, optionally substituted haloalkynyl, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

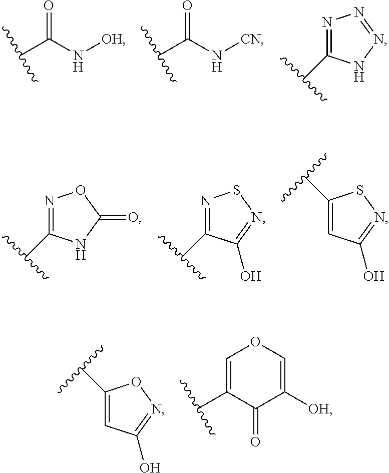

and the like.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

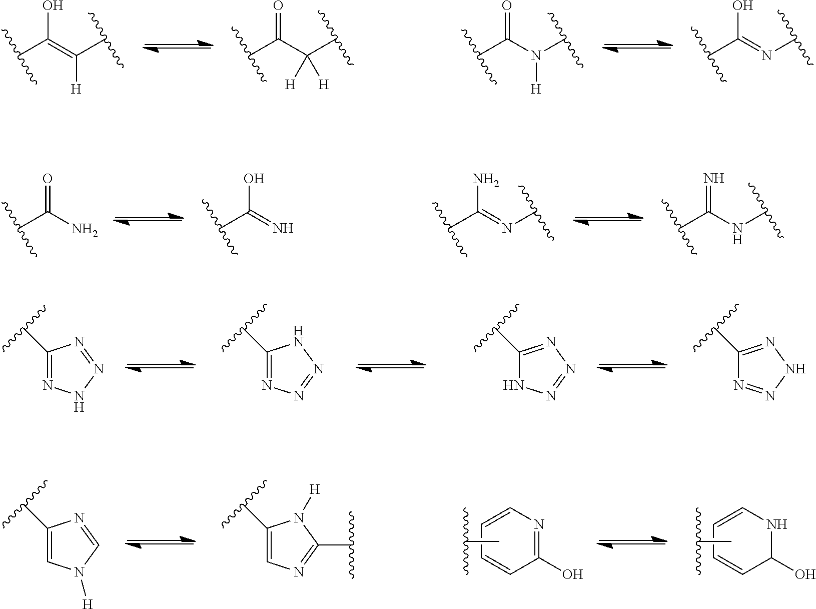

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

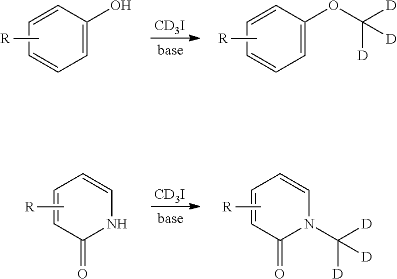

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

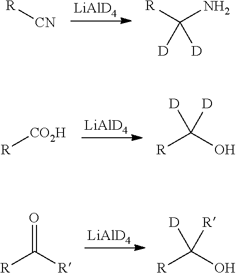

Deuterium gas and palladium catalyst are employed to reduce unsaturated carbon-carbon linkages and to perform a reductive substitution of aryl carbon-halogen bonds as illustrated, by way of example only, in the reaction schemes below.

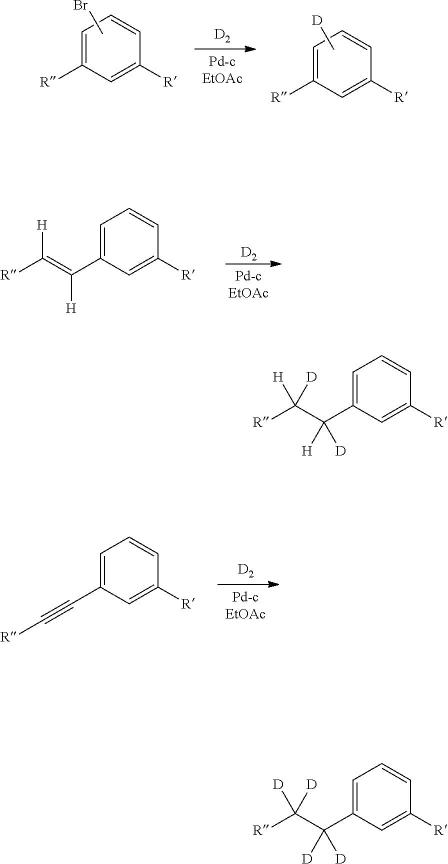

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the TNF-α inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein exist in either unsolvated or solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### Tumor Necrosis Factor Alpha (TNFα) Protein and Function

Tumor necrosis factor alpha (TNFα) proteins are members of the TNF superfamily, comprising various transmembrane proteins with a homologous TNF domain forming trimers.

The TNF superfamily comprises 19 family members, including, but not limited to tumor necrosis factor alpha (also known as tumor necrosis factor, or TNF), lymphotoxin alpha (TNFβ), lymphotoxin beta (TNFγ), OX40 ligand, CD40 ligand, Fas ligand, CD27 ligand, CD30 ligand, CD137 ligand, CD137 ligand, and TNF-related apoptosis-inducing ligand. TNFα proteins are cytokines and adipokines (cytokines secreted by adipose tissue).

TNFα is a transmembrane protein, with soluble TNFα (sTNFα) released via protein cleavage. The sTNFα can propagate signaling by binding to two receptors, TNFR1 and TNFR2. TNFα is a regulator of immune responses for cell signaling and can mediate cell survival and cell death inducing signaling. There are two receptors for TNF signaling, TNFR1 and TNFR2. sTNFα-TNFR1 signaling promotes immune cell activation and drives acute and chronic inflammation. Membrane TNFα-TNFR2 signaling promotes inflammation resolution, immune cell regulatory functions and cell survival.

The extracellular region of both TNFR1 and TNFR2 have four homologous cysteine-rich domains, but they have structurally different intracellular regions. TNFR1 has a protein binding region called a death domain which allows homo- and hetero-typic interactions with other death domain-containing proteins. In contrast, TNFR2 has a TNF Receptor Associated Factor TRAF) that interacts with TRAF family of signaling adaptors. The distinct profiles and differences of the two TNF receptors influence the cellular activity and physiological roles. TNFR1 can activate NF-κB and MAPK signaling, and cell death, and is important to regulate for inflammatory diseases. TNFR2 is highly regulated and restricted to specific cell types such as endothelial cells and T cells. TNFR1 primarily promotes tissue degeneration and inflammation and TNFR2 typically mediates local homestatic effects such as tissue regeneration and cell survival (D. Fresegna et al., Cells, 2020, 9, 2290).

Binding of TNFα to TNFR1 can activate NF-κB for mediating transcription of various proteins involved in cell survival and proliferation, anti-apoptotic factors, and inflammatory response. Further, the MAPK pathway can also be activated by binding of TNFα to TNFR1, which is involved in cell differentiation and proliferation. When TNF binds to TNFR1, it triggers receptor trimerization, leading to the assembly of a TNFR1-associated signaling complex. This complex recruits the receptor interacting protein 1 (RIP2) and TNF receptor associated death domain (TRADD) to the TNFR1 through the receptive death domains. TRADD then recruits adaptor proteins TRAF2 and TRAF5, which can engage the E3 ligases cellular inhibitors of apoptosis (c-IAP1, c-IAP2). C-IAP1/2 are important for TNFR1 complex signaling, which can eventually lead to the recruitment of the signaling kinase complexes of kinase IKKα and IKKβ, which are inhibitors of kappa B kinase 1 and 2, and transforming growth factor beta-activated kinase 1 (TAK1) leading to activation of NF-κB and MAPK signaling. Activation of these signaling pathways can result in gene activation and expression of pro-inflammatory cytokines and pro-survival proteins.

TNF signaling is regulated by post-translational ubiquitination, which is essential for my biological processes. Post-translational modifications of TNFR1-associated signaling complexes can result in a change from inflammatory gene signaling to cell death. This switch is dependent upon the ubiquitination status of RIP1, which is formed as part of the TNFR1-associated signaling complex from TNFα binding.

TNF has long been known to be a key regulator of the inflammatory response, and recently has been known to be involved n brain functioning (D. Fresegna et al., Cells, 2020, 9, 2290). As a regulator of the inflammatory response, TNF can regulate many aspects of T cell biology including, but not limited to proliferation, survival, priming, and apoptotic fate. TNF is also known to play a role in conclusion of lymphocyte response, by the ability to promote cell death in both CD4 and CD8P T cells, through TNFR1. Specific inflammatory conditions can also result in TNFR2 promoting or supporting T cell apoptosis.

In normal adult brains, TNF is expressed at low levels, and it is believed that the expression could be influenced by presence or absence of cytokines that can cross the blood brain barrier. TNFRs in the brain are expressed by glia and neurons cells, and have regulatory functions, including, but not limited to homeostatic synaptic plasticity, astrocyte-mediated synaptic transmission, and neurogenesis. These functions are useful for regulating learning and memory functions amongst other roles.

TNF is recognized to be physiological gliotransmitter for the communication between neurons and glial cells, which in turn affects synaptic regulation. Glial TNF is important for maintenance of normal surface expression of AMPA receptors, and for homeostatic synaptic scaling, which allows for adjustment of the strength of all synapses on a neuron.

### Prior Art Small Molecules Inhibitors

Diseases treated with biologic TNFα inhibitors include, but are not limited to rheumatoid arthritis, inflammatory bowel disease, psoriatic arthritis, psoriasis, and ankylosing spondylitis.

Patients with neuroinflammatory conditions and degenerative disease, including, but not limited to Alzheimer's disease, Parkinson's disease, multiple sclerosis, treatment resistant depression, and tinnitus, may benefit from treatment with oral CNS sTNFα inhibitors by disrupting the sTNFα signaling and sparing the mTNFα signaling. Previous reports have also indicated targeting TNFR2 for treating Alzheimer's Disease (N. Orti-Casañ et al., Front Neurosci. 2019; 13: 49).

Small molecules have been developed for treatment of rheumatoid arthritis as some patients have responded poorly to monotherapy of approved anti-TNFα drugs (J. D. Dietrich et al., J. Med. Chem. 2021, 64, 417-429). Anti-TNFα drugs have also been expanded for use in other chronic autoimmune diseases, including, but not limited to, Crohn's disease, psoriasis, psoriatic arthritis, ulcerative colitis inflammatory bowel disease, ankylosing spondylitis, and juvenile rheumatoid arthritis. Small molecules have been developed as an alternative to anti-TNFα biologics since the long-term clinical response rate is generally around 60-70% for rheumatoid arthritis.

Previous research has also indicated that TNFα inhibitors can be therapeutic for treatment of multiple sclerosis (D. Fresegna et al., Cells, 2020, 9, 2290). There has been evidence of the involvement of TNF in various pathological issues of multiple sclerosis, including immune dysregulation, demylination, synaptopathy, and neuroinflammation. TNFα inhibitors have the potential for treatment of multiple sclerosis, other potential chronic neurodegenerative diseases of the central nervous system.

More than 50 million Americans struggle with tinnitus, which is the hearing of a sound with no external source. It has been shown that TNFα is necessary for noise-induced neuroinflammation and synaptic imbalance (W. Wang et al., PLoS Biol. 2019 Jun. 18; 17(6):e3000307; A. Shulman et al., Curr Top Behav Neurosci. 2021; 51:161-174). It is believed that certain inhibitors of TNFα have activities for treating tinnitus.

Recent reports also indicate that TNFα inhibitors can be used alone or in combination for treatment with inflammatory bowel disease (S. F. Fowler Braga and K. J. Clark, US Pharm. 2021; 46(5):34-37). TNFα is a mediator of the abnormal immune response of inflammatory bowel disease, which leads to disruption of the intestinal mucosa and epithelial wall barrier. The anti-TNF agents can block TNF-mediated activation of the proinflammatory pathways to result in decreased immune-mediated inflammation.

Small molecule sTNFα inhibitors are active in pharmacology models of sTNFα/TNFR1 signaling in addition to demonstrating efficacy in a model of collagen antibody induced arthritis. There is currently limited data in the public domain for small molecule sTNF α inhibitors. Some TNFα inhibitors include, but are not limited to XPro1595, Etanercept, Infliximab, Adalimumab, Certolizumab pegol, Golimumamb, and other inhibitors described in “TNF-α: The Shape of Small Molecules to Come?” (A. Dömling and X. Li, Drug Discov Today 2022 January; 27(1):3-7) and “Small Molecules that Inhibit TNF Signalling by Stabilising an Asymmetric Form of the Trimer (J. O'Connell et al., Nature Communications 10, 5795 (2019)). Additional small molecule inhibitors of TNFα include, but are not limited to the inhibitors described in “Biologic-like In Vivo Efficacy with Small Molecule Inhibitors of TNFα Identified Using Scaffold Hopping and Structure-Based Drug Design Approaches” (H-Y Xiao et al., J. Med. Chem. 2020, 15050-15071), “Development of Orally Efficacious Allosteric Inhibitors of TNFα via Fragment-Based Drug Design” (J. D. Dietrich et al., J. Med. Chem. 2021, 64, 417-429), and “Small-Molecule Inhibition of TNF-α” (M. M. He et al., Science, 310 (2015), 1022-1025).

Small molecule sTNFα inhibitors have potential as a valuable therapy for patients currently treated with biologic TNFα inhibitors which affect mTNFα with the ability to fine tune oral dosing requirements and avoid anti-drug antibody responses, thereby improving short and long responses (A. Dömling and X. Li, Drug Discov Today 2022 January; 27(1):3-7).

### Novel Compounds Inhibiting TNF-α

In one aspect, provided herein are TNF-α inhibitory compounds.

One embodiment provides a compound of Formula (I), or pharmaceutically acceptable salt, solvate, or N-oxide thereof:

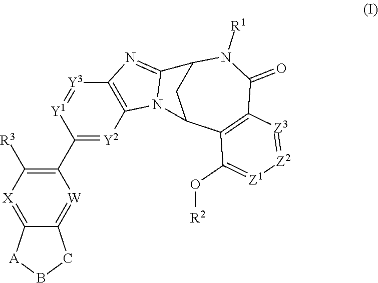

wherein,
W is N or C—R⁴;
X is N, C—F, or C—H;
Y¹ is N, C—F, or C—H;
Y² is N, C—F, or C—H;
Y³ is N, C—F, or C—H;
Z¹ is N or C—H;
Z² is N or C—H;
Z³ is N or C—H;
A is CR⁵R⁶, or N—R⁹;
B is CR⁷R⁸, or N—R¹⁰;
C is —CH₂—, —CH₂CH₂—, —NH—, —O—, —OCH₂—, or —CH₂O—;
R¹ is selected from hydrogen, optionally substituted C1-C6 alkyl, optionally substituted C3-C6 cycloalkyl, or optionally substituted C4-C7 cycloalkylalkyl;
R² is hydrogen, or optionally substituted C1-C4 alkyl;
R³ is hydrogen, methyl, or halogen;
R⁴ is hydrogen, halogen, or optionally substituted C1 alkyl;
   R⁵ is —OH, —NH₂, or —NH(optionally substituted C1-C6 alkyl); or optionally, R⁵ and R⁶ are both —OH and join to form an oxo group;
R⁶ is hydrogen, —OH, or optionally substituted C1-C6 alkyl; or optionally, R⁶ and R⁸ are both optionally substituted C1-C6 alkyl and join to form a ring;
R⁷ is hydrogen, —OH, or optionally substituted C1-C6 alkyl; or optionally, R⁷ and R⁸ are both —OH and join to form an oxo group;
R⁸ is hydrogen, —OH, or optionally substituted C1-C6 alkyl;
   R⁹ is hydrogen, or optionally substituted C1-C6 alkyl; and
R¹⁰ is hydrogen, or optionally substituted C1-C6 alkyl.

One embodiment provides a compound of Formula (Ia), or pharmaceutically acceptable salt, solvate, or N-oxide thereof:

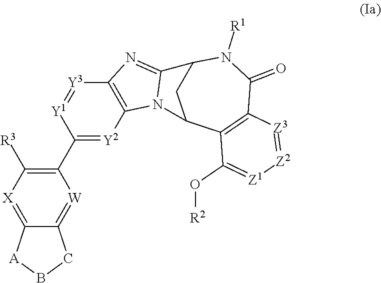

wherein,
W is N or C—R⁴;
X is N, C—F, or C—H;
Y¹ is N, C—F, or C—H;
Y² is N, C—F, or C—H;
Y³ is N, C—F, or C—H;
Z¹ is N or C—H;
Z² is N or C—H;
Z³ is N or C—H;
A is CR⁵R⁶, or N—R⁹;
B is CR⁷R⁸, or N—R¹⁰;
C is —CH₂—, —CH₂CH₂—, —NH—, —O—, or —CH₂O—;
R¹ is selected from hydrogen, optionally substituted C1-C6 alkyl, optionally substituted C3-C6 cycloalkyl, or optionally substituted C4-C7 cycloalkylalkyl;
R² is hydrogen, or optionally substituted C1-C4 alkyl;
R³ is hydrogen or halogen;
R⁴ is hydrogen, halogen, or optionally substituted C1 alkyl;
   R⁵ is —OH, —NH₂, or —NH(optionally substituted C1-C6 alkyl); or optionally, R⁵ and R⁶ are both —OH and join to form an oxo group;
R⁶ is hydrogen, —OH, or optionally substituted C1-C6 alkyl; or optionally, R⁶ and R⁸ are both optionally substituted C1-C6 alkyl and join to form a ring;
R⁷ is hydrogen, —OH, or optionally substituted C1-C6 alkyl; or optionally, R⁷ and R⁸ are both —OH and join to form an oxo group;
R⁸ is hydrogen, —OH, or optionally substituted C1-C6 alkyl;
   R⁹ is hydrogen, or optionally substituted C1-C6 alkyl; and
   R¹⁰ is hydrogen, or optionally substituted C1-C6 alkyl.

Another embodiment provides the compound of Formula (I) or (Ia), or pharmaceutically acceptable salt, solvate, or N-oxide thereof, wherein X is N. Another embodiment provides the compound of Formula (I) or (Ia), or pharmaceutically acceptable salt, solvate, or N-oxide thereof, wherein X is C—F, or C—H.

Another embodiment provides the compound of Formula (I) or (Ia), or pharmaceutically acceptable salt, solvate, or N-oxide thereof, wherein Z¹ is N. Another embodiment provides the compound of Formula (I) or (Ia), or pharmaceutically acceptable salt, solvate, or N-oxide thereof, wherein Z¹ is C—H.

Another embodiment provides the compound of Formula (I) or (Ia), or pharmaceutically acceptable salt, solvate, or N-oxide thereof, wherein Z² is N. Another embodiment provides the compound of Formula (I) or (Ia), or pharmaceutically acceptable salt, solvate, or N-oxide thereof, wherein Z² is C—H.

Another embodiment provides the compound of Formula (I) or (Ia), or pharmaceutically acceptable salt, solvate, or N-oxide thereof, wherein Z³ is N. Another embodiment provides the compound of Formula (I) or (Ia), or pharmaceutically acceptable salt, solvate, or N-oxide thereof, wherein Z³ is C—H.

Another embodiment provides the compound of Formula (I) or (Ia), or pharmaceutically acceptable salt, solvate, or N-oxide thereof, wherein Y¹ is N. Another embodiment provides the compound of Formula (I) or (Ia), or pharmaceutically acceptable salt, solvate, or N-oxide thereof, wherein Y¹ is C—F or C—H.

Another embodiment provides the compound of Formula (I) or (Ia), or pharmaceutically acceptable salt, solvate, or N-oxide thereof, wherein Y² is N. Another embodiment provides the compound of Formula (I) or (Ia), or pharmaceutically acceptable salt, solvate, or N-oxide thereof, wherein Y² is C—F or C—H.

Another embodiment provides the compound of Formula (I) or (Ia), or pharmaceutically acceptable salt, solvate, or N-oxide thereof, wherein Y³ is N. Another embodiment provides the compound of Formula (I) or (Ia), or pharmaceutically acceptable salt, solvate, or N-oxide thereof, wherein Y³ is C—F or C—H.

Another embodiment provides the compound of Formula (I) or (Ia), or pharmaceutically acceptable salt, solvate, or N-oxide thereof, wherein W is N. Another embodiment provides the compound of Formula (I) or (Ia), or pharmaceutically acceptable salt, solvate, or N-oxide thereof, wherein W is C—R⁴.

Another embodiment provides the compound of Formula (I) or (Ia), or pharmaceutically acceptable salt, solvate, or N-oxide thereof, wherein A is CR⁵R⁶; B is CR⁷R⁸; and C is —CH₂— or —CH₂CH₂—.

Another embodiment provides the compound of Formula (I) or (Ia), or pharmaceutically acceptable salt, solvate, or N-oxide thereof, wherein A is CR⁵R⁶; B is CR⁷R⁸; and C is —O—, —OCH₂—, or —CH₂O—.

Another embodiment provides the compound of Formula (I) or (Ia), or pharmaceutically acceptable salt, solvate, or N-oxide thereof, wherein A is CR⁵R⁶; B is CR⁷R⁸; and C is —O—, —OCH₂—, or —CH₂O—.

Another embodiment provides the compound of Formula (I) or (Ia), or pharmaceutically acceptable salt, solvate, or N-oxide thereof, wherein A is CR⁵R⁶, and R⁵ and R⁶ are both —OH joined to form an oxo group; and B is N—R¹⁰.

Another embodiment provides the compound of Formula (I) or (Ia), or pharmaceutically acceptable salt, solvate, or N-oxide thereof, wherein A is N—R⁹; and B is CR⁷R⁸ and R⁷ and R⁸ are both —OH joined to form an oxo group.

Another embodiment provides the compound of Formula (I) or (Ia), or pharmaceutically acceptable salt, solvate, or N-oxide thereof, wherein A is CR⁵R⁶, and R⁵ and R⁶ are both —OH joined to form an oxo group.

Another embodiment provides the compound of Formula (I) or (Ia), or pharmaceutically acceptable salt, solvate, or N-oxide thereof, wherein R⁶ and R⁸ are both optionally substituted C1-C6 alkyl and join to form a ring.

Another embodiment provides the compound of Formula (I) or (Ia), or pharmaceutically acceptable salt, solvate, or N-oxide thereof, wherein R¹ is selected from hydrogen or optionally substituted C1-C6 alkyl. Another embodiment provides the compound of Formula (I) or (Ia), or pharmaceutically acceptable salt, solvate, or N-oxide thereof, wherein R¹ is selected from CH₃ or CD₃.

Another embodiment provides the compound of Formula (I) or (Ia), or pharmaceutically acceptable salt, solvate, or N-oxide thereof, wherein R² is selected from optionally substituted C1-C6 alkyl. Another embodiment provides the compound of Formula (I) or (Ia), or pharmaceutically acceptable salt, solvate, or N-oxide thereof, wherein R² is selected from optionally substituted C1 alkyl. Another embodiment provides the compound of Formula (I) or (Ia), or pharmaceutically acceptable salt, solvate, or N-oxide thereof, wherein R² is —CHF₂.

Another embodiment provides the compound of Formula (I) or (Ia), or pharmaceutically acceptable salt, solvate, or N-oxide thereof, wherein R³ is hydrogen.

Another embodiment provides the compound of Formula (I) or (Ia), or pharmaceutically acceptable salt, solvate, or N-oxide thereof, wherein R⁴ is hydrogen.

Another embodiment provides the compound of Formula (I) or (Ia), or pharmaceutically acceptable salt, solvate, or N-oxide thereof, wherein R⁵ is —OH. Another embodiment provides the compound of Formula (I) or (Ia), or pharmaceutically acceptable salt, solvate, or N-oxide thereof, wherein R⁵ is —NH₂.

Another embodiment provides the compound of Formula (I) or (Ia), or pharmaceutically acceptable salt, solvate, or N-oxide thereof, wherein R⁶ is hydrogen. Another embodiment provides the compound of Formula (I) or (Ia), or pharmaceutically acceptable salt, solvate, or N-oxide thereof, wherein R⁶ is optionally substituted C1-C6 alkyl. Another embodiment provides the compound of Formula (I) or (Ia), or pharmaceutically acceptable salt, solvate, or N-oxide thereof, wherein R⁶ is —CH₃.

One embodiment provides a TNF-α inhibitory compound, or a pharmaceutically acceptable salt, solvate, or N-oxide thereof, having a structure presented in Table 1.

TABLE 1

| Synthetic | | |
|---|---|---|
| Chemistry | | |
| Example | Compound Structure | Compound Name |
| 1 | 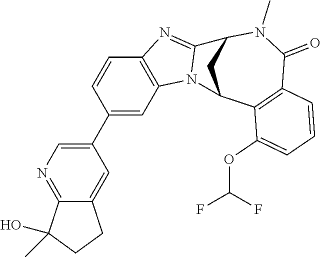 [SMILES: CN1C(=O)c2cccc(OC(F)F)c2[C@H]2C[C@@H]1c1nc3ccc(-c4cnc5c(c4)CCC5(C)O)cc3n12] | (1R,11R)-18-(difluoromethoxy)-5-{7- hydroxy-7-methyl-5H,6H- cyclopenta[b]pyridin-3-yl}-12-methyl- 2,9,12-triazapentacy- clo[9.8.1.0\hat{ }{2,10}.0\hat{ }{3,8}.0\hat{ }{14,19}] icosa-3(8),4,6,9,14(19),15,17- heptaen-13-one |
| 2 | 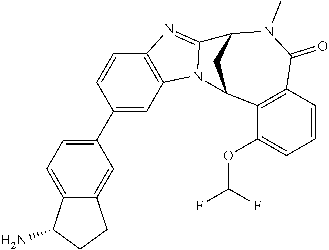 [SMILES: CN1C(=O)c2cccc(OC(F)F)c2[C@H]2C[C@@H]1c1nc3ccc(-c4ccc5c(c4)CC[C@@H]5N)cc3n12] | (1R,11R)-5-[(1S)-1-amino-2,3-dihydro- 1H-inden-5-yl]-18-(difluoromethoxy)- 12-methyl-2,9,12-triazapentacy- clo[9.8.1.0\hat{ }{2,10}.0\hat{ }{3,8}.0\hat{ }{14,19}] icosa-3(8),4,6,9,14(19),15,17- heptaen-13-one |
| 3 | 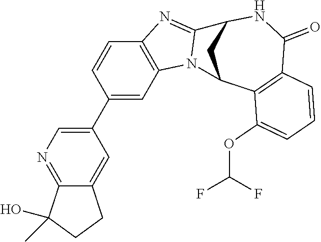 [SMILES: CC1(O)CCc2cc(-c3ccc4nc5n(c4c3)[C@@H]3C[C@H]5NC(=O)c4cccc(OC(F)F)c43)cnc21] | (1R,11R)-18-(difluoromethoxy)-5-{7- hydroxy-7-methyl-5H,6H- cyclopenta[b]pyridin-3-yl}-2,9,12-triazapenta- cyclo[9.8.1.0\hat{ }{2,10}.0\hat{ }{3,8}.0\hat{ }{14,19}] icosa-3(8),4,6,9,14(19),15,17- heptaen-13-one |
| 4 | 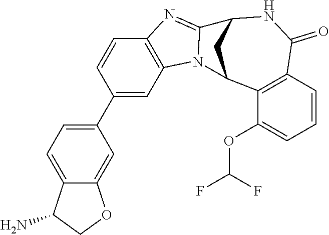 [SMILES: N[C@H]1COc2cc(-c3ccc4nc5n(c4c3)[C@@H]3C[C@H]5NC(=O)c4cccc(OC(F)F)c43)ccc21] | (1R,11R)-5-[(3R)-3-amino-2,3-dihydro- 1-benzofuran-6-yl]-18- (difluoromethoxy)-2,9,12-triazapentacyclo [9.8.1.0\hat{ }{2,10}.0\hat{ }{3,8}.0\hat{ }{14,19}]icosa- 3(8),4,6,9,14(19),15,17-heptaen-13-one |
| 5 | 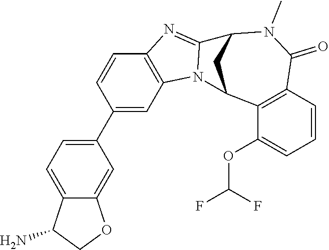 [SMILES: CN1C(=O)c2cccc(OC(F)F)c2[C@H]2C[C@@H]1c1nc3ccc(-c4ccc5c(c4)OC[C@@H]5N)cc3n12] | (1R,11R)-5-[(3R)-3-amino-2,3-dihydro- 1-benzofuran-6-yl]-18-(difluoro- methoxy)-12-methyl-2,9,12-triazapenta- cyclo[9.8.1.0\hat{ }{2,10}.0\hat{ }{3,8}.0\hat{ }{14,19}] icosa-3(8),4,6,9,14(19),15,17- heptaen-13-one |
| 6 | 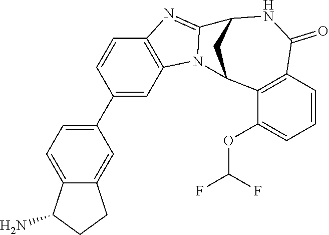 [SMILES: N[C@H]1CCc2cc(-c3ccc4nc5n(c4c3)[C@@H]3C[C@H]5NC(=O)c4cccc(OC(F)F)c43)ccc21] | (1R,11R)-5-[(1S)-1-amino-2,3-dihydro- 1H-inden-5-yl]-18-(difluoromethoxy)- 2,9,12-triazapentacy- clo[9.8.1.0\hat{ }{2,10}.0\hat{ }{3,8}.0\hat{ }{14,19}] icosa-3(8),4,6,9,14(19),15,17- heptaen-13-one |
| 7 | 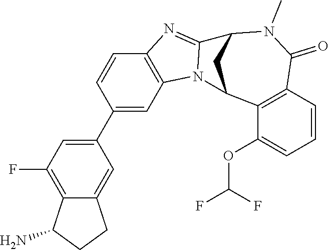 [SMILES: CN1C(=O)c2cccc(OC(F)F)c2[C@H]2C[C@@H]1c1nc3ccc(-c4cc(F)c5c(c4)CC[C@@H]5N)cc3n12] | (1R,11R)-5-[(1S)-1-amino-7-fluoro-2,3- dihydro-1H-inden-5-yl]-18-(difluoro- methoxy)-12-methyl-2,9,12-triazapenta- cyclo[9.8,1.0\hat{ }{2,10}.0\hat{ }{3,8}.0\hat{ }{14,19}] icosa-3(8),4,6,9,14(19),15,17- heptaen-13-one |
| 8 | 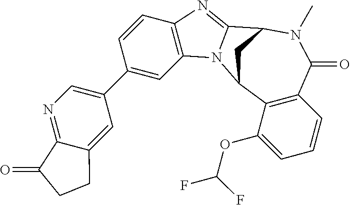 [SMILES: CN1C(=O)c2cccc(OC(F)F)c2[C@H]2C[C@@H]1c1nc3ccc(-c4cnc5c(c4)CCC5=O)cc3n12] | (1R,11R)-18-(difluoromethoxy)-12- methyl-5-{7-oxo-5H,6H-cyclo- penta[b]pyridin-3-yl}-2,9,12-triazapenta- cyclo[9.8.1.0\hat{ }{2,10}.0\hat{ }{3,8}.0\hat{ }{14,19}] icosa-3(8),4,6,9,14(19),15,17- heptaen-13-one |
| 9 | 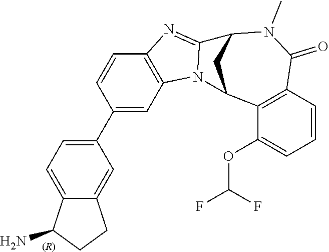 | (1R,11R)-5-[(1R)-1-amino-2,3-dihydro- 1H-inden-5-yl]-18-(difluoromethoxy)- 12-methyl-2,9,12-triazapentacy- clo[9.8.1.0\hat{ }{2,10}.0\hat{ }{3,8}.0\hat{ }\hat{ }{14,19} icosa-3(8),4,6,9,14(19),15,17- heptaen-13-one |
| 10 | 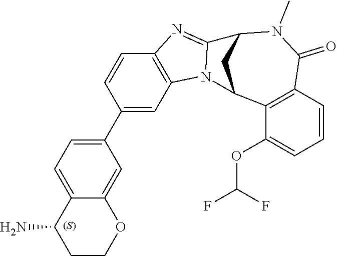 | (1R,11R)-5-[(4S)-4-amino-3,4-dihydro- 2H-1-benzopyran-7-yl]-18-(difluorometh- oxy)-12-methyl-2,9,12-triazapentacy- clo[9.8.1.0\hat{ }{2,10}.0\hat{ }{3,8}.0\hat{ }{14,19}] icosa-3(8),4,6,9,14(19),15,17- heptaen-13-one |
| 11, 12 | 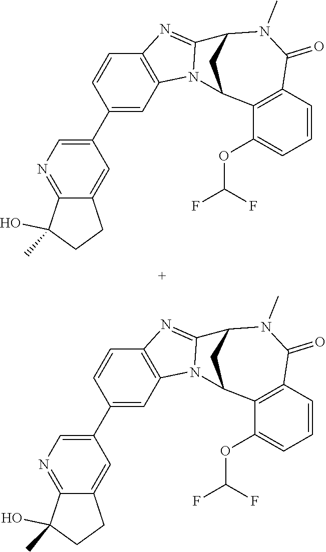 [SMILES: CN1C(=O)c2cccc(OC(F)F)c2[C@H]2C[C@@H]1c1nc3ccc(-c4cnc5c(c4)CC[C@@]5(C)O)cc3n12 ; CN1C(=O)c2cccc(OC(F)F)c2[C@H]2C[C@@H]1c1nc3ccc(-c4cnc5c(c4)CC[C@]5(C)O)cc3n12] | (7R,14R)-1-(difluoromethoxy)-11-((R)- 7-hydroxy-7-methyl-6,7-dihydro-5H- cyclopenta[b]pyridin-3-yl)-6-methyl-6,7- dihydro-7,14- methanobenzo[f]benzo[4.5]imidazo[1,2- a][1,4]diazocin-5(14H)-one and (7R,14R)-1-(difluoromethoxy)-11-((S)- 7-hydroxy-7-methyl-6,7-dihydro-5H- cyclopenta[b]pyridin-3-yl)-6-methyl-6,7- dihydro-7,14- methanobenzo[f]benzo[4,5]imidazo[1,2- a][1,4]diazocin-5(14H)-one |
| 13 | 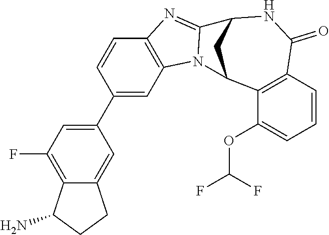 [SMILES: N[C@H]1CCc2cc(-c3ccc4nc5n(c4c3)[C@@H]3C[C@H]5NC(=O)c4cccc(OC(F)F)c43)cc(F)c21] | (1R,11R)-5-[(1S)-1-amino-7-fluoro-2,3- dihydro-1H-inden-5-yl]-18-(difluorometh- oxy)-2,9,12-triazapentacy- clo[9.8,1.0\hat{ }{2,10}.0\hat{ }{3,8}.0\hat{ }{14,19}] icosa-3(8),4,6,9,14(19),15,17- heptaen-13-one |
| 14 | 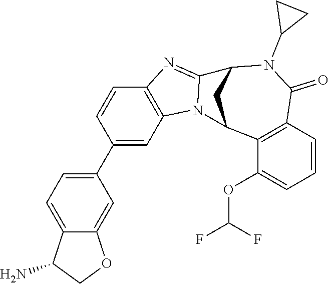 [SMILES: N[C@H]1COc2cc(-c3ccc4nc5n(c4c3)[C@@H]3C[C@H]5N(C4CC4)C(=O)c4cccc(OC(F)F)c43)ccc21] | (1R,11R)-5-[(3R)-3-amino-2,3-dihydro- 1-benzofuran-6-yl]-12-cyclopropyl-18- (difluoromethoxy)-2,9,12-triazapentacy- clo[9.8.1.0\hat{ }{2,10}.0\hat{ }{3,8}.0\hat{ }{14,19}] icosa-3(8),4,6,9,14(19),15,17- heptaen-13-one |
| 15 | 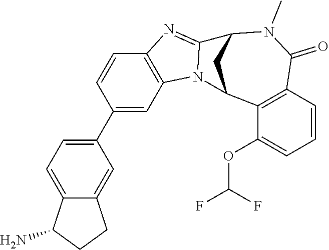 [SMILES: CN1C(=O)c2cccc(OC(F)F)c2[C@H]2C[C@@H]1c1nc3ccc(-c4ccc5c(c4)CC[C@@H]5N)cc3n12] | (1R,11R)-18-(difluoromethoxy)-5-[(1S)- 1-hydroxy-2,3-dihydro-1H-inden-5-yl]- 12-methyl-2,9,12-triazapentacy- clo[9.8.1.0\hat{ }{2,10}.0\hat{ }{3,8}.0\hat{ }{14,19}] icosa-3(8),4,6,9,14(19),15,17- heptaen-13-one |
| 16, 17 | 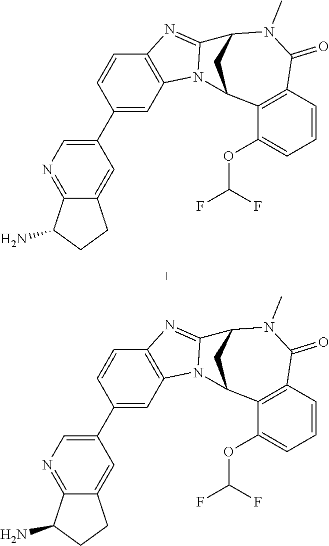 [SMILES: CN1C(=O)c2cccc(OC(F)F)c2[C@H]2C[C@@H]1c1nc3ccc(-c4cnc5c(c4)CC[C@@H]5N)cc3n12 ; CN1C(=O)c2cccc(OC(F)F)c2[C@H]2C[C@@H]1c1nc3ccc(-c4cnc5c(c4)CC[C@H]5N)cc3n12] | (7R,14R)-11-((S)-7-amino-6,7-dihydro- 5H-cyclopenta[b]pyridin-3-yl)-1- (difluoromethoxy)-6-methyl-6,7-dihydro- 7,14- methanobenzo[f]benzo[4,5]imidazo[1,2- a][1,4]diazocin-5(14H)-one And (7R,14R)-11-((R)-7-amino-6,7- dihydro-5H-cyclopenta[b]pyridin-3-yl)- 1-(difluoromethoxy)-6-methyl-6,7- dihydro-7,14- methanobenzo[f]benzo[4,5]imidazo[1,2- a][1,4]diazocin-5(14H)-one |
| 18 | 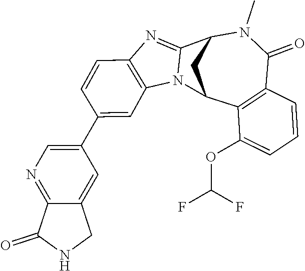 [SMILES: CN1C(=O)c2cccc(OC(F)F)c2[C@H]2C[C@@H]1c1nc3ccc(-c4cnc5c(c4)CNC5=O)cc3n12] | (1R,11R)-18-(difluoromethoxy)-12- methyl-5-{7-oxo-5H,6H-pyrrolo[3,4- b]pyridin-3-yl}-2,9,12-triazapentacy- clo[9.8.1.0\hat{ }{2,10}.0\hat{ }{3,8}.0\hat{ }{14,19}] icosa-3(8),4,6,9,14(19),15,17- heptaen-13-one |
| 19 | 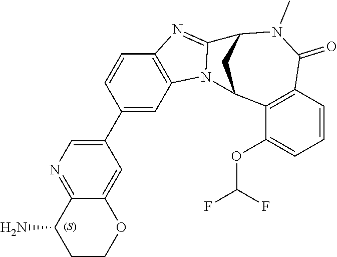 | (1R,11R)-5-[(4S)-4-amino-2H,3H,4H- pyrano[3,2-b]pyridin-7-yl]-18-(difluoro- methoxy)-12-methyl-2,9,12-triazapentacy- clo[9.8.1.0\hat{ }{2,10}.0\hat{ }{3,8}.0\hat{ }{14,19}] icosa-3(8),4,6,9,14(19),15,17- heptaen-13-one |
| 20 | 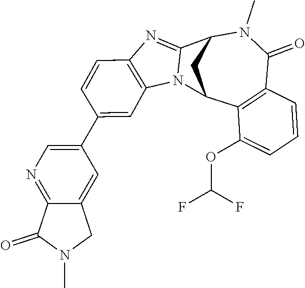 [SMILES: CN1Cc2cc(-c3ccc4nc5n(c4c3)[C@@H]3C[C@H]5N(C)C(=O)c4cccc(OC(F)F)c43)cnc2C1=O] | (1R,11R)-18-(difluoromethoxy)-12- methyl-5-{6-methyl-7-oxo-5H- pyrrolo[3,4-b]pyridin-3-yl}-2,9,12-triazapenta- cyclo[9.8.1.0\hat{ }{2,10}.0\hat{ }{3,8}.0\hat{ }{14,19}] icosa-3(8),4,6,9,14(19),15,17- heptaen-13-one |
| 21 | 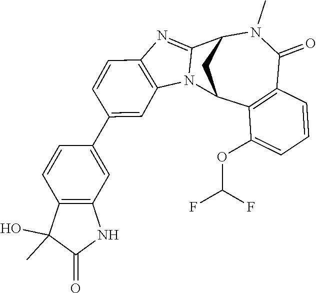 [SMILES: CN1C(=O)c2cccc(OC(F)F)c2[C@H]2C[C@@H]1c1nc3ccc(-c4ccc5c(c4)NC(=O)C5(C)O)cc3n12] | (1R,11R)-18-(difluoromethoxy)-5-(3- hydroxy-3-methyl-2-oxo-1H-indol-6-yl)- 12-methyl-2,9,12-triazapentacy- clo[9.8.1.0\hat{ }{2,10}.0\hat{ }{3,8}.0\hat{ }{14,19}] icosa-3(8),4,6,9,14(19),15,17- heptaen-13-one |
| 22, 23 | 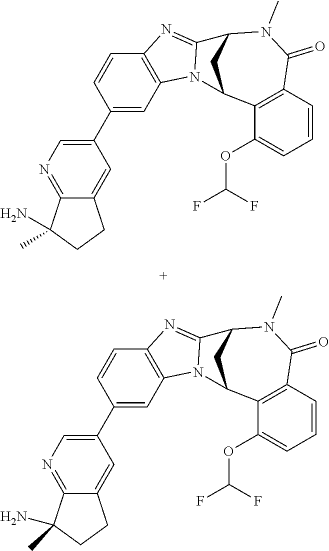 [SMILES: CN1C(=O)c2cccc(OC(F)F)c2[C@H]2C[C@@H]1c1nc3ccc(-c4cnc5c(c4)CC[C@@]5(C)N)cc3n12 ; CN1C(=O)c2cccc(OC(F)F)c2[C@H]2C[C@@H]1c1nc3ccc(-c4cnc5c(c4)CC[C@]5(C)N)cc3n12] | (7R,14R)-11-((R)-7-amino-7-methyl-6,7- dihydro-5H-cyclopenta[b]pyridin-3-yl)- 1-(difluoromethoxy)-6-methyl-6,7- dihydro-7,14- methanobenzo[f]benzo[4,5]imidazo[1,2- a][1,4]diazocin-5(14H)-one and (7R,14R)-11-((S)-7-amino-7-methyl-6,7- dihydro-5H-cyclopenta[b]pyridin-3-yl)- 1-(difluoromethoxy)-6-methyl-6,7- dihydro-7,14- methanobenzo[f]benzo[4,5]imidazo[1,2- a][1,4]diazocin-5(14H)-one |
| 24 | 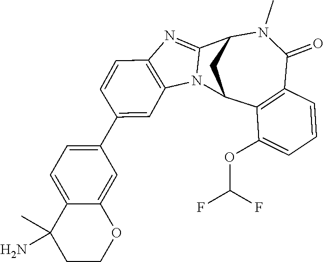 [SMILES: CN1C(=O)c2cccc(OC(F)F)c2[C@H]2C[C@@H]1c1nc3ccc(-c4ccc5c(c4)OCCC5(C)N)cc3n12] | (1R,11R)-5-(4-amino-4-methyl-2,3- dihydro-1-benzopyran-7-yl)-18-(di- fluoromethoxy)-12-methyl-2,9,12-triazapenta- cyclo[9.8.1.0\hat{ }{2,10}.0\hat{ }{3,8}.0\hat{ }{14,19}] icosa-3(8),4,6,9,14(19),15,17- heptaen-13-one |
| 25, 26 | 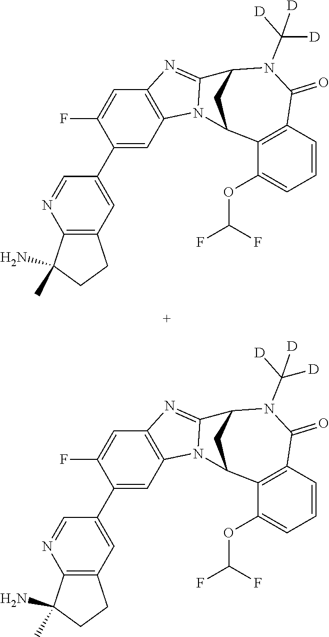 [SMILES: [2H]C([2H])([2H])N1C(=O)c2cccc(OC(F)F)c2[C@H]2C[C@@H]1c1nc3cc(F)c(-c4cnc5c(c4)CC[C@]5(C)N)cc3n12 ; [2H]C([2H])([2H])N1C(=O)c2cccc(OC(F)F)c2[C@H]2C[C@@H]1c1nc3cc(F)c(-c4cnc5c(c4)CC[C@@]5(C)N)cc3n12] | (7R,14R)-11-((S)-7-amino-7-methyl-6,7- dihydro-5H-cyclopenta[b]pyridin-3-yl)- 1-(difluoromethoxy)-10-fluoro-6- (methyl-d3)-6,7-dihydro-7,14- methanobenzo[f]benzo[4,5]imidazo[1,2- a][1,4]diazocin-5(14H)-one and (7R,14R)-11-((R)-7-amino-7-methyl-6,7- dihydro-5H-cyclopenta[b]pyridin-3-yl)- 1-(difluoromethoxy)-10-fluoro-6- (methyl-d3)-6,7-dihydro-7,14- methanobenzo[f]benzo[4,5]imidazo[1,2- a][1,4]diazocin-5(14H)-one |
| 27 | 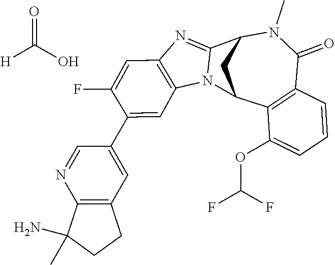 | (1R,11R)-5-{7-amino-7-methyl-5H,6H- cyclopenta[b]pyridin-3-yl}-18- (difluoromethoxy)-6-fluoro-12-methyl- 2,9,12-triazapentacy- clo[9.8.1.0\hat{ }{2,10}.0\hat{ }{3,8}.0\hat{ }{14,19}] icosa-3(8),4,6,9,14(19),15,17- heptaen-13-one; formic acid |
| 28, 29 | 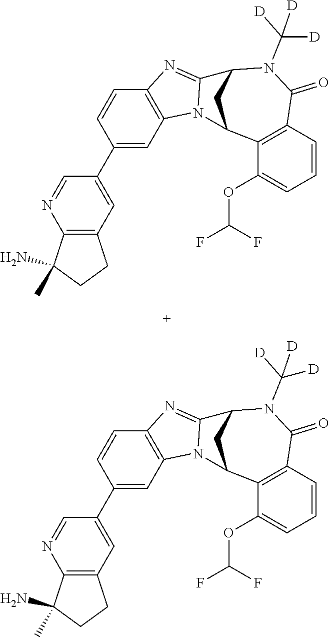 [SMILES: [2H]C([2H])([2H])N1C(=O)c2cccc(OC(F)F)c2[C@H]2C[C@@H]1c1nc3ccc(-c4cnc5c(c4)CC[C@]5(C)N)cc3n12 ; [2H]C([2H])([2H])N1C(=O)c2cccc(OC(F)F)c2[C@H]2C[C@@H]1c1nc3ccc(-c4cnc5c(c4)CC[C@@]5(C)N)cc3n12] | (7R,14R)-11-((S)-7-amino-7-methyl-6,7- dihydro-5H-cyclopenta[b]pyridin-3-yl)- 1-(difluoromethoxy)-6-(methyl-d3)-6,7- dihydro-7,14- methanobenzo[f]benzo[4,5]imidazo[1,2- a][1,4]diazocin-5(14H)-one and (7R,14R)-11-((R)-7-amino-7-methyl-6,7- dihydro-5H-cyclopenta[b]pyridin-3-yl)- 1-(difluoromethoxy)-6-(methyl-d3)-6,7- dihydro-7,14- methanobenzo[f]benzo[4,5]imidazo[1,2- a][1,4]diazocin-5(14H)-one |
| 30 | 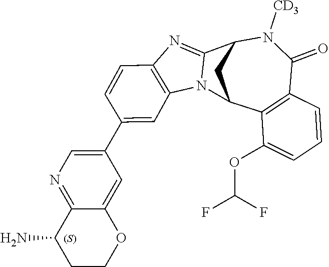 | (7R,14R)-11-((S)-4-amino-3,4-dihydro- 2H-pyrano[3,2-b]pyridin-7-yl)-1- (difluoromethoxy)-6-(methyl-d3)-6,7- dihydro-7,14- methanobenzo[f]benzo[4,5]imidazo[1,2- a][1,4]diazocin-5(14H)-one |
| 31 | 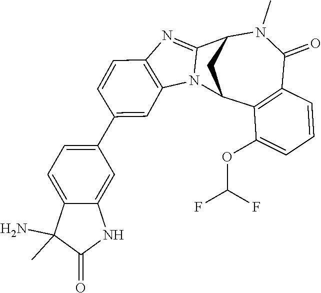 [SMILES: CN1C(=O)c2cccc(OC(F)F)c2[C@H]2C[C@@H]1c1nc3ccc(-c4ccc5c(c4)NC(=O)C5(C)N)cc3n12] | (7R,14R)-11-(3-amino-3-methyl-2- oxoindolin-6-yl)-1-(difluoromethoxy)-6- methyl-6,7-dihydro-7,14- methanobenzo[f]benzo[4,5]imidazo[1,2- a][1,4]diazocin-5(14H)-one |
| 32, 33 | 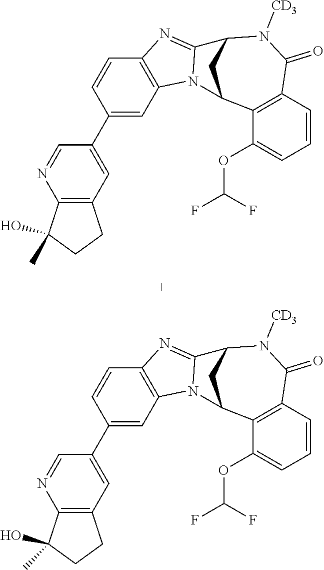 [SMILES: [2H]C([2H])([2H])N1C(=O)c2cccc(OC(F)F)c2[C@H]2C[C@@H]1c1nc3ccc(-c4cnc5c(c4)CC[C@]5(C)O)cc3n12 ; [2H]C([2H])([2H])N1C(=O)c2cccc(OC(F)F)c2[C@H]2C[C@@H]1c1nc3ccc(-c4cnc5c(c4)CC[C@@]5(C)O)cc3n12] | (7R,14R)-1-(difluoromethoxy)-11-((S)-7- hydroxy-7-methyl-6,7-dihydro-5H- cyclopenta[b]pyridin-3-yl)-6-(methyl- d3)-6,7-dihydro-7,14- methanobenzo[f]benzo[4,5]imidazo[1,2- a][1,4]diazocin-5(14H)-one and (7R,14R)-1-(difluoromethoxy)-11-((R)- 7-hydroxy-7-methyl-6,7-dihydro-5H- cyclopenta[b]pyridin-3-yl)-6-(methyl- d3)-6,7-dihydro-7,14- methanobenzo[f]benzo[4,5]imidazo[1,2- a][1,4]diazocin-5(14H)-one |
| 34, 35 | 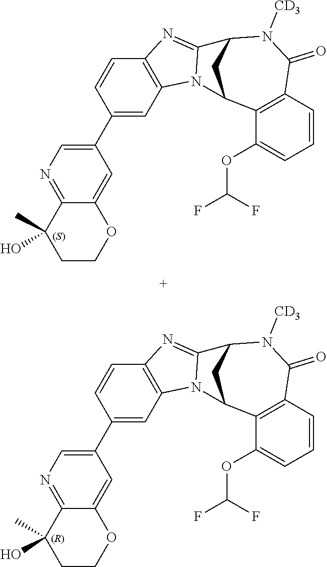 | (7R,14R)-1-(difluoromethoxy)-11-((S)-4- hydroxy-4-methyl-3,4-dihydro-2H- pyrazno[3,2-b]pyridin-7-yl)-6-(methyl- d3)-6,7-dihydro-7,14- methanobenzo[f]benzo[4,5]imidazo[1,2- a][1,4]diazocin-5(14H)-one and (7R,14R)-1-(difluoromethoxy)-11-((R)- 4-hydroxy-4-methyl-3,4-dihydro-2H- pyrano[3,2-b]pyridin-7-yl)-6-(methyl- d3)-6,7-dihydro-7,14- methanobenzo[f]benzo[4,5]imidazo[1,2- a][1,4]diazocin-5(14H)-one |
| 36, 37 | 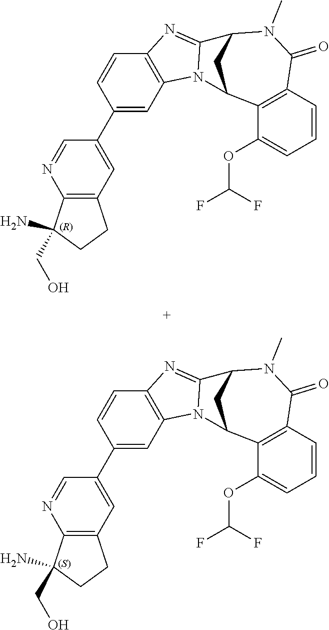 | (7R,14R)-11-((R)-7-amino-7- (hydroxymethyl)-6,7-dihydro-5H- cyclopenta[b]pyridin-3-yl)-1- (difluoromethoxy)-6-methyl-6,7-dihydro- 7,14- methanobenzo[f]benzo[4,5]imidazo[1,2- a][1,4]diazocin-5(14H)-one and (7R,14R)-11-((S)-7-amino-7- (hydroxymethyl)-6,7-dihydro-5H- cyclopenta[b]pyridin-3-yl)-1- (difluoromethoxy)-6-methyl-6,7-dihydro- 7,14- methanobenzo[f]benzo[4,5]imidazo[1,2- a][1,4]diazocin-5(14H)-one |
| 38, 39 | 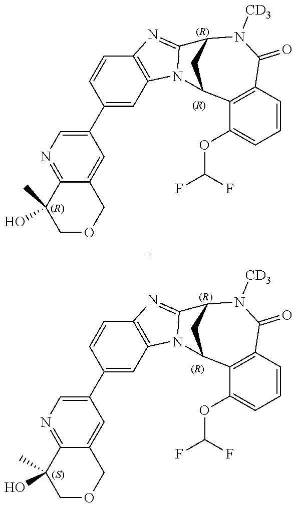 | (7R,14R)-1-(difluoromethoxy)-11-((R)- 8-hydroxy-8-methyl-7,8-dihydro-5H- pyrano[4,3-b]pyridin-3-yl)-6-(methyl- d3)-6,7-dihydro-7,14- methanobenzo[f]benzo[4,5]imidazo[1,2- a][1,4]diazocin-5(14H)-one and (7R,14R)-1-(difluoromethoxy)-11-((S)-8- hydroxy-8-methyl-7,8-dihydro-5H- pyrano[4,3-b]pyridin-3-yl)-6-(methyl- d3)-6,7-dihydro-7,14- methanobenzo[f]benzo[4,5]imidazo[1,2- a][1,4]diazocin-5(14H)-one |
| 40, 41 | 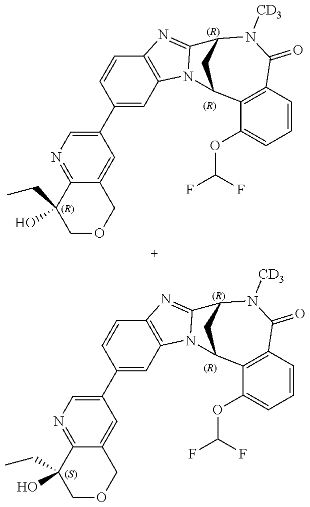 | (7R,14R)-1-(difluoromethoxy)-11-((R)- 8-ethyl-8-hydroxy-7,8-dihydro-5H- pyrano[4,3-b]pyridin-3-yl)-6-(methyl- d3)-6,7-dihydro-7,14- methanobenzo[f]benzo[4,5]imidazo[1,2- a][1,4]diazocin-5(14H)-one and (7R,14R)-1-(difluoromethoxy)-11-((S)-8- ethyl-8-hydroxy-7,8-dihydro-5H- pyrano[4,3-b]pyridin-3-yl)-6-(methyl- d3)-6,7-dihydro-7,14- methanobenzo[f]benzo[4,5]imidazo[1,2- a][1,4]diazocin-5(14H)-one |
| 42, 43 | 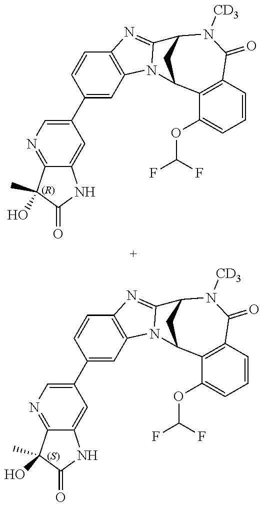 | (7R,14R)-1-(difluoromethoxy)-11-((R)- 3-hydroxy-3-methyl-2-oxo-2,3-dihydro- 1H-pyrrolo[3,2-b]pyridin-6-yl)-6- (methyl-d3)-6,7-dihydro-7,14- methanobenzo[f]benzo[4,5]imidazo[1,2- a][1,4]diazocin-5(14H)-one and (7R,14R)-1-(difluoromethoxy)-11-((S)-3- hydroxy-3-methyl-2-oxo-2,3-dihydro- 1H-pyrrolo[3,2-b]pyridin-6-yl)-6- (methyl-d3)-6,7-dihydro-7,14- methanobenzo[f]benzo[4,5]imidazo[1,2- a][1,4]diazocin-5(14H)-one |
| 44 | 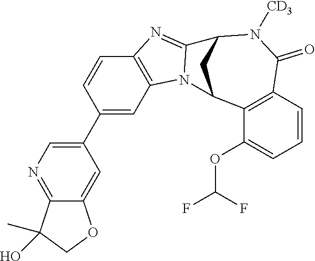 [SMILES: [2H]C([2H])([2H])N1C(=O)c2cccc(OC(F)F)c2[C@H]2C[C@@H]1c1nc3ccc(-c4cnc5c(c4)OCC5(C)O)cc3n12] | (7R,14R)-1-(difluoromethoxy)-11-(3- hydroxy-3-methyl-2,3-dihydrofuro[3,2- b]pyridin-6-yl)-6-(methyl-d3)-6,7- dihydro-7,14- methanobenzo[f]benzo[4,5]imidazo[1,2- a][1,4]diazocin-5(14H)-one |
| 45 | 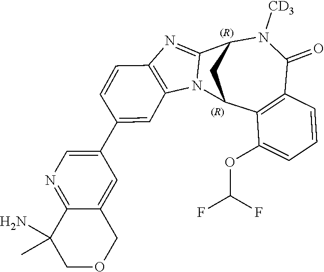 | (7R,14R)-11-(8-amino-8-methyl-7,8- dihydro-5H-pyrano[4,3-b]pyridin-3-yl)- 1-(difluoromethoxy)-6-(methyl-d3)-6,7- dihydro-7,14- methanobenzo[f]benzo[4,5]imidazo[1,2- a][1,4]diazocin-5(14H)-one |
| 46, 47 | 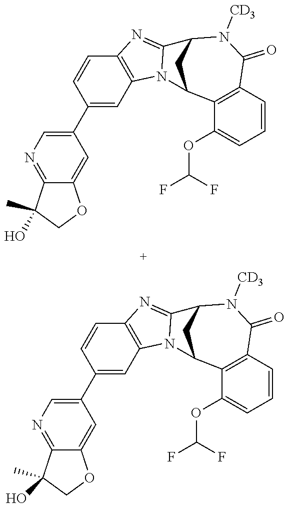 [SMILES: [2H]C([2H])([2H])N1C(=O)c2cccc(OC(F)F)c2[C@H]2C[C@@H]1c1nc3ccc(-c4cnc5c(c4)OC[C@]5(C)O)cc3n12 ; [2H]C([2H])([2H])N1C(=O)c2cccc(OC(F)F)c2[C@H]2C[C@@H]1c1nc3ccc(-c4cnc5c(c4)OC[C@@]5(C)O)cc3n12] | (7R,14R)-1-(difluoromethoxy)-11-((R)- 3-hydroxy-3-methyl-2,3-dihydrofuro[3,2- b]pyridin-6-yl)-6-(methyl-d3)-6,7- dihydro-7,14- methanobenzo[f]benzo[4,5]imidazo[1,2- a][1,4]diazocin-5(14H)-one and (7R,14R)-1-(difluoromethoxy)-11-((S)-3- hydroxy-3-methyl-2,3-dihydrofuro[3,2- b]pyridin-6-yl)-6-(methyl-d3)-6,7- dihydro-7,14- methanobenzo[f]benzo[4,5]imidazo[1,2- a][1,4]diazocin-5(14H)-one |
| 48 | 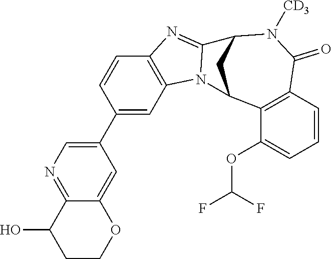 [SMILES: [2H]C([2H])([2H])N1C(=O)c2cccc(OC(F)F)c2[C@H]2C[C@@H]1c1nc3ccc(-c4cnc5c(c4)OCCC5O)cc3n12] | (7R,14R)-1-(difluoromethoxy)-11-(4- hydroxy-3,4-dihydro-2H-pyrano[3,2- b]pyridin-7-yl)-6-(methyl-d3)-6,7- dihydro-7,14- methanobenzo[f]benzo[4,5]imidazo[1,2- a][1,4]diazocin-5(14H)-one |
| 49, 50 | 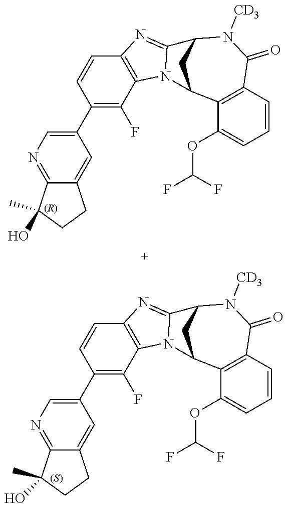 | (7R,14R)-1-(difluoromethoxy)-12- fluoro-11-((R)-7-hydroxy-7-methyl-6,7- dihydro-5H-cyclopenta[b]pyridin-3-yl)- 6-(methyl-d3)-6,7-dihydro-7,14- methanobenzo[f]benzo[4,5]imidazo[1,2- a][1,4]diazocin-5(14H)-one and (7R,14R)-1-(difluoromethoxy)-12- fluoro-11-((S)-7-hydroxy-7-methyl-6,7- dihydro-5H-cyclopenta[b]pyridin-3-yl)- 6-(methyl-d3)-6,7-dihydro-7,14- methanobenzo[f]benzo[4,5]imidazo[1,2- a][1,4]diazocin-5(14H)-one |
| 51, 52 | 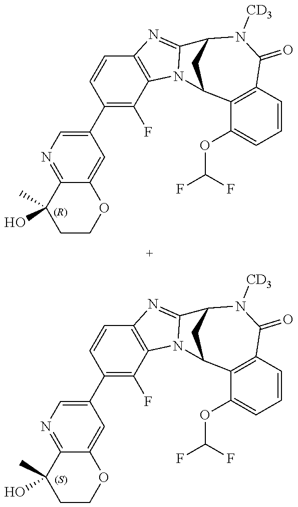 | (7R,14R)-1-(difluoromethoxy)-12- fluoro-11-((R)-4-hydroxy-4-methyl-3,4- dihydro-2H-pyrano[3,2-b]pyridin-7-yl)- 6-(methyl-d3)-6,7-dihydro-7,14- methanobenzo[f]benzo[4,5]imidazo[1,2- a][1,4]diazocin-5(14H)-one and (7R,14R)-1-(difluoromethoxy)-12- fluoro-11-((S)-4-hydroxy-4-methyl-3,4- dihydro-2H-pyrano[3,2-b]pyridin-7-yl)- 6-(methyl-d3)-6,7-dihydro-7,14- methanobenzo[f]benzo[4,5]imidazo[1,2- a][1,4]diazocin-5(14H)-one |
| 53, 54 | 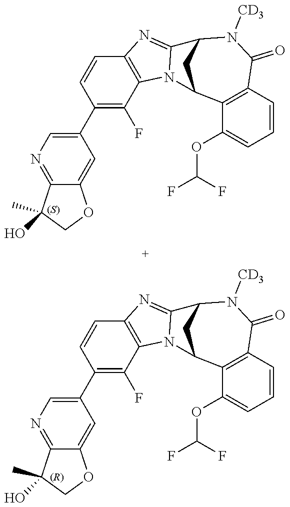 | (7R,14R)-1-(difluoromethoxy)-12- fluoro-11-((S)-3-hydroxy-3-methyl-2,3- dihydrofuro[3,2-b]pyridin-6-yl)-6- (methyl-d3)-6,7-dihydro-7,14- methanobenzo[f]benzo[4,5]imidazo[1,2- a][1,4]diazocin-5(14H)-one and (7R,14R)-1-(difluoromethoxy)-12- fluoro-11-((R)-3-hydroxy-3-methyl-2,3- dihydrofuro[3,2-b]pyridin-6-yl)-6- (methyl-d3)-6,7-dihydro-7,14- methanobenzo[f]benzo[4,5]imidazo[1,2- a][1,4]diazocin-5(14H)-one |
| 55, 56 | 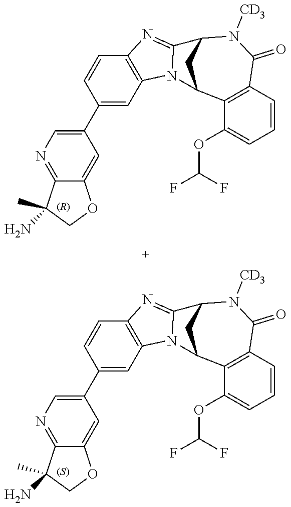 | (7R,14R)-11-((R)-3-amino-3-methyl-2,3- dihydrofuro[3,2-b]pyridin-6-yl)-1- (difluoromethoxy)-6-(methyl-d3)-6,7- dihydro-7,14- methanobenzo[f]benzo[4,5]imidazo[1,2- a][1,4]diazocin-5(14H)-one and (7R,14R)-11-((S)-3-amino-3-methyl-2,3- dihydrofuro[3,2-b]pyridin-6-yl)-1- (difluoromethoxy)-6-(methyl-d3)-6,7- dihydro-7,14- methanobenzo[f]benzo[4,5]imidazo[1,2- a][1,4]diazocin-5(14H)-one |
| 57, 58 | 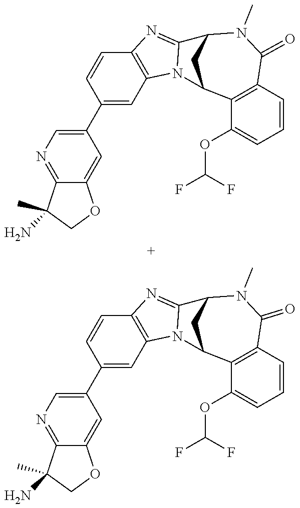 [SMILES: CN1C(=O)c2cccc(OC(F)F)c2[C@H]2C[C@@H]1c1nc3ccc(-c4cnc5c(c4)OC[C@]5(C)N)cc3n12 ; CN1C(=O)c2cccc(OC(F)F)c2[C@H]2C[C@@H]1c1nc3ccc(-c4cnc5c(c4)OC[C@@]5(C)N)cc3n12] | (7R,14R)-11-((R)-3-amino-3-methyl-2,3- dihydrofuro[3,2-b]pyridin-6-yl)-1- (difluoromethoxy)-6-methyl-6,7-dihydro- 7,14- methanobenzo[f]benzo[4,5]imidazo[1,2- a][1,4]diazocin-5(14H)-one and (7R,14R)-11-((S)-3-amino-3-methyl-2,3- dihydrofuro[3,2-b]pyridin-6-yl)-1- (difluoromethoxy)-6-methyl-6,7-dihydro- 7,14- methanobenzo[f]benzo[4,5]imidazo[1,2- a][1,4]diazocin-5(14H)-one |
| 59, 60 | 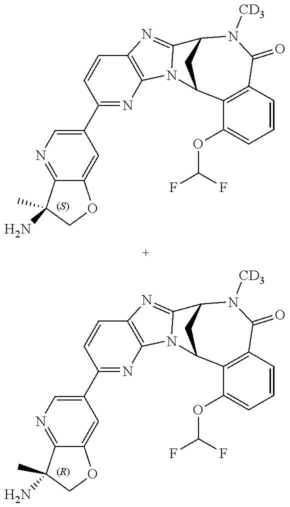 | (7R,14R)-11-((S)-3-amino-3-methyl-2,3- dihydrofuro[3,2-b]pyridin-6-yl)-1- (difluoromethoxy)-6-(methyl-d3)-6,7- dihydro-7,14- methanobenzo[f]pyrido[3′,2′:4,5]imidazo [1,2-a][1,4]diazocin-5(14H)-one and (7R,14R)-11-((R)-3-amino-3-methyl-2,3- dihydrofuro[3,2-b]pyridin-6-yl)-1- (difluoromethoxy)-6-(methyl-d3)-6,7- dihydro-7,14- methanobenzo[f]pyrido[3′,2′:4,5]imidazo [1,2-a][1,4]diazocin-5(14H)-one |
| 61, 62 | 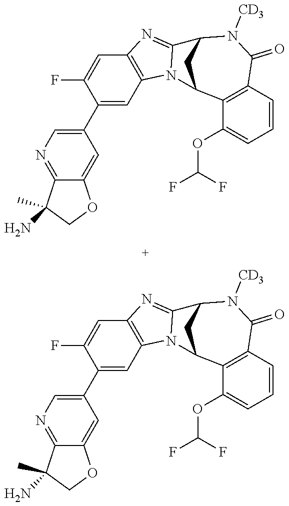 [SMILES: [2H]C([2H])([2H])N1C(=O)c2cccc(OC(F)F)c2[C@H]2C[C@@H]1c1nc3cc(F)c(-c4cnc5c(c4)OC[C@@]5(C)N)cc3n12 ; [2H]C([2H])([2H])N1C(=O)c2cccc(OC(F)F)c2[C@H]2C[C@@H]1c1nc3cc(F)c(-c4cnc5c(c4)OC[C@]5(C)N)cc3n12] | (7R,14R)-11-((S)-3-amino-3-methyl-2,3- dihydrofuro[3,2-b]pyridin-6-yl)-1- (difluoromethoxy)-10-fluoro-6-(methyl- d3)-6,7-dihydro-7,14- methanobenzo[f]benzo[4,5]imidazo[1,2- a][1,4]diazocin-5(14H)-one and (7R,14R)-11-((R)-3-amino-3-methyl-2,3- dihydrofuro[3,2-b]pyridin-6-yl)-1- (difluoromethoxy)-10-fluoro-6-(methyl- d3)-6,7-dihydro-7,14- methanobenzo[f]benzo[4,5]imidazo[1,2- a][1,4]diazocin-5(14H)-one |
| 63, 64 | 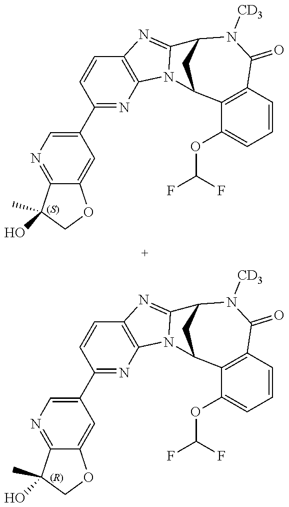 | (7R,14R)-1-(difluoromethoxy)-11-((S)-3- hydroxy-3-methyl-2,3-dihydrofuro[3,2- b]pyridin-6-yl)-6-(methyl-d3)-6,7- dihydro-7,14- methanobenzo[f]pyrido[3′,2′:4,5]imidazo [1,2-a][1,4]diazocin-5(14H)-one and (7R,14R)-1-(difluoromethoxy)-11-((R)- 3-hydroxy-3-methyl-2,3-dihydrofuro[3,2- b]pyridin-6-yl)-6-(methyl-d3)-6,7- dihydro-7,14- methanobenzo[f]pyrido[3′,2′:4,5]imidazo [1,2-a][1,4]diazocin-5(14H)-one |
| 65, 66 | 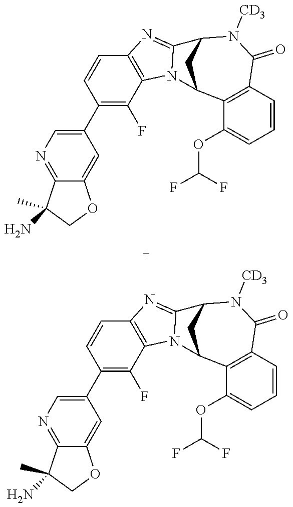 [SMILES: [2H]C([2H])([2H])N1C(=O)c2cccc(OC(F)F)c2[C@H]2C[C@@H]1c1nc3ccc(-c4cnc5c(c4)OC[C@@]5(C)N)c(F)c3n12 ; [2H]C([2H])([2H])N1C(=O)c2cccc(OC(F)F)c2[C@H]2C[C@@H]1c1nc3ccc(-c4cnc5c(c4)OC[C@]5(C)N)c(F)c3n12] | (7R,14R)-11-((S)-3-amino-3-methyl-2,3- dihydrofuro[3,2-b]pyridin-6-yl)-1- (difluoromethoxy)-12-fluoro-6-(methyl- d3)-6,7-dihydro-7,14- methanobenzo[f]benzo[4,5]imidazo[1,2- a][1,4]diazocin-5(14H)-one and (7R,14R)-11-((R)-3-amino-3-methyl-2,3- dihydrofuro[3,2-b]pyridin-6-yl)-1- (difluoromethoxy)-12-fluoro-6-(methyl- d3)-6,7-dihydro-7,14- methanobenzo[f]benzo[4,5]imidazo[1,2- a][1,4]diazocin-5(14H)-one |
| 67, 68 | 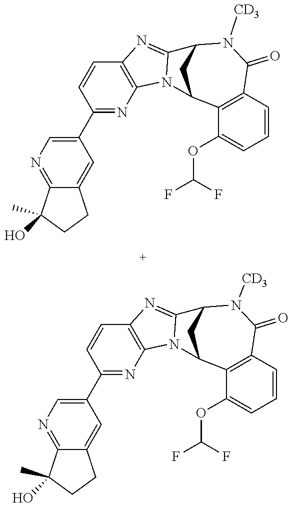 [SMILES: [2H]C([2H])([2H])N1C(=O)c2cccc(OC(F)F)c2[C@H]2C[C@@H]1c1nc3ccc(-c4cnc5c(c4)CC[C@@]5(C)O)nc3n12 ; [2H]C([2H])([2H])N1C(=O)c2cccc(OC(F)F)c2[C@H]2C[C@@H]1c1nc3ccc(-c4cnc5c(c4)CC[C@]5(C)O)nc3n12] | (7R,14R)-1-(difluoromethoxy)-11-((R)- 7-hydroxy-7-methyl-6,7-dihydro-5H- cyclopenta[b]pyridin-3-yl)-6-(methyl- d3)-6,7-dihydro-7,14- methanobenzo[f]pyrido[3′,2′:4,5]imidazo [1,2-a][1,4]diazocin-5(14H)-one and (7R,14R)-1-(difluoromethoxy)-11-((S)-7- hydroxy-7-methyl-6,7-dihydro-5H- cyclopenta[b]pyridin-3-yl)-6-(methyl- d3)-6,7-dihydro-7,14- methanobenzo[f]pyrido[3′,2′:4,5]imidazo [1,2-a][1,4]diazocin-5(14H)-one |
| 69, 70 | 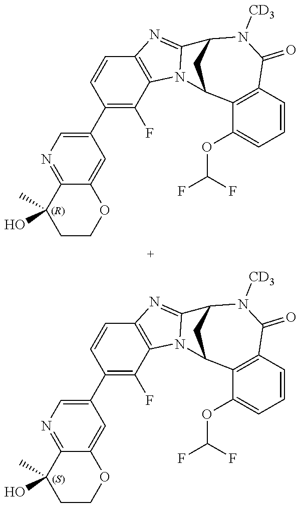 | (7R,14R)-1-(difluoromethoxy)-11-((R)- 4-hydroxy-4-methyl-3,4-dihydro-2H- pyrano[3,2-b]pyridin-7-yl)-6-(methyl- d3)-6,7-dihydro-7,14- methanobenzo[f]pyrido[3′,2′:4,5]imidazo [1,2-a][1,4]diazocin-5(14H)-one and (7R,14R)-1-(difluoromethoxy)-11-((S)-4- hydroxy-4-methyl-3,4-dihydro-2H- pyrano[3,2-b]pyridin-7-yl)-6-(methyl- d3)-6,7-dihydro-7,14- methanobenzo[f]pyrido[3′,2′:4,5]imidazo [1,2-a][1,4]diazocin-5(14H)-one |

Another embodiment provides a TNF-α inhibitory compound, or a pharmaceutically acceptable salt, solvate, or N-oxide thereof, having a structure presented in Table 2.

TABLE 2

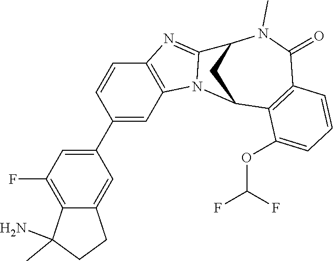 [SMILES: CN1C(=O)c2cccc(OC(F)F)c2[C@H]2C[C@H]1c1nc3ccc(-c4cc(F)c5c(c4)CCC5(C)N)cc3n12]

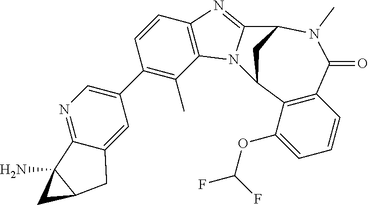 [SMILES: Cc1c(-c2cnc3c(c2)C[C@H]2C[C@@]32N)ccc2nc3n(c12)[C@@H]1C[C@H]3N(C)C(=O)c2cccc(OC(F)F)c21]

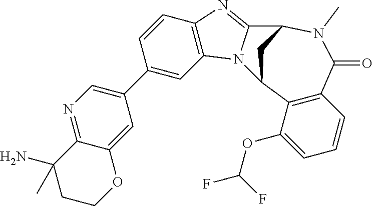 [SMILES: CN1C(=O)c2cccc(OC(F)F)c2[C@H]2C[C@@H]1c1nc3ccc(-c4cnc5c(c4)OCCC5(C)N)cc3n12]

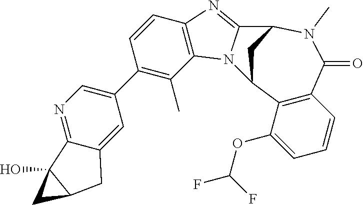 [SMILES: Cc1c(-c2cnc3c(c2)C[C@H]2C[C@@]32O)ccc2nc3n(c12)[C@@H]1C[C@H]3N(C)C(=O)c2cccc(OC(F)F)c21]

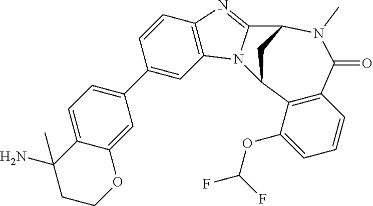 [SMILES: CN1C(=O)c2cccc(OC(F)F)c2[C@H]2C[C@@H]1c1nc3ccc(-c4ccc5c(c4)OCCC5(C)N)cc3n12]

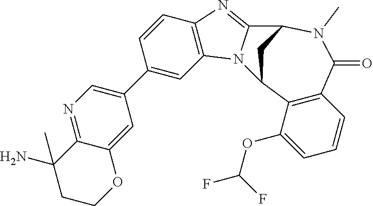 [SMILES: CN1C(=O)c2cccc(OC(F)F)c2[C@H]2C[C@@H]1c1nc3ccc(-c4cnc5c(c4)OCCC5(C)N)cc3n12]

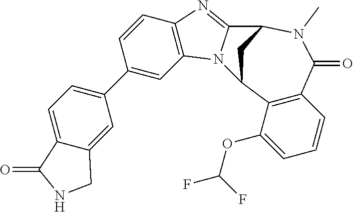 [SMILES: CN1C(=O)c2cccc(OC(F)F)c2[C@H]2C[C@@H]1c1nc3ccc(-c4ccc5c(c4)CNC5=O)cc3n12]

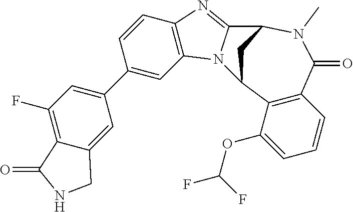 [SMILES: CN1C(=O)c2cccc(OC(F)F)c2[C@H]2C[C@@H]1c1nc3ccc(-c4cc(F)c5c(c4)CNC5=O)cc3n12]

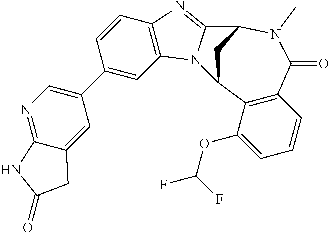 [SMILES: CN1C(=O)c2cccc(OC(F)F)c2[C@H]2C[C@@H]1c1nc3ccc(-c4cnc5c(c4)CC(=O)N5)cc3n12]

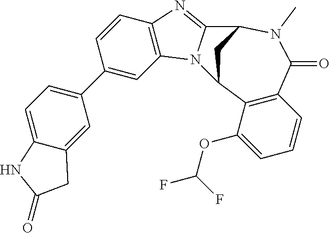 [SMILES: CN1C(=O)c2cccc(OC(F)F)c2[C@H]2C[C@@H]1c1nc3ccc(-c4ccc5c(c4)CC(=O)N5)cc3n12]

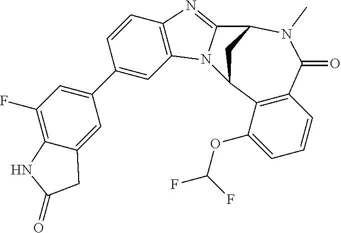 [SMILES: CN1C(=O)c2cccc(OC(F)F)c2[C@H]2C[C@@H]1c1nc3ccc(-c4cc(F)c5c(c4)CC(=O)N5)cc3n12]

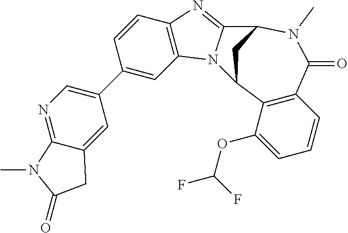 [SMILES: CN1C(=O)Cc2cc(-c3ccc4nc5n(c4c3)[C@@H]3C[C@H]5N(C)C(=O)c4cccc(OC(F)F)c43)cnc21]

### Preparation of Compounds

The compounds used in the synthetic chemistry reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the TNF-α inhibitory compound described herein is administered as a pure chemical. In other embodiments, the TNF-α inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one TNF-α inhibitory compound as described herein, or pharmaceutically acceptable salt, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I) or (Ia), or pharmaceutically acceptable salt, solvate, or N-oxide thereof.

One embodiment provides a method of preparing a pharmaceutical composition comprising mixing a compound of Formula (I) or (Ia), or a pharmaceutically acceptable salt, solvate, or N-oxide thereof, and a pharmaceutically acceptable carrier.

In certain embodiments, the TNF-α inhibitory compound as described by Formula (I) or (Ia), or a pharmaceutically acceptable salt, solvate, or N-oxide thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

One embodiment provides a pharmaceutical composition comprising a pharmaceutically acceptable excipient and a compound of Table 1 or Table 2, or a pharmaceutically acceptable salt, solvate, or N-oxide thereof.

One embodiment provides a method of preparing a pharmaceutical composition comprising mixing a compound of Table 1 or Table 2, or a pharmaceutically acceptable salt, solvate, or N-oxide thereof, and a pharmaceutically acceptable carrier.

In certain embodiments, the TNF-α inhibitory compound as described by Table 1 or Table 2, or a pharmaceutically acceptable salt, solvate, or N-oxide thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the TNF-α inhibitory compound as described by Formula (I) or (Ia), or Table 1 or Table 2, or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one TNF-α inhibitory compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a compound of Formula (I) or (Ia), or a pharmaceutically acceptable salt, solvate, or N-oxide thereof, for use in a method of treatment of the human or animal body.

One embodiment provides a compound of Formula (I) or (Ia), or a pharmaceutically acceptable salt, solvate, or N-oxide thereof, for use in a method of treatment of inflammatory or autoimmune disease or disorder. Another embodiment provides a compound of Formula (I) or (Ia), or a pharmaceutically acceptable salt, solvate, or N-oxide thereof, for use in a method of treatment of inflammatory disease or disorder. Yet another embodiment provides a compound of Formula (I) or (Ia), or a pharmaceutically acceptable salt, solvate, or N-oxide thereof, for use in a method of treatment of autoimmune disease or disorder.

One embodiment provides a pharmaceutical composition comprising a compound of Formula (I) or (Ia), or a pharmaceutically acceptable salt, solvate, or N-oxide thereof, and a pharmaceutically acceptable excipient for use in a method of treatment of inflammatory or autoimmune disease or disorder.

One embodiment provides a use of a compound of Formula (I) or (Ia), or a pharmaceutically acceptable salt, solvate, or N-oxide thereof, in the manufacture of a medicament for the treatment of inflammatory or autoimmune disease or disorder.

In some embodiments is provided a method of treating an inflammatory or autoimmune disease or disorder, in a patient in need thereof, comprising administering to the patient a compound of Formula (I) or (Ia), or a pharmaceutically acceptable salt, solvate, or N-oxide thereof. In some embodiments is provided a method of treating inflammatory or autoimmune disease or disorder, in a patient in need thereof, comprising administering to the patient a pharmaceutical composition comprising a compound of Formula (I) or (Ia), or a pharmaceutically acceptable salt, solvate, or N-oxide thereof, and a pharmaceutically acceptable excipient. One embodiment provides a method of treating an inflammatory disease or disorder. Another embodiment provides a method of treating an autoimmune disease or disorder.

One embodiment provides a compound of Table 1 or Table 2, or a pharmaceutically acceptable salt, solvate, or N-oxide thereof, for use in a method of treatment of the human or animal body.

One embodiment provides a compound of Table 1 or Table 2, or a pharmaceutically acceptable salt, solvate, or N-oxide thereof, for use in a method of treatment of inflammatory or autoimmune disease or disorder.

One embodiment provides a pharmaceutical composition comprising a compound of Table 1 or Table 2, or a pharmaceutically acceptable salt, solvate, or N-oxide thereof, and a pharmaceutically acceptable excipient for use in a method of treatment of inflammatory or autoimmune disease or disorder.

One embodiment provides a use of a compound of Table 1 or Table 2, or a pharmaceutically acceptable salt, solvate, or N-oxide thereof, in the manufacture of a medicament for the treatment of inflammatory or autoimmune disease or disorder.

In some embodiments is provided a method of treating an inflammatory or autoimmune disease or disorder in a patient in need thereof, comprising administering to the patient a compound of Table 1 or Table 2, or a pharmaceutically acceptable salt, solvate, or N-oxide thereof. In some embodiments is provided a method of treating an inflammatory or autoimmune disease or disorder, in a patient in need thereof, comprising administering to the patient a pharmaceutical composition comprising a compound of Table 1 or Table 2, or a pharmaceutically acceptable salt, solvate, or N-oxide thereof, and a pharmaceutically acceptable excipient.

In some embodiments the inflammatory and autoimmune disease or disorder is selected from, but are not limited to: rheumatoid arthritis, psoriatic arthritis, systemic onset juvenile idiopathic arthritis, multiple sclerosis, lupus nephritis, systemic lupus erythematosus, psoriasis, Crohn's disease, colitis, asthma, graft versus host disease, allograft rejection, chronic obstructive pulmonary disease, multiple sclerosis, Alzheimer's disease, Graves' disease, cutaneous lupus, ankylosing spondylitis, cryopyrin-associated periodic syndromes (CAPS), gout, and gouty arthritis, ulcerative TNF receptor associated periodic syndrome (TRAPS), Wegener's granulomatosis, sarcoidosis, familial Mediterranean fever (FMF), neuropathic pain, and adult onset stills.

Provided herein is the method wherein the pharmaceutical composition is administered orally. Provided herein is the method wherein the pharmaceutical composition is administered by injection.

One embodiment provides a method of inhibiting TNF-α activity comprising contacting the TNF-α protein with a compound of Formula (I) or (Ia), or Table 1, or Table 2. Another embodiment provides the method of inhibiting TNF-α activity, wherein the TNF-α protein is contacted in an in vivo setting. Another embodiment provides the method of inhibiting TNF-α activity, wherein the TNF-α protein is contacted in an in vitro setting.

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

In some embodiments, the TNF-α inhibitory compounds disclosed herein are synthesized according to the following examples. As used below, and throughout the description of the invention, the following abbreviations, unless otherwise indicated, shall be understood to have the following meanings:
ACN acetonitrile
° C. degrees Celsius
δ_{H} chemical shift in parts per million downfield from tetramethylsilane
DCM dichloromethane (CH₂Cl₂)
DIAD diisopropyl azodicarboxylate
DIEA diisopropylethylamine
DMF dimethylformamide
DMSO dimethylsulfoxide
EA ethyl acetate
EtOAc ethyl acetate
ESI electrospray ionization
Et ethyl
g gram(s)
h hour(s)
HPLC high performance liquid chromatography
Hz hertz
J coupling constant (in NMR spectrometry)
LCMS liquid chromatography mass spectrometry
μ micro
m multiplet (spectral); meter(s); milli
M molar
M⁺ parent molecular ion
Me methyl
MsCl methanesulfonyl chloride
MHz megahertz
min minute(s)
mol mole(s); molecular (as in mol wt)
mL milliliter
MS mass spectrometry
nm nanometer(s)
NMR nuclear magnetic resonance
pH potential of hydrogen; a measure of the acidity or basicity of an aqueous solution
PE petroleum ether
RT room temperature
s singlet (spectral)
t triplet (spectral)
SFC Supercritical fluid chromatography
T temperature
TFA trifluoroacetic acid
THF tetrahydrofuran
TPP Triphenylphosphine

### Intermediate 1: (1R,11R)-18-(difluoromethoxy)-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

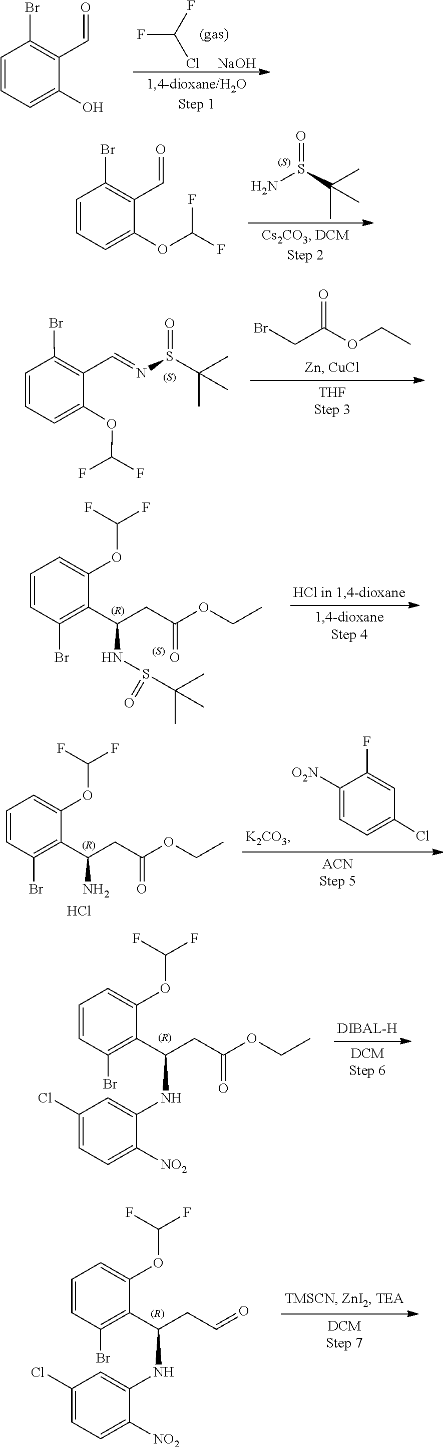

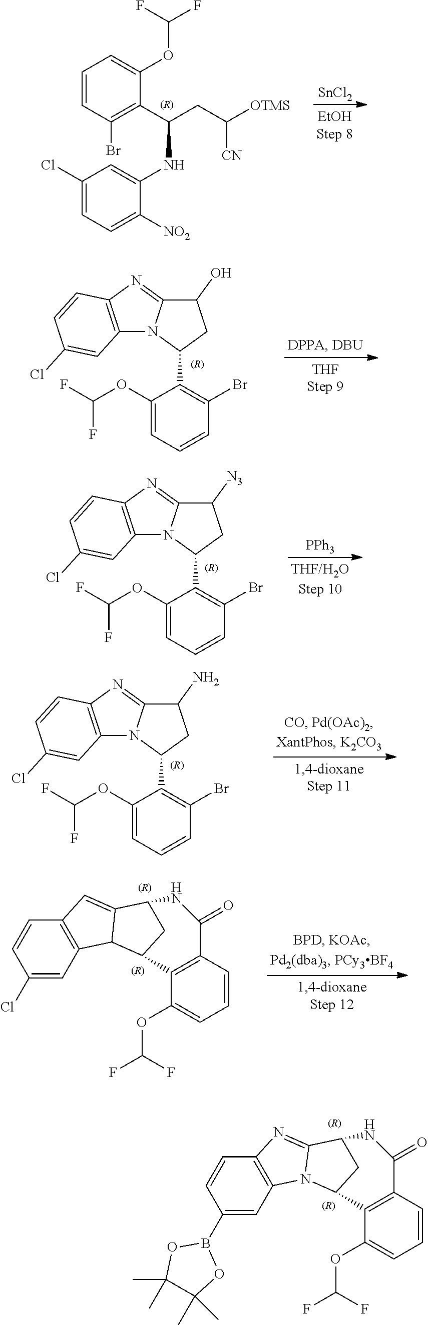

### Preparation 1A: 2-bromo-6-(difluoromethoxy)benzaldehyde

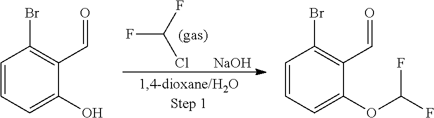

To a stirred solution of 2-bromo-6-hydroxybenzaldehyde (90.00 g, 447.719 mmol) in 1,4-dioxane (900 mL) was added a solution of NaOH (107.44 g, 2686.314 mmol) in H₂O (900 mL) dropwise at room temperature. The mixture was heated at 65° C. and chlorodifluoromethane (gas) was passed through the solution. The mixture was stirred for 1 h at 65° C. The reaction mixture was allowed to cool down to room temperature. The resulting mixture was filtered, and the filter cake was washed with EtOAc (3×100 mL). The filtrate was extracted with EtOAc (3×500 mL) and combined organic layers were dried over anhydrous Na₂SO₄. After filtration, the filtrate was concentrated under reduced pressure. The residue was purified by silica gel column chromatography, eluted with PE/EA (20:1) to afford 2-bromo-6-(difluoromethoxy)benzaldehyde (80.00 g, 71%) as a yellow oil. ¹H NMR (300 MHz, Chloroform-d) δ 10.35 (s, 1H), 7.58 (d, J=8.1 Hz, 1H), 7.41 (t, J=8.1 Hz, 1H), 7.26 (d, J=7.9 Hz, 1H), 6.61 (t, J=73.4 Hz, 1H).

### Preparation 1B: (S)—N-{[2-bromo-6-(difluoromethoxy)phenyl]methylidene}-2-methylpropane-2-sulfinamide

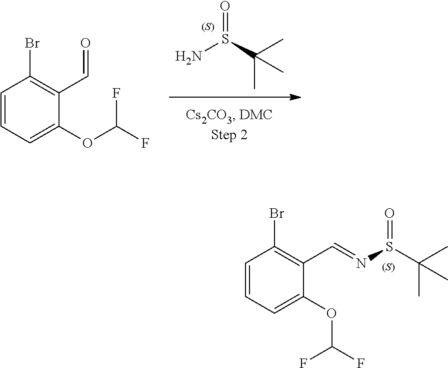

To a stirred solution of 2-bromo-6-(difluoromethoxy)benzaldehyde (80.00 g, 318.691 mmol) and (S)-2-methylpropane-2-sulfinamide (38.63 g, 318.691 mmol) in CH₂Cl₂ (800 mL) was added Cs₂CO₃ (207.67 g, 637.382 mmol) at room temperature. The mixture was stirred for 16 h at room temperature. The resulting mixture was diluted with water (1 L) and extracted with EtOAc (3×500 mL). The combined organic layers were washed with brine (1×1 L), dried over anhydrous Na₂SO₄. After filtration, the filtrate was concentrated under reduced pressure. The residue was purified by silica gel column chromatography, eluted with PE/EA (10:1) to afford (S)—N-{[2-bromo-6-(difluoromethoxy)phenyl]methylidene}-2-methylpropane-2-sulfinamide (90.00 g, 80%) as a yellow oil. MS ESI calculated for C₁₂H₁₄BrF₂NO₂S [M+H]⁺ 353.99 355.99, found 353.80 355.75. ¹H NMR (300 MHz, Chloroform-d) δ 8.84 (s, 1H), 7.59-7.57 (m, 1H), 7.35-7.31 (m, 1H), 7.26-7.24 (m, 1H), 6.57 (t, J=73.8 Hz, 1H), 1.30 (s, 9H).

### Preparation 1C: ethyl (3R)-3-[2-bromo-6-(difluoromethoxy)phenyl]-3-{[(S)-2-methylpropane-2-sulfinyl]amino}propanoate

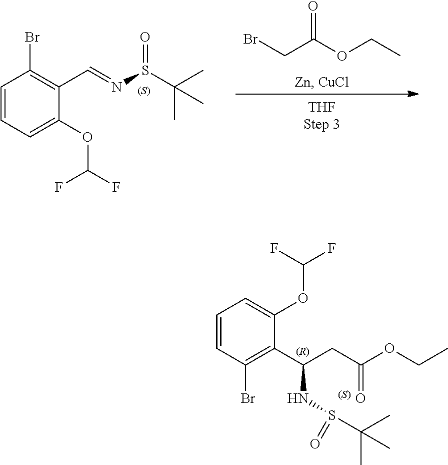

To a stirred mixture of active Zn powder (36.92 g, 564.640 mmol) in THF (200 mL) was added anhydrous CuCl (5.59 g, 56.464 mmol) at room temperature. The mixture was stirred for 0.5 h at 70° C. The mixture was allowed to cool down to room temperature. To the above mixture was added solution of ethyl bromoacetate (23.57 g, 141.160 mmol) in THF (200 mL) dropwise at room temperature. The resulting mixture was stirred for additional 0.5 h at 50° C. The resulting mixture was filtered. To the above filtrate was added solution of (S)—N-{[2-bromo-6-(difluoromethoxy)phenyl]methylidene}-2-methylpropane-2-sulfinamide (20.00 g, 56.464 mmol) in THF (20 mL) dropwise at 0° C. The resulting mixture was stirred for additional 2 h at room temperature. This reaction was quenched with sat. NH₄Cl (aq.) at room temperature. The resulting mixture was filtered, and the filter cake was washed with ethyl acetate (3×100 mL). The filtrate was extracted with EtOAc (3×200 mL). The combined organic layers were washed with brine (1×500 mL), dried over anhydrous Na₂SO₄. After filtration, the filtrate was concentrated under reduced pressure. The residue was purified by silica gel column chromatography, eluted with CH₂Cl₂/MeOH (10:1) to afford ethyl (3R)-3-[2-bromo-6-(difluoromethoxy)phenyl]-3-{[(S)-2-methylpropane-2-sulfinyl]amino}propanoate (21.00 g, 84%) as a yellow oil. MS ESI calculated for C₁₆H₂₂BrF₂NO₄S [M+H]⁺ 442.04 444.4, found 441.90 443.90. ¹H NMR (300 MHz, Chloroform-d) δ 7.50-7.42 (m, 1H), 7.23-7.00 (m, 2H), 6.62 (t, J=73.0 Hz, 1H), 5.68-5.55 (m, 1H), 4.18-4.03 (m, 2H), 3.36-2.92 (m, 2H), 1.22 (t, J=7.0 Hz, 3H), 1.16 (s, 9H).

### Preparation 1D: ethyl (3R)-3-amino-3-[2-bromo-6-(difluoromethoxy)phenyl]propanoate hydrochloride

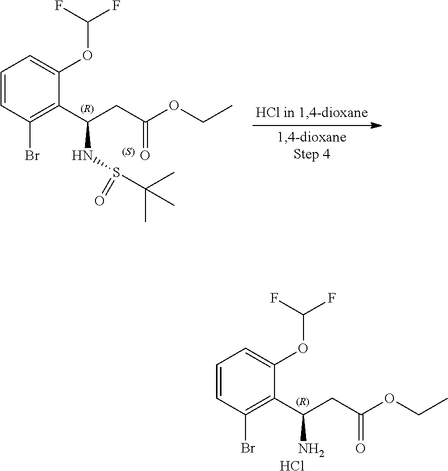

To a stirred solution of ethyl (3R)-3-[2-bromo-6-(difluoromethoxy)phenyl]-3-{[(S)-2-methylpropane-2-sulfinyl]amino}propanoate (24.00 g, 54.259 mmol) in Et₂O (50 mL) and EtOH (25 mL) was added 4 N HCl(gas) in 1,4-dioxane (75 mL) at room temperature. The mixture was stirred for 3 h at room temperature. The resulting mixture was concentrated under reduced pressure. This resulting in ethyl (3R)-3-amino-3-[2-bromo-6-(difluoromethoxy)phenyl]propanoate hydrochloride (20.00 g, 98%) as a yellow oil. MS ESI calculated for C₁₂H₁₄BrF₂NO₃ [M+H]⁺ 338.01 340.01, found 338.00 340.00.

### Preparation 1E: ethyl (3R)-3-[2-bromo-6-(difluoromethoxy)phenyl]-3-[(5-chloro-2-nitrophenyl)amino]propanoate

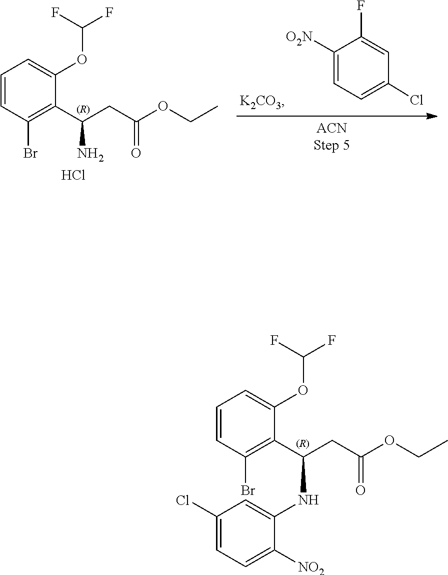

To a stirred solution of ethyl (3R)-3-amino-3-[2-bromo-6-(difluoromethoxy)phenyl]propanoate hydrochloride (20.00 g, 53.389 mmol) and 4-chloro-2-fluoro-1-nitrobenzene (11.25 g, 64.067 mmol) in ACN (200 mL) was added potassium carbonate (22.30 g, 160.167 mmol) at room temperature. The mixture was stirred for 16 h at 80° C. The resulting mixture was diluted with water (200 mL), and extracted with EtOAc (3×500 mL). The combined organic layers were washed with brine (1×500 mL), dried over anhydrous Na₂SO₄. After filtration, the filtrate was concentrated under reduced pressure. The residue was purified by silica gel column chromatography, eluted with PE/EA (10:1) to afford ethyl (3R)-3-[2-bromo-6-(difluoromethoxy)phenyl]-3-[(5-chloro-2-nitrophenyl)amino]propanoate (20.00 g, 76%) as a yellow oil. MS ESI calculated for C₁₈H₁₆BrClF₂N₂O₅ [M+H]⁺ 492.99 494.99, found 492.95 494.85. ¹H NMR (400 MHz, Chloroform-d) δ 8.92 (d, J=8.8 Hz, 1H), 8.08 (d, J=9.1 Hz, 1H), 7.48-7.43 (m, 1H), 7.23-7.11 (m, 2H), 7.11-7.04 (m, 1H), 6.65 (t, J=73.0 Hz, 1H), 6.63-6.57 (m, 1H), 5.87-5.77 (m, 1H), 4.18-4.09 (m, 2H), 3.23-3.17 (m, 1H), 3.02-2.85 (m, 1H), 1.22 (t, J=7.1 Hz, 3H).

### Preparation 1F: (3R)-3-[2-bromo-6-(difluoromethoxy)phenyl]-3-[(5-chloro-2-nitrophenyl)amino]propanal

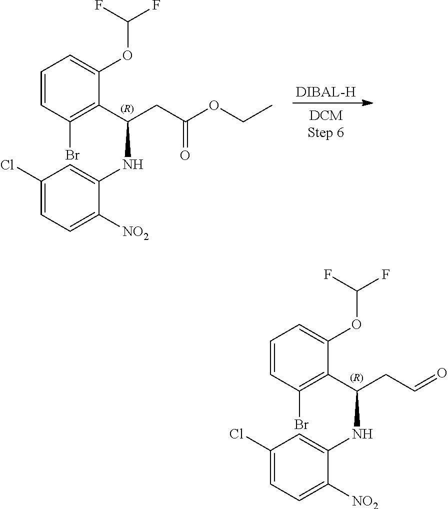

To a stirred solution of ethyl (3R)-3-[2-bromo-6-(difluoromethoxy)phenyl]-3-[(5-chloro-2-nitrophenyl)amino]propanoate (18.00 g, 36.460 mmol) in CH₂Cl₂ (200 mL) was added 1N DIBAl-H (73 mL, 73.000 mmol) in THF dropwise at −78° C. under nitrogen atmosphere. The reaction was stirred for 3 h at −78° C. under nitrogen atmosphere. The resulting mixture was quenched with sat. NH₄Cl (aq.) at −78° C., and extracted with CH₂Cl₂ (3×500 mL). The combined organic layers were dried over anhydrous Na₂SO₄. After filtration, the filtrate was concentrated under reduced pressure. The residue was purified by silica gel column chromatography, eluted with PE/EA (10:1) to afford (3R)-3-[2-bromo-6-(difluoromethoxy)phenyl]-3-[(5-chloro-2-nitrophenyl)amino]propanal (12.00 g, 73%) as a yellow oil. MS ESI calculated for C₁₆H₁₂BrClF₂N₂O₄ [M+H]⁺ 448.96 450.96, found 448.95 450.95. ¹H NMR (400 MHz, Chloroform-d) δ 9.81 (s, 1H), 8.84 (d, J=9.0 Hz, 1H), 8.08 (d, J=9.1 Hz, 1H), 7.49-7.43 (m, 1H), 7.22-7.14 (m, 2H), 7.13-7.06 (m, 1H), 6.67 (t, J=73.0 Hz, 1H), 6.65-6.60 (m, 1H), 5.97-5.88 (m, 1H), 3.56-3.41 (m, 1H), 3.22-2.93 (m, 1H).

### Preparation 1G: (4R)-4-[2-bromo-6-(difluoromethoxy)phenyl]-4-[(5-chloro-2-nitrophenyl)amino]-2-[(trimethylsilyl)oxy]butanenitrile

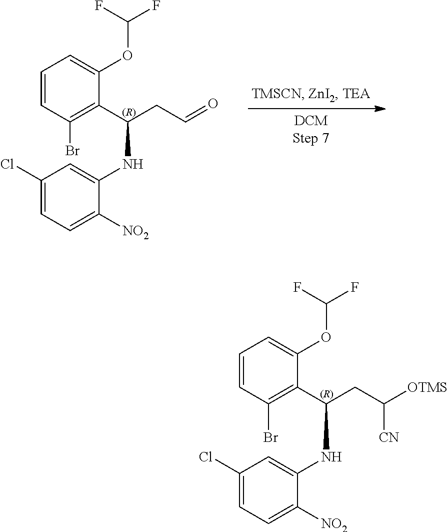

To a stirred solution of (3R)-3-[2-bromo-6-(difluoromethoxy)phenyl]-3-[(5-chloro-2-nitrophenyl)amino]propanal (12.00 g, 26.689 mmol) in CH₂Cl₂ (120 mL) were added ZnI₂ (852 mg, 2.669 mmol), TMSCN (5.30 g, 53.378 mmol) and TEA (0.37 mL, 2.669 mmol) at room temperature. The mixture was stirred for 16 h at room temperature. The resulting mixture was diluted with water (50 mL), and extracted with CH₂Cl₂ (3×50 mL). The combined organic layers were dried over anhydrous Na₂SO₄. After filtration, the filtrate was concentrated under reduced pressure to afford (4R)-4-[2-bromo-6-(difluoromethoxy)phenyl]-4-[(5-chloro-2-nitrophenyl)amino]-2-[(trimethylsilyl)oxy]butanenitrile (13.00 g, 89%) as a yellow oil. The crude product was used in the next step directly without further purification. MS ESI calculated for C₂₀H₂₁BrClF₂N₃O₄Si [M+H]⁺ 548.01 550.01, found 548.05 550.00.

### Preparation 1H: (3R)-3-[2-bromo-6-(difluoromethoxy)phenyl]-11-chloro-2,7-diazatricyclo[6.4.0.0\hat{ }{2,6}]dodeca-1(8),6,9,11-tetraen-5-ol

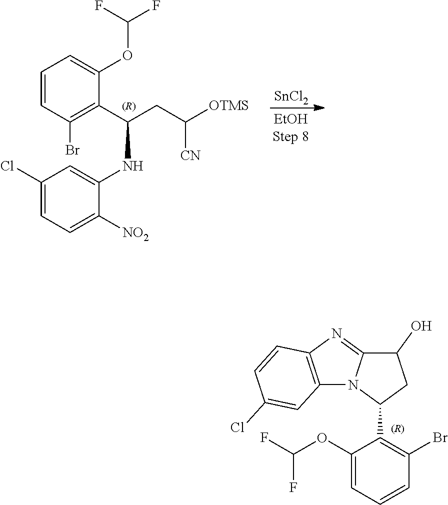

To a stirred solution of (4R)-4-[2-bromo-6-(difluoromethoxy)phenyl]-4-[(5-chloro-2-nitrophenyl)amino]-2-[(trimethylsilyl)oxy]butanenitrile (13.00 g, 23.686 mmol) in EtOH (100 mL) was added SnCl₂·2H₂O (26.96 g, 118.430 mmol) at room temperature. The mixture was stirred for 16 h at 80° C. The reaction was quenched by the addition of water (50 mL) at room temperature, and basified to pH ˜8 with 1N KOH (aq.). The mixture was filtered, the filter cake was washed with EtOAc (3×50 mL), and extracted with EtOAc (3×100 mL). The combined organic layers were washed with brine (1×100 mL), dried over anhydrous Na₂SO₄. After filtration, the filtrate was concentrated under reduced pressure. The residue was purified by silica gel column chromatography, eluted with PE/EA (1:2) to afford (3R)-3-[2-bromo-6-(difluoromethoxy)phenyl]-11-chloro-2,7-diazatricyclo[6.4.0.0\hat{ }{2,6}]dodeca-1(8),6,9,11-tetraen-5-ol (7.00 g, 69%) as a yellow solid. MS ESI calculated for C₁₇H₁₂BrClF₂N₂O₂ [M+H]⁺ 428.97 430.97, found 429.00 431.00.

### Preparation 1I: (3R)-5-azido-3-[2-bromo-6-(difluoromethoxy)phenyl]-11-chloro-2,7-diazatricyclo[6.4.0.0\hat{ }{2,6}]dodeca-1(8),6,9,11-tetraene

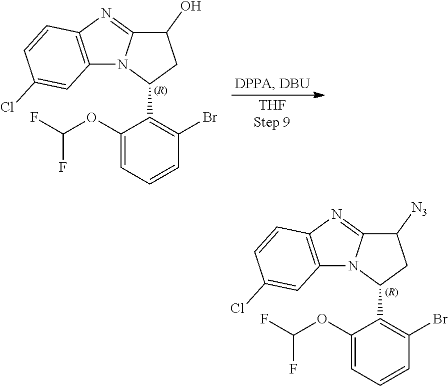

To a stirred solution of (3R)-3-[2-bromo-6-(difluoromethoxy)phenyl]-11-chloro-2,7-diazatricyclo[6.4.0.0\hat{ }{2,6}]dodeca-1(8),6,9,11-tetraen-5-ol (22.00 g, 51.204 mmol) in THF (200 mL) were added DPPA (16.91 g, 61.445 mmol) and DBU (15.59 g, 102.408 mmol) at 0° C. The mixture was stirred for 16 h at room temperature. The reaction was quenched by the addition of water (200 mL) at room temperature, and extracted with EtOAc (3×500 mL). The combined organic layers were washed with brine (3×200 mL), dried over anhydrous Na₂SO₄. After filtration, the filtrate was concentrated under reduced pressure. The residue was purified by silica gel column chromatography, eluted with CH₂Cl₂/MeOH (20:1) to afford desired mixture of cis (3R)-5-azido-3-[2-bromo-6-(difluoromethoxy)phenyl]-11-chloro-2,7-diazatricyclo[6.4.0.0\hat{ }{2,6}]dodeca-1(8),6,9,11-tetraene (15.00 g, 64%) as a green oil. MS ESI calculated for C₁₇H₁₁BrClF₂N₅O [M+H]⁺ 453.98 455.98, found 454.05 456.05.

### Preparation 1J: (3R)-3-[2-bromo-6-(difluoromethoxy)phenyl]-11-chloro-2,7-diazatricyclo[6.4.0.0\hat{ }{2,6}]dodeca-1(8),6,9,11-tetraen-5-amine

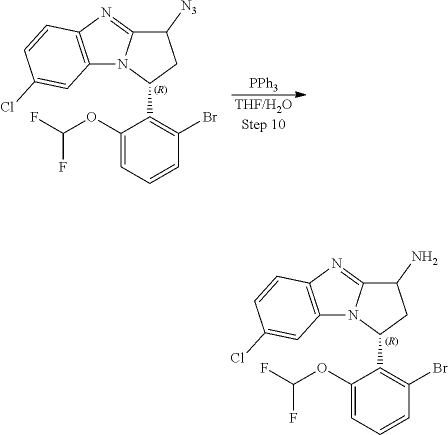

To a stirred solution of (3R)-5-azido-3-[2-bromo-6-(difluoromethoxy)phenyl]-11-chloro-2,7-diazatricyclo[6.4.0.0\hat{ }{2,6}]dodeca-1(8),6,9,11-tetraene (15.00 g, 32.992 mmol) in THF (50 mL) and H₂O (5 mL) was added PPh₃ (12.98 g, 49.488 mmol) at room temperature. The mixture was stirred for 16 h at room temperature. The resulting mixture was concentrated under reduced pressure. The residue was purified by silica gel column chromatography, eluted with CH₂Cl₂/MeOH (12:1) to afford (3R)-3-[2-bromo-6-(difluoromethoxy)phenyl]-11-chloro-2,7-diazatricyclo[6.4.0.0\hat{ }{2,6}]dodeca-1(8),6,9,11-tetraen-5-amine (10 g, 71%) as a yellow solid. MS ESI calculated for C₁₇H₁₃BrClF₂N₃O [M+H]⁺ 427.99 429.99, found 428.00 430.00. ¹H NMR (400 MHz, Chloroform-d) δ 7.70-7.58 (m, 2H), 7.35-7.28 (m, 1H), 7.23-7.00 (m, 2H), 6.90-6.46 (m, 1H), 6.17-6.06 (m, 1H), 5.98-5.58 (m, 1H), 4.77-4.57 (m, 1H), 3.60-3.40 (m 1H), 2.82-2.55 (m, 1H).

### Preparation 1K: (1R,11R)-5-chloro-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

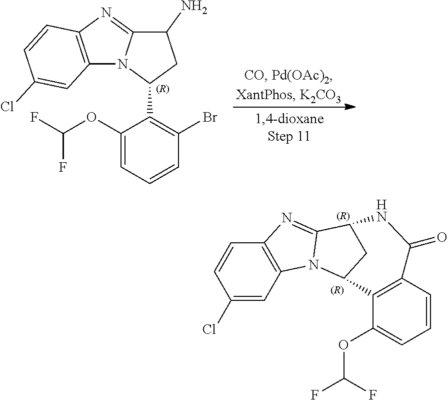

To a solution of (3R)-3-[2-bromo-6-(difluoromethoxy)phenyl]-11-chloro-2,7-diazatricyclo[6.4.0.0\hat{ }{2,6}]dodeca-1(8),6,9,11-tetraen-5-amine (500 mg, 1.166 mmol) in 1,4-dioxane (10 mL) were added K₂CO₃ (806 mg, 5.830 mmol), XantPhos (34 mg, 0.058 mmol) and Pd(OAc)₂ (13 mg, 0.058 mmol) in a pressure tank. The mixture was purged with nitrogen for 5 min and then was pressurized to 1 atm with carbon monoxide at 100° C. for 16 h. The resulting mixture was concentrated under reduced pressure. The residue was purified by silica gel column chromatography, eluted with CH₂Cl₂/MeOH (10:1) to afford a mixture of (1R,11R)-5-chloro-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one and (1S,11S)-5-chloro-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (200 mg). The mixture was purified by Chiral-Prep-HPLC with the following conditions (Column: CHIRAL ART Cellulose-SB, 2*25 cm, 5 μm; Mobile Phase A: Hex (10 mM NH₃-MeOH), Mobile Phase B: EtOH; Flow rate: 20 mL/min; Gradient: 30% B; 210/286 nm). The first peak afforded 160 mg of (1R,11R)-5-chloro-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one as a white solid. MS ESI calculated for C₁₈H₁₂ClF₂N₃O₂ [M+H]⁺ 376.06, found 375.95. ¹H NMR (400 MHz, Chloroform-d) δ 8.44 (d, J=8.0 Hz, 1H), 7.64 (d, J=8.7 Hz, 1H), 7.52-7.35 (m, 4H), 7.24-7.19 (m, 1H), 6.85 (t, J=72.6 Hz, 1H), 6.29 (d, J=7.3 Hz, 1H), 4.94 (t, J=6.6 Hz, 1H), 3.53-3.41 (m, 1H), 2.85 (d, J=13.3 Hz, 1H).

### Intermediate 1: (1R,11R)-18-(difluoromethoxy)-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

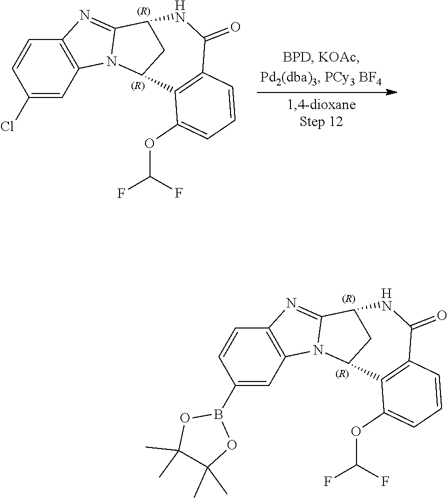

To a stirred solution of (1R,11R)-5-chloro-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (50 mg, 0.133 mmol) and BPD (51 mg, 0.200 mmol) in 1,4-dioxane (2 mL) were added KOAc (39 mg, 0.399 mmol,), Pd₂(dba)₃ (12 mg, 0.013 mmol) and PCy₃·HBF₄ (5 mg, 0.013 mmol) at room temperature under nitrogen atmosphere. The mixture was stirred for 16 h at 140° C. under nitrogen atmosphere. The resulting mixture was concentrated under reduced pressure. The residue was purified by silica gel column chromatography, eluted with CH₂Cl₂/MeOH (10:1) to afford (1R,11R)-18-(difluoromethoxy)-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (22 mg, 35%) as a yellow oil. MS ESI calculated for C₂₄H₂₄BF₂N₃O₄ [M+H]⁺ 468.18, found 467.95.

### Intermediate 2: (1R,11R)-18-(difluoromethoxy)-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

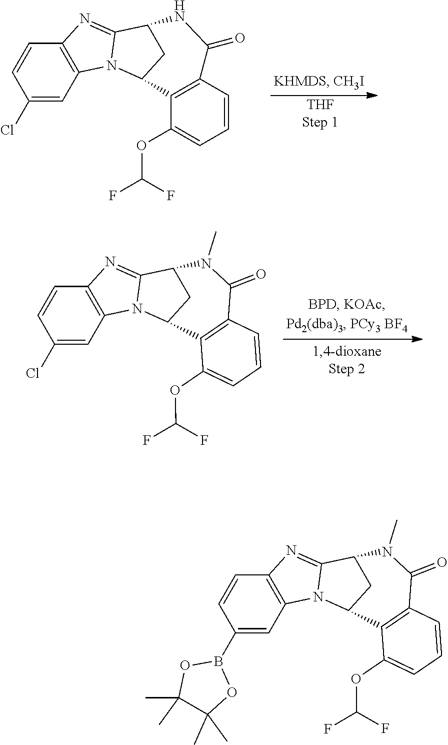 [SMILES: O=C1N[C@@H]2C[C@H](c3c(OC(F)F)cccc31)n1c2nc2ccc(Cl)cc21 ; CN1C(=O)c2cccc(OC(F)F)c2[C@H]2C[C@@H]1c1nc3ccc(Cl)cc3n12 ; CN1C(=O)c2cccc(OC(F)F)c2[C@H]2C[C@@H]1c1nc3ccc(B4OC(C)(C)C(C)(C)O4)cc3n12]

### Preparation 2A: (1R,11R)-5-chloro-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

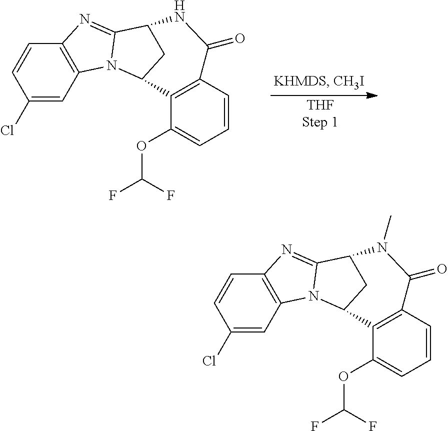 [SMILES: O=C1N[C@@H]2C[C@H](c3c(OC(F)F)cccc31)n1c2nc2ccc(Cl)cc21 ; CN1C(=O)c2cccc(OC(F)F)c2[C@H]2C[C@@H]1c1nc3ccc(Cl)cc3n12]

To a stirred solution of (1R,11R)-5-chloro-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (120 mg, 0.319 mmol) in dry THF (2 mL) was added 1N KHMDS (0.4 mL, 0.40 mmol) in THF dropwise at −78° C. under nitrogen atmosphere. This reaction was stirred for 1 h at −78° C. under nitrogen atmosphere. To the above solution was added CH₃I (68 mg, 0.479 mmol) dropwise over 2 min at −78° C. The mixture was stirred for 1 h at room temperature. The reaction was quenched by the addition of sat. NH₄Cl (aq.) (1 mL) at room temperature. The resulting mixture was concentrated under reduced pressure. The residue was purified by silica gel column chromatography, eluted with CH₂Cl₂/MeOH (10:1) to afford (1R,11R)-5-chloro-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (85 mg, 68%) as a yellow solid. MS ESI calculated for C₁₉H₁₄ClF₂N₃O₂ [M+H]⁺ 390.07 392.07, found 390.25 392.25. ¹H NMR (300 MHz, Chloroform-d) δ 8.50 (d, J=8.2 Hz, 1H), 7.65 (d, J=8.7 Hz, 1H), 7.48-7.42 (m, 2H), 7.38-7.32 (m, 1H), 7.25-7.22 (m, 1H), 6.84 (t, J=72.6 Hz, 1H), 6.24-6.21 (m, 1H), 5.02-5.00 (m, 1H), 3.53 (s, 3H), 3.49-3.41 (m, 1H), 2.90-2.86 (m, 1H).

### Intermediate 2: (1R,11R)-18-(difluoromethoxy)-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

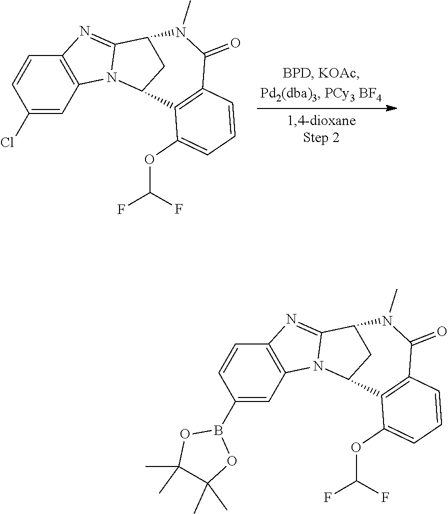 [SMILES: CN1C(=O)c2cccc(OC(F)F)c2[C@H]2C[C@@H]1c1nc3ccc(Cl)cc3n12 ; CN1C(=O)c2cccc(OC(F)F)c2[C@H]2C[C@@H]1c1nc3ccc(B4OC(C)(C)C(C)(C)O4)cc3n12]

To a stirred mixture of (1R,11R)-5-chloro-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (85 mg, 0.218 mmol), KOAc (64 mg, 0.654 mmol) and BPD (83 mg, 0.327 mmol) in 1,4-dioxane (3 mL) were added PCy₃·HBF₄ (8 mg, 0.022 mmol) and Pd₂(dba)₃ (20 mg, 0.022 mmol) at room temperature under nitrogen atmosphere. The mixture was stirred for 16 h at 140° C. under nitrogen atmosphere. The resulting mixture was concentrated under reduced pressure. The residue was purified by silica gel column chromatography, eluted with CH₂Cl₂/MeOH (10:1) to afford (1R,11R)-18-(difluoromethoxy)-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (65 mg, 62%) as a yellow oil. MS ESI calculated for C₂₅H₂₆BF₂N₃O₄ [M+H]⁺ 482.20, found 481.90.

### Example 1: (7R,14R)-1-(difluoromethoxy)-11-(7-hydroxy-7-methyl-6,7-dihydro-5H-cyclopenta[b]pyridin-3-yl)-6-methyl-6,7-dihydro-7,14-methanobenzo[f]benzo[4,5]imidazo[1,2-a][1,4]diazocin-5(14H)-one

### Synthetic Scheme

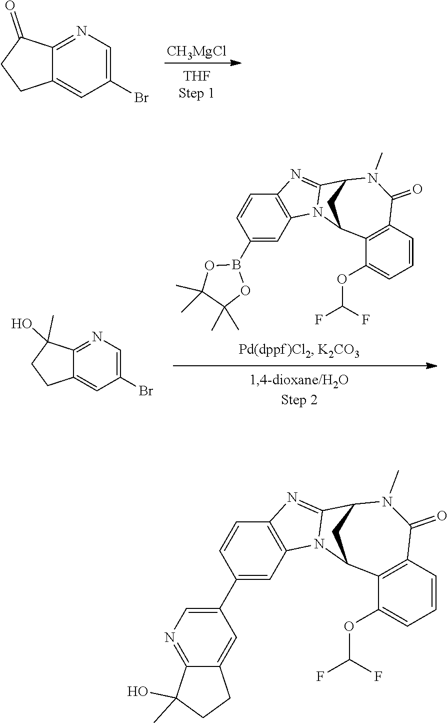 [SMILES: O=C1CCc2cc(Br)cnc21 ; "CH3MgCl" ; CC1(O)CCc2cc(Br)cnc21 ; CN1C(=O)c2cccc(OC(F)F)c2[C@H]2C[C@@H]1c1nc3ccc(B4OC(C)(C)C(C)(C)O4)cc3n12 ; CN1C(=O)c2cccc(OC(F)F)c2[C@H]2C[C@@H]1c1nc3ccc(-c4cnc5c(c4)CCC5(C)O)cc3n12]

### Preparation 1A: 3-bromo-7-methyl-5H,6H-cyclopenta[b]pyridin-7-ol

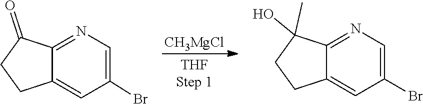

To a stirred solution of 3-bromo-5H,6H-cyclopenta[b]pyridin-7-one (300 mg, 1.415 mmol) in THF (3 mL) was added methylmagnesium chloride (3M in THF) (1.40 mL, 4.245 mmol) dropwise at 0° C. under nitrogen atmosphere. The mixture was stirred for 4 h at room temperature under nitrogen atmosphere. The reaction was quenched by the addition of sat. NH₄Cl (aq.) (10 mL) at 0° C. The resulting mixture was extracted with EtOAc (3×30 mL). The combined organic layers were washed with brine (3×25 mL), dried over anhydrous Na₂SO₄. After filtration, the filtrate was concentrated under reduced pressure. The residue was purified by reversed-phase flash chromatography with the following conditions: column, C18 silica gel; mobile phase, CH₃CN in water (10 mmol/L NH₄HCO₃), 20% to 50% gradient in 30 min; detector, 254 nm to afford 3-bromo-7-methyl-5H,6H-cyclopenta[b]pyridin-7-ol (148 mg, 45%) as a light-grey solid. MS ESI calculated for C₉H₁₀BrNO [M+H]⁺, 227.99 229.99, found 227.95 229.95. ¹H NMR (400 MHz, Chloroform-d) δ 8.51-8.46 (m, 1H), 7.73-7.67 (m, 1H), 3.06-2.93 (m, 1H), 2.88-2.75 (m, 1H), 2.37-2.19 (m, 2H), 1.58 (s, 3H).

### Example 1: (1R,11R)-18-(difluoromethoxy)-5-{7-hydroxy-7-methyl-5H,6H-cyclopenta[b]pyridin-3-yl}-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

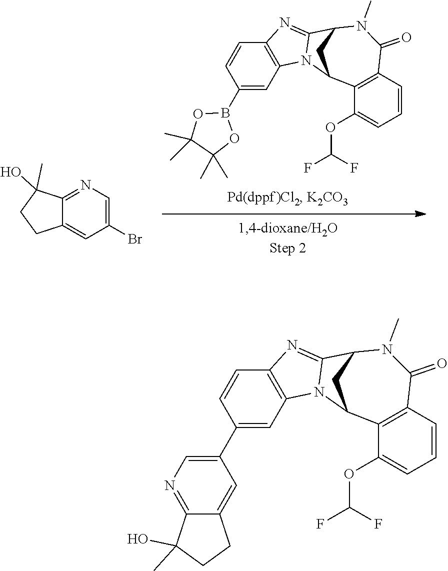 [SMILES: CC1(O)CCc2cc(Br)cnc21 ; CN1C(=O)c2cccc(OC(F)F)c2[C@H]2C[C@@H]1c1nc3ccc(B4OC(C)(C)C(C)(C)O4)cc3n12 ; CN1C(=O)c2cccc(OC(F)F)c2[C@H]2C[C@@H]1c1nc3ccc(-c4cnc5c(c4)CCC5(C)O)cc3n12]

Into a 8 mL vial were added (1R,11R)-18-(difluoromethoxy)-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (50 mg, 0.104 mmol), 3-bromo-7-methyl-5H,6H-cyclopenta[b]pyridin-7-ol (26 mg, 0.114 mmol), 1,1′-bis(diphenylphosphino)ferrocene-palladium(II)dichloride dichloromethane complex (8 mg, 0.010 mmol), K₂CO₃ (36 mg, 0.260 mmol), 1,4-dioxane (0.5 mL) and H₂O (0.1 mL) at room temperature. The resulting mixture was stirred for 2 h at 80° C. under nitrogen atmosphere. The mixture was allowed to cool down to room temperature. The resulting mixture was concentrated under reduced pressure. The residue was purified by Prep-TLC (CH₂Cl₂/CH₃OH 10:1) followed by reversed-phase flash chromatography with the following conditions: column, C18 silica gel; mobile phase, CH₃CN in water (10 mmol/L NH₄HCO₃), 20% to 50% gradient in 30 min; detector, 254 nm to afford (1R,11R)-18-(difluoromethoxy)-5-{7-hydroxy-7-methyl-5H,6H-cyclopenta[b]pyridin-3-yl}-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (30 mg, 57%) as a white solid. MS ESI calculated for C₂₈H₂₄F₂N₄O₃ [M+H]⁺, 503.18, found 503.15. ¹H NMR (400 MHz, Chloroform-d) δ 8.71-8.63 (m, 1H), 8.49 (d, J=8.2 Hz, 1H), 7.90-7.75 (m, 2H), 7.66 (d, J=1.6 Hz, 1H), 7.42 (t, J=8.1 Hz, 2H), 7.35-7.29 (m, 1H), 6.84 (t, J=72.8 Hz, 1H), 6.28 (d, J=7.1 Hz, 1H), 4.98 (d, J=7.1 Hz, 1H), 3.53 (s, 3H), 3.52-3.44 (m, 1H), 3.16-3.01 (m, 1H), 2.94-2.80 (m, 2H), 2.41-2.23 (m, 2H), 1.68 (s, 3H). ¹⁹F NMR (377 MHz, Chloroform-d) δ −80.19, −80.21, −80.63, −80.65, −80.79, −81.23.

### Example 2: (1R,11R)-5-[(1S)-1-amino-2,3-dihydro-1H-inden-5-yl]-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

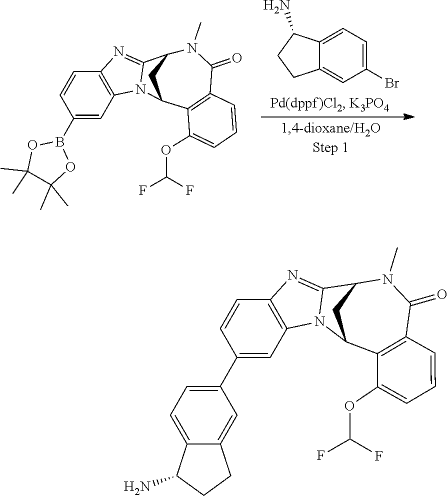 [SMILES: CN1C(=O)c2cccc(OC(F)F)c2[C@H]2C[C@@H]1c1nc3ccc(B4OC(C)(C)C(C)(C)O4)cc3n12 ; N[C@H]1CCc2cc(Br)ccc21 ; CN1C(=O)c2cccc(OC(F)F)c2[C@H]2C[C@@H]1c1nc3ccc(-c4ccc5c(c4)CC[C@@H]5N)cc3n12]

To a solution of (1S)-5-bromo-2,3-dihydro-1H-inden-1-amine (24 mg, 0.114 mmol) and (1R,11R)-18-(difluoromethoxy)-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (50 mg, 0.104 mmol) in 1,4-dioxane (0.5 mL) and H₂O (0.1 mL) were added K₃PO₄ (66 mg, 0.312 mmol) and Pd(dppf)Cl₂·CH₂Cl₂ (8 mg, 0.010 mmol). After stirring for 2 h at 80° C. under a nitrogen atmosphere, the resulting mixture was concentrated under reduced pressure. The residue was purified by silica gel column chromatography, eluted with CH₂Cl₂/MeOH (5:1) followed by reversed-phase flash chromatography with the following conditions: column, C18 silica gel; mobile phase, CH₃CN in water (10 mmol/L NH₄HCO₃), 20% to 50% gradient in 30 min; detector, 254 nm. This resulted in (1R,11R)-5-[(1S)-1-amino-2,3-dihydro-1H-inden-5-yl]-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (22 mg, 42%) as a white solid. MS ESI calculated for C₂₈H₂₄F₂N₄O₂ [M+H]⁺, 487.19, found 487.30. ¹H NMR (400 MHz, Chloroform-d) δ 8.49 (d, J=8.3 Hz, 1H), 7.75 (d, J=8.5 Hz, 1H), 7.68 (d, J=1.7 Hz, 1H), 7.51-7.32 (m, 5H), 7.32-7.27 (m, 1H), 6.82 (t, J=72.9 Hz, 1H), 6.27 (d, J=7.2 Hz, 1H), 4.96 (d, J=7.1 Hz, 1H), 4.42 (t, J=7.4 Hz, 1H), 3.52 (s, 3H), 3.50-3.40 (m, 1H), 3.08-2.98 (m, 1H), 2.94-2.74 (m, 2H), 2.61-2.50 (m, 1H), 1.83-1.68 (m, 1H). ¹⁹F NMR (377 MHz, Chloroform-d) δ −80.30, −80.48, −80.88, −81.32.

### Example 3: (1R,11R)-18-(difluoromethoxy)-5-{7-hydroxy-7-methyl-5H,6H-cyclopenta[b]pyridin-3-yl}-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

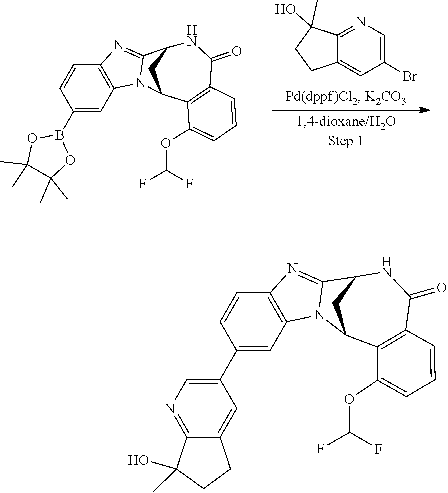 [SMILES: CC1(C)OB(c2ccc3nc4n(c3c2)[C@@H]2C[C@H]4NC(=O)c3cccc(OC(F)F)c32)OC1(C)C ; CC1(O)CCc2cc(Br)cnc21 ; CC1(O)CCc2cc(-c3ccc4nc5n(c4c3)[C@@H]3C[C@H]5NC(=O)c4cccc(OC(F)F)c43)cnc21]

Into a 8 mL vial were added (1R,11R)-18-(difluoromethoxy)-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (50 mg, 0.107 mmol), 3-bromo-7-methyl-5H,6H-cyclopenta[b]pyridin-7-ol (27 mg, 0.118 mmol), 1,1′-bis(diphenylphosphino)ferrocene-palladium(II)dichloride dichloromethane complex (9 mg, 0.011 mmol), K₃PO₄ (68 mg, 0.321 mmol), 1,4-dioxane (0.5 mL) and H₂O (0.1 mL) at room temperature. The resulting mixture was stirred for 2 h at 80° C. under nitrogen atmosphere. The mixture was allowed to cool down to room temperature. The resulting mixture was concentrated under reduced pressure. The residue was purified by Prep-TLC (CH₂Cl₂/CH₃OH 9:1) followed by reversed-phase flash chromatography with the following conditions: column, C18 silica gel; mobile phase, CH₃CN in water (10 mmol/L NH₄HCO₃), 20% to 50% gradient in 30 min; detector, 254 nm to afford (1R,11R)-18-(difluoromethoxy)-5-{7-hydroxy-7-methyl-5H,6H-cyclopenta[b]pyridin-3-yl}-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (15 mg, 30%) as a white solid. MS ESI calculated for C₂₇H₂₂F₂N₄O₃ [M+H]⁺ 489.17, found 489.10. ¹H NMR (400 MHz, Chloroform-d) δ 8.65 (s, 1H), 8.47-8.40 (m, 1H), 7.80 (t, J=6.7 Hz, 2H), 7.64 (s, 1H), 7.49-7.40 (m, 2H), 7.44-7.34 (m, 1H), 7.24 (d, J=6.6 Hz, 1H), 6.86 (t, J=72.7 Hz, 1H), 6.38 (d, J=7.2 Hz, 1H), 4.97 (t, J=6.6 Hz, 1H), 3.56-3.45 (m, 1H), 3.16-3.03 (m, 1H), 2.98-2.85 (m, 2H), 2.44-2.27 (m, 2H), 1.69 (s, 3H). ¹⁹F NMR (377 MHz, Chloroform-d) δ −80.38, −80.39, −80.82, −80.83, −80.89, −81.34.

### Example 4: (1R,11R)-5-[(3R)-3-amino-2,3-dihydro-1-benzofuran-6-yl]-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

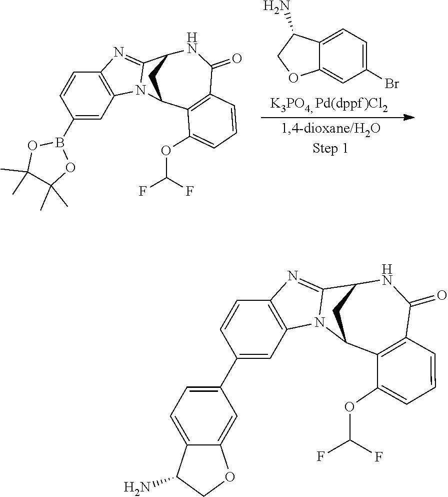 [SMILES: CC1(C)OB(c2ccc3nc4n(c3c2)[C@@H]2C[C@H]4NC(=O)c3cccc(OC(F)F)c32)OC1(C)C ; N[C@H]1COc2cc(Br)ccc21 ; N[C@H]1COc2cc(-c3ccc4nc5n(c4c3)[C@@H]3C[C@H]5NC(=O)c4cccc(OC(F)F)c43)ccc21]

A solution of (3R)-6-bromo-2,3-dihydro-1-benzofuran-3-amine (41 mg, 0.192 mmol), (1R,11R)-18-(difluoromethoxy)-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (60 mg, 0.128 mmol), K₃PO₄ (81 mg, 0.384 mmol) and Pd(dppf)Cl₂·CH₂Cl₂ (10 mg, 0.013 mmol) in 1,4-dioxane (1 mL) and H₂O (0.2 mL) was stirred for 2 h at 80° C. under nitrogen atmosphere. The mixture was allowed to cool down to room temperature. The resulting mixture was concentrated under vacuum. The residue was purified by silica gel column chromatography, eluted with DCM/MeOH (10:1) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (1R,11R)-5-[(3R)-3-amino-2,3-dihydro-1-benzofuran-6-yl]-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (26 mg, 41%) as a white solid. MS ESI calculated for C₂₆H₂₀F₂N₄O₃ [M+H]⁺, 475.15, found 475.20. ¹H NMR (400 MHz, Chloroform-d) δ 8.43 (d, J=8.0 Hz, 1H), 7.75 (d, J=8.4 Hz, 1H), 7.64 (d, J=1.8 Hz, 1H), 7.49-7.38 (m, 2H), 7.41-7.33 (m, 2H), 7.23 (d, J=6.6 Hz, 1H), 7.18-7.14 (m, 1H), 7.04 (d, J=1.6 Hz, 1H), 6.84 (t, J=72.7 Hz, 1H), 6.36 (d, J=7.3 Hz, 1H), 4.95 (t, J=6.6 Hz, 1H), 4.77-4.69 (m, 1H), 4.72-4.65 (m, 1H), 4.27-4.22 (m, 1H), 3.52-3.43 (m, 1H), 2.86 (d, J=13.3 Hz, 1H). ¹⁹F NMR (377 MHz, Chloroform-d) δ −80.21 −80.66 −80.95 −81.40.

### Example 5: (1R,11R)-5-[(3R)-3-amino-2,3-dihydro-1-benzofuran-6-yl]-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

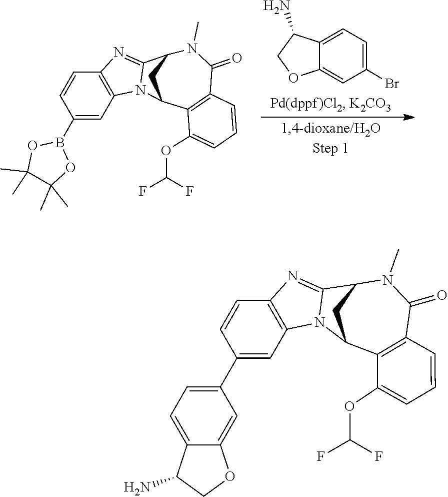 [SMILES: CN1C(=O)c2cccc(OC(F)F)c2[C@H]2C[C@@H]1c1nc3ccc(B4OC(C)(C)C(C)(C)O4)cc3n12 ; N[C@H]1COc2cc(Br)ccc21 ; CN1C(=O)c2cccc(OC(F)F)c2[C@H]2C[C@@H]1c1nc3ccc(-c4ccc5c(c4)OC[C@@H]5N)cc3n12]

To a stirred solution of (1R,11R)-18-(difluoromethoxy)-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3,5,7,9,14,16,18-heptaen-13-one (50 mg, 0.104 mmol) and (3R)-6-bromo-2,3-dihydro-1-benzofuran-3-amine (29 mg, 0.135 mmol) in 1,4-dioxane (1 mL) and H₂O (0.2 mL) were added K₂CO₃ (36 mg, 0.260 mmol) and Pd(dppf)Cl₂·CH₂Cl₂ (9 mg, 0.011 mmol) at room temperature under nitrogen atmosphere. The resulting mixture was stirred for 3 h at 80° C. under nitrogen atmosphere. The resulting mixture was concentrated under reduced pressure. The residue was purified by silica gel column chromatography, eluted with CH₂Cl₂/CH₃OH (12/1) followed by reversed-phase flash chromatography with the following conditions: column, C18 silica gel; mobile phase, CH₃CN in water (10 mmol/L NH₄HCO₃), 30% to 45% gradient in 30 min; detector, 254 nm. This resulted in (1R,11R)-5-[(3R)-3-amino-2,3-dihydro-1-benzofuran-6-yl]-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (27 mg, 54%) as a white solid. MS ESI calculated for C₂₇H₂₂F₂N₄O₃ [M+H]⁺, 489.17, found 489.15. ¹H NMR (400 MHz, Chloroform-d) δ 8.49 (d, J=8.2 Hz, 1H), 7.74 (d, J=8.5 Hz, 1H), 7.66 (s, 1H), 7.50-7.35 (m, 3H), 7.33-7.29 (m, 1H), 7.16 (d, J=7.8 Hz, 1H), 7.04 (s, 1H), 6.83 (t, J=72.8 Hz, 1H), 6.27 (d, J=7.1 Hz, 1H), 4.95 (d, J=7.1 Hz, 1H), 4.78-4.64 (m, 2H), 4.31-4.22 (m, 1H), 3.52 (s, 3H), 3.50-3.42 (m, 1H), 2.87 (d, J=13.5 Hz, 1H). ¹⁹F NMR (377 MHz, Chloroform-d) δ −80.04, −80.49, −80.89, −81.34.

### Example 6: (1R,11R)-5-[(1S)-1-amino-2,3-dihydro-1H-inden-5-yl]-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

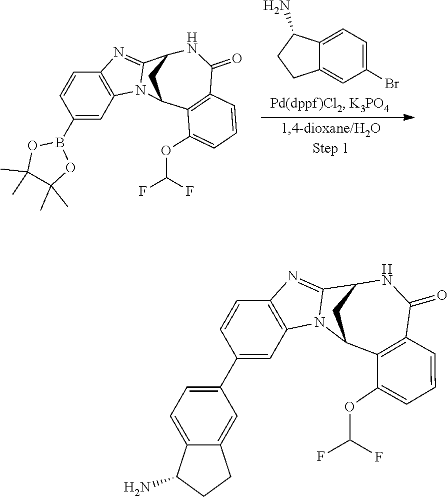 [SMILES: CC1(C)OB(c2ccc3nc4n(c3c2)[C@@H]2C[C@H]4NC(=O)c3cccc(OC(F)F)c32)OC1(C)C ; N[C@H]1CCc2cc(Br)ccc21 ; N[C@H]1CCc2cc(-c3ccc4nc5n(c4c3)[C@@H]3C[C@H]5NC(=O)c4cccc(OC(F)F)c43)ccc21]

To a solution of (1S)-5-bromo-2,3-dihydro-1H-inden-1-amine (25 mg, 0.118 mmol) and (1R,11R)-18-(difluoromethoxy)-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (50 mg, 0.107 mmol) in 1,4-dioxane (0.5 mL) and H₂O (0.1 mL) were added K₃PO₄ (68 mg, 0.321 mmol) and Pd(dppf)Cl₂·CH₂Cl₂ (9 mg, 0.011 mmol). After stirring for 2 h at 80° C. under a nitrogen atmosphere, the resulting mixture was concentrated under reduced pressure. The residue was purified by silica gel column chromatography, eluted with CH₂Cl₂/MeOH (5:1) followed by reversed-phase flash chromatography with the following conditions: column, C18 silica gel; mobile phase, CH₃CN in water (10 mmol/L NH₄HCO₃), 20% to 50% gradient in 30 min; detector, 254 nm. This resulted in (1R,11R)-5-[(1S)-1-amino-2,3-dihydro-1H-inden-5-yl]-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (19 mg, 36%) as a white solid. MS ESI calculated for C₂₇H₂₂F₂N₄O₂ [M+H]⁺, 473.17, found 473.15. ¹H NMR (400 MHz, Chloroform-d) δ 8.42 (d, J=8.1 Hz, 1H), 7.75 (d, J=8.5 Hz, 1H), 7.65 (d, J=1.7 Hz, 1H), 7.51-7.38 (m, 5H), 7.38-7.32 (m, 1H), 7.03-6.99 (m, 1H), 6.74 (d, J=72.7 Hz, 1H), 6.37 (d, J=7.2 Hz, 1H), 4.92 (t, J=6.6 Hz, 1H), 4.42 (t, J=7.4 Hz, 1H), 3.53-3.43 (m, 1H), 3.08-2.98 (m, 1H), 2.93-2.82 (m, 2H), 2.63-2.50 (m, 1H), 1.83-7.71 (m, 1H). ¹⁹F NMR (377 MHz, Chloroform-d) δ −80.20, −80.65, −80.94, −81.38.

### Example 7: (1R,11R)-5-[(1S)-1-amino-7-fluoro-2,3-dihydro-1H-inden-5-yl]-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

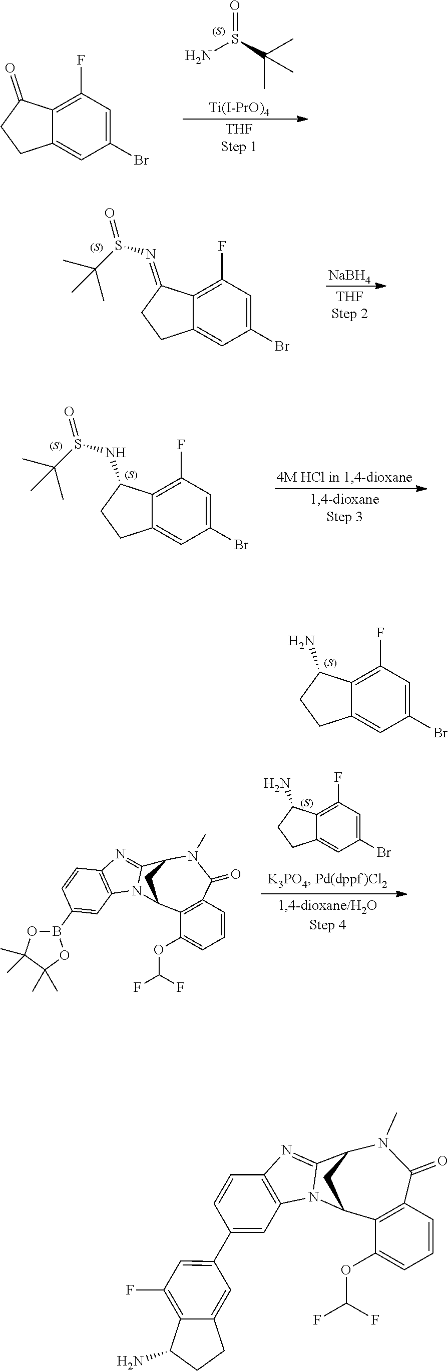

### Preparation 7A: (S)—N-[(1E)-5-bromo-7-fluoro-2,3-dihydroinden-1-ylidene]-2-methylpropane-2-sulfinamide

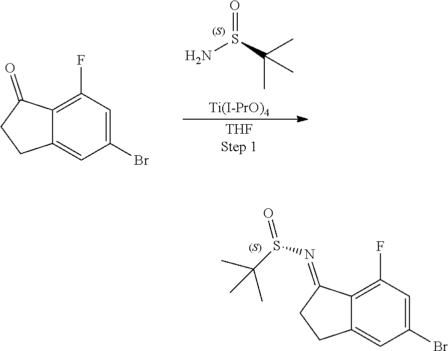

To a stirred solution of 5-bromo-7-fluoro-2,3-dihydroinden-1-one (4.50 g, 19.647 mmol) and Ti(OEₜ)₄ (8.96 g, 39.294 mmol) in THF (45 mL) was added (S)-2-methylpropane-2-sulfinamide (4.76 g, 39.294 mmol) at room temperature. The resulting mixture was stirred for 16 h at 60° C. The reaction was quenched with water. The aqueous layer was extracted with CH₂C12 (3×100 mL). The resulting mixture was concentrated under vacuum. The residue was purified by silica gel column chromatography, eluted with PE/EA (5:1) to afford (S)—N-[(1E)-5-bromo-7-fluoro-2,3-dihydroinden-1-ylidene]-2-methylpropane-2-sulfinamide (2.00 g, 30%) as a green solid. MS ESI calculated for C₁₃H₁₅BrFNOS [M+H]⁺, 332.00 334.00, found 332.00 333.95. ¹H NMR (300 MHz, Chloroform-d) δ 7.35 (s, 1H), 7.16 (d, J=9.1 Hz, 1H), 3.56-3.46 (m, 1H), 3.19-3.11 (m, 2H), 3.15-3.03 (m, 1H), 1.32 (s, 9H).

### Preparation 7B: (S)—N-[(1S)-5-bromo-7-fluoro-2,3-dihydro-1H-inden-1-yl]-2-methylpropane-2-sulfinamide

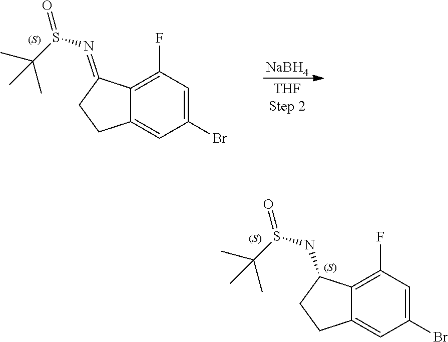

A solution of (S)—N-[(1E)-5-bromo-7-fluoro-2,3-dihydroinden-1-ylidene]-2-methylpropane-2-sulfinamide (1.00 g, 3.010 mmol) and NaBH₄ (171 mg, 4.515 mmol) in THF (10 mL) was stirred for 30 min at −50° C. under nitrogen atmosphere. The resulting mixture was stirred for 2 h at 0° C. under nitrogen atmosphere. The resulting mixture was concentrated under reduced pressure. The residue was purified by silica gel column chromatography, eluted with CH₂Cl₂/MeOH (20:1) to afford (S)—N-[(1S)-5-bromo-7-fluoro-2,3-dihydro-1H-inden-1-yl]-2-methylpropane-2-sulfinamide (500 mg, 50%) as a green oil. MS ESI calculated for C₁₃H₁₇BrFNOS [M+H]⁺, 334.02 336.02, found 334.00 336.00. ¹H NMR (400 MHz, Chloroform-d) δ 7.22-7.19 (m, 1H), 7.11-7.04 (m, 1H), 5.10-5.05 (m, 1H), 3.71-3.65 (m, 1H), 3.18-3.07 (m, 1H), 2.94-2.84 (m, 1H), 2.52-2.41 (m, 1H), 2.30-2.18 (m, 1H), 1.19 (s, 9H).

### Preparation 7C: (1S)-5-bromo-7-fluoro-2,3-dihydro-1H-inden-1-amine

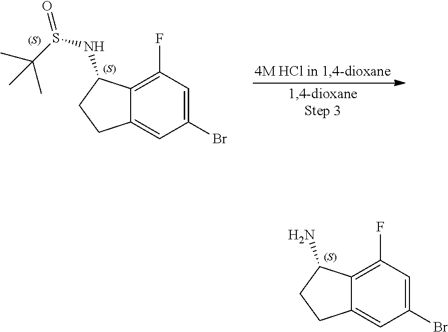

A solution of (S)—N-(5-bromo-7-fluoro-2,3-dihydro-1H-inden-1-yl)-2-methylpropane-2-sulfinamide (500 mg, 1.496 mmol) and HCl(gas) in 1,4-dioxane (3 mL, 12.000 mmol) in 1,4-dioxane (6 mL) was stirred for 2 h at 10° C. The resulting mixture was concentrated under vacuum. The mixture was basified to pH 8 with saturated NaHCO₃ (aq.). The aqueous layer was extracted with EtOAc (2×50 mL). The resulting mixture was concentrated under vacuum. The residue was purified by silica gel column chromatography, eluted with CH₂Cl₂/MeOH (20:1) followed by reversed-phase flash chromatography with the following conditions: column, C18 silica gel; mobile phase, CH₃CN in water (10 mmol/L NH₄HCO₃), 25% to 55% gradient in 25 min; detector, 254 nm. This resulted in 5-bromo-7-fluoro-2,3-dihydro-1H-inden-1-amine (250 mg) as a colorless oil. The mixture was purified by Chiral HPLC with the following conditions, Column: CHIRALPAK IG, 3*25 cm, 5 μm; Mobile Phase A: Hex (0.5% 2M NH₃-MeOH), Mobile Phase B: EtOH; Flow rate: 40 mL/min; Gradient: isocratic 10; Wave Length: 215/256 nm; RT1(min): 10.5; RT2(min): 13; Sample Solvent: DCM. The first peak was concentrated under vacuum. This resulted in (1S)-5-bromo-7-fluoro-2,3-dihydro-1H-inden-1-amine (170 mg, 49%) as a colorless oil. MS ESI calculated for C₉H₉BrFN [M+H]⁺, 229.99 231.99, found 230.05 232.05. ¹H NMR (400 MHz, Chloroform-d) δ 7.16 (s, 1H), 7.08-7.02 (m, 1H), 4.60 (t, J=7.5 Hz, 1H), 3.10-3.00 (m, 1H), 2.87-2.78 (m, 1H), 2.54-2.44 (m, 1H), 1.86-1.75 (m, 1H).

### Example 7: (1R,11R)-5-[(1S)-1-amino-7-fluoro-2,3-dihydro-1H-inden-5-yl]-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

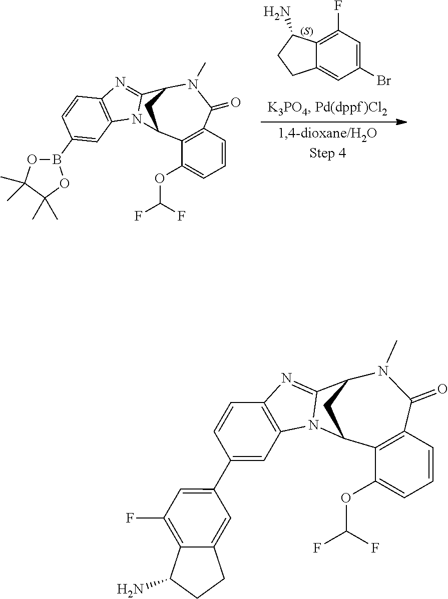

To a stirred solution of (1S)-5-bromo-7-fluoro-2,3-dihydro-1H-inden-1-amine (24 mg, 0.104 mmol) and (1R,11R)-18-(difluoromethoxy)-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (50 mg, 0.104 mmol) in 1,4-dioxane (2 mL) and H₂O (0.4 mL) were added K₃PO₄ (66 mg, 0.312 mmol) and Pd(dppf)Cl₂ (8 mg, 0.010 mmol) at room temperature. The resulting mixture was stirred for 16 h at 80° C. under nitrogen atmosphere. The resulting mixture was concentrated under vacuum. The residue was purified by silica gel column chromatography, eluted with CH₂Cl₂/MeOH (10:1) followed by reversed-phase flash chromatography with the following conditions: column, C18 silica gel; mobile phase, CH₃CN in water (10 mmol/L NH₄HCO₃), 30% to 60% gradient in 25 min; detector, 254 nm. The resulting mixture was concentrated under reduced pressure. This resulted in (1R,11R)-5-[(1S)-1-amino-7-fluoro-2,3-dihydro-1H-inden-5-yl]-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (30 mg, 58%) as a white solid. MS ESI calculated for C₂₈H₂₃F₃N₄O₂ [M+H]⁺, 505.18, found 505.15. ¹H NMR (400 MHz, DMSO-d₆) δ 8.32-8.25 (m, 1H), 7.93-7.64 (m, 3H), 7.56-7.42 (m, 3H), 7.30 (s, 1H), 7.22-7.13 (m, 1H), 6.28 (d, J=7.0 Hz, 1H), 5.23 (d, J=7.1 Hz, 1H), 4.56-4.49 (m, 1H), 3.57-3.47 (m, 1H), 3.42-3.35 (m, 4H), 3.13-3.02 (m, 1H), 2.88-2.74 (m, 2H), 2.41-2.29 (m, 1H), 2.04-1.85 (m, 1H), 1.82-1.70 (m, 1H). ¹⁹F NMR (377 MHz, DMSO-d₆) δ −82.23-−82.26 (1F), −121.15 (1F).

### Example 8: (1R,11R)-18-(difluoromethoxy)-12-methyl-5-{7-oxo-5H,6H-cyclopenta[b]pyridin-3-yl}-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

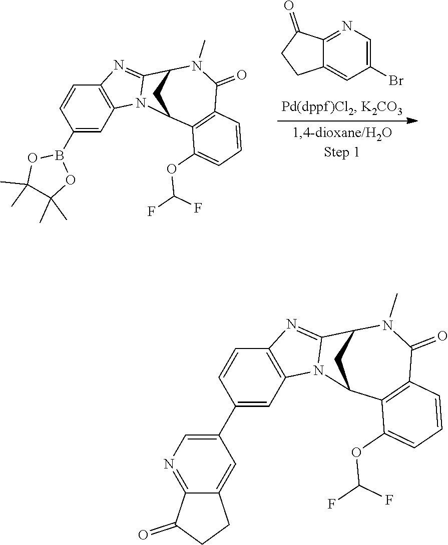 [SMILES: CN1C(=O)c2cccc(OC(F)F)c2[C@H]2C[C@@H]1c1nc3ccc(B4OC(C)(C)C(C)(C)O4)cc3n12 ; O=C1CCc2cc(Br)cnc21 ; CN1C(=O)c2cccc(OC(F)F)c2[C@H]2C[C@@H]1c1nc3ccc(-c4cnc5c(c4)CCC5=O)cc3n12]

A solution of (1R,11R)-18-(difluoromethoxy)-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (50 mg, 0.104 mmol), 3-bromo-5H,6H-cyclopenta[b]pyridin-7-one (22 mg, 0.104 mmol), Pd(dppf)Cl₂·CH₂Cl₂ (8 mg, 0.010 mmol) and K₂CO₃ (36 mg, 0.260 mmol) in 1,4-dioxane (0.8 mL) and H₂O (0.2 mL) was stirred for 2 h at 80° C. under nitrogen atmosphere. The resulting mixture was concentrated under vacuum. The residue was purified by silica gel column chromatography, eluted with CH₂Cl₂/MeOH (9:1) followed by reversed-phase flash chromatography with the following conditions: column, C18 silica gel; mobile phase, CH₃CN in water (10 mmol/L NH₄HCO₃), 20% to 45% gradient in 20 min; detector, 254 nm to afford (1R,11R)-18-(difluoromethoxy)-12-methyl-5-{7-oxo-5H,6H-cyclopenta[b]pyridin-3-yl}-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (13 mg, 26%) as a white solid. MS ESI calculated for C₂₇H₂₀F₂N₄O₃ [M+H]⁺, 487.15 found 487.10. ¹H NMR (400 MHz, Chloroform-d) δ 9.00 (d, J=2.0 Hz, 1H), 8.53-8.48 (m, 1H), 8.02 (d, J=2.0 Hz, 1H), 7.87 (d, J=8.5 Hz, 1H), 7.81 (d, J=1.7 Hz, 1H), 7.60-7.53 (m, 1H), 7.45 (t, J=8.2 Hz, 1H), 7.34-7.29 (m, 1H), 6.86 (t, J=72.8 Hz, 1H), 6.35 (d, J=7.0 Hz, 1H), 5.08 (d, J=6.9 Hz, 1H), 3.56 (s, 3H), 3.57-3.47 (m, 1H), 3.27-3.20 (m, 2H), 2.94 (d, J=13.5 Hz, 1H), 2.87-2.79 (m, 2H). ¹⁹F NMR (377 MHz, Chloroform-d) δ −80.29, −80.74, −80.84, −81.28.

### Example 9: (1R,11R)-5-[(1R)-1-amino-2,3-dihydro-1H-inden-5-yl]-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

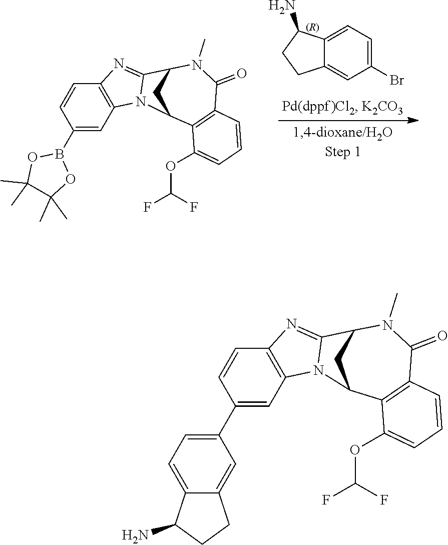

To a stirred solution of (1R,11R)-18-(difluoromethoxy)-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3,5,7,9,14,16,18-heptaen-13-one (50 mg, 0.104 mmol) and (1R)-5-bromo-2,3-dihydro-1H-inden-1-amine (29 mg, 0.135 mmol) in 1,4-dioxane (1 mL) and H₂O (0.2 mL) were added K₂CO₃ (36 mg, 0.260 mmol) and Pd(dppf)Cl₂·CH₂Cl₂ (9 mg, 0.010 mmol) at room temperature under nitrogen atmosphere. The resulting mixture was stirred for 3 h at 80° C. under nitrogen atmosphere. The resulting mixture was concentrated under reduce pressure. The residue was purified by silica gel column chromatography, eluted with CH₂Cl₂/MeOH (12/1) followed by reversed-phase flash chromatography with the following conditions: column, C18 silica gel; mobile phase, CH₃CN in water (10 mmol/L NH₄HCO₃), 30% to 45% gradient in 30 min; detector, 254 nm. This resulted in (1R,11R)-5-[(1R)-1-amino-2,3-dihydro-1H-inden-5-yl]-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (26 mg, 51%) as a white solid. MS ESI calculated for C₂₈H₂₄F₂N₄O₂ [M+H]⁺, 487.19, found 487.30. ¹H NMR (400 MHz, Chloroform-d) δ 8.51-8.46 (m, 1H), 7.74 (d, J=8.5 Hz, 1H), 7.69-7.64 (m, 1H), 7.50-7.36 (m, 5H), 7.32-7.26 (m, 1H), 6.81 (t, J=73.3 Hz, 1H), 6.25 (d, J=7.2 Hz, 1H), 4.96 (d, J=7.1 Hz, 1H), 4.46 (t, J=7.3 Hz, 1H), 3.52 (s, 3H), 3.51-3.40 (m, 1H), 3.10-3.00 (m, 1H), 2.94-2.82 (m, 2H), 2.63-2.50 (m, 1H), 1.86-1.72 (m, 1H). ¹⁹F NMR (377 MHz, Chloroform-d) δ −80.04, −80.49, −80.89, −81.34.

### Example 10: (1R,11R)-5-[(4S)-4-amino-3,4-dihydro-2H-1-benzopyran-7-yl]-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

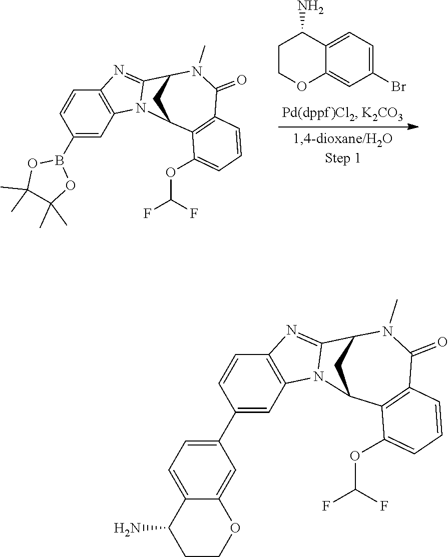

To a stirred solution of (1R,11R)-18-(difluoromethoxy)-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3,5,7,9,14,16,18-heptaen-13-one (50 mg, 0.104 mmol) and (4S)-7-bromo-3,4-dihydro-2H-1-benzopyran-4-amine (31 mg, 0.135 mmol) in 1,4-dioxane (1 mL) and H₂O (0.2 mL) were added K₂CO₃ (36 mg, 0.260 mmol) and Pd(dppf)Cl₂·CH₂Cl₂ (9 mg, 0.010 mmol) at room temperature under nitrogen atmosphere. The resulting mixture was stirred for 3 h at 80° C. under nitrogen atmosphere. The residue was purified by silica gel column chromatography, eluted with CH₂Cl₂/MeOH (12/1) to followed by reversed-phase flash chromatography with the following conditions: column, C18 silica gel; mobile phase, CH₃CN in water (10 mmol/L NH₄HCO₃), 30% to 45% gradient in 30 min; detector, 254 nm. This resulted in (1R,11R)-5-[(4S)-4-amino-3,4-dihydro-2H-1-benzopyran-7-yl]-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (29 mg, 56%) as a white solid. MS ESI calculated for C₂₈H₂₄F₂N₄O₃ [M+H]⁺, 503.18, found 503.05. ¹H NMR (400 MHz, Chloroform-d) δ 8.52-8.44 (m, 1H), 7.73 (d, J=8.5 Hz, 1H), 7.65 (d, J=1.7 Hz, 1H), 7.50-7.43 (m, 1H), 7.45-7.36 (m, 2H), 7.30 (d, J=8.2 Hz, 1H), 7.17-7.11 (m, 1H), 7.06 (d, J=1.9 Hz, 1H), 6.83 (t, J=73.3 Hz, 1H), 6.25 (d, J=7.2 Hz, 1H), 4.95 (d, J=7.1 Hz, 1H), 4.40-4.22 (m, 2H), 4.13 (t, J=5.3 Hz, 1H), 3.52 (s, 3H), 3.50-3.40 (m, 1H), 2.87 (d, J=13.5 Hz, 1H), 2.27-2.16 (m, 1H), 1.98-1.85 (m, 1H). ¹⁹F NMR (377 MHz, Chloroform-d) δ −79.96, −80.41, −80.96, −81.40.

### Example 11 and 12: (7R,14R)-1-(difluoromethoxy)-11-((S)-7-hydroxy-7-methyl-6,7-dihydro-5H-cyclopenta[b]pyridin-3-yl)-6-methyl-6,7-dihydro-7,14-methanobenzo[f]benzo[4,5]imidazo[1,2-a][1,4]diazocin-5(14H)-one and (7R,14R)-1-(difluoromethoxy)-11-((S)-7-hydroxy-7-methyl-6,7-dihydro-5H-cyclopenta[b]pyridin-3-yl)-6-methyl-6,7-dihydro-7,14-methanobenzo[f]benzo[4,5]imidazo[1,2-a][1,4]diazocin-5(14H)-one

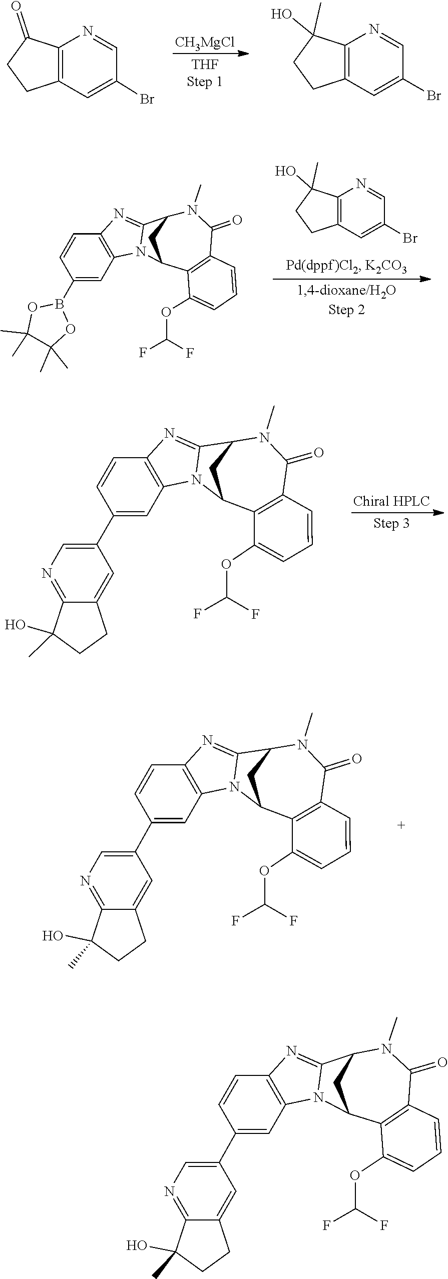

### Preparation 11A: 3-bromo-7-methyl-5H,6H-cyclopenta[b]pyridin-7-ol

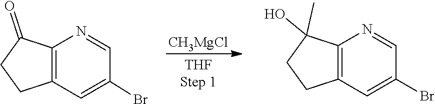

To a stirred solution of 3-bromo-5H,6H-cyclopenta[b]pyridin-7-one (300 mg, 1.415 mmol) in THF (3 mL) was added CH₃MgCl (3M in THF) (1.4 mL, 4.245 mmol) dropwise at 0° C. under nitrogen atmosphere. The mixture was stirred for 4 h at room temperature under nitrogen atmosphere. The reaction was quenched by the addition of sat. NH₄Cl (aq.) (10 mL) at 0° C. The resulting mixture was extracted with EtOAc (3×30 mL). The combined organic layers were washed with brine (3×25 mL), dried over anhydrous Na₂SO₄. After filtration, the filtrate was concentrated under reduced pressure. The residue was purified by reversed-phase flash chromatography with the following conditions: column, C18 silica gel; mobile phase, CH₃CN in water (10 mmol/L NH₄HCO₃), 20% to 50% gradient in 30 min; detector, 254 nm to afford 3-bromo-7-methyl-5H,6H-cyclopenta[b]pyridin-7-ol (148 mg, 46%) as a light-grey solid. MS ESI calculated for C₉H₁₀BrNO [M+H]⁺, 227.99 229.99, found 227.90 229.90. ¹H NMR (400 MHz, Chloroform-d) δ 8.51-8.46 (m, 1H), 7.73-7.67 (m, 1H), 3.06-2.93 (m, 1H), 2.88-2.75 (m, 1H), 2.37-2.19 (m, 2H), 1.58 (s, 3H).

### Preparation 11B: (1R,11R)-18-(difluoromethoxy)-5-{7-hydroxy-7-methyl-5H,6H-cyclopenta[b]pyridin-3-yl}-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

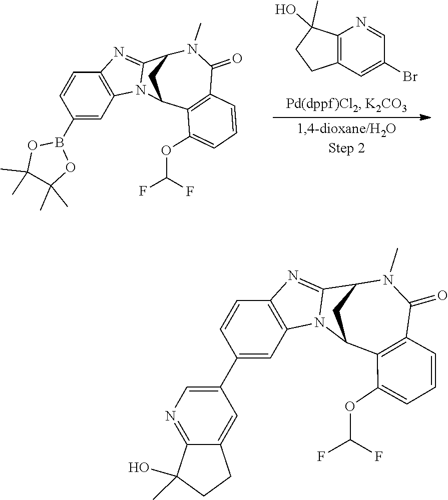 [SMILES: CN1C(=O)c2cccc(OC(F)F)c2[C@H]2C[C@@H]1c1nc3ccc(B4OC(C)(C)C(C)(C)O4)cc3n12 ; CC1(O)CCc2cc(Br)cnc21 ; CN1C(=O)c2cccc(OC(F)F)c2[C@H]2C[C@@H]1c1nc3ccc(-c4cnc5c(c4)CCC5(C)O)cc3n12]

Into a 8 mL vial were added (1R,11R)-18-(difluoromethoxy)-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (50 mg, 0.104 mmol), 3-bromo-7-methyl-5H,6H-cyclopenta[b]pyridin-7-ol (26 mg, 0.114 mmol), Pd(dppf)Cl₂·CH₂Cl₂ (8 mg, 0.010 mmol) and K₂CO₃ (36 mg, 0.260 mmol), 1,4-dioxane (0.5 mL) and H₂O (0.1 mL) at room temperature. The resulting mixture was stirred for 2 h at 80° C. under nitrogen atmosphere. The resulting mixture was concentrated under reduced pressure. The residue was purified by Prep-TLC (CH₂Cl₂/MeOH 10:1) followed by reversed-phase flash chromatography with the following conditions: column, C18 silica gel; mobile phase, CH₃CN in water (10 mmol/L NH₄HCO₃), 20% to 50% gradient in 30 min; detector, 254 nm to afford (1R,11R)-18-(difluoromethoxy)-5-{7-hydroxy-7-methyl-5H,6H-cyclopenta[b]pyridin-3-yl}-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (30 mg, 57%) as a white solid. MS ESI calculated for C₂₈H₂₄F₂N₄O₃ [M+H]⁺, 503.18, found 503.15. ¹H NMR (400 MHz, Chloroform-d) δ 8.67-8.62 (m, 1H), 8.49 (d, J=8.2 Hz, 1H), 7.83-7.73 (m, 2H), 7.66 (d, J=1.7 Hz, 1H), 7.46-7.38 (m, 2H), 7.31 (d, J=8.1 Hz, 1H), 6.84 (t, J=72.8 Hz, 1H), 6.28 (d, J=7.1 Hz, 1H), 4.98 (d, J=7.1 Hz, 1H), 3.53 (s, 3H), 3.52-3.43 (m, 1H), 3.13-3.02 (m, 1H), 2.96-2.84 (m, 2H), 2.43-2.26 (m, 2H), 1.68 (s, 3H). ¹⁹F NMR (377 MHz, Chloroform-d) δ −80.19, −80.21, −80.63, −80.65, −80.79, −81.23.

### Example 11 and 12: (7R,14R)-1-(difluoromethoxy)-11-((R)-7-hydroxy-7-methyl-6,7-dihydro-5H-cyclopenta[b]pyridin-3-yl)-6-methyl-6,7-dihydro-7,14-methanobenzo[f]benzo[4,5]imidazo[1,2-a][1,4]diazocin-5(14H)-one and (7R,14R)-1-(difluoromethoxy)-11-((S)-7-hydroxy-7-methyl-6,7-dihydro-5H-cyclopenta[b]pyridin-3-yl)-6-methyl-6,7-dihydro-7,14-methanobenzo[f]benzo[4,5]imidazo[1,2-a][1,4]diazocin-5(14H)-one

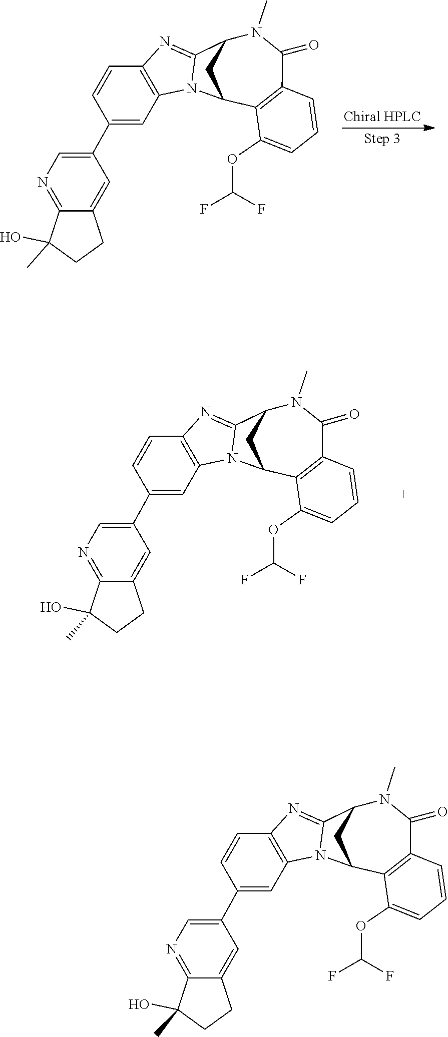 [SMILES: CN1C(=O)c2cccc(OC(F)F)c2[C@H]2C[C@@H]1c1nc3ccc(-c4cnc5c(c4)CCC5(C)O)cc3n12 ; CN1C(=O)c2cccc(OC(F)F)c2[C@H]2C[C@@H]1c1nc3ccc(-c4cnc5c(c4)CC[C@@]5(C)O)cc3n12 ; CN1C(=O)c2cccc(OC(F)F)c2[C@H]2C[C@@H]1c1nc3ccc(-c4cnc5c(c4)CC[C@]5(C)O)cc3n12]

The mixture (60 mg) was purified by Prep-Chiral HPLC with the following conditions (Column: CHIRAL ART Cellulose-SB, 2*25 cm, 5 μm; Mobile Phase A: Hex (10 mM NH₃-MeOH), Mobile Phase B: EtOH; Flow rate: 20 mL/min; Gradient: isocratic 50; Wave Length: 222/293 nm; RT1(min): 6.5; RT2(min): 9.5). The first peak afford 24 mg (41%) as a white solid. MS ESI calculated for C₂₈H₂₄F₂N₄O₃ [M+H]⁺, 503.18, found 503.05. ¹H NMR (400 MHz, Chloroform-d) δ 8.65 (d, J=2.0 Hz, 1H), 8.52-8.46 (m, 1H), 7.79 (d, J=8.5 Hz, 1H), 7.75 (d, J=2.0 Hz, 1H), 7.66 (d, J=1.8 Hz, 1H), 7.46-7.38 (m, 2H), 7.31 (d, J=8.2 Hz, 1H), 6.83 (t, J=72.9 Hz, 1H), 6.29 (d, J=7.2 Hz, 1H), 4.97 (d, J=7.1 Hz, 1H), 3.53 (s, 3H), 3.52-3.43 (m, 1H), 3.12-3.01 (m, 1H), 2.96-2.83 (m, 2H), 2.43-2.24 (m, 2H), 1.67 (s, 3H). ¹⁹F NMR (376 MHz, Chloroform-d) δ −80.21, −80.66, −80.81, −81.26.

The second peak afford 23 mg (39%) as a white solid. MS ESI calculated for C₂₈H₂₄F₂N₄O₃ [M+H]⁺, 503.18, found 503.15. ¹H NMR (400 MHz, Chloroform-d) δ 8.64 (d, J=2.1 Hz, 1H), 8.52-8.47 (m, 1H), 7.83-7.76 (m, 2H), 7.66 (d, J=1.7 Hz, 1H), 7.46-7.38 (m, 2H), 7.31 (d, J=8.1 Hz, 1H), 6.84 (t, J=72.9 Hz, 1H), 6.28 (d, J=7.2 Hz, 1H), 4.98 (d, J=7.1 Hz, 1H), 3.53 (s, 3H), 3.52-3.42 (m, 1H), 3.14-3.03 (m, 1H), 2.97-2.85 (m, 2H), 2.44-2.26 (m, 2H), 1.69 (s, 3H). ¹⁹F NMR (376 MHz, Chloroform-d) δ −80.22, −80.67, −80.79, −81.24.

### Example 13: (1R,11R)-5-[(1S)-1-amino-7-fluoro-2,3-dihydro-1H-inden-5-yl]-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

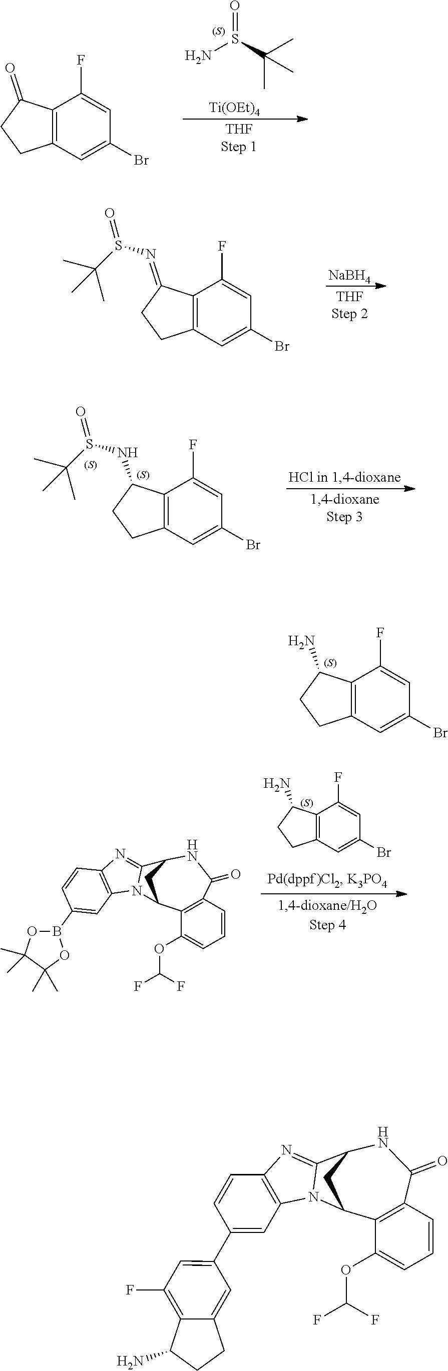

### Preparation 13A: (S)—N-[(1E)-5-bromo-7-fluoro-2,3-dihydroinden-1-ylidene]-2-methylpropane-2-sulfinamide

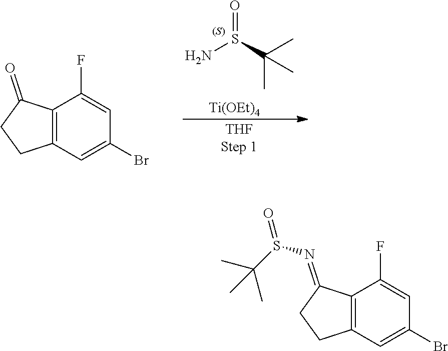

To a stirred solution of 5-bromo-7-fluoro-2,3-dihydroinden-1-one (4.50 g, 19.647 mmol) and Ti(OEₜ)₄ (8.96 g, 39.294 mmol) in THF (45 mL) was added (S)-2-methylpropane-2-sulfinamide (4.76 g, 39.294 mmol) at room temperature. The resulting mixture was stirred for 16 h at 60° C. The reaction was diluted with water. The aqueous layer was extracted with CH₂Cl₂ (3×100 mL). The resulting mixture was concentrated under vacuum. The residue was purified by silica gel column chromatography, eluted with PE/EA (5:1) to afford (S)—N-[(1E)-5-bromo-7-fluoro-2,3-dihydroinden-1-ylidene]-2-methylpropane-2-sulfinamide (2.00 g, 30%) as a green solid. MS ESI calculated for C₁₃H₁₅BrFNOS [M+H]⁺, 332.00 334.00, found 332.00 333.95. ¹H NMR (300 MHz, Chloroform-d) δ 7.35 (s, 1H), 7.16 (d, J=9.1 Hz, 1H), 3.56-3.46 (m, 1H), 3.19-3.11 (m, 2H), 3.15-3.03 (m, 1H), 1.32 (s, 9H).

### Preparation 13B: (S)—N-[(1S)-5-bromo-7-fluoro-2,3-dihydro-1H-inden-1-yl]-2-methylpropane-2-sulfinamide

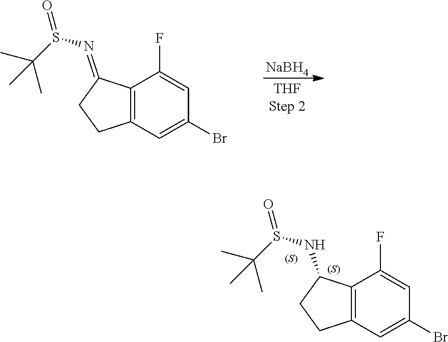

To a solution of (S)—N-[(1E)-5-bromo-7-fluoro-2,3-dihydroinden-1-ylidene]-2-methylpropane-2-sulfinamide (1.00 g, 3.010 mmol) in THF (10 mL) was added NaBH₄ (171 mg, 4.515 mmol) in ports at −50° C. The solution was stirred for 2 h at −50˜0° C. under nitrogen atmosphere. The resulting mixture was concentrated under reduced pressure. The residue was purified by silica gel column chromatography, eluted with CH₂Cl₂/MeOH (20:1) to afford (S)—N-[(1S)-5-bromo-7-fluoro-2,3-dihydro-1H-inden-1-yl]-2-methylpropane-2-sulfinamide (500 mg, 50%) as a green oil. MS ESI calculated for C₁₃H₁₇BrFNOS [M+H]⁺, 334.02 336.02, found 334.00 336.00. ¹H NMR (400 MHz, Chloroform-d) δ 7.22-7.19 (m, 1H), 7.11-7.04 (m, 1H), 5.10-5.05 (m, 1H), 3.71-3.65 (m, 1H), 3.18-3.07 (m, 1H), 2.94-2.84 (m, 1H), 2.52-2.41 (m, 1H), 2.30-2.18 (m, 1H), 1.19 (s, 9H).

### Preparation 13C: (1S)-5-bromo-7-fluoro-2,3-dihydro-1H-inden-1-amine

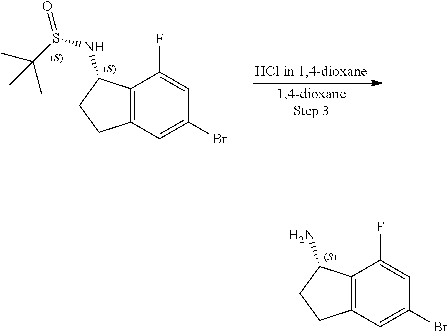

To a solution of (S)—N-(5-bromo-7-fluoro-2,3-dihydro-1H-inden-1-yl)-2-methylpropane-2-sulfinamide (500 mg, 1.496 mmol) in 1,4-dioxane (6 mL) was added 4M HCl in 1,4-dioxane (3 mL) at 0° C. The mixture was stirred for 2 h at 10° C. The resulting mixture was concentrated under vacuum. The mixture was basified to pH 8 with saturated NaHCO₃ (aq.). The aqueous layer was extracted with EtOAc (2×50 mL). The resulting mixture was concentrated under vacuum. The residue was purified by silica gel column chromatography, eluted with CH₂Cl₂/MeOH (20:1) followed by reversed-phase flash chromatography with the following conditions: column, C18 silica gel; mobile phase, CH₃CN in water (10 mmol/L NH₄HCO₃), 25% to 55% gradient in 25 min; detector, 254 nm. This resulted in 5-bromo-7-fluoro-2,3-dihydro-1H-inden-1-amine (250 mg) as a colorless oil. The mixture was purified by Chiral HPLC with the following conditions, Column: CHIRALPAK IG, 3*25 cm, 5 μm; Mobile Phase A: Hex (0.5% 2M NH₃-MeOH), Mobile Phase B: EtOH; Flow rate: 40 mL/min; Gradient: isocratic 10; Wave Length: 215/256 nm; RT1(min): 10.5; RT2(min): 13; Sample Solvent: DCM. The first peak was concentrated under vacuum. This resulted in (1S)-5-bromo-7-fluoro-2,3-dihydro-1H-inden-1-amine (170 mg, 49%) as a colorless oil. MS ESI calculated for C₉H₉BrFN [M+H]⁺, 229.99 231.99, found 230.05 232.05. ¹H NMR (400 MHz, Chloroform-d) δ 7.16 (s, 1H), 7.08-7.02 (m, 1H), 4.60 (t, J=7.5 Hz, 1H), 3.10-3.00 (m, 1H), 2.87-2.78 (m, 1H), 2.54-2.44 (m, 1H), 1.86-1.75 (m, 1H).

### Example 13: (1R,11R)-5-[(1S)-1-amino-7-fluoro-2,3-dihydro-1H-inden-5-yl]-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

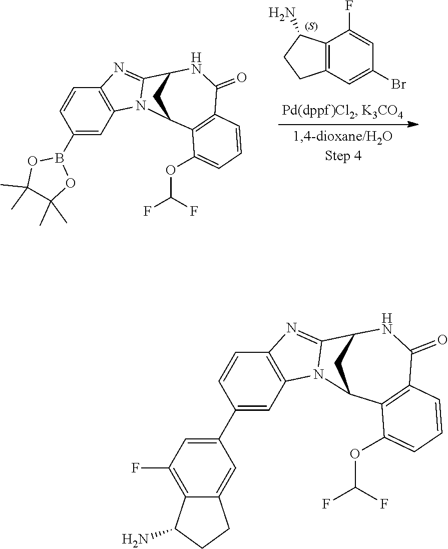

To a stirred solution of (1S)-5-bromo-7-fluoro-2,3-dihydro-1H-inden-1-amine (24 mg, 0.107 mmol) and (1R,11R)-18-(difluoromethoxy)-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (50 mg, 0.107 mmol) in 1,4-dioxane (2 mL) and H₂O (0.4 mL) were added K₃PO₄ (68 mg, 0.321 mmol) and Pd(dppf)Cl₂·CH₂Cl₂ (8 mg, 0.011 mmol) at room temperature. The resulting mixture was stirred for 16 h at 100° C. under nitrogen atmosphere. The resulting mixture was concentrated under vacuum. The residue was purified by silica gel column chromatography, eluted with CH₂Cl₂/MeOH (10:1) followed by reversed-phase flash chromatography with the following conditions: column, C18 silica gel; mobile phase, CH₃CN in water (10 mmol/L NH₄HCO₃), 30% to 60% gradient in 25 min; detector, 254 nm. This resulted in (1R,11R)-5-[(1S)-1-amino-7-fluoro-2,3-dihydro-1H-inden-5-yl]-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (20 mg, 37%) as a white solid. MS ESI calculated for C₂₇H₂₁F₃N₄O₂ [M+H]⁺, 491.16, found 491.15. ¹H NMR (400 MHz, DMSO-d₆) δ 9.14 (d, J=7.4 Hz, 1H), 8.24-8.21 (m, 1H), 7.90-7.45 (m, 6H), 7.29 (s, 1H), 7.18-7.15 (m, 1H), 6.34 (d, J=6.9 Hz, 1H), 4.88 (t, J=6.6 Hz, 1H), 4.52-4.50 (m, 1H), 3.51-3.45 (m, 1H), 3.12-3.04 (m, 1H), 2.84-2.72 (m, 2H), 2.42-2.28 (m, 1H), 2.15-1.70 (m, 3H). ¹⁹F NMR (377 MHz, DMSO-d₆) δ −82.33, −121.15.

### Example 14: (1R,11R)-5-[(3R)-3-amino-2,3-dihydro-1-benzofuran-6-yl]-12-cyclopropyl-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

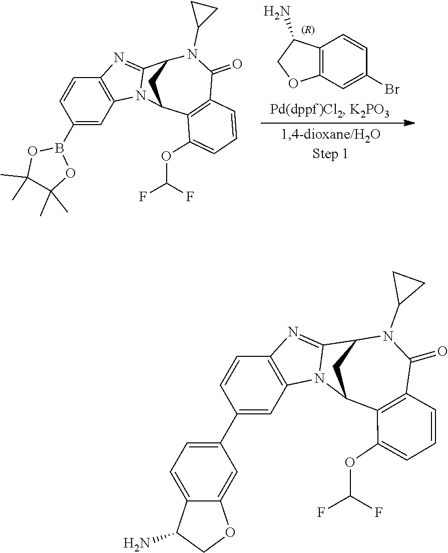

A mixture of (3R)-6-bromo-2,3-dihydro-1-benzofuran-3-amine (25 mg, 0.118 mmol), (1R,11R)-12-cyclopropyl-18-(difluoromethoxy)-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (40 mg, 0.079 mmol), K₃PO₄ (50 mg, 0.237 mmol) and Pd(dppf)Cl₂·CH₂Cl₂ (6 mg, 0.008 mmol) in 1,4-dioxane (1 mL) and H₂O (0.2 mL) was stirred for overnight at 80° C. under nitrogen atmosphere. The resulting mixture was concentrated under vacuum. The residue was purified by silica gel column chromatography, eluted with DCM/MeOH (10:1) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (1R,11R)-5-[(3R)-3-amino-2,3-dihydro-1-benzofuran-6-yl]-12-cyclopropyl-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (8 mg, 19%) as a white solid. MS ESI calculated for C₂₉H₂₄F₂N₄O₃ [M+H]⁺, 515.18, found 515.15. ¹H NMR (400 MHz, Chloroform-d) δ 8.36 (d, J=8.0 Hz, 1H), 7.74 (d, J=8.4 Hz, 1H), 7.67 (s, 1H), 7.49-7.42 (m, 1H), 7.41-7.37 (m, 2H), 7.29 (s, 1H), 7.15 (d, J=7.4 Hz, 1H), 7.04 (s, 1H), 6.81 (t, J=72.9 Hz, 1H), 6.24 (d, J=7.1 Hz, 1H), 5.22 (d, J=7.3 Hz, 1H), 4.77-4.65 (m, 2H), 4.27-4.24 (m, 1H), 3.49-3.41 (m, 1H), 3.29-3.20 (m, 1H), 2.84 (d, J=13.5 Hz, 1H), 1.58-1.48 (m, 1H), 1.13-1.05 (m, 2H), 0.70-0.66 (m, 1H); ¹⁹F NMR (377 MHz, Chloroform-d) δ −80.20, −80.65, −80.83, −81.28.

### Example 15: (1R,11R)-18-(difluoromethoxy)-5-[(1S)-1-hydroxy-2,3-dihydro-1H-inden-5-yl]-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

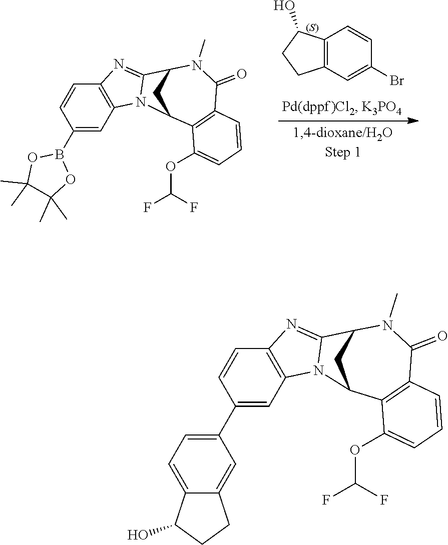

A mixture of (1R,11R)-18-(difluoromethoxy)-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (50 mg, 0.104 mmol), (1S)-5-bromo-2,3-dihydro-1H-inden-1-ol (29 mg, 0.135 mmol), K₃PO₄ (66 mg, 0.312 mmol) and Pd(dppf)Cl₂·CH₂Cl₂ (9 mg, 0.010 mmol) in 1,4-dioxane (1 mL) and H₂O (0.2 mL) was stirred for 16 h at 80° C. under nitrogen atmosphere. The resulting mixture was concentrated under reduced pressure. The residue was purified by silica gel column chromatography, eluted with CH₂Cl₂/MeOH (5/1) followed by reversed-phase flash chromatography with the following conditions: column, C18 silica gel; mobile phase, CH₃CN in water (10 mmol/L NH₄HCO₃), 20% to 50% gradient in 30 min; detector, 254 nm. This resulted in (1R,11R)-18-(difluoromethoxy)-5-[(1S)-1-hydroxy-2,3-dihydro-1H-inden-5-yl]-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (15 mg, 29%) as a white solid. MS ESI calculated for C₂₈H₂₃F₂N₃O₃ [M+H]⁺, 488.17, found 488.15. ¹H NMR (400 MHz, Chloroform-d) δ 8.52-8.46 (m, 1H), 7.78-7.72 (m, 1H), 7.69 (d, J=1.7 Hz, 1H), 7.53-7.43 (m, 4H), 7.41 (t, J=8.2 Hz, 1H), 7.33-7.26 (m, 1H), 6.82 (t, J=72.9 Hz, 1H), 6.28 (d, J=7.2 Hz, 1H), 5.34-5.27 (m, 1H), 4.98 (d, J=7.0 Hz, 1H), 3.53 (s, 3H), 3.51-3.41 (m, 1H), 3.19-3.07 (m, 1H), 2.95-2.83 (m, 2H), 2.61-2.50 (m, 1H), 2.07-1.96 (m, 1H). ¹⁹F NMR (377 MHz, Chloroform-d) δ −80.12, −80.57, −80.83, −81.28.

### Example 16 and 17: (7R,14R)-11-((S)-7-amino-6,7-dihydro-5H-cyclopenta[b]pyridin-3-yl)-1-(difluoromethoxy)-6-methyl-6,7-dihydro-7,14-methanobenzo[f]benzo[4,5]imidazo[1,2-a][1,4]diazocin-5(14H)-one and (7R,14R)-11-((R)-7-amino-6,7-dihydro-5H-cyclopenta[b]pyridin-3-yl)-1-(difluoromethoxy)-6-methyl-6,7-dihydro-7,14-methanobenzo[f]benzo[4,5]imidazo[1,2-a][1,4]diazocin-5(14H)-one

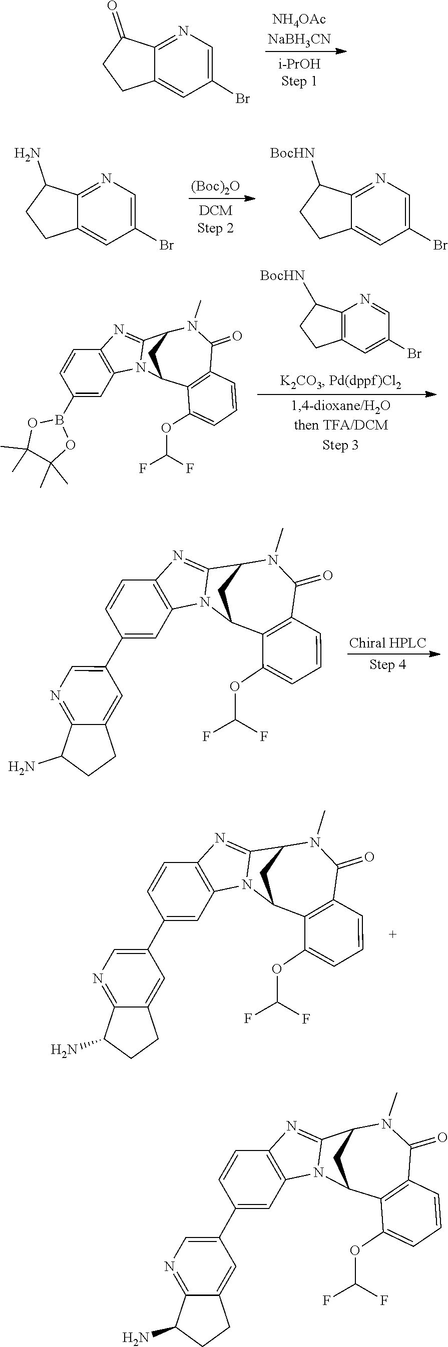

### Preparation 16A: 3-bromo-5H,6H,7H-cyclopenta[b]pyridin-7-amine

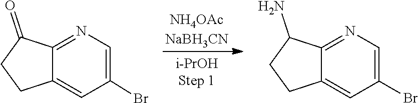 [SMILES: O=C1CCc2cc(Br)cnc21 ; "NH4OAcNaBH3CN" ; NC1CCc2cc(Br)cnc21]

A solution of 3-bromo-5H,6H-cyclopenta[b]pyridin-7-one (150 mg, 0.707 mmol), NH₄OAc (1.31 g, 16.968 mmol) and NaBH₃CN (444 mg, 7.070 mmol) in 2-Propanol (6 mL) was stirred for 16 h at 60° C. The resulting mixture was diluted with water (10 mL). The resulting mixture was extracted with CH₂Cl₂ (3×30 mL). The combined organic layers were washed with brine (3×20 mL), dried over anhydrous Na₂SO₄. After filtration, the filtrate was concentrated under reduced pressure to afford 3-bromo-5H,6H,7H-cyclopenta[b]pyridin-7-amine (190 mg) as a green solid. The crude product was used in the next step directly without further purification. MS ESI calculated for C₈H₉BrN₂ [M+H]⁺, 212.99, 214.99, found 212.85, 214.85.

### Preparation 16B: tert-butyl N-{3-bromo-5H,6H,7H-cyclopenta[b]pyridin-7-yl}carbamate

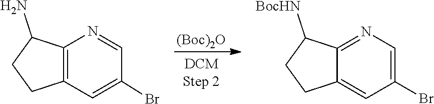 [SMILES: "(Boc)2O" ; NC1CCc2cc(Br)cnc21 ; CC(C)(C)OC(=O)NC1CCc2cc(Br)cnc21]

To a stirred solution of 3-bromo-5H,6H,7H-cyclopenta[b]pyridin-7-amine (180 mg, 0.845 mmol) in DCM (4 mL) was added Boc₂O (202 mg, 0.929 mmol) at room temperature. The resulting mixture was stirred for 30 min at room temperature. The resulting mixture was concentrated under reduced pressure. The residue was purified by silica gel column chromatography, eluted with PE/EA (5/1) to afford tert-butyl N-{3-bromo-5H,6H,7H-cyclopenta[b]pyridin-7-yl}carbamate (110 mg, 41%) as a white solid. MS ESI calculated for C₁₃H₁₇BrN₂O₂ [M+H]⁺, 313.05, 315.05, found 313.10, 315.10. ¹H NMR (400 MHz, Chloroform-d) δ 8.48 (s, 1H), 7.71 (s, 1H), 5.20 (s, 1H), 4.96 (s, 1H), 3.02-2.73 (m, 3H), 2.00-1.90 (m, 1H), 1.46 (s, 9H).

### Preparation 16C: (1R,11R)-5-{7-amino-5H,6H,7H-cyclopenta[b]pyridin-3-yl}-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

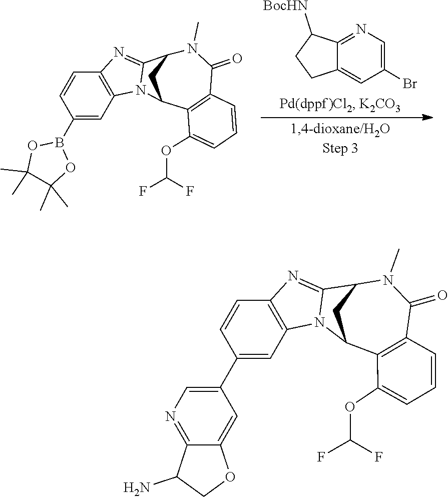 [SMILES: CN1C(=O)c2cccc(OC(F)F)c2[C@H]2C[C@@H]1c1nc3ccc(B4OC(C)(C)C(C)(C)O4)cc3n12 ; CC(C)(C)OC(=O)NC1CCc2cc(Br)cnc21 ; CN1C(=O)c2cccc(OC(F)F)c2[C@H]2C[C@@H]1c1nc3ccc(-c4cnc5c(c4)OCC5N)cc3n12]

A solution of tert-butyl N-{3-bromo-5H,6H,7H-cyclopenta[b]pyridin-7-yl}carbamate (78 mg, 0.250 mmol), (1R,11R)-18-(difluoromethoxy)-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (100 mg, 0.208 mmol), K₂CO₃ (71 mg, 0.520 mmol) and Pd(dppf)Cl₂·CH₂Cl₂ (16 mg, 0.021 mmol) in 1,4-dioxane (1 mL) and H₂O (0.2 mL) was stirred for 16 h at 80° C. under nitrogen atmosphere. The mixture was allowed to cool down to room temperature. The resulting mixture was concentrated under reduced pressure. The residue was purified by silica gel column chromatography, eluted with CH₂Cl₂/MeOH (12/1) to afford a yellow solid. The yellow solid was dissolved in DCM (2 mL). To the above solution was added TFA (1 mL) dropwise at room temperature. The resulting mixture was stirred for 2 h at room temperature. The resulting mixture was concentrated under vacuum. The residue was purified by reversed-phase flash chromatography with the following conditions: column, C18 silica gel; mobile phase, CH₃CN in water (10 mmol/L NH₄HCO₃), 20% to 50% gradient in 30 min; detector, 254 nm to afford (1R,11R)-5-{7-amino-5H,6H,7H-cyclopenta[b]pyridin-3-yl}-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (44 mg, 43%) as a light yellow solid. MS ESI calculated for C₂₇H₂₃F₂N₅O₂ [M+H]⁺, 488.18, found 488.20.

### Example 16 and 17: (7R,14R)-11-((S)-7-amino-6,7-dihydro-5H-cyclopenta[b]pyridin-3-yl)-1-(difluoromethoxy)-6-methyl-6,7-dihydro-7,14-methanobenzo[f]benzo[4,5]imidazo[1,2-a][1,4]diazocin-5(14H)-one and (7R,14R)-11-((R)-7-amino-6,7-dihydro-5H-cyclopenta[b]pyridin-3-yl)-1-(difluoromethoxy)-6-methyl-6,7-dihydro-7,14-methanobenzo[f]benzo[4,5]imidazo[1,2-a][1,4]diazocin-5(14H)-one

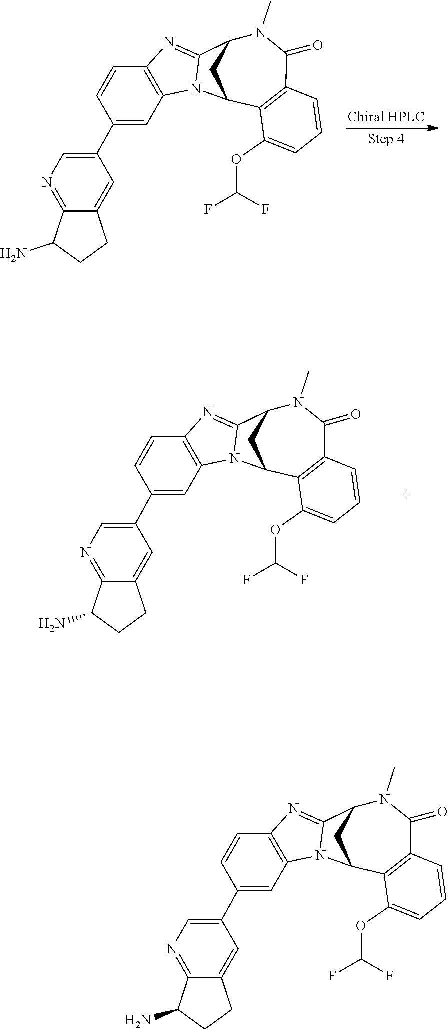 [SMILES: CN1C(=O)c2cccc(OC(F)F)c2[C@H]2C[C@@H]1c1nc3ccc(-c4cnc5c(c4)CCC5N)cc3n12 ; CN1C(=O)c2cccc(OC(F)F)c2[C@H]2C[C@@H]1c1nc3ccc(-c4cnc5c(c4)CC[C@@H]5N)cc3n12 ; CN1C(=O)c2cccc(OC(F)F)c2[C@H]2C[C@@H]1c1nc3ccc(-c4cnc5c(c4)CC[C@H]5N)cc3n12]

The mixture (44 mg) was purified by Chiral-HPLC with the following conditions (Column: CHIRAL ART Cellulose-SB, 2*25 cm, 5 μm; Mobile Phase A: Hex (10 mM NH₃-MeOH), Mobile Phase B: EtOH; Flow rate: 20 mL/min; Gradient: isocratic 50; Wave Length: 215/294 nm; RT1(min): 9.3; RT2(min): 13.5). The first peak afforded 12 mg (28%) as a white solid. MS ESI calculated for C₂₇H₂₃F₂N₅O₂ [M+H]⁺, 488.18, found 488.15. ¹H NMR (400 MHz, Chloroform-d) δ 8.63 (s, 1H), 8.49 (d, J=8.1 Hz, 1H), 7.79 (d, J=8.5 Hz, 1H), 7.71 (s, 1H), 7.67 (s, 1H), 7.46-7.39 (m, 2H), 7.31 (d, J=8.1 Hz, 1H), 6.83 (t, J=72.8 Hz, 1H), 6.29 (d, J=7.2 Hz, 1H), 4.98 (d, J=7.1 Hz, 1H), 4.38 (t, J=7.9 Hz, 1H), 3.53 (s, 3H), 3.51-3.42 (m, 1H), 3.06-2.98 (m, 1H), 2.94-2.84 (m, 2H), 2.69-2.58 (m, 1H), 1.90-1.77 (m, 1H); ¹⁹F NMR (377 MHz, Chloroform-d) δ −80.22, −80.66, −80.82, −81.24.

The second peak afforded 9 mg (19%) as a white solid. MS ESI calculated for C₂₇H₂₃F₂N₅O₂ [M+H]⁺, 488.18, found 488.15. ¹H NMR (400 MHz, Chloroform-d) δ 8.63 (s, 1H), 8.49 (d, J=8.1 Hz, 1H), 7.78 (d, J=8.5 Hz, 1H), 7.71 (s, 1H), 7.66 (s, 1H), 7.46-7.38 (m, 2H), 7.31 (d, J=8.1 Hz, 1H), 6.83 (t, J=72.8 Hz, 1H), 6.29 (d, J=7.2 Hz, 1H), 4.98 (d, J=7.1 Hz, 1H), 4.38 (t, J=7.9 Hz, 1H), 3.53 (s, 3H), 3.51-3.43 (m, 1H), 3.06-2.98 (m, 1H), 2.94-2.84 (m, 2H), 2.69-2.58 (m, 1H), 1.90-1.77 (m, 1H); ¹⁹F NMR (377 MHz, Chloroform-d) δ −80.21, −80.66, −80.83, −81.28.

### Example 18: (1R,11R)-18-(difluoromethoxy)-12-methyl-5-{7-oxo-5H,6H-pyrrolo[3,4-b]pyridin-3-yl}-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

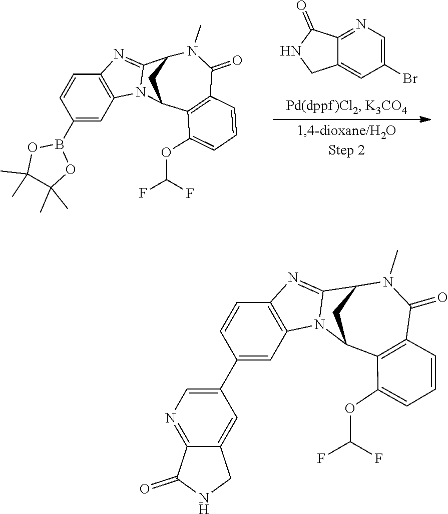 [SMILES: CN1C(=O)c2cccc(OC(F)F)c2[C@H]2C[C@@H]1c1nc3ccc(B4OC(C)(C)C(C)(C)O4)cc3n12 ; O=C1NCc2cc(Br)cnc21 ; CN1C(=O)c2cccc(OC(F)F)c2[C@H]2C[C@@H]1c1nc3ccc(-c4cnc5c(c4)CNC5=O)cc3n12]

To a solution of (1R,11R)-18-(difluoromethoxy)-12-methyl-5-(3,3,4,4-tetramethylborolan-1-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (50 mg, 0.105 mmol) and 3-bromo-5H,6H-pyrrolo[3,4-b]pyridin-7-one (24 mg, 0.114 mmol) in 1,4-dioxane (0.5 mL) and H₂O (0.1 mL) were added K₃PO₄ (66 mg, 0.312 mmol) and Pd(dppf)Cl₂·CH₂Cl₂ (8 mg, 0.010 mmol). After stirring for 2 h at 80° C. under a nitrogen atmosphere, the resulting mixture was concentrated under reduced pressure. The resulting mixture was filtered, and the filter cake was washed with MeOH (3×5 mL). The filtrate was concentrated under reduced pressure. The residue was purified by reversed-phase flash chromatography with the following conditions: column, C18 silica gel; mobile phase, CH₃CN in water (10 mmol/L NH₄HCO₃), 20% to 50% gradient in 30 min; detector, 254 nm. This resulted in (1R,11R)-18-(difluoromethoxy)-12-methyl-5-{7-oxo-5H,6H-pyrrolo[3,4-b]pyridin-3-yl}-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (9 mg, 18%) as a white solid. MS ESI calculated for C₂₆H₁₉F₂N₅O₃ [M+H]⁺, 488.15, found 488.00. ¹H NMR (400 MHz, DMSO-d₆) δ 9.03-8.93 (m, 2H), 8.32-8.21 (m, 2H), 7.89-7.45 (m, 6H), 6.32 (d, J=7.1 Hz, 1H), 5.26 (d, J=7.1 Hz, 1H), 4.46 (s, 2H), 3.58-3.50 (m, 1H), 3.33 (s, 3H), 2.85 (d, J=13.7 Hz, 1H). ¹⁹F NMR (377 MHz, DMSO-d₆) δ −81.55, −82.00, −82.35, −82.80.

### Example 19: (1R,11R)-5-[(4S)-4-amino-2H,3H,4H-pyrano[3,2-b]pyridin-7-yl]-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

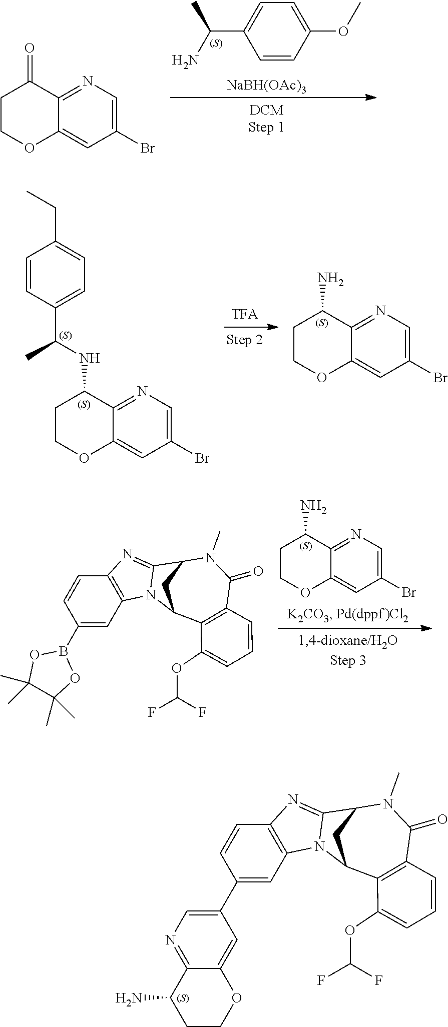

### Preparation 19A: (4S)-7-bromo-N-[(1S)-1-(4-methoxyphenyl)ethyl]-2H,3H,4H-pyrano[3,2-b]pyridin-4-amine

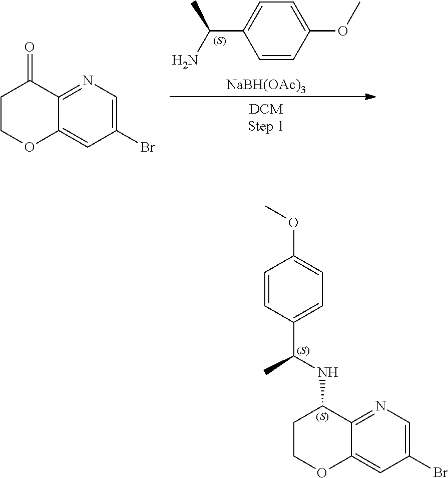

To a stirred mixture of 7-bromo-2H,3H-pyrano[3,2-b]pyridin-4-one (300 mg, 1.316 mmol) and (1S)-1-(4-methoxyphenyl)ethanamine (191 mg, 1.263 mmol) in DCM (4 mL) was added NaBH(OAc)₃ (502 mg, 2.369 mmol) in portions at room temperature under air atmosphere. The resulting mixture was stirred for 24 h at room temperature. The mixture was basified to pH 11 with NaOH (1 M). The resulting mixture was extracted with CH₂Cl₂ (3×100 mL). The combined organic layers were dried over anhydrous Na₂SO₄. After filtration, the filtrate was concentrated under reduced pressure. The residue was purified by silica gel column chromatography, eluted with PE/EA (3/1) to afford (4S)-7-bromo-N-[(1S)-1-(4-methoxyphenyl)ethyl]-2H,3H,4H-pyrano[3,2-b]pyridin-4-amine (288 mg, 60%) as a yellow oil. MS ESI calculated for C₁₇H₁₉BrN₂O₂ [M+H]⁺, 363.06 365.06, found 363.00 365.00. ¹H NMR (400 MHz, Chloroform-d) δ 8.20 (d, J=2.0 Hz, 1H), 7.35 (d, J=8.2 Hz, 2H), 7.26 (d, J=1.9 Hz, 1H), 6.90-6.82 (m, 2H), 4.26-4.16 (m, 1H), 4.16-3.98 (m, 2H), 3.85 (t, J=5.6 Hz, 1H), 3.80 (s, 3H), 1.81-1.61 (m, 2H), 1.44 (d, J=6.6 Hz, 3H).

### Preparation 19B: (4S)-7-bromo-2H,3H,4H-pyrano[3,2-b]pyridin-4-amine

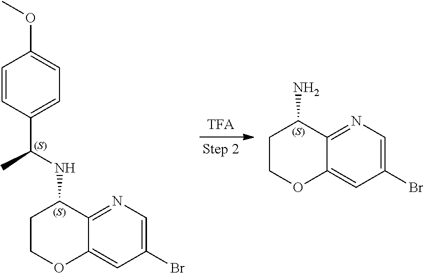

A solution of (4S)-7-bromo-N-[(1S)-1-(4-methoxyphenyl)ethyl]-2H,3H,4H-pyrano[3,2-b]pyridin-4-amine (280 mg, 0.771 mmol) in trifluoroacetaldehyde (3 mL) was stirred for 24 h at room temperature. The resulting mixture concentrated under reduced pressure. The residue was purified by reversed-phase flash chromatography with the following conditions: column, C18 silica gel; mobile phase, CH₃CN in water (10 mmol/L NH₄HCO₃), 10% to 50% gradient in 30 min; detector, 254 nm. This resulted in (4S)-7-bromo-2H,3H,4H-pyrano[3,2-b]pyridin-4-amine (150 mg, 84%) as a yellow solid. MS ESI calculated for C₈H₉BrN₂O [M+H]⁺, 228.99 230.99, found 228.90 230.90. ¹H NMR (400 MHz, Chloroform-d) δ 8.21 (d, J=2.0 Hz, 1H), 7.29 (d, J=2.0 Hz, 1H), 4.38-4.27 (m, 1H), 4.27-4.17 (m, 1H), 4.12-4.02 (m, 1H), 2.34-2.21 (m, 1H), 2.03-1.88 (m, 1H).

### Example 19: (1R,11R)-5-[(4S)-4-amino-2H,3H,4H-pyrano[3,2-b]pyridin-7-yl]-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

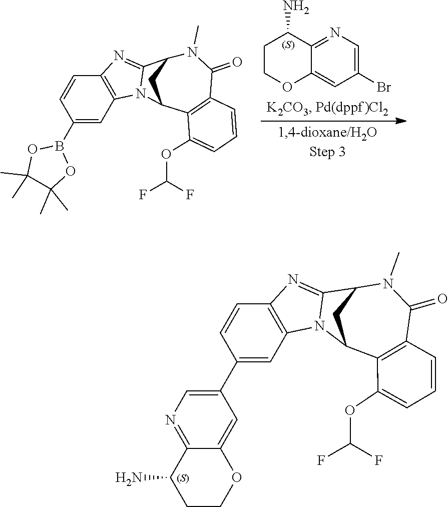

A mixture of (4S)-7-bromo-2H,3H,4H-pyrano[3,2-b]pyridin-4-amine (31 mg, 0.135 mmol), (1R,11R)-18-(difluoromethoxy)-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (50 mg, 0.104 mmol), K₂CO₃ (36 mg, 0.260 mmol) and Pd(dppf)Cl₂·CH₂Cl₂ (8 mg, 0.010 mmol) in 1,4-dioxane (1 mL) and H₂O (0.2 mL) was stirred for 2 h at 80° C. under nitrogen atmosphere. The resulting mixture was concentrated under reduced pressure. The residue was purified by silica gel column chromatography, eluted with CH₂Cl₂/MeOH (5/1) followed by reversed-phase flash chromatography with the following conditions: column, C18 silica gel; mobile phase, CH₃CN in water (10 mmol/L NH₄HCO₃), 25% to 50% gradient in 30 min; detector, 254 nm. This resulted in (1R,11R)-5-[(4S)-4-amino-2H,3H,4H-pyrano[3,2-b]pyridin-7-yl]-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (13 mg, 24%) as a white solid. MS ESI calculated for C₂₇H₂₃F₂N₅O₃ [M+H]⁺, 504.18, found 504.20. ¹H NMR (400 MHz, Chloroform-d) δ 8.51-8.46 (m, 1H), 8.42-8.37 (m, 1H), 7.81-7.75 (m, 1H), 7.65 (d, J=1.8 Hz, 1H), 7.46-7.37 (m, 2H), 7.34-7.29 (m, 2H), 6.84 (t, J=73.2 Hz, 1H), 6.28 (d, J=7.2 Hz, 1H), 4.97 (d, J=7.2 Hz, 1H), 4.43-4.34 (m, 1H), 4.32-4.17 (m, 2H), 3.52 (s, 3H), 3.51-3.42 (m, 1H), 2.88 (d, J=13.6 Hz, 1H), 2.41-2.32 (m, 1H), 2.09-1.99 (m, 1H). ¹⁹F NMR (377 MHz, Chloroform-d) δ −80.14, −80.58, −80.89, −81.34.

### Example 20: (1R,11R)-18-(difluoromethoxy)-12-methyl-5-{6-methyl-7-oxo-5H-pyrrolo[3,4-b]pyridin-3-yl}-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

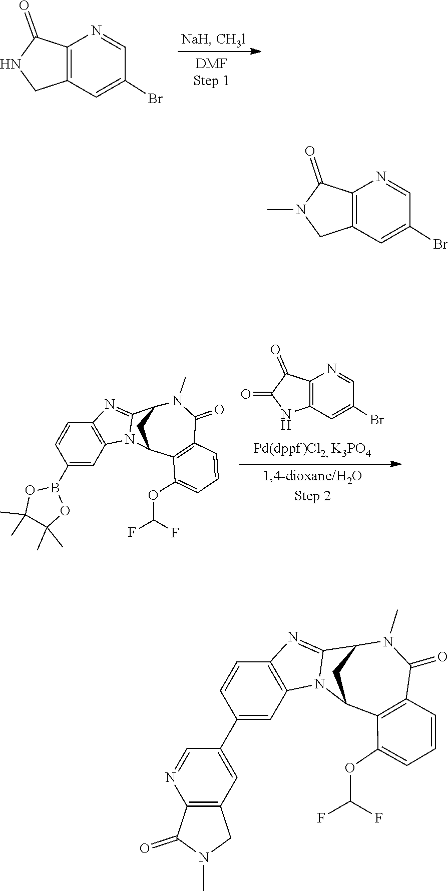

### Preparation 20A: 3-bromo-6-methyl-5H-pyrrolo[3,4-b]pyridin-7-one

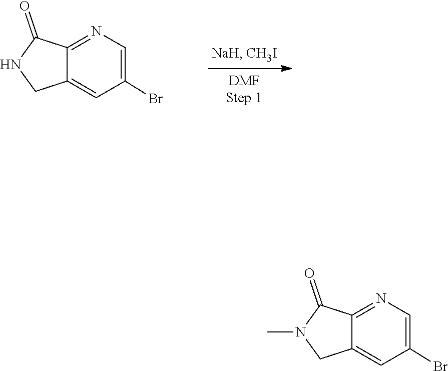

A mixture of 3-bromo-5H,6H-pyrrolo[3,4-b]pyridin-7-one (100 mg, 0.469 mmol) and NaH (23 mg, 0.563 mmol, 60%) in DMF (2 mL) was stirred for 10 min at 20° C. under nitrogen atmosphere. To the above mixture was added CH₃I (80 mg, 0.563 mmol) dropwise at 20° C. The resulting mixture was stirred for additional 16 h at 20° C. The reaction was quenched with water (1 mL) at 0° C. The resulting solution was purified by reversed-phase flash chromatography with the following conditions: column, C18 silica gel; mobile phase, CH₃CN in water (10 mmol/L NH₄HCO₃), 30% to 70% gradient in 30 min; detector, 254 nm. This resulted in 3-bromo-6-methyl-5H-pyrrolo[3,4-b]pyridin-7-one (51 mg, 48%) as a yellow solid. MS ESI calculated for C₈H₇BrN₂O [M+H]⁺, 226.97 228.97, found 226.90 228.90. ¹H NMR (400 MHz, Chloroform-d) δ 8.84 (d, J=2.0 Hz, 1H), 7.98 (d, J=2.0 Hz, 1H), 4.40 (s, 2H), 3.26 (s, 3H).

### Example 20: (1R,11R)-18-(difluoromethoxy)-12-methyl-5-{6-methyl-7-oxo-5H-pyrrolo[3,4-b]pyridin-3-yl}-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

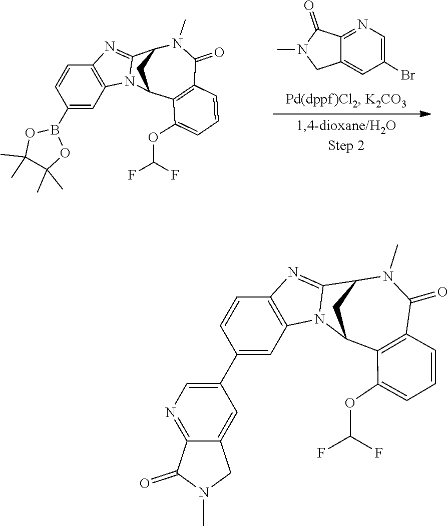 [SMILES: CN1C(=O)c2cccc(OC(F)F)c2[C@H]2C[C@@H]1c1nc3ccc(B4OC(C)(C)C(C)(C)O4)cc3n12 ; CN1Cc2cc(Br)cnc2C1=O ; CN1Cc2cc(-c3ccc4nc5n(c4c3)[C@@H]3C[C@H]5N(C)C(=O)c4cccc(OC(F)F)c43)cnc2C1=O]

To a solution of (1R,11R)-18-(difluoromethoxy)-12-methyl-5-(3,3,4,4-tetramethylborolan-1-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (50 mg, 0.105 mmol) and 3-bromo-6-methyl-5H-pyrrolo[3,4-b]pyridin-7-one (26 mg, 0.116 mmol) in 1,4-dioxane (0.5 mL) and H₂O (0.1 mL) were added K₃PO₄ (67 mg, 0.315 mmol) and Pd(dppf)Cl₂·CH₂Cl₂ (9 mg, 0.011 mmol). After stirring for 2 h at 80° C. under a nitrogen atmosphere, the resulting mixture was concentrated under reduced pressure. The resulting mixture was filtered, and the filter cake was washed with MeOH (3×5 mL). The filtrate was concentrated under reduced pressure. The residue was purified by reversed-phase flash chromatography with the following conditions: column, C18 silica gel; mobile phase, CH₃CN in water (10 mmol/L NH₄HCO₃), 30% to 60% gradient in 30 min; detector, 254 nm. This resulted in (1R,11R)-18-(difluoromethoxy)-12-methyl-5-{6-methyl-7-oxo-5H-pyrrolo[3,4-b]pyridin-3-yl}-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (32 mg, 59%) as a white solid. MS ESI calculated for C₂₇H₂₁F₂N₅O₃ [M+H]⁺, 502.16, found 502.10. ¹H NMR (400 MHz, DMSO-d₆) δ 8.96-8.91 (m, 1H), 8.32-8.21 (m, 2H), 7.89-7.66 (m, 3H), 7.64-7.57 (m, 1H), 7.55-7.46 (m, 2H), 6.30 (d, J=7.0 Hz, 1H), 5.26 (d, J=7.1 Hz, 1H), 4.53 (s, 2H), 3.59-3.48 (m, 1H), 3.37 (s, 3H), 3.14 (s, 3H), 2.84 (d, J=13.8 Hz, 1H). ¹⁹F NMR (377 MHz, DMSO-d₆) δ −81.54, −81.99, −82.35, −82.80.

### Example 21: (1R,11R)-18-(difluoromethoxy)-5-(3-hydroxy-3-methyl-2-oxo-1H-indol-6-yl)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

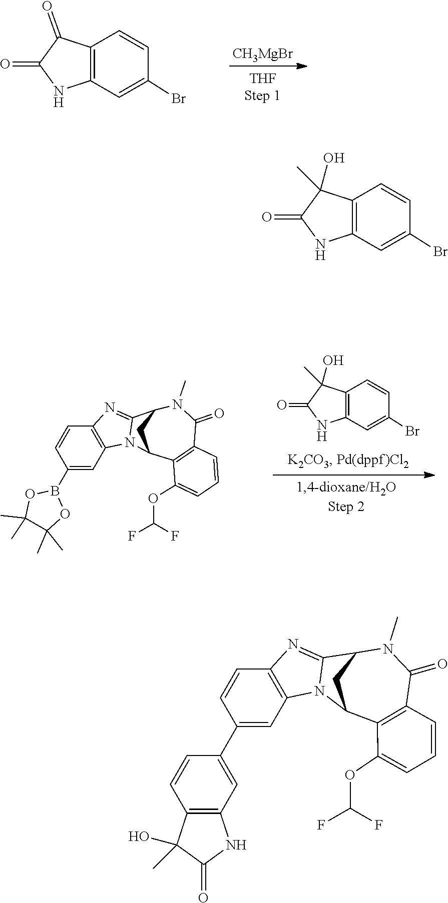 [SMILES: O=C1Nc2cc(Br)ccc2C1=O ; "CH3MgBr" ; CC1(O)C(=O)Nc2cc(Br)ccc21 ; CN1C(=O)c2cccc(OC(F)F)c2[C@H]2C[C@@H]1c1nc3ccc(B4OC(C)(C)C(C)(C)O4)cc3n12 ; CC1(O)C(=O)Nc2cc(Br)ccc21 ; CN1C(=O)c2cccc(OC(F)F)c2[C@H]2C[C@@H]1c1nc3ccc(-c4ccc5c(c4)NC(=O)C5(C)O)cc3n12]

### Preparation 21A: 6-bromo-3-hydroxy-3-methyl-1H-indol-2-one

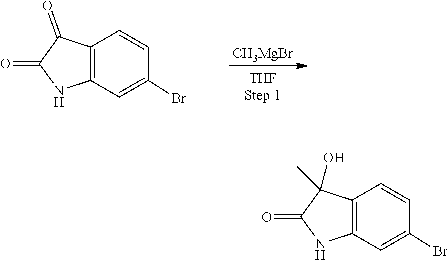

To a stirred mixture of 6-bromo-1H-indole-2,3-dione (10.00 g, 44.242 mmol) in THF (150 mL) was added CH₃MgBr (15 mL, 44.242 mmol, 3M in THF) in portions over 30 min at −78° C. under nitrogen atmosphere. The resulting mixture was stirred for 16 h at −78° C. under nitrogen atmosphere. The reaction was quenched by the addition of sat. NH₄Cl (aq.) (50 mL) at −78° C. The aqueous layer was extracted with EtOAc (2×200 mL). The resulting mixture was concentrated under reduced pressure. This resulted in 6-bromo-3-hydroxy-3-methyl-1H-indol-2-one (700 mg, 6%) as a white oil. MS ESI calculated for C₉H₈BrNO₂ [M−H]⁻, 239.97 241.97, found 239.95 241.85. ¹H NMR (400 MHz, DMSO-d₆) δ 10.37 (s, 1H), 7.24 (d, J=7.9 Hz, 1H), 7.16-7.14 (m, 1H), 6.95 (d, J=1.7 Hz, 1H), 5.95 (s, 1H), 1.35 (s, 3H).

### Example 21: (1R,11R)-18-(difluoromethoxy)-5-(3-hydroxy-3-methyl-2-oxo-1H-indol-6-yl)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

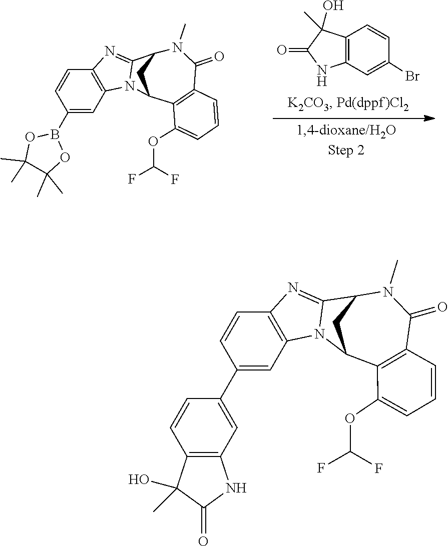 [SMILES: CN1C(=O)c2cccc(OC(F)F)c2[C@H]2C[C@@H]1c1nc3ccc(B4OC(C)(C)C(C)(C)O4)cc3n12 ; CC1(O)C(=O)Nc2cc(Br)ccc21 ; CN1C(=O)c2cccc(OC(F)F)c2[C@H]2C[C@@H]1c1nc3ccc(-c4ccc5c(c4)NC(=O)C5(C)O)cc3n12]

To a stirred mixture of (1R,11R)-18-(difluoromethoxy)-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (50 mg, 0.104 mmol) and 6-bromo-3-hydroxy-3-methyl-1H-indol-2-one (30 mg, 0.125 mmol) in 1,4-dioxane (1 mL) and H₂O (0.2 mL) were added K₂CO₃ (43 mg, 0.312 mmol) and Pd(dppf)Cl₂·CH₂Cl₂ (8 mg, 0.010 mmol) in portions at room temperature under nitrogen atmosphere. The resulting mixture was stirred for 2 h at 80° C. under nitrogen atmosphere. The resulting mixture was concentrated under reduced pressure. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (1R,11R)-18-(difluoromethoxy)-5-(3-hydroxy-3-methyl-2-oxo-1H-indol-6-yl)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (13 mg, 24%) as a white solid. MS ESI calculated for C₂₈H₂₂F₂N₄O₄ [M+H]⁺, 517.16, found 517.25. ¹H NMR (300 MHz, DMSO-d₆) δ 10.36 (s, 1H), 8.32-8.25 (m, 1H), 7.88-7.60 (m, 3H), 7.49 (q, J=4.7, 3.6 Hz, 2H), 7.44-7.41 (m, 1H), 7.37 (d, J=7.7 Hz, 1H), 7.19 (d, J=7.7 Hz, 1H), 6.99 (s, 1H), 6.29 (d, J=7.1 Hz, 1H), 5.90 (s, 1H), 5.24 (d, J=7.1 Hz, 1H), 3.59-3.47 (m, 1H), 3.36 (s, 3H), 2.83 (d, J=13.8 Hz, 1H), 1.41 (s, 3H); ¹⁹F NMR (282 MHz, DMSO-d₆) δ −81.26, −81.86, −81.89, −81.91, −82.51.

### Example 22 and 23: (7R,14R)-11-((R)-7-amino-7-methyl-6,7-dihydro-5H-cyclopenta[b]pyridin-3-yl)-1-(difluoromethoxy)-6-methyl-6,7-dihydro-7,14-methanobenzo[f]benzo[4,5]imidazo[1,2-a][1,4]diazocin-5(14H)-one and (7R,14R)-11-((S)-7-amino-7-methyl-6,7-dihydro-5H-cyclopenta[b]pyridin-3-yl)-1-(difluoromethoxy)-6-methyl-6,7-dihydro-7,14-methanobenzo[f]benzo[4,5]imidazo[1,2-a][1,4]diazocin-5(14H)-one

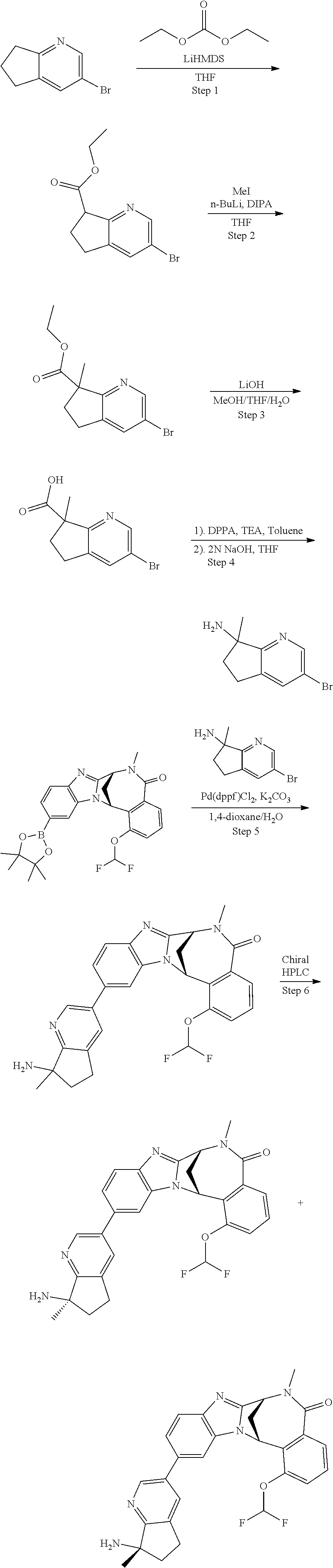

### Preparation 22A: ethyl 3-bromo-5H,6H,7H-cyclopenta[b]pyridine-7-carboxylate

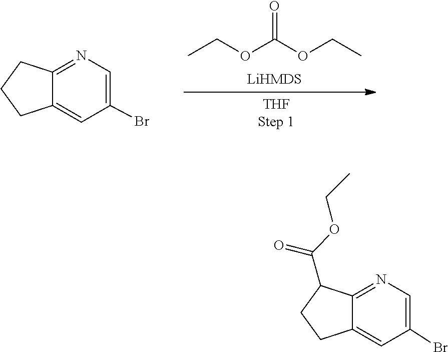

To a stirred solution of 3-bromo-5H,6H,7H-cyclopenta[b]pyridine (9.00 g, 45.440 mmol) in THF (150 mL) was added LiHMDS (114 mL, 113.600 mmol, 1M in THF) dropwise at −78° C. under nitrogen atmosphere. The resulting mixture was stirred for 1 h at −78° C. under nitrogen atmosphere. To the above mixture was added diethyl carbonate (16.10 g, 136.320 mmol) dropwise over 5 min at −78° C. The resulting mixture was stirred for additional overnight at room temperature. The reaction was quenched by the addition of sat. NH₄Cl (aq.) (150 mL) at room temperature. The resulting mixture was diluted with EtOAc (300 mL) and extracted with EtOAc (2×300 mL). The combined organic layers were washed with brine (300 mL), dried over anhydrous Na₂SO₄. After filtration, the filtrate was concentrated under reduced pressure. The residue was purified by silica gel column chromatography, eluted with PE/EA (3/1) to afford ethyl 3-bromo-5H,6H,7H-cyclopenta[b]pyridine-7-carboxylate (11.10 g, 89%) as a yellow oil. MS ESI calculated for C₁₁H₁₂BrNO₂ [M+H]⁺, 270.01 272.01, found 269.80 271.80. ¹H NMR (400 MHz, Chloroform-d) δ 8.49-8.43 (m, 1H), 7.71-7.66 (m, 1H), 4.22 (q, J=7.2 Hz, 2H), 4.09-3.99 (m, 1H), 3.19-3.06 (m, 1H), 2.99-2.87 (m, 1H), 2.55-2.36 (m, 2H), 1.28 (t, J=7.2 Hz, 3H).

### Preparation 22B: ethyl 3-bromo-7-methyl-5H,6H-cyclopenta[b]pyridine-7-carboxylate

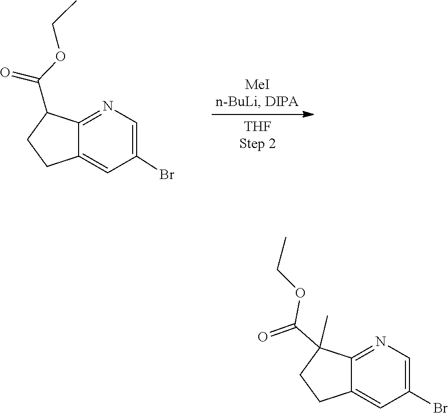 [SMILES: CCOC(=O)C1CCc2cc(Br)cnc21 ; CCOC(=O)C1(C)CCc2cc(Br)cnc21]

To a stirred solution of DIPA (3.77 mL, 26.654 mmol) in THF (50 mL) was added n-BuLi in hexanes (10.6 mL, 26.654 mmol, 2.5M in hex) dropwise at −78° C. under nitrogen atmosphere. The resulting mixture was stirred for 30 min at −78° C. under nitrogen atmosphere. To a stirred solution of ethyl 3-bromo-5H,6H,7H-cyclopenta[b]pyridine-7-carboxylate (6.00 g, 22.212 mmol) in THF (50 mL) was added the above mixture (fresh LDA) dropwise at −78° C. under nitrogen atmosphere. The reaction mixture was allowed to stir for 10 min then warmed to 0° C. over 30 min. The mixture was then cooled to −78° C. and a solution of methyl iodide (3.15 g, 22.212 mmol) in 5 mL THE was added. The resulting mixture was allowed to warm to ambient temperature and was stirred for 2 h. The reaction was quenched with 100 mL of a saturated ammonium chloride solution and extracted with EtOAc (3×100 mL). The organic layer was then separated and concentrated. The residue was purified by silica gel column chromatography, eluted with PE/EA (5/1) to afford ethyl 3-bromo-7-methyl-5H,6H-cyclopenta[b]pyridine-7-carboxylate (5.80 g, 91%) as a yellow oil. MS ESI calculated for C₁₂H₁₄BrNO₂ [M+H]⁺, 284.02 286.02, found 283.90 285.90. ¹H NMR (400 MHz, Chloroform-d) δ 8.48-8.42 (m, 1H), 7.68-7.63 (m, 1H), 4.20-4.04 (m, 2H), 3.12-2.99 (m, 1H), 2.95-2.83 (m, 1H), 2.76-2.64 (m, 1H), 2.08-1.96 (m, 1H), 1.56 (s, 3H), 1.18 (t, J=7.2 Hz, 3H).

### Preparation 22C: 3-bromo-7-methyl-5H,6H-cyclopenta[b]pyridine-7-carboxylic acid

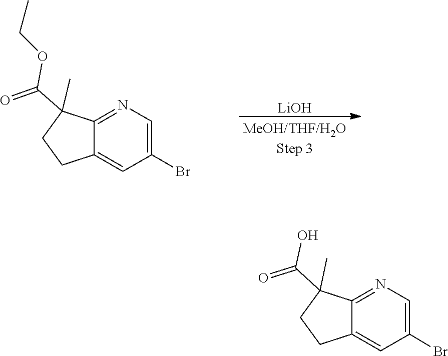 [SMILES: CCOC(=O)C1(C)CCc2cc(Br)cnc21 ; "LiOH" ; CC1(C(=O)O)CCc2cc(Br)cnc21]

To a stirred solution of ethyl 3-bromo-7-methyl-5H,6H-cyclopenta[b]pyridine-7-carboxylate (5.80 g, 20.412 mmol) in MeOH (30 mL) and THE (30 mL) was added a solution of LiOH (1.47 g, 61.378 mmol) in H₂O (30 mL) at room temperature under nitrogen atmosphere. The resulting mixture was stirred for 1 h at room temperature. The mixture was acidified to pH 2 with citric acid and extracted with EtOAc (3×200 mL). The combined organic layers were dried over anhydrous Na₂SO₄. After filtration, the filtrate was concentrated under reduced pressure to afford 3-bromo-7-methyl-5H,6H-cyclopenta[b]pyridine-7-carboxylic acid (4.90 g, 84%) as a white solid. MS ESI calculated for C₁₀H₁₀BrNO₂ [M+H]⁺,255.99 257.99, found 255.90 257.90. ¹H NMR (400 MHz, Chloroform-d) δ 11.53 (s, 1H), 8.50-8.45 (m, 1H), 7.73-7.67 (m, 1H), 3.09-2.96 (m, 1H), 2.96-2.86 (m, 1H), 2.76-7.65 (m, 1H), 2.16-2.04 (m, 1H), 1.56 (s, 3H).

### Preparation 22D: 3-bromo-7-methyl-5H,6H-cyclopenta[b]pyridin-7-amine

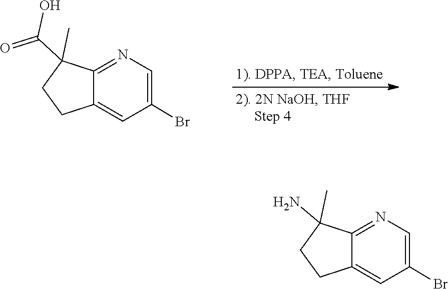

A solution of 3-bromo-7-methyl-5H,6H-cyclopenta[b]pyridine-7-carboxylic acid (500 mg, 1.952 mmol), TEA (237 mg, 2.342 mmol) and DPPA (591 mg, 2.147 mmol) in Toluene (20 mL) was stirred for 3 h at 110° C. under nitrogen atmosphere. The mixture was allowed to cool down to room temperature. The resulting mixture was concentrated under vacuum. The resulting mixture was diluted with THF (50 mL) and 20% aq. NaOH (50 mL). The resulting mixture was stirred for additional 30 min at room temperature. The resulting mixture was extracted with EtOAc (3×60 mL). The combined organic layers were washed with brine (100 mL), dried over anhydrous Na₂SO₄. After filtration, the filtrate was concentrated under reduced pressure. The residue was purified by silica gel column chromatography, eluted with CH₂Cl₂/MeOH (8/1) to afford 3-bromo-7-methyl-5H,6H-cyclopenta[b]pyridin-7-amine (120 mg, 27%) as a colorless solid. MS ESI calculated for C₉H₁₁BrN₂ [M+H]⁺, 227.01 229.01, found 227.05 229.10. ¹H NMR (400 MHz, DMSO-d₆) δ 8.51-8.36 (m, 1H), 7.93-7.80 (m, 1H), 2.95-2.83 (m, 1H), 2.83-2.70 (m, 1H), 2.22 (s, 2H), 2.07-1.91 (m, 2H), 1.28 (s, 3H).

### Preparation 22E: (1R,11R)-5-{7-amino-7-methyl-5H,6H-cyclopenta[b]pyridin-3-yl}-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

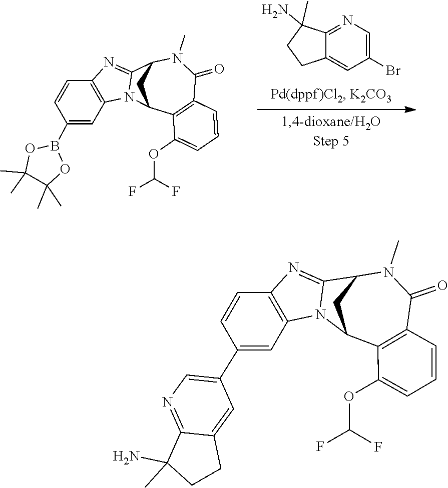 [SMILES: CN1C(=O)c2cccc(OC(F)F)c2[C@H]2C[C@@H]1c1nc3ccc(B4OC(C)(C)C(C)(C)O4)cc3n12 ; CC1(N)CCc2cc(Br)cnc21 ; CN1C(=O)c2cccc(OC(F)F)c2[C@H]2C[C@@H]1c1nc3ccc(-c4cnc5c(c4)CCC5(C)N)cc3n12]

To a solution of (1R,11R)-18-(difluoromethoxy)-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (100 mg, 0.208 mmol) and 3-bromo-7-methyl-5H,6H-cyclopenta[b]pyridin-7-amine (61 mg, 0.270 mmol) in 1,4-dioxane (1.6 mL) and H₂O (0.4 mL) were added K₂CO₃ (86 mg, 0.624 mmol) and Pd(dppf)Cl₂·CH₂Cl₂ (17 mg, 0.021 mmol). After stirring for 2 h at 80° C. under a nitrogen atmosphere, the resulting mixture was concentrated under reduced pressure. The residue was purified by silica gel column chromatography, eluted with CH₂Cl₂/MeOH (5/1) followed by reversed-phase flash chromatography with the following conditions: column, C18 silica gel; mobile phase, CH₃CN in water (10 mmol/L NH₄HCO₃), 25% to 50% gradient in 25 min; detector, 254 nm to afford (1R,11R)-5-{7-amino-7-methyl-5H,6H-cyclopenta[b]pyridin-3-yl}-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (100 mg, 94%) as a white solid. MS ESI calculated for C₂₈H₂₅F₂N₅O₂ [M+H]⁺, 502.20, found 502.20. ¹H NMR (400 MHz, DMSO-d₆) δ 8.58 (d, J=2.0 Hz, 1H), 8.31-8.24 (m, 1H), 7.86-7.64 (m, 4H), 7.53-7.44 (m, 3H), 6.29 (d, J=7.2 Hz, 1H), 5.24 (d, J=7.2 Hz, 1H), 3.59-3.44 (m, 1H), 3.36 (s, 3H), 2.99-2.88 (m, 1H), 2.87-2.77 (m, 2H), 2.12-1.96 (m, 2H), 1.91 (s, 2H), 1.33 (s, 3H). ¹⁹F NMR (377 MHz, DMSO-d₆) δ −81.56, −81.57, −82.01, −82.02, −82.15, −82.16, −82.59, −82.61.

### Example 22 and 23: (7R,14R)-11-((R)-7-amino-7-methyl-6,7-dihydro-5H-cyclopenta[b]pyridin-3-yl)-1-(difluoromethoxy)-6-methyl-6,7-dihydro-7,14-methanobenzo[f]benzo[4,5]imidazo[1,2-a][1,4]diazocin-5(14H)-one and (7R,14R)-11-((S)-7-amino-7-methyl-6,7-dihydro-5H-cyclopenta[b]pyridin-3-yl)-1-(difluoromethoxy)-6-methyl-6,7-dihydro-7,14-methanobenzo[f]benzo[4,5]imidazo[1,2-a][1,4]diazocin-5(14H)-one

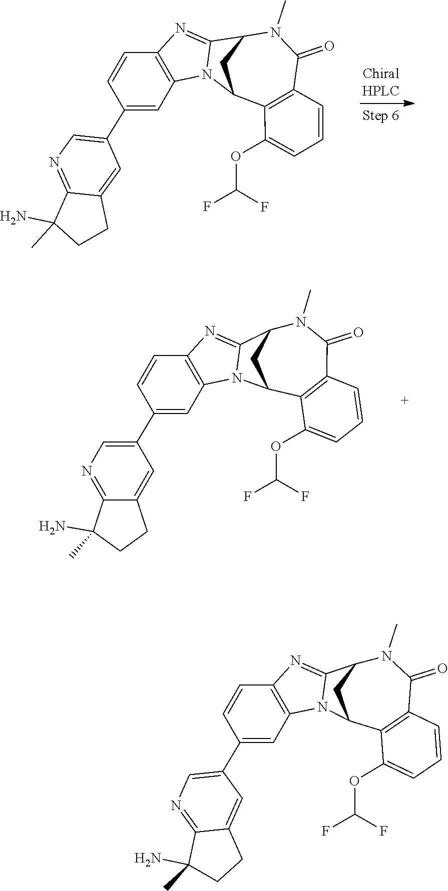 [SMILES: CN1C(=O)c2cccc(OC(F)F)c2[C@H]2C[C@@H]1c1nc3ccc(-c4cnc5c(c4)CCC5(C)N)cc3n12 ; CN1C(=O)c2cccc(OC(F)F)c2[C@H]2C[C@@H]1c1nc3ccc(-c4cnc5c(c4)CC[C@@]5(C)N)cc3n12 ; CN1C(=O)c2cccc(OC(F)F)c2[C@H]2C[C@@H]1c1nc3ccc(-c4cnc5c(c4)CC[C@]5(C)N)cc3n12]

The mixture (90 mg) was purified by Prep-Chiral HPLC with the following conditions (Column: CHIRAL ART Cellulose-SB, 2*25 cm, 5 μm; Mobile Phase A: Hex (10 mM NH₃-MeOH), Mobile Phase B: EtOH; Flow rate: 20 mL/min; Gradient: isocratic 50; Wave Length: 210/234 nm; RT1(min): 7.3; RT2(min): 16.3;). The first peak afford 35 mg (39%) as an off-white solid. MS ESI calculated for C₂₈H₂₅F₂N₅O₂ [M+H]⁺, 502.20, found 502.15. ¹H NMR (400 MHz, DMSO-d₆) δ 8.58 (d, J=2.0 Hz, 1H), 8.33-8.23 (m, 1H), 7.87-7.65 (m, 4H), 7.53-7.44 (m, 3H), 6.29 (d, J=7.2 Hz, 1H), 5.24 (d, J=7.2 Hz, 1H), 3.60-3.44 (m, 1H), 3.36 (s, 3H), 2.99-2.89 (m, 1H), 2.88-2.77 (m, 2H), 2.16-1.83 (m, 4H), 1.34 (s, 3H). ¹⁹F NMR (377 MHz, DMSO-d₆) δ −81.57, −82.02, −82.15, −82.60.

The second peak afford 30 mg (33%) as an off-white solid. MS ESI calculated for C₂₈H₂₅F₂N₅O₂ [M+H]⁺, 502.20, found 502.15. ¹H NMR (400 MHz, DMSO-d₆) δ 8.58 (d, J=2.0 Hz, 1H), 8.31-8.24 (m, 1H), 7.86-7.64 (m, 4H), 7.53-7.44 (m, 3H), 6.29 (d, J=7.2 Hz, 1H), 5.24 (d, J=7.2 Hz, 1H), 3.59-3.44 (m, 1H), 3.36 (s, 3H), 2.99-2.88 (m, 1H), 2.87-2.77 (m, 2H), 2.12-1.96 (m, 2H), 1.91 (s, 2H), 1.33 (s, 3H). ¹⁹F NMR (377 MHz, DMSO-d₆) δ −81.55, −82.00, −82.17, −82.62.

### Example 24: (1R,11R)-5-(4-amino-4-methyl-2,3-dihydro-1-benzopyran-7-yl)-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

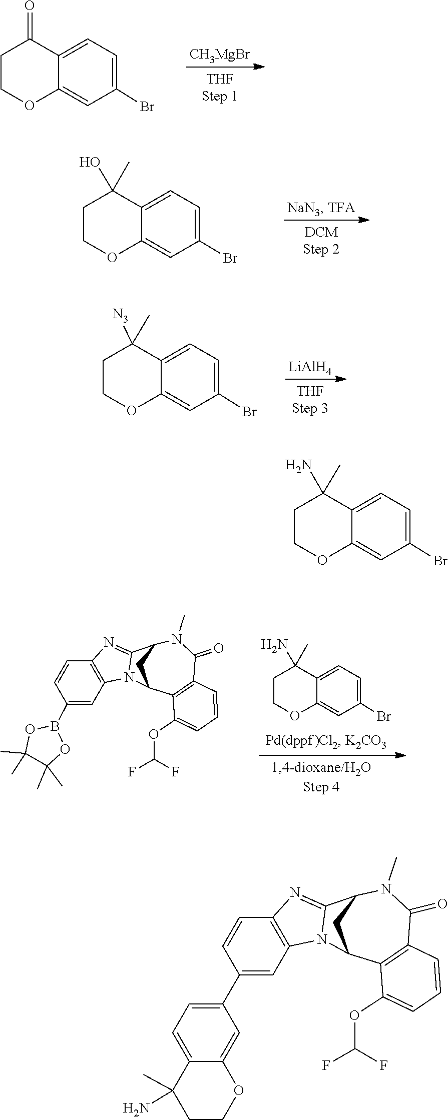

### Preparation 24A: 7-bromo-4-methyl-2,3-dihydro-1-benzopyran-4-ol

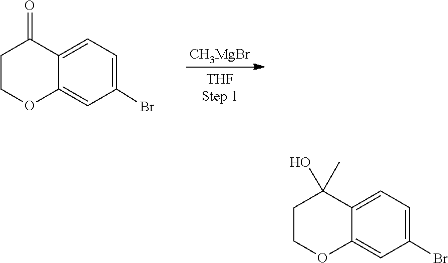

To a solution of 7-bromo-2,3-dihydro-1-benzopyran-4-one (5.00 g, 22.021 mmol) in THF (88 mL) was added CH₃MgBr (22 mL, 66.063 mmol, 3 M in THF) in dropwise at −58° C. under argon atmosphere. The resulting mixture was stirred for 16 h at room temperature under argon atmosphere. The resulting solution was quenched by the addition of sat. NH₄Cl (aq.) (80 mL) at room temperature. The aqueous layer was extracted with EtOAc (2×100 mL). The resulting mixture was concentrated under reduced pressure. This resulted in 7-bromo-4-methyl-2,3-dihydro-1-benzopyran-4-ol (3.00 g, 53%) as a colorless oil. ¹H NMR (300 MHz, Chloroform-d) δ 7.36 (d, J=8.3 Hz, 1H), 7.09-7.05 (m, 1H), 7.02 (d, J=2.0 Hz, 1H), 4.36-4.20 (m, 2H), 2.09-2.05 (m, 2H), 1.63 (s, 3H).

### Preparation 24B: 4-azido-7-bromo-4-methyl-2,3-dihydro-1-benzopyran

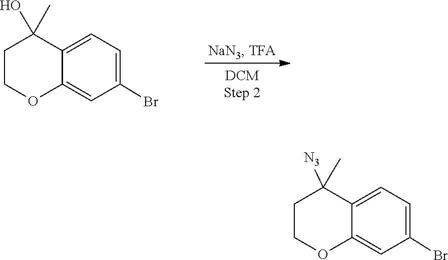

To a stirred solution of 7-bromo-4-methyl-2,3-dihydro-1-benzopyran-4-ol (1.00 g, 4.114 mmol) in DCM (15 mL) was added NaN₃ (0.80 g, 12.342 mmol) and TFA (1.41 g, 12.342 mmol) in portions at 0° C. The resulting mixture was stirred for 3 h at room temperature. The residue was basified to pH 8 with saturated NaHCO₃ (aq.). The aqueous layer was extracted with EtOAc (2×200 mL). The resulting mixture was concentrated under reduced pressure. This resulted in 4-azido-7-bromo-4-methyl-2,3-dihydro-1-benzopyran (600 mg, 52%) as a white solid. ¹H NMR (300 MHz, Chloroform-d) δ 7.15 (d, J=8.3 Hz, 1H), 7.04-6.96 (m, 2H), 4.22-4.15 (m, 2H), 2.00-1.93 (m, 2H), 1.59 (s, 3H).

### Preparation 24C: 7-bromo-4-methyl-2,3-dihydro-1-benzopyran-4-amine

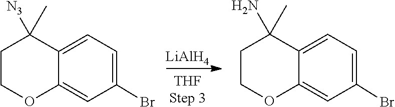

To a stirred solution of 4-azido-7-bromo-4-methyl-2,3-dihydro-1-benzopyran (500 mg, 1.865 mmol) in tetrahydrofuran (10 mL) was added LiAlH₄ (1.86 mL, 1.865 mmol) in portions at −10° C. under nitrogen atmosphere. The resulting mixture was stirred for 3 h at −10° C. under nitrogen atmosphere. The reaction was quenched by the addition of 15% NaOH (0.5 mL) at −10° C. The aqueous layer was extracted with EtOAc (2×100 mL). The organic layers were concentrated under reduced pressure. This resulted in 7-bromo-4-methyl-2,3-dihydro-1-benzopyran-4-amine (400 mg, 80%) as a yellow oil. ¹HNMR (400 MHz, Chloroform-d) δ 7.23 (d, J=8.3 Hz, 1H), 6.97-6.94 (m, 1H), 6.89 (d, J=2.0 Hz, 1H), 4.19-4.15 (m, 2H), 1.96-1.85 (m, 2H), 1.41 (s, 3H).

### Example 24: (1R,11R)-5-(4-amino-4-methyl-2,3-dihydro-1-benzopyran-7-yl)-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

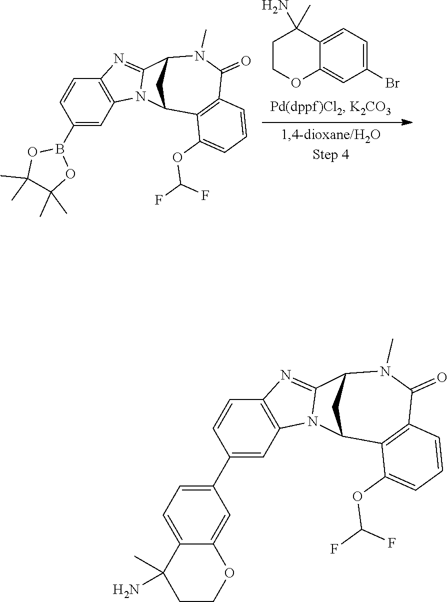

To a stirred solution of (1R,11R)-18-(difluoromethoxy)-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaene (50 mg, 0.107 mmol) and 7-bromo-4-methyl-2,3-dihydro-1-benzopyran-4-amine (31 mg, 0.128 mmol) in 1,4-dioxane (1.5 mL) and H₂O (0.3 mL) were added K₂CO₃ (44 mg, 0.321 mmol) and Pd(dppf)Cl₂·CH₂Cl₂ (8 mg, 0.011 mmol) in portions at room temperature under nitrogen atmosphere. The resulting mixture was concentrated under reduced pressure. The residue was purified by silica gel column chromatography, eluted with CH₂Cl₂/MeOH (10:1) followed by reversed phase flash with the following conditions (column, C18 silica gel; mobile phase, CH₃CN in water (10 mmol/L NH₄HCO₃), 30% to 70% gradient in 20 min; detector, 254 nm) to afford (1R,11R)-5-(4-amino-4-methyl-2,3-dihydro-1-benzopyran-7-yl)-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (17 mg) as a white solid. MS ESI calculated for C₂₉H₂₆F₂N₄O₃ [M+H]⁺, 517.20, found 517.30. ¹H NMR (400 MHz, DMSO-d₆) δ 8.30-8.25 (m, 1H), 7.84-7.41 (m, 7H), 7.13-7.10 (m, 1H), 6.92 (s, 1H), 6.27 (d, J=7.1 Hz, 1H), 5.22 (d, J=7.1 Hz, 1H), 4.30-4.25 (m, 1H), 4.21-4.15 (m, 1H), 3.55-3.47 (m, 1H), 3.32 (s, 3H), 2.81 (d, J=13.7 Hz, 1H), 2.04 (s, 2H), 1.89 (t, J=5.3 Hz, 2H), 1.40 (s, 3H). ¹⁹F NMR (376 MHz, DMSO-d₆) δ −81.96, −81.98.

### Example 25 and 26: (7R,14R)-11-((S)-7-amino-7-methyl-6,7-dihydro-5H-cyclopenta[b]pyridin-3-yl)-1-(difluoromethoxy)-10-fluoro-6-(methyl-d3)-6,7-dihydro-7,14-methanobenzo[f]benzo[4,5]imidazo[1,2-a][1,4]diazocin-5(14H)-one and (7R,14R)-11-((R)-7-amino-7-methyl-6,7-dihydro-5H-cyclopenta[b]pyridin-3-yl)-1-(difluoromethoxy)-10-fluoro-6-(methyl-d3)-6,7-dihydro-7,14-methanobenzo[f]benzo[4,5]imidazo[1,2-a][1,4]diazocin-5(14H)-one

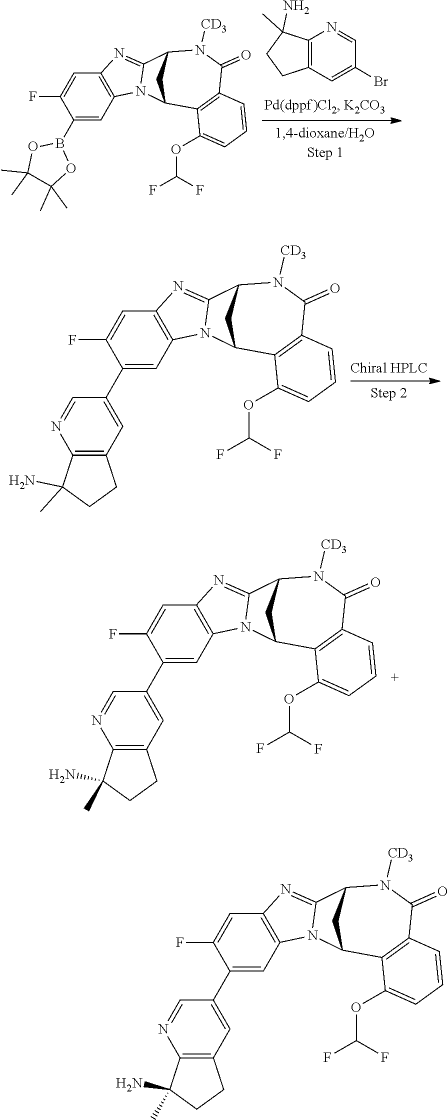

### Preparation 25A: (7R,14R)-11-(7-amino-7-methyl-6,7-dihydro-5H-cyclopenta[b]pyridin-3-yl)-1-(difluoromethoxy)-10-fluoro-6-(methyl-d3)-6,7-dihydro-7,14-methanobenzo[f]benzo[4,5]imidazo[1,2-a][1,4]diazocin-5(14H)-one

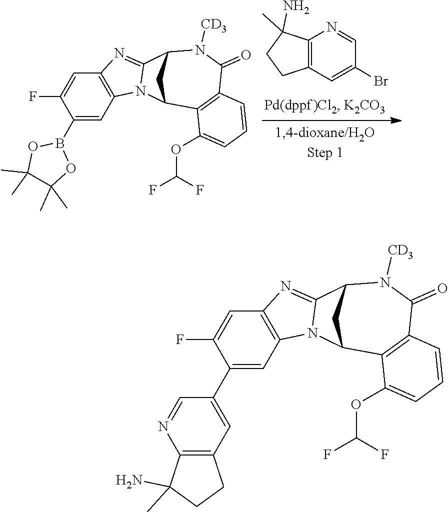

To a stirred solution of (7R,14R)-1-(difluoromethoxy)-10-fluoro-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (120 mg, 0.239 mmol) and 3-bromo-7-methyl-5H,6H-cyclopenta[b]pyridin-7-amine (81 mg, 0.358 mmol) in 1,4-dioxane (2 mL) and H₂O (0.4 mL) were added K₂CO₃ (83 mg, 0.597 mmol) and Pd(dppf)Cl₂·CH₂Cl₂ (19 mg, 0.024 mmol) at room temperature under nitrogen atmosphere. The resulting mixture was stirred for 3 h at 80° C. under nitrogen atmosphere. The resulting mixture was concentrated under reduced pressure. The residue was purified by silica gel column chromatography, eluted with CH₂Cl₂/MeOH (15/1) followed by reversed-phase flash chromatography with the following conditions: column, C18 silica gel; mobile phase, CH₃CN in water (10 mmol/L NH₄HCO₃), 25% to 40% gradient in 30 min; detector, 254 nm. This resulted in (7R,14R)-11-(7-amino-7-methyl-6,7-dihydro-5H-cyclopenta[b]pyridin-3-yl)-1-(difluoromethoxy)-10-fluoro-6-(methyl-d3)-6,7-dihydro-7,14-methanobenzo[f]benzo[4,5]imidazo[1,2-a][1,4]diazocin-5(14H)-one (65 mg, 52%) as a white solid. MS ESI calculated for C₂₈H₂₁D₃F₃N₅O₂ [M+H]⁺, 523.21, found 523.25. ¹H NMR (400 MHz, Chloroform-d) δ 8.56-8.46 (m, 2H), 7.68 (d, J=7.7 Hz, 1H), 7.52-7.39 (m, 3H), 7.31 (d, J=8.1 Hz, 1H), 6.82 (t, J=72.7 Hz, 1H), 6.24 (d, J=7.2 Hz, 1H), 4.96 (d, J=7.1 Hz, 1H), 3.51-3.42 (m, 1H), 3.07-2.98 (m, 1H), 2.96-2.85 (m, 2H), 2.35-2.20 (m, 2H), 1.55 (s, 3H). ¹⁹F NMR (377 MHz, Chloroform-d) δ −80.32, −80.34, −80.77, −80.78, −81.00, −81.05, −81.45, −81.49, −123.05, −123.07.

### Example 25 and 26: (7R,14R)-11-((S)-7-amino-7-methyl-6,7-dihydro-5H-cyclopenta[b]pyridin-3-yl)-1-(difluoromethoxy)-10-fluoro-6-(methyl-d3)-6,7-dihydro-7,14-methanobenzo[f]benzo[4,5]imidazo[1,2-a][1,4]diazocin-5(14H)-one and (7R,14R)-11-((R)-7-amino-7-methyl-6,7-dihydro-5H-cyclopenta[b]pyridin-3-yl)-1-(difluoromethoxy)-10-fluoro-6-(methyl-d3)-6,7-dihydro-7,14-methanobenzo[f]benzo[4,5]imidazo[1,2-a][1,4]diazocin-5(14H)-one

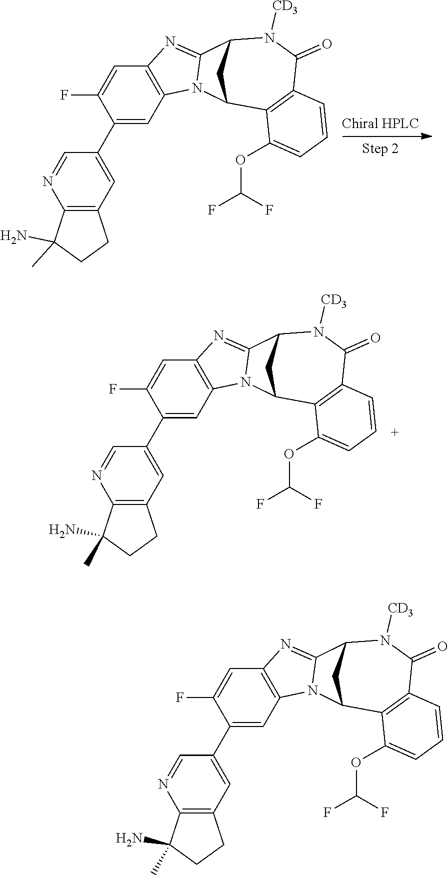 [SMILES: [2H]C([2H])([2H])N1C(=O)c2cccc(OC(F)F)c2[C@H]2C[C@@H]1c1nc3cc(F)c(-c4cnc5c(c4)CCC5(C)N)cc3n12 ; [2H]C([2H])([2H])N1C(=O)c2cccc(OC(F)F)c2[C@H]2C[C@@H]1c1nc3cc(F)c(-c4cnc5c(c4)CC[C@]5(C)N)cc3n12 ; [2H]C([2H])([2H])N1C(=O)c2cccc(OC(F)F)c2[C@H]2C[C@@H]1c1nc3cc(F)c(-c4cnc5c(c4)CC[C@@]5(C)N)cc3n12]

The mixture (55 mg) was purified by Chiral-Prep-HPLC with the following conditions (Column: CHIRAL ART Cellulose-SB, 2*25 cm, 5 μm; Mobile Phase A: Hex (10 mM NH₃-MeOH), Mobile Phase B: EtOH; Flow rate: 20 mL/min; Gradient: isocratic 30; Wave Length: 222/213 nm; RT1(min): 11; RT2(min): 17;). The first peak afforded 22 mg (40%) as a white solid. MS ESI calculated for C₂₈H₂₁D₃F₃N₅O₂ [M+H]⁺, 523.21, found 523.10. ¹H NMR (400 MHz, Chloroform-d) δ 8.54 (s, 1H), 8.51-8.47 (m, 1H), 7.68 (t, J=2.1 Hz, 1H), 7.49 (d, J=11.0 Hz, 1H), 7.47-7.43 (m, 1H), 7.41 (d, J=8.2 Hz, 1H), 7.33-7.29 (m, 1H), 6.82 (t, J=72.7 Hz, 1H), 6.24 (d, J=7.2 Hz, 1H), 4.96 (d, J=7.1 Hz, 1H), 3.52-3.43 (m, 1H), 3.07-2.97 (m, 1H), 2.97-2.84 (m, 2H), 2.36-2.21 (m, 2H), 1.56 (s, 3H). ¹⁹F NMR (377 MHz, Chloroform-d) δ −80.36, −80.80, −81.07, −81.52, −123.07.

The second peak afforded 20 mg (36%) as a white solid. MS ESI calculated for C₂₈H₂₁D₃F₃N₅O₂ [M+H]⁺, 523.21, found 523.15. ¹H NMR (400 MHz, Chloroform-d) δ 8.53-8.46 (m, 2H), 7.70 (t, J=2.1 Hz, 1H), 7.49 (d, J=11.0 Hz, 1H), 7.46-7.38 (m, 2H), 7.33-7.29 (m, 1H), 6.83 (t, J=72.7 Hz, 1H), 6.24 (d, J=7.2 Hz, 1H), 4.96 (d, J=7.1 Hz, 1H), 3.52-3.43 (m, 1H), 3.10-2.98 (m, 1H), 2.96-2.86 (m, 2H), 2.38-2.26 (m, 2H), 1.56 (s, 3H). ¹⁹F NMR (377 MHz, Chloroform-d) δ −80.34, −80.79, −81.08, −81.52, −123.05.

### Example 27: (1R,11R)-5-{7-amino-7-methyl-5H,6H-cyclopenta[b]pyridin-3-yl}-18-(difluoromethoxy)-6-fluoro-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one; formic acid

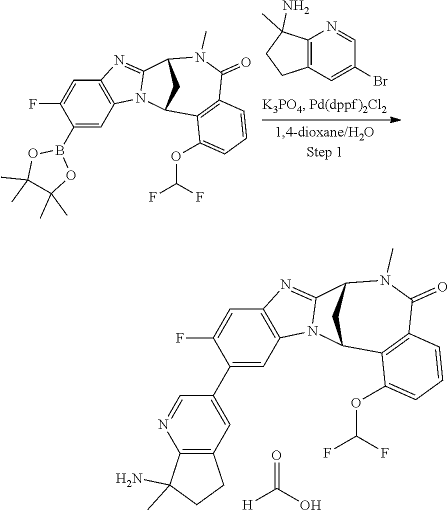

To a stirred solution of (1R,11R)-18-(difluoromethoxy)-6-fluoro-12-methyl-5-(4,4,5,5-tetramethyl-1,3,2-dioxaborolan-2-yl)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (70 mg, 0.140 mmol) and 3-bromo-7-methyl-5H,6H-cyclopenta[b]pyridin-7-amine (31 mg, 0.140 mmol) in 1,4-dioxane (2 mL) was added solution of K₃PO₄ (89 mg, 0.420 mmol) in H₂O (0.5 mL) at room temperature under nitrogen atmosphere. To the above solution was added Pd(dppf)Cl₂·CH₂Cl₂ (9 mg, 0.011 mmol) at room temperature under nitrogen atmosphere. The resulting mixture was stirred for additional 2 h at 80° C. The mixture was allowed to cool down to room temperature. The resulting mixture was concentrated under vacuum. The residue was purified by silica gel column chromatography, eluted with DCM/MeOH (0% to 10%) followed by Prep-HPLC with the following conditions: Column: C18 Column 120 g; Mobile Phase A: water (0.1% FA), Mobile Phase B: CH₃CN; Flow rate: 50 mL/min; Gradient: 20 B to 40 B in 40 min; 254/220 nm to afford (1R,11R)-5-{7-amino-7-methyl-5H,6H-cyclopenta[b]pyridin-3-yl}-18-(difluoromethoxy)-6-fluoro-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one; formic acid (30 mg, 38%) as a white solid. MS ESI calculated for C₂₈H₂₄F₃N₅O₂ [M+H]⁺, 520.19, found 520.20. ¹H NMR (400 MHz, DMSO-d₆) δ 8.70-8.62 (m, 4H), 8.30-8.27 (m, 1H), 7.93 (s, 1H), 7.82-7.19 (m, 5H), 6.30-6.28 (m, 1H), 5.30-5.28 (m, 1H), 3.57-3.50 (m, 1H), 3.36 (s, 3H), 3.17-2.99 (m, 2H), 2.87-2.83 (m, 1H), 2.45-2.28 (m, 2H), 1.62 (s, 3H). ¹⁹F NMR (377 MHz, DMSO-d₆) δ −81.94, −81.97, −81.99, −82.01, −123.93, −123.94.

### Example 28 and 29: (7R,14R)-11-((S)-7-amino-7-methyl-6,7-dihydro-5H-cyclopenta[b]pyridin-3-yl)-1-(difluoromethoxy)-6-(methyl-d3)-6,7-dihydro-7,14-methanobenzo[f]benzo[4,5]imidazo[1,2-a][1,4]diazocin-5(14H)-one and (7R,14R)-11-((R)-7-amino-7-methyl-6,7-dihydro-5H-cyclopenta[b]pyridin-3-yl)-1-(difluoromethoxy)-6-(methyl-d3)-6,7-dihydro-7,14-methanobenzo[f]benzo[4,5]imidazo[1,2-a][1,4]diazocin-5(14H)-one

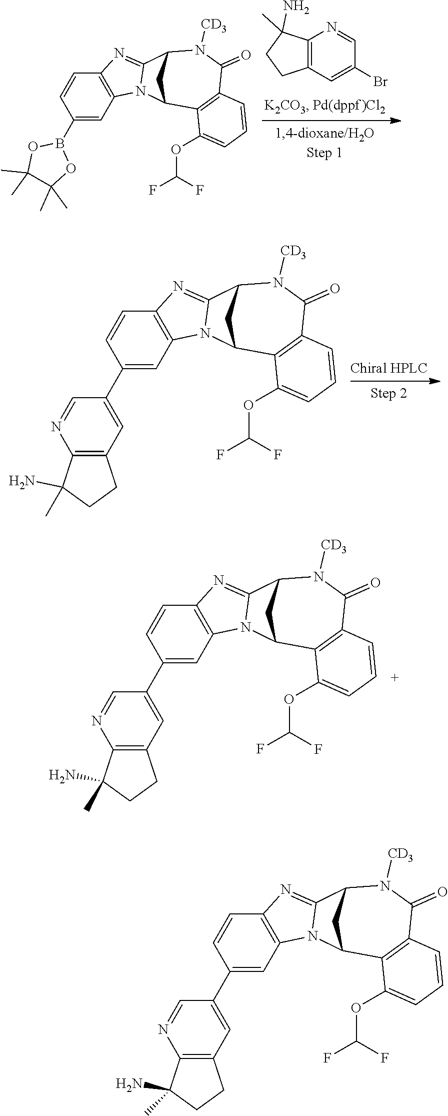

### Preparation 28A: (7R,14R)-11-(7-amino-7-methyl-6,7-dihydro-5H-cyclopenta[b]pyridin-3-yl)-1-(difluoromethoxy)-6-(methyl-d3)-6,7-dihydro-7,14-methanobenzo[f]benzo[4,5]imidazo[1,2-a][1,4]diazocin-5(14H)-one

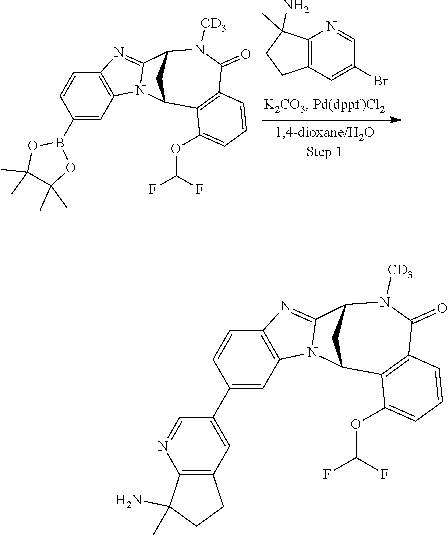

To a stirred mixture of (7R,14R)-1-(difluoromethoxy)-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (150 mg, 0.310 mmol) and 3-bromo-7-methyl-5H,6H-cyclopenta[b]pyridin-7-amine (84 mg, 0.372 mmol) in 1,4-dioxane (2 mL) were added K₂CO₃ (128 mg, 0.930 mmol) in H₂O (0.5 mL) and Pd(dppf)Cl₂·CH₂Cl₂ (25 mg, 0.031 mmol) at room temperature under nitrogen atmosphere. The resulting mixture was stirred for 2 h at 80° C. under nitrogen atmosphere. The resulting mixture was concentrated under vacuum. The residue was purified by silica gel column chromatography, eluted with DCM/MeOH (0% to 10%) followed by Prep-HPLC with the following conditions: Column: C18 Column 120 g; Mobile Phase A: water (0.1% NH₄HCO₃), Mobile Phase B: CH₃CN; Flow rate: 60 mL/min; Gradient: 30 B to 60 B in 30 min; 254/220 nm to afford (7R,14R)-11-(7-amino-7-methyl-6,7-dihydro-5H-cyclopenta[b]pyridin-3-yl)-1-(difluoromethoxy)-6-(methyl-d3)-6,7-dihydro-7,14-methanobenzo[f]benzo[4,5]imidazo[1,2-a][1,4]diazocin-5(14H)-one (90 mg, 56%) as a white solid. MS ESI calculated for C₂₈H₂₂D₃F₂N₅O₂ [M+H]⁺, 505.22, found 505.20. ¹H NMR (400 MHz, DMSO-d₆) δ 8.59 (d, J=2.1 Hz, 1H), 8.32-8.22 (m, 1H), 7.83-7.65 (m, 4H), 7.55-7.46 (m, 3H), 6.29 (d, J=7.1 Hz, 1H), 5.24 (d, J=7.2 Hz, 1H), 3.57-3.46 (m, 1H), 3.00-2.89 (m, 1H), 2.88-2.77 (m, 2H), 2.28-2.13 (m, 2H), 2.14-1.95 (m, 2H), 1.34 (s, 3H). ¹⁹F NMR (377 MHz, DMSO-d₆) δ −81.55, −81.57, −82.00, −82.02, −82.14, −82.16, −82.59, −82.61.

### Example 28 and 29: (7R,14R)-11-((S)-7-amino-7-methyl-6,7-dihydro-5H-cyclopenta[b]pyridin-3-yl)-1-(difluoromethoxy)-6-(methyl-d3)-6,7-dihydro-7,14-methanobenzo[f]benzo[4,5]imidazo[1,2-a][1,4]diazocin-5(14H)-one and (7R,14R)-11-((R)-7-amino-7-methyl-6,7-dihydro-5H-cyclopenta[b]pyridin-3-yl)-1-(difluoromethoxy)-6-(methyl-d3)-6,7-dihydro-7,14-methanobenzo[f]benzo[4,5]imidazo[1,2-a][1,4]diazocin-5(14H)-one

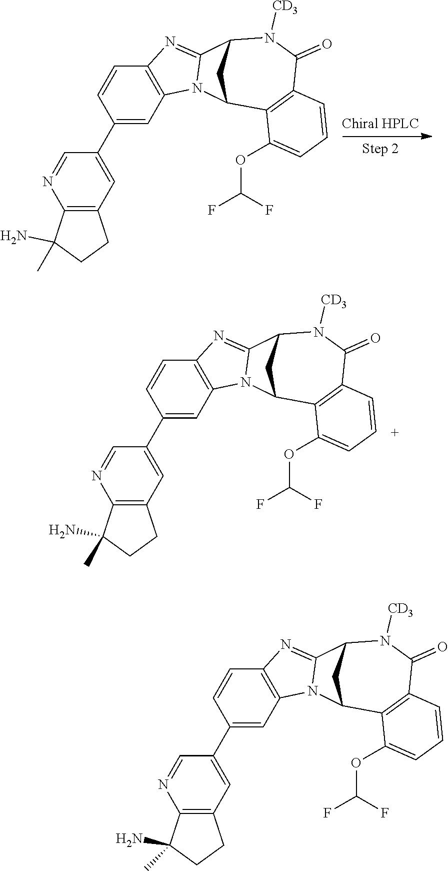 [SMILES: [2H]C([2H])([2H])N1C(=O)c2cccc(OC(F)F)c2[C@H]2C[C@@H]1c1nc3ccc(-c4cnc5c(c4)CCC5(C)N)cc3n12 ; [2H]C([2H])([2H])N1C(=O)c2cccc(OC(F)F)c2[C@H]2C[C@@H]1c1nc3ccc(-c4cnc5c(c4)CC[C@]5(C)N)cc3n12 ; [2H]C([2H])([2H])N1C(=O)c2cccc(OC(F)F)c2[C@H]2C[C@@H]1c1nc3ccc(-c4cnc5c(c4)CC[C@@]5(C)N)cc3n12]

(7R,14R)-11-(7-amino-7-methyl-6,7-dihydro-5H-cyclopenta[b]pyridin-3-yl)-1-(difluoromethoxy)-6-(methyl-d3)-6,7-dihydro-7,14-methanobenzo[f]benzo[4,5]imidazo[1,2-a][1,4]diazocin-5(14H)-one (85 mg) was purified by Chiral-Prep-PLC with the following conditions (Column: CHIRAL ART Cellulose-SB, 2*25 cm, 5 μm; Mobile Phase A: Hex (10 mM NH3-MeOH), Mobile Phase B: EtOH; Flow rate: 20 mL/min; Gradient: isocratic 50; Wave Length: 220/293 nm; RT1(min): 7.5; RT2(min): 14.2;). The first peak afforded 25 mg (28%) as a white solid. MS ESI calculated for C₂₈H₂₂D₃F₂N₅O₂ [M+H], 505.22, found 505.20. ¹H NMR (400 MHz, Chloroform-d) δ 8.63 (d, J=2.1 Hz, 1H), 8.52-8.47 (m, 1H), 7.81-7.75 (m, 1H), 7.71-7.62 (m, 2H), 7.46-7.37 (m, 2H), 7.34-7.28 (m, 1H), 6.84 (t, J=72.8 Hz, 1H), 6.28 (d, J=7.2 Hz, 1H), 4.97 (d, J=7.1 Hz, 1H), 3.53-3.42 (m, 1H), 3.04-2.85 (m, 3H), 2.34-2.25 (m, 1H), 2.21-2.10 (m, 1H), 1.51 (s, 3H). ¹⁹F NMR (377 MHz, Chloroform-d) δ −80.17, −80.61, −80.83, −81.28.

The second peak afforded 25 mg (29%) as a white solid. MS ESI calculated for C₂₈H₂₂D₃F₂N₅O₂ [M+H]⁺, 505.22, found 505.15. ¹H NMR (400 MHz, Chloroform-d) δ 8.61 (d, J=2.1 Hz, 1H), 8.52-8.46 (m, 1H), 7.78 (d, J=8.4 Hz, 1H), 7.73-7.68 (m, 1H), 7.64 (d, J=1.7 Hz, 1H), 7.46-7.37 (m, 2H), 7.31 (d, J=8.2 Hz, 1H), 6.84 (t, J=72.9 Hz, 1H), 6.27 (d, J=7.2 Hz, 1H), 4.96 (d, J=7.1 Hz, 1H), 3.54-3.41 (m, 1H), 3.06-2.97 (m, 1H), 2.96-2.84 (m, 2H), 2.35-2.26 (m, 1H), 2.22-2.12 (m, 1H), 1.52 (s, 3H). ¹⁹F NMR (377 MHz, Chloroform-d) 6-80.14, −80.63, −80.84, −81.29.

### Example 30: (7R,14R)-11-((S)-4-amino-3,4-dihydro-2H-pyrano[3,2-b]pyridin-7-yl)-1-(difluoromethoxy)-6-(methyl-d3)-6,7-dihydro-7,14-methanobenzo[f]benzo[4,5]imidazo[1,2-a][1,4]diazocin-5(14H)-one

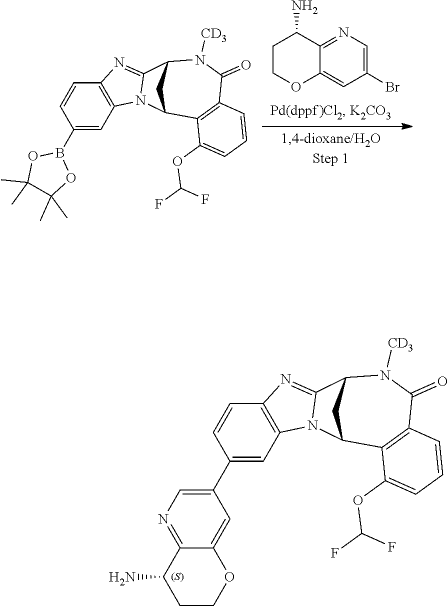

A mixture of (7R,14R)-1-(difluoromethoxy)-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (70 mg, 0.145 mmol), (4S)-7-bromo-2H,3H,4H-pyrano[3,2-b]pyridin-4-amine (39 mg, 0.174 mmol), K₂CO₃ (69 mg, 0.507 mmol) and Pd(dppf)Cl₂·CH₂Cl₂ (23 mg, 0.029 mmol) in 1,4-dioxane (1 mL) and H₂O (0.2 mL) was stirred for 2 h at 80° C. under nitrogen atmosphere. The resulting mixture was concentrated under vacuum and purified by silica gel column chromatography, eluted with CH₂Cl₂/MeOH (10/1) followed by reversed-phase flash chromatography with the following conditions: column, C18 silica gel; mobile phase, CH₃CN in water (10 mmol/L NH₄HCO₃), 30% to 70% gradient in 20 min; detector, 254/220 nm to afford (7R,14R)-11-((S)-4-amino-3,4-dihydro-2H-pyrano[3,2-b]pyridin-7-yl)-1-(difluoromethoxy)-6-(methyl-d3)-6,7-dihydro-7,14-methanobenzo[f]benzo[4,5]imidazo[1,2-a][1,4]diazocin-5(14H)-one (24 mg, 32%) as a white solid. MS ESI calculated for C₂₇H₂₀D₃F₂N₅O₃ [M+H]⁺, 507.20, found 507.30. ¹H NMR (400 MHz, Chloroform-d) δ 8.49 (d, J=8.9 Hz, 1H), 8.42-8.38 (m, 1H), 7.78 (d, J=8.5 Hz, 1H), 7.65 (s, 1H), 7.45-7.38 (m, 2H), 7.32-7.30 (m, 2H), 6.84 (t, J=72.8 Hz, 1H), 6.28 (d, J=7.2 Hz, 1H), 4.96 (d, J=7.1 Hz, 1H), 4.43-4.35 (m, 1H), 4.33-4.19 (m, 2H), 3.52-3.44 (m, 1H), 2.88 (d, J=13.5 Hz, 1H), 2.41-2.32 (m, 1H), 2.10-2.08 (m, 1H). ¹⁹F NMR (377 MHz, Chloroform-d) δ −80.12, −80.56, −80.88, −81.32.

### Example 31: (7R,14R)-11-(3-amino-3-methyl-2-oxoindolin-6-yl)-1-(difluoromethoxy)-6-methyl-6,7-dihydro-7,14-methanobenzo[f]benzo[4,5]imidazo[1,2-a][1,4]diazocin-5(14H)-one

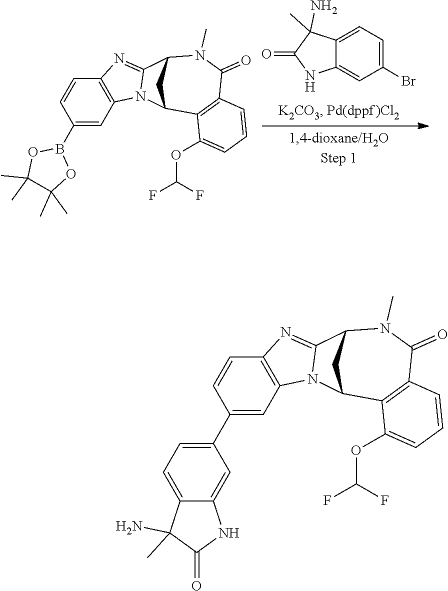

A solution of (7R,14R)-1-(difluoromethoxy)-6-methyl-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (80 mg, 0.166 mmol), 3-amino-6-bromo-3-methyl-1H-indol-2-one (48 mg, 0.199 mmol), K₂CO₃ (68 mg, 0.498 mmol) and Pd(dppf)Cl₂ (13 mg, 0.017 mmol) in 1,4-dioxane (1 mL) and H₂O (0.2 mL) was stirred for 2 h at 80° C. under nitrogen atmosphere. The resulting mixture was concentrated under vacuum and purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-11-(3-amino-3-methyl-2-oxoindolin-6-yl)-1-(difluoromethoxy)-6-methyl-6,7-dihydro-7,14-methanobenzo[f]benzo[4,5]imidazo[1,2-a][1,4]diazocin-5(14H)-one (25 mg, 29%) as a white solid. MS ESI calculated for C₂₈H₂₃F₂N₅O₃ [M+H]⁺, 516.18, found 516.10. ¹H NMR (400 MHz, Chloroform-d) δ 8.49 (d, J=8.3 Hz, 1H), 7.75 (d, J=8.5 Hz, 1H), 7.71-7.61 (m, 2H), 7.51-7.37 (m, 3H), 7.35-7.28 (m, 2H), 7.08 (d, J=5.8 Hz, 1H), 6.84 (t, J=73.0 Hz, 1H), 6.27 (d, J=7.0 Hz, 1H), 4.97 (d, J=6.9 Hz, 1H), 3.52 (s, 3H), 3.51-3.40 (m, 1H), 2.88 (d, J=13.3 Hz, 1H), 1.53 (s, 3H), ¹⁹F NMR (377 MHz, Chloroform-d) δ −80.12, −80.58, −80.60, −81.04.

### Example 32 and 33: (7R,14R)-1-(difluoromethoxy)-11-((S)-7-hydroxy-7-methyl-6,7-dihydro-5H-cyclopenta[b]pyridin-3-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((R)-7-hydroxy-7-methyl-6,7-dihydro-5H-cyclopenta[b]pyridin-3-yl)-6-(methyl-d3)-6,7-dihydro-7,14-methanobenzo[f]benzo[4,5]imidazo[1,2-a][1,4]diazocin-5(14H)-one

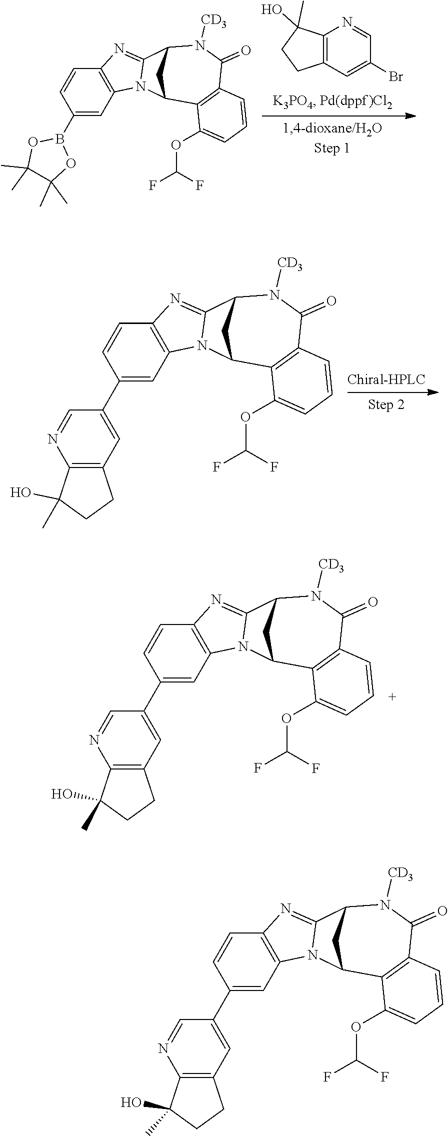

### Preparation 32A: (7R,14R)-1-(difluoromethoxy)-11-(7-hydroxy-7-methyl-6,7-dihydro-5H-cyclopenta[b]pyridin-3-yl)-6-(methyl-d3)-6,7-dihydro-7,14-methanobenzo[f]benzo[4,5]imidazo[1,2-a][1,4]diazocin-5(14H)-one

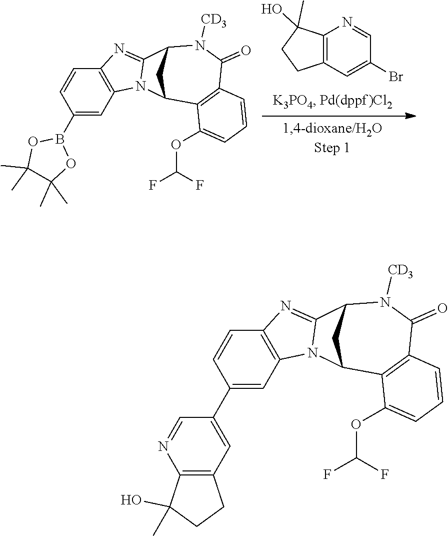

To a solution of (7R,14R)-1-(difluoromethoxy)-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (150 mg, 0.310 mmol) and 3-bromo-7-methyl-5H,6H-cyclopenta[b]pyridin-7-ol (85 mg, 0.372 mmol) in 1,4-dioxane (2 mL) and H₂O (0.4 mL) were added K₂CO₃ (129 mg, 0.930 mmol) and Pd(dppf)Cl₂ (25 mg, 0.031 mmol) at room temperature. After stirring for overnight at 100° C. under a nitrogen atmosphere, the resulting mixture was concentrated under reduced pressure and purified by silica gel column chromatography, eluted with CH₂Cl₂/MeOH (10/1) followed by reversed-phase flash chromatography with the following conditions: column, C18 silica gel; mobile phase, CH₃CN in Water (0.1% NH₃H₂O), 25% to 50% gradient in 25 min to afford (7R,14R)-1-(difluoromethoxy)-11-(7-hydroxy-7-methyl-6,7-dihydro-5H-cyclopenta[b]pyridin-3-yl)-6-(methyl-d3)-6,7-dihydro-7,14-methanobenzo[f]benzo[4,5]imidazo[1,2-a][1,4]diazocin-5(14H)-one (95 mg, 60%) as a white solid. MS ESI calculated for C₂₈H₂₁D₃F₂N₄O₃ [M+H]⁺, 506.20, found 506.15.

### Example 32 and 33: (7R,14R)-1-(difluoromethoxy)-11-((S)-7-hydroxy-7-methyl-6,7-dihydro-5H-cyclopenta[b]pyridin-3-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((R)-7-hydroxy-7-methyl-6,7-dihydro-5H-cyclopenta[b]pyridin-3-yl)-6-(methyl-d3)-6,7-dihydro-7,14-methanobenzo[f]benzo[4,5]imidazo[1,2-a][1,4]diazocin-5(14H)-one

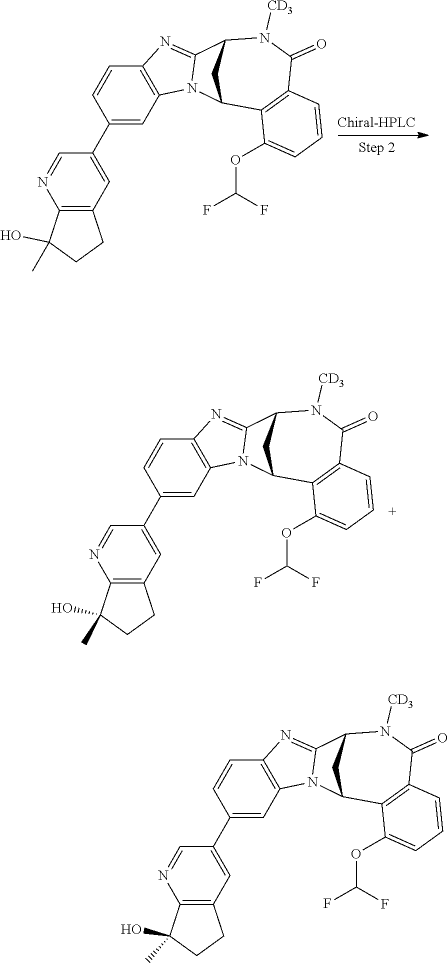 [SMILES: [2H]C([2H])([2H])N1C(=O)c2cccc(OC(F)F)c2[C@H]2C[C@@H]1c1nc3ccc(-c4cnc5c(c4)CCC5(C)O)cc3n12 ; [2H]C([2H])([2H])N1C(=O)c2cccc(OC(F)F)c2[C@H]2C[C@@H]1c1nc3ccc(-c4cnc5c(c4)CC[C@]5(C)O)cc3n12 ; [2H]C([2H])([2H])N1C(=O)c2cccc(OC(F)F)c2[C@H]2C[C@@H]1c1nc3ccc(-c4cnc5c(c4)CC[C@@]5(C)O)cc3n12]

The mixture (94 mg) was separated by Chiral Separation with following Conditions: (Column: CHIRAL ART Cellulose-SZ, 2.0*25 cm, 5 um; Mobile Phase A: Hex (10 mM NH₃-MeOH), Mobile Phase B: EtOH; Flow rate: 20 mL/min; Gradient: isocratic 50; Wave Length: 210/220 nm; RT1(min): 8.4; RT2(min): 10.6). The first peak afforded 37 mg (39%) as a white solid. MS ESI calculated for C₂₈H₂₁D₃F₂N₄O₃ [M+H]⁺, 506.20, found 506.15. ¹H NMR (400 MHz, Chloroform-d) δ 8.67-8.61 (m, 1H), 8.52-8.47 (m, 1H), 7.89-7.84 (m, 1H), 7.81 (d, J=8.4 Hz, 1H), 7.66 (d, J=1.8 Hz, 1H), 7.47-7.38 (m, 2H), 7.33 (d, J=1.2 Hz, 1H), 6.86 (t, J=72.8 Hz, 1H), 6.29 (d, J=7.2 Hz, 1H), 4.98 (d, J=7.1 Hz, 1H), 3.55-3.41 (m, 1H), 3.16-3.05 (m, 1H), 2.97-2.85 (m, 2H), 2.46-2.29 (m, 2H), 1.73 (s, 3H). ¹⁹F NMR (376 MHz, Chloroform-d) δ −80.33, −80.78, −80.83, −81.27. The second peak afford 36 mg (38%) as a white solid. MS ESI calculated for C₂₈H₂₁D₃F₂N₄O₃ [M+H]⁺, 506.20, found 506.10. ¹H NMR (400 MHz, Chloroform-d) δ 8.67-8.61 (m, 1H), 8.52-8.47 (m, 1H), 7.89-7.84 (m, 1H), 7.81 (d, J=8.4 Hz, 1H), 7.66 (d, J=1.8 Hz, 1H), 7.47-7.38 (m, 2H), 7.33 (d, J=1.2 Hz, 1H), 6.86 (t, J=72.8 Hz, 1H), 6.29 (d, J=7.2 Hz, 1H), 4.98 (d, J=7.1 Hz, 1H), 3.55-3.41 (m, 1H), 3.16-3.05 (m, 1H), 2.97-2.85 (m, 2H), 2.46-2.29 (m, 2H), 1.68 (s, 3H). ¹⁹F NMR (376 MHz, Chloroform-d) δ −80.33, −80.78, −80.83, −81.27.

### Example 34 and 35: (7R,14R)-1-(difluoromethoxy)-11-((S)-4-hydroxy-4-methyl-3,4-dihydro-2H-pyrano[3,2-b]pyridin-7-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((R)-4-hydroxy-4-methyl-3,4-dihydro-2H-pyrano[3,2-b]pyridin-7-yl)-6-(methyl-d3)-6,7-dihydro-7,14-methanobenzo[f]benzo[4,5]imidazo[1,2-a][1,4]diazocin-5(14H)-one

### Synthetic Scheme

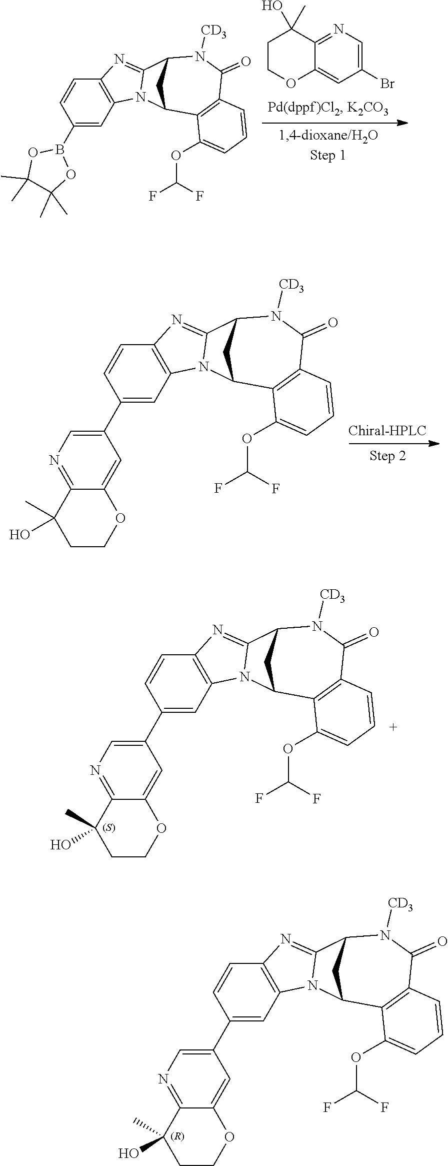

### Preparation 34A: (7R,14R)-1-(difluoromethoxy)-11-(4-hydroxy-4-methyl-3,4-dihydro-2H-pyrano[3,2-b]pyridin-7-yl)-6-(methyl-d3)-6,7-dihydro-7,14-methanobenzo[f]benzo[4,5]imidazo[1,2-a][1,4]diazocin-5(14H)-one

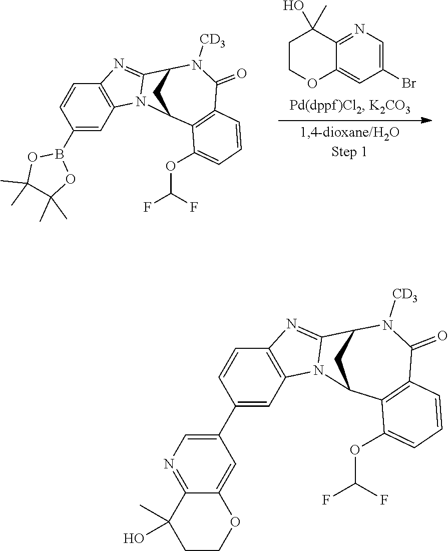 [SMILES: [2H]C([2H])([2H])N1C(=O)c2cccc(OC(F)F)c2[C@H]2C[C@@H]1c1nc3ccc(B4OC(C)(C)C(C)(C)O4)cc3n12 ; CC1(O)CCOc2cc(Br)cnc21 ; [2H]C([2H])([2H])N1C(=O)c2cccc(OC(F)F)c2[C@H]2C[C@@H]1c1nc3ccc(-c4cnc5c(c4)OCCC5(C)O)cc3n12]

A mixture of (7R,14R)-1-(difluoromethoxy)-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (120 mg, 0.248 mmol), 7-bromo-4-methyl-2H,3H-pyrano[3,2-b]pyridin-4-ol (72 mg, 0.298 mmol), Pd(dppf)Cl₂ (40 mg, 0.050 mmol) and K₂CO₃ (119 mg, 0.868 mmol) in 1,4-dioxane (1 mL) and H₂O (0.2 mL) were stirred for 2 h at 80° C. under nitrogen atmosphere. The resulting mixture was concentrated under vacuum and purified by silica gel column chromatography, eluted with CH₂Cl₂/MeOH (10/1) followed by reversed-phase flash chromatography with the following conditions: column, C18 silica gel; mobile phase, CH₃CN in water (10 mmol/L NH₄HCO₃), 30% to 70% gradient in 20 min; detector, 254/220 nm to afford (7R,14R)-1-(difluoromethoxy)-11-(4-hydroxy-4-methyl-3,4-dihydro-2H-pyrano[3,2-b]pyridin-7-yl)-6-(methyl-d3)-6,7-dihydro-7,14-methanobenzo[f]benzo[4,5]imidazo[1,2-a][1,4]diazocin-5(14H)-one (100 mg, 77%) as a white solid. MS ESI calculated for C₂₈H₂₁D₃F₂N₄O₄ [M+H]⁺, 522.20 found 522.20.

### Example 34 and 35: (7R,14R)-1-(difluoromethoxy)-11-((S)-4-hydroxy-4-methyl-3,4-dihydro-2H-pyrano[3,2-b]pyridin-7-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((R)-4-hydroxy-4-methyl-3,4-dihydro-2H-pyrano[3,2-b]pyridin-7-yl)-6-(methyl-d3)-6,7-dihydro-7,14-methanobenzo[f]benzo[4,5]imidazo[1,2-a][1,4]diazocin-5(14H)-one

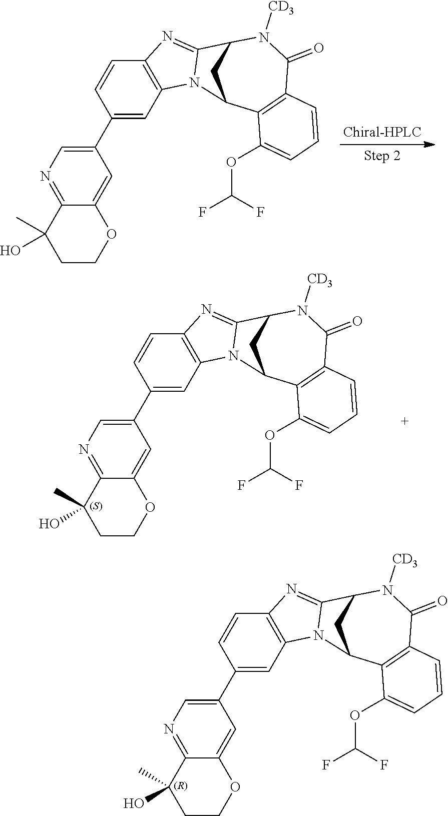

The mixture (100 mg) was purified by Prep-HPLC with the following conditions (Column: CHIRAL ART Cellulose-SZ, 2.0*25 cm, 5 um; Mobile Phase A: Hex (10 mM NH₃-MeOH), Mobile Phase B: EtOH; Flow rate: 20 mL/min; Gradient: isocratic 25; Wave Length: 220/301 nm; RT1(min): 12.7; RT2(min): 15.5). The first peak afford 23 mg (23%) as a white solid. MS ESI calculated for C₂₈H₂₁D₃F₂N₄O₄ [M+H]⁺, 522.20, found 522.10. ¹H NMR (400 MHz, Chloroform-d) δ 8.54-8.47 (m, 1H), 8.41 (d, J=1.9 Hz, 1H), 7.79 (d, J=8.5 Hz, 1H), 7.66 (d, J=1.7 Hz, 1H), 7.45-7.39 (m, 2H), 7.37 (d, J=1.9 Hz, 1H), 7.32 (d, J=8.2 Hz, 1H), 6.85 (t, J=72.7 Hz, 1H), 6.29 (d, J=7.2 Hz, 1H), 4.97 (d, J=7.1 Hz, 1H), 4.47-4.38 (m, 1H), 4.34-4.26 (m, 1H), 3.55-3.41 (m, 2H), 2.89 (d, J=13.6 Hz, 1H), 2.38-2.27 (m, 1H), 2.23-2.13 (m, 1H), 1.74 (s, 3H). ¹⁹F NMR (377 MHz, Chloroform-d) δ −80.16, −80.60, −80.88, −81.32. The second peak afford 30 mg (30%) as a white solid. MS ESI calculated for C₂₈H₂₁D₃F₂N₄O₄ [M+H]⁺, 522.20 found 522.15. ¹H NMR (400 MHz, Chloroform-d) δ 8.55-8.47 (m, 1H), 8.40 (d, J=1.9 Hz, 1H), 7.79 (d, J=8.5 Hz, 1H), 7.66 (d, J=1.7 Hz, 1H), 7.47-7.41 (m, 3H), 7.37-7.31 (m, 1H), 6.86 (t, J=72.8 Hz, 1H), 6.29 (d, J=7.2 Hz, 1H), 4.98 (d, J=7.1 Hz, 1H), 4.48-4.39 (m, 1H), 4.37-4.26 (m, 1H), 3.72 (s, 1H), 3.54-3.38 (m, 1H), 2.89 (d, J=13.5 Hz, 1H), 2.40-2.28 (m, 1H), 2.25-2.15 (m, 1H), 1.77 (s, 3H). ¹⁹F NMR (377 MHz, Chloroform-d) δ −80.23, −80.67, −80.87, −81.31.

### Example 36 and 37: (7R,14R)-11-((R)-7-amino-7-(hydroxymethyl)-6,7-dihydro-5H-cyclopenta[b]pyridin-3-yl)-1-(difluoromethoxy)-6-methyl-6,7-dihydro-7,14-methanobenzo[f]benzo[4,5]imidazo[1,2-a][1,4]diazocin-5(14H)-one and (7R,14R)-11-((S)-7-amino-7-(hydroxymethyl)-6,7-dihydro-5H-cyclopenta[b]pyridin-3-yl)-1-(difluoromethoxy)-6-methyl-6,7-dihydro-7,14-methanobenzo[f]benzo[4,5]imidazo[1,2-a][1,4]diazocin-5(14H)-one

### Synthetic Scheme

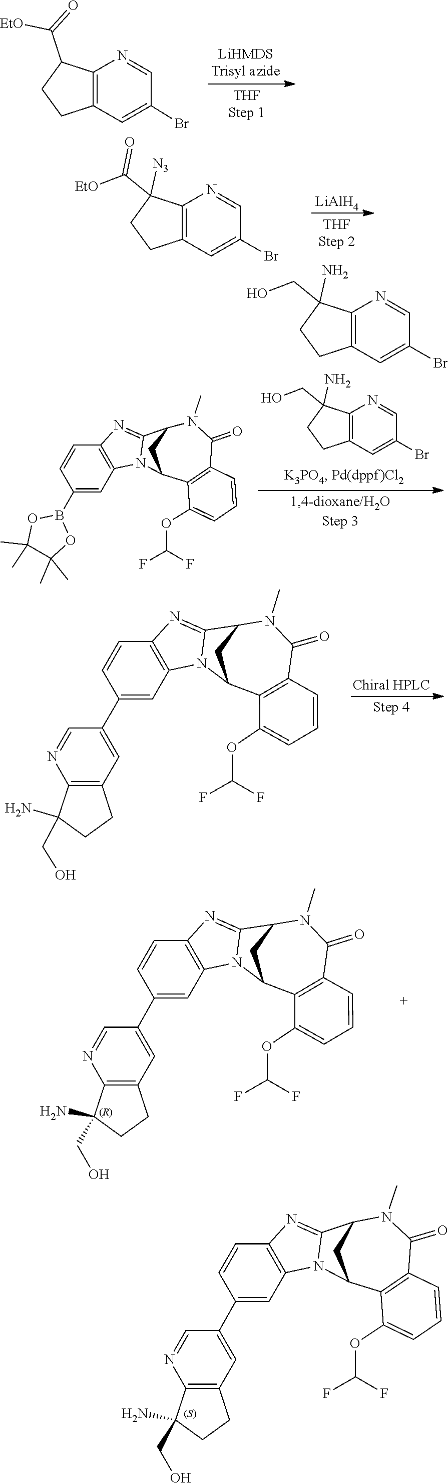

### Preparation 36A: ethyl 7-azido-3-bromo-5H,6H-cyclopenta[b]pyridine-7-carboxylate

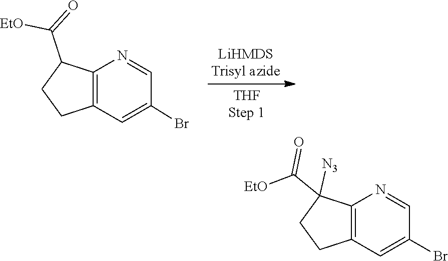 [SMILES: CCOC(=O)C1CCc2cc(Br)cnc21 ; "LiHMDSTrisyl azide" ; CCOC(=O)C1(N=[N+]=[N-])CCc2cc(Br)cnc21]

To a stirred solution of ethyl 3-bromo-5H,6H,7H-cyclopenta[b]pyridine-7-carboxylate (1.00 g, 3.702 mmol) in THF (12 mL) was added LiHMDS (4 mL, 4.072 mmol, 1 M in THF) dropwise at −20° C. under nitrogen atmosphere. The resulting mixture was stirred for 15 min at −20° C. under nitrogen atmosphere. To the above mixture was added 2,4,6-tris(propan-2-yl)benzene-1-sulfonyl azide (1.26 g, 4.072 mmol) in THF (5 mL) dropwise at −78° C. The resulting mixture was stirred for additional 10 min at −78° C. The reaction was quenched with water at 0° C. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with CH₂Cl₂/MeOH (10/1) to afford ethyl 7-azido-3-bromo-5H,6H-cyclopenta[b]pyridine-7-carboxylate (0.89 g, 77%) as a colorless oil. MS ESI calculated for C₁₁H₁₁BrN₄O₂ [M+H]⁺, 311.01 313.01 found 311.00 313.00. ¹H NMR (400 MHz, Chloroform-d) δ 8.53 (s, 1H), 7.78 (s, 1H), 4.37-4.23 (m, 2H), 3.15-2.99 (m, 2H), 2.80-2.73 (m, 1H), 2.29-2.22 (m, 1H), 1.30 (t, J=7.1 Hz, 3H).

### Preparation 36B: {7-amino-3-bromo-5H,6H-cyclopenta[b]pyridin-7-yl}methanol

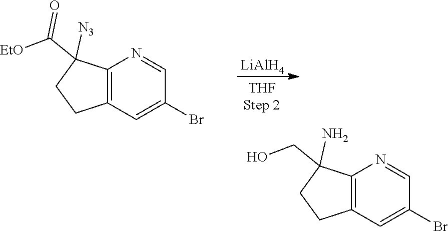 [SMILES: CCOC(=O)C1(N=[N+]=[N-])CCc2cc(Br)cnc21 ; "LiAlH4" ; NC1(CO)CCc2cc(Br)cnc21]

To a stirred solution of ethyl 7-azido-3-bromo-5H,6H-cyclopenta[b]pyridine-7-carboxylate (400 mg, 1.286 mmol) in THF (20 mL) was added LiAlH₄ (2.57 mL, 2.572 mmol, 1M in THF) dropwise at −10° C. under nitrogen atmosphere. The resulting mixture was stirred for 2 h at −10° C. under nitrogen atmosphere. The reaction was quenched with NaOH (0.6 mL, 15%) and H₂O (2.4 mL) at −10° C. The resulting mixture was extracted with EtOAc (3×30 mL). The combined organic layers were washed with brine (3×50 mL), dried over anhydrous Na₂SO₄. After filtration, the filtrate was concentrated under reduced pressure. The residue was purified by silica gel column chromatography, eluted with CH₂Cl₂/MeOH (5/1) to afford {7-amino-3-bromo-5H,6H-cyclopenta[b]pyridin-7-yl}methanol (69 mg, 22%) as a yellow oil. MS ESI calculated for C₉H₁₁BrN₂O [M+H]⁺, 243.01 245.01 found 243.05 245.05. ¹H NMR (400 MHz, Chloroform-d) δ 8.41 (s, 1H), 7.69 (s, 1H), 3.76-3.54 (m, 2H), 3.06-2.80 (m, 2H), 2.45-2.39 (m, 1H), 2.06-1.98 (m, 1H).

### Preparation 36C: (7R,14R)-11-(7-amino-7-(hydroxymethyl)-6,7-dihydro-5H-cyclopenta[b]pyridin-3-yl)-1-(difluoromethoxy)-6-methyl-6,7-dihydro-7,14-methanobenzo[f]benzo[4,5]imidazo[1,2-a][1,4]diazocin-5(14H)-one

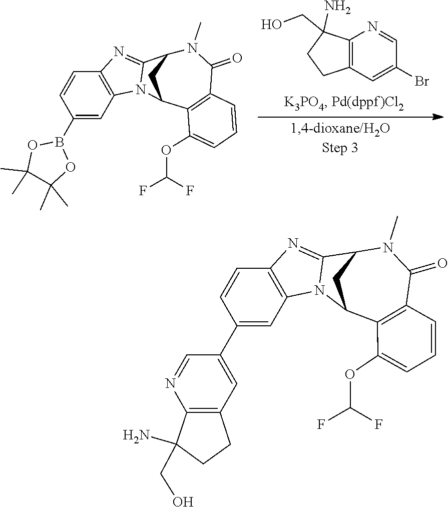 [SMILES: CN1C(=O)c2cccc(OC(F)F)c2[C@H]2C[C@@H]1c1nc3ccc(B4OC(C)(C)C(C)(C)O4)cc3n12 ; NC1(CO)CCc2cc(Br)cnc21 ; CN1C(=O)c2cccc(OC(F)F)c2[C@H]2C[C@@H]1c1nc3ccc(-c4cnc5c(c4)CCC5(N)CO)cc3n12]

A mixture of {7-amino-3-bromo-5H,6H-cyclopenta[b]pyridin-7-yl}methanol (48 mg, 0.199 mmol), (7R,14R)-1-(difluoromethoxy)-6-methyl-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (80 mg, 0.166 mmol), K₃PO₄ (105 mg, 0.498 mmol) and Pd(dppf)Cl₂ (13 mg, 0.017 mmol) in 1,4-dioxane (1 mL) and H₂O (0.2 mL) was stirred for 2 h at 80° C. under nitrogen atmosphere. The resulting mixture was concentrated under vacuum and purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-11-(7-amino-7-(hydroxymethyl)-6,7-dihydro-5H-cyclopenta[b]pyridin-3-yl)-1-(difluoromethoxy)-6-methyl-6,7-dihydro-7,14-methanobenzo[f]benzo[4,5]imidazo[1,2-a][1,4]diazocin-5(14H)-one (40 mg, 46%) as a white solid. MS ESI calculated for C₂₈H₂₅F₂N₅O₃ [M+H]⁺, 518.19, found 518.15.

### Example 36 and 37: (7R,14R)-11-((R)-7-amino-7-(hydroxymethyl)-6,7-dihydro-5H-cyclopenta[b]pyridin-3-yl)-1-(difluoromethoxy)-6-methyl-6,7-dihydro-7,14-methanobenzo[f]benzo[4,5]imidazo[1,2-a][1,4]diazocin-5(14H)-one and (7R,14R)-11-((S)-7-amino-7-(hydroxymethyl)-6,7-dihydro-5H-cyclopenta[b]pyridin-3-yl)-1-(difluoromethoxy)-6-methyl-6,7-dihydro-7,14-methanobenzo[f]benzo[4,5]imidazo[1,2-a][1,4]diazocin-5(14H)-one

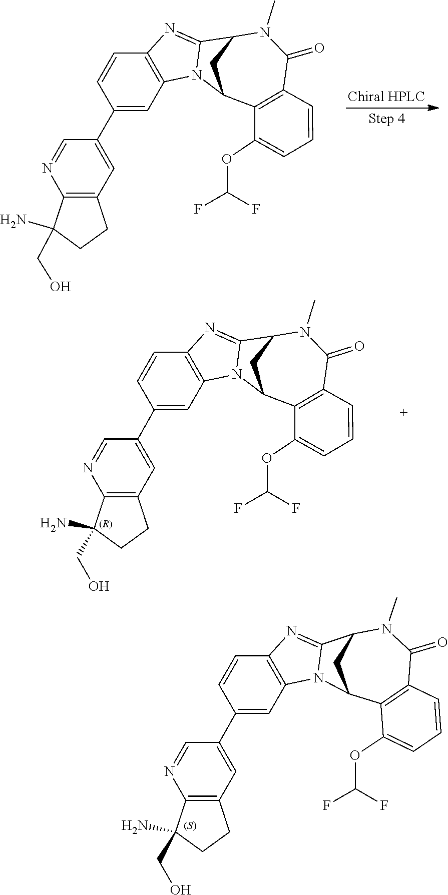

The mixture (40 mg) was resolved by Chiral-HPLC with the following conditions (Column: CHIRAL ART Cellulose-SB, 3*25 cm, 5 μm; Mobile Phase A: Hex (10 mM NH₃-MeOH), Mobile Phase B: EtOH; Flow rate: 40 mL/min; Gradient: isocratic 30; Wave Length: 200/230 nm; RT1(min): 17.86; RT2(min): 26.75). The first peak afforded (10 mg, 25%) as a white solid. MS ESI calculated for C₂₈H₂₅F₂N₅O₃ [M+H]⁺, 518.19, found 518.10. ¹H NMR (400 MHz, Chloroform-d) δ 8.55 (s, 1H), 8.47 (d, J=8.1 Hz, 1H), 7.77 (d, J=8.5 Hz, 1H), 7.70 (s, 1H), 7.58 (s, 1H), 7.42-7.36 (m, 2H), 7.29 (d, J=8.1 Hz, 1H), 6.85 (t, J=72.8 Hz, 1H), 6.24 (d, J=7.2 Hz, 1H), 4.95 (d, J=7.1 Hz, 1H), 3.84 (d, J=11.0 Hz, 1H), 3.77 (d, J=11.0 Hz, 1H), 3.52 (s, 3H), 3.50-3.43 (m, 1H), 3.12-3.05 (m, 1H), 2.99-2.91 (m, 1H), 2.86 (d, J=13.5 Hz, 1H), 2.46-2.39 (m, 1H), 2.16-2.08 (m, 1H). ¹⁹F NMR (377 MHz, Chloroform-d) δ −80.30, −80.75, −80.90, −81.35. The second peak afforded (10 mg, 25%) as a white solid. MS ESI calculated for C₂₈H₂₅F₂N₅O₃ [M+H]⁺, 518.19, found 518.15. ¹H NMR (400 MHz, Chloroform-d) δ 8.51 (s, 1H), 8.48 (d, J=8.1 Hz, 1H), 7.77 (d, J=8.5 Hz, 1H), 7.73 (s, 1H), 7.59 (s, 1H), 7.42-7.36 (m, 2H), 7.29 (d, J=8.1 Hz, 1H), 6.85 (t, J=72.8 Hz, 1H), 6.24 (d, J=7.2 Hz, 1H), 4.95 (d, J=7.1 Hz, 1H), 3.83 (d, J=11.0 Hz, 1H), 3.74 (d, J=11.0 Hz, 1H), 3.52 (s, 3H), 3.50-3.43 (m, 1H), 3.14-3.06 (m, 1H), 2.99-2.91 (m, 1H), 2.86 (d, J=13.5 Hz, 1H), 2.45-2.38 (m, 1H), 2.16-2.08 (m, 1H). ¹⁹F NMR (377 MHz, Chloroform-d) δ −80.23, −80.67, −80.86, −81.31.

### Example 38 and 39: (7R,14R)-1-(difluoromethoxy)-11-((R)-8-hydroxy-8-methyl-7,8-dihydro-5H-pyrano[4,3-b]pyridin-3-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((S)-8-hydroxy-8-methyl-7,8-dihydro-5H-pyrano[4,3-b]pyridin-3-yl)-6-(methyl-d3)-6,7-dihydro-7,14-methanobenzo[f]benzo[4,5]imidazo[1,2-a][1,4]diazocin-5(14H)-one

### Synthetic Scheme

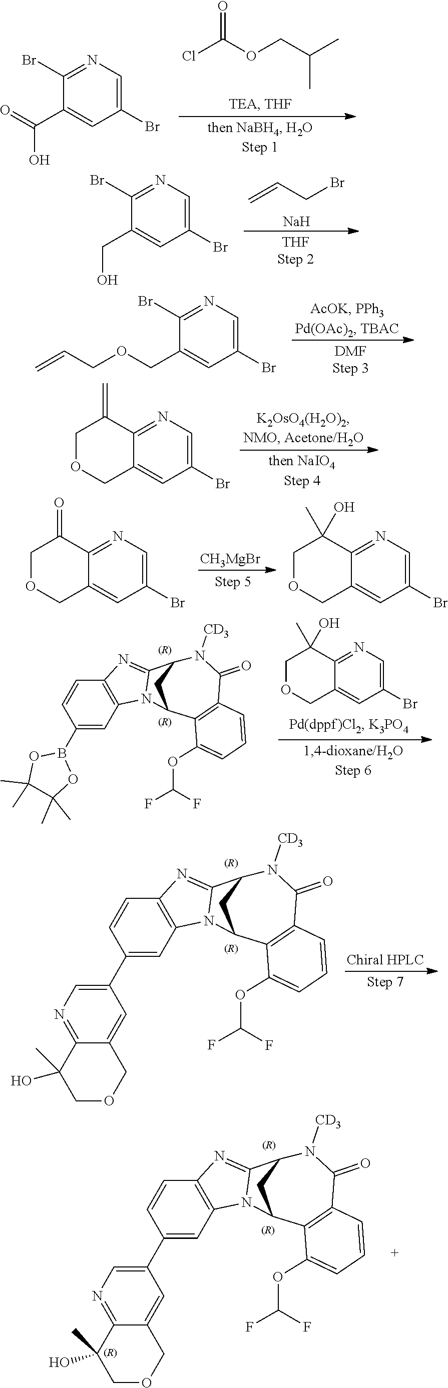

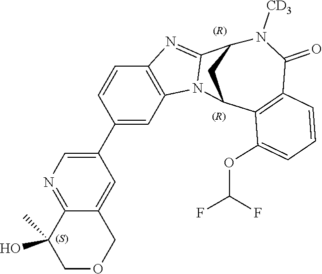

### Preparation 38A: (2,5-dibromopyridin-3-yl)methanol

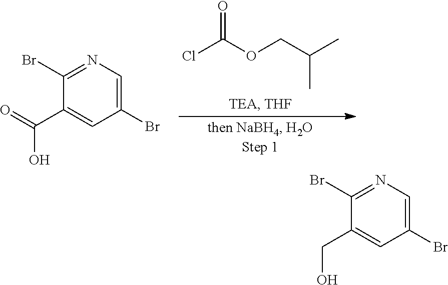

To a solution of 2,5-dibromopyridine-3-carboxylic acid (35.00 g, 124.598 mmol) and TEA (15.13 g, 149.518 mmol) in THF (350 mL) was added 2-methylpropyl carbonochloridate (20.42 g, 149.518 mmol) at 0° C. The mixture was stirred at 0° C. for 10 min and filtered. To this filtrate was added a suspension of NaBH₄ (9.43 g, 249.196 mmol) in water H₂O (35 mL) at 0° C. The mixture was stirred for 3 h at room temperature and concentrated to dryness. The residue was purified by silica gel column chromatography, eluted with CH₂Cl₂/MeOH (12/1) to afford (2,5-dibromopyridin-3-yl)methanol (27.01 g, 81%) as a white solid. MS ESI calculated for C₆H₅Br₂NO [M+H]⁺, 265.87 267.87 found 266.00 268.00. ¹H NMR (400 MHz, Chloroform-d) δ 8.34 (d, J=2.4 Hz, 1H), 8.04-7.98 (m, 1H), 4.72 (s, 2H), 2.51 (s, 1H).

### Preparation 38B: 2,5-dibromo-3-[(prop-2-en-1-yloxy)methyl]pyridine

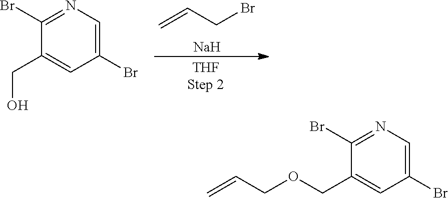

To a solution of (2,5-dibromopyridin-3-yl)methanol (27.01 g, 101.154 mmol) in THF (270 mL) was added NaH (6.07 g, 151.731 mmol, 60%)) at 0° C. The mixture was stirred for 15 min at 0° C. allyl bromide (14.68 g, 121.385 mmol) was added and the mixture was allowed to warm to RT and stirred for 1 h. The reaction mixture was quenched by water (500 mL and extracted with EA (3×500 mL). The combined organic layers were washed with brine (500 mL), dried over anhydrous Na₂SO₄. After filtration, the filtrate was concentrated under reduced pressure. The residue was purified by silica gel column chromatography, eluted with CH₂Cl₂/PE (5/1) to afford 2,5-dibromo-3-[(prop-2-en-1-yloxy)methyl]pyridine (25.00 g, 80%) as a colorless oil. MS ESI calculated for C₉H₉Br₂NO [M+H]⁺, 305.91 307.91, found 305.95 307.95. ¹H NMR (400 MHz, Chloroform-d) δ 8.35 (d, J=2.4 Hz, 1H), 7.96 (d, J=2.4 Hz, 1H), 6.05-5.90 (m, 1H), 5.43-5.33 (m, 1H), 5.32-5.24 (m, 1H), 4.50 (s, 2H), 4.121-4.11 (m, 2H).

### Preparation 38C: 3-bromo-8-methylidene-5H,7H-pyrano[4,3-b]pyridine

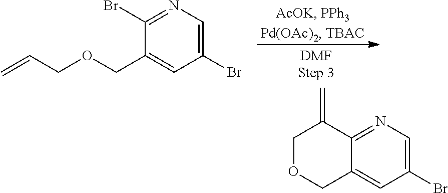

Into a 40 mL vial were added 2,5-dibromo-3-[(prop-2-en-1-yloxy)methyl]pyridine (1.00 g, 3.257 mmol), KOAc (1.60 g, 16.285 mmol), tetraethylazanium chloride (1.08 g, 6.514 mmol), PPh₃ (256 mg, 0.977 mmol), Pd(OAc)₂ (73 mg, 0.326 mmol) and DMF (10 mL) at room temperature. The mixture was purged with nitrogen for 5 min and then was stirred for overnight at 100° C. under nitrogen atmosphere. The mixture was allowed to cool down to room temperature and diluted with ethyl acetate (40 mL). The resulting mixture was filtered, the filter cake was washed with ethyl acetate (2×10 mL). The filtrate diluted with water (50 mL) and extracted with ethyl acetate (3×50 mL). The combined organic layers were washed with brine (3×150 mL), dried over anhydrous Na₂SO₄. After filtration, the filtrate was concentrated under reduced pressure. The residue was purified by silica gel column chromatography, eluted with CH₂Cl₂/PE (5/1) to afford 3-bromo-8-methylidene-5H,7H-pyrano[4,3-b]pyridine (195 mg, 26%) as a white solid. MS ESI calculated for C₉H₈BrNO [M+H]⁺, 225.98 227.98 found 226.10 228.10. ¹H NMR (400 MHz, Chloroform-d) δ 8.55 (d, J=2.4 Hz, 1H), 7.54-7.49 (m, 1H), 6.31 (s, 1H), 5.21 (q, J=1.6 Hz, 1H), 4.79 (s, 2H), 4.56 (t, J=1.6 Hz, 2H).

### Preparation 38D: 3-bromo-5H,7H-pyrano[4,3-b]pyridin-8-one

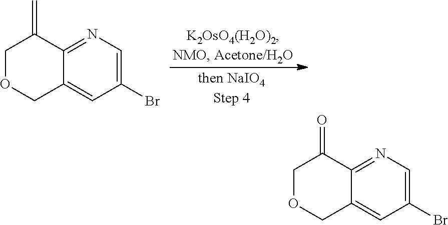

To a solution of 3-bromo-8-methylidene-5H,7H-pyrano[4,3-b]pyridine (1.00 g, 4.423 mmol) and NMO (621 mg, 5.308 mmol) in acetone (16 mL) and water H₂O (2 mL) was added K₂OsO₄·2H₂O (33 mg, 0.088 mmol) at room temperature. This mixture was stirred for 6 h at room temperature before NaIO₄ (1.89 g, 8.846 mmol) was added. This mixture was stirred for 18 h at room temperature, and then EtOAc (150 mL) and saturated sodium thiosulfate (100 mL) were added. This mixture was stirred vigorously for 5 min and then the layers were separated. The organic layer was then dried over anhydrous MgSO4, filtered, and concentrated in vacuum. The residue was purified by silica gel column chromatography, eluted with PE/EA (5/1) to afford 3-bromo-5H,7H-pyrano[4,3-b]pyridin-8-one (700 mg, 69%) as a yellow solid. MS ESI calculated for C₈H₆BrNO₂ [M+H]⁺, 227.96 229.96 found 228.05 230.05. ¹H NMR (400 MHz, Chloroform-d) δ 8.84 (d, J=2.4 Hz, 1H), 7.82 (d, J=2.4 Hz, 1H), 4.95 (s, 2H), 4.50 (s, 2H).

### Preparation 38E: 3-bromo-8-methyl-5H,7H-pyrano[4,3-b]pyridin-8-ol

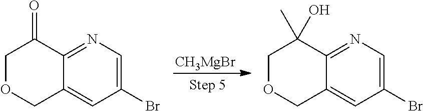

To a stirred solution of 3-bromo-5H,7H-pyrano[4,3-b]pyridin-8-one (110 mg, 0.482 mmol) in THF (5 mL) was added CH₃MgBr (0.24 mL, 0.723 mmol, 3M in THF) dropwise portions at 0° C. The resulting mixture was stirred for 1 h at 0° C. under nitrogen atmosphere. The reaction was quenched with 15 mL sat. NH₄Cl (aq.) at room temperature and extracted with EtOAc (3×15 mL). The combined organic layers were washed with brine (3×20 mL), dried over anhydrous Na₂SO₄. After filtration, the filtrate was concentrated under reduced pressure to afford 3-bromo-8-methyl-5H,7H-pyrano[4,3-b]pyridin-8-ol (95 mg, 80%) as a yellow solid. MS ESI calculated for C₉H₁₀BrNO₂ [M+H]⁺, 243.99 245.99 found 243.70 245.70. ¹H NMR (400 MHz, Chloroform-d) δ 8.59 (d, J=2.0 Hz, 1H), 7.62 (s, 1H), 4.81 (s, 2H), 3.93 (d, J=11.2 Hz, 1H), 3.85 (d, J=11.6 Hz, 1H), 1.65 (s, 3H).

### Preparation 38F: (7R,14R)-1-(difluoromethoxy)-11-(8-hydroxy-8-methyl-7,8-dihydro-5H-pyrano[4,3-b]pyridin-3-yl)-6-(methyl-d3)-6,7-dihydro-7,14-methanobenzo[f]benzo[4,5]imidazo[1,2-a][1,4]diazocin-5(14H)-one

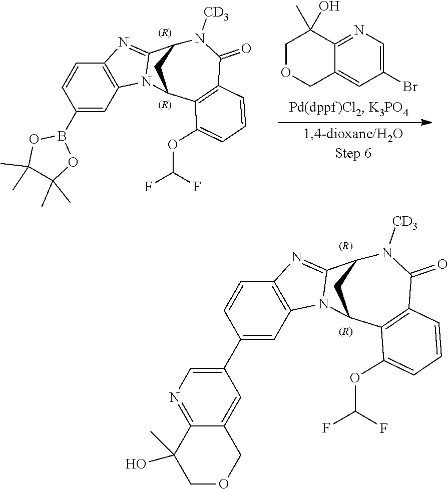

To a solution of (7R,14R)-1-(difluoromethoxy)-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (160 mg, 0.330 mmol) and 3-bromo-8-methyl-5H,7H-pyrano[4,3-b]pyridin-8-ol (96 mg, 0.396 mmol) in 1,4-dioxane (3 mL) and H₂O (0.6 mL) were added K₃PO₄ (210 mg, 0.990 mmol) and Pd(dppf)Cl₂ (27 mg, 0.033 mmol) at room temperature. After stirring for 2 h at 80° C. under a nitrogen atmosphere, the resulting mixture was concentrated under reduced pressure and purified by silica gel column chromatography, eluted with DCM/MeOH (0% to 10%) followed by Prep-HPLC with the following conditions: column, C18 silica gel; mobile phase, CH₃CN in water (10 mmol/L NH₄HCO₃), 35% to 70% gradient in 25 min; detector, 254 nm to afford (7R,14R)-1-(difluoromethoxy)-11-(8-hydroxy-8-methyl-7,8-dihydro-5H-pyrano[4,3-b]pyridin-3-yl)-6-(methyl-d3)-6,7-dihydro-7,14-methanobenzo[f]benzo[4,5]imidazo[1,2-a][1,4]diazocin-5(14H)-one (80 mg, 46%) as a white solid. MS ESI calculated for C₂₈H₂₁D₃F₂N₄O₄ [M+H]⁺, 522.20, found 522.20.

### Example 38 and 39: (7R,14R)-1-(difluoromethoxy)-11-((R)-8-hydroxy-8-methyl-7,8-dihydro-5H-pyrano[4,3-b]pyridin-3-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((S)-8-hydroxy-8-methyl-7,8-dihydro-5H-pyrano[4,3-b]pyridin-3-yl)-6-(methyl-d3)-6,7-dihydro-7,14-methanobenzo[f]benzo[4,5]imidazo[1,2-a][1,4]diazocin-5(14H)-one

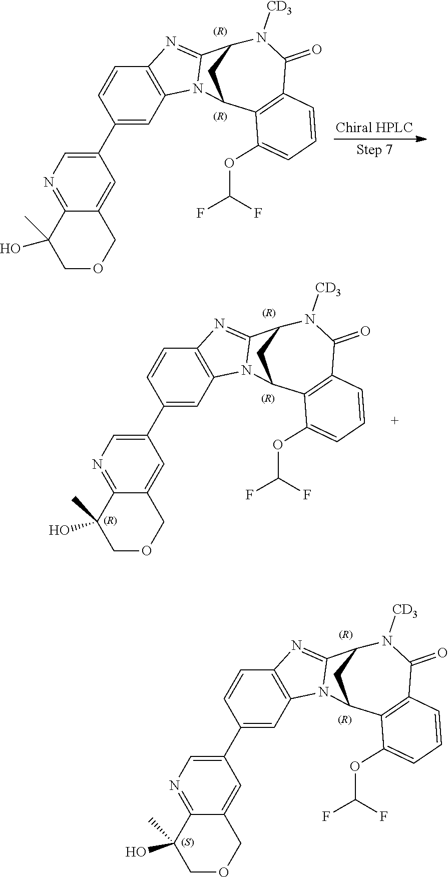

The mixture (77 mg) was purified by Prep-Chiral HPLC with the following conditions (Column: CHIRAL ART Cellulose-SB, 2*25 cm, 5 μm; Mobile Phase A: Hex (10 mM NH₃-MeOH), Mobile Phase B: EtOH; Flow rate: 20 mL/min; Gradient: isocratic 50; Wave Length: 206/232 nm; RT1(min): 11.05; RT2(min): 16.34). The first peak afford 25 mg (31%) as a white solid. MS ESI calculated for C₂₈H₂₁D₃F₂N₄O₄ [M+H]⁺, 522.20 found 522.20. ¹H NMR (400 MHz, Chloroform-d) δ 8.75 (d, J=2.0 Hz, 1H), 8.53-8.45 (m, 1H), 7.81 (d, J=8.4 Hz, 1H), 7.68 (d, J=1.6 Hz, 1H), 7.59 (d, J=2.0 Hz, 1H), 7.46-7.38 (m, 2H), 7.31 (d, J=8.0 Hz, 1H), 6.86 (t, J=72.8 Hz, 1H), 6.29 (d, J=7.2 Hz, 1H), 4.98 (d, J=7.2 Hz, 1H), 4.89 (s, 2H), 4.01-3.86 (m, 2H), 3.55-3.42 (m, 1H), 2.90 (d, J=13.6 Hz, 1H), 1.71 (s, 3H). ¹⁹F NMR (376 MHz, Chloroform-d) δ −80.79. The second peak afford 26 mg (33%) as a white solid. MS ESI calculated for C₂₈H₂₁D₃F₂N₄O₄ [M+H]⁺, 522.20 found 522.25 ¹H NMR (400 MHz, Chloroform-d) δ 8.74 (d, J=2.0 Hz, 1H), 8.53-8.45 (m, 1H), 7.82 (d, J=8.4 Hz, 1H), 7.73-7.63 (m, 2H), 7.46-7.38 (m, 2H), 7.32 (d, J=8.0 Hz, 1H), 6.88 (t, J=72.8 Hz, 1H), 6.29 (d, J=7.2 Hz, 1H), 5.00 (d, J=7.2 Hz, 1H), 4.91 (s, 2H), 4.02-3.86 (m, 2H), 3.55-3.42 (m, 1H), 2.91 (d, J=13.6 Hz, 1H), 1.75 (s, 3H). ¹⁹F NMR (376 MHz, Chloroform-d) δ −80.35, −80.80, −80.83, −81.28.

### Example 40 and 41: (7R,14R)-1-(difluoromethoxy)-11-((R)-8-ethyl-8-hydroxy-7,8-dihydro-5H-pyrano[4,3-b]pyridin-3-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((S)-8-ethyl-8-hydroxy-7,8-dihydro-5H-pyrano[4,3-b]pyridin-3-yl)-6-(methyl-d3)-6,7-dihydro-7,14-methanobenzo[f]benzo[4,5]imidazo[1,2-a][1,4]diazocin-5(14H)-one

### Synthetic Scheme

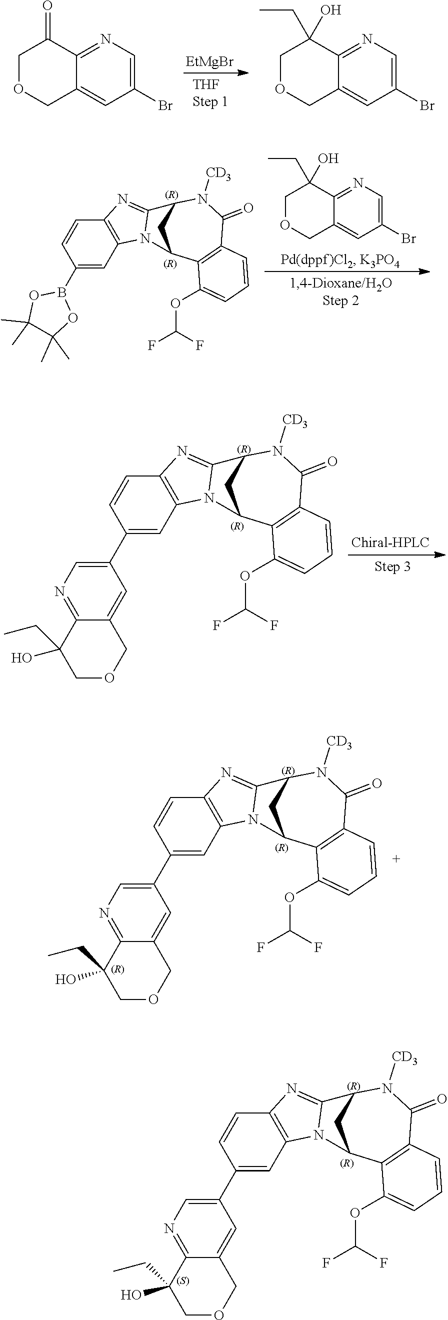

### Preparation 40A: 3-bromo-8-ethyl-5H,7H-pyrano[4,3-b]pyridin-8-ol

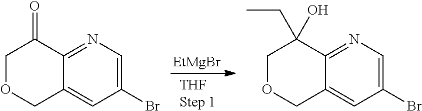

To a stirred solution of 3-bromo-5H,7H-pyrano[4,3-b]pyridin-8-one (150 mg, 0.658 mmol) in THF (3 mL) was added EtMgBr (0.33 mL, 0.987 mmol, 3 M in THF) dropwise at 0° C. under nitrogen atmosphere. The resulting mixture was stirred for 2 h at room temperature. The reaction was quenched with sat. NH₄Cl (aq.) at 0° C. and was extracted with EtOAc (3×20 mL). The combined organic layers were washed with brine (3×30 mL), dried over anhydrous Na₂SO₄. After filtration, the filtrate was concentrated under reduced pressure. The residue was purified by reversed-phase flash chromatography with the following conditions: column, C18 silica gel; mobile phase, CH₃CN in Water (0.1% FA), 10% to 60% gradient in 25 min; detector, 254 nm to afford 3-bromo-8-ethyl-5H,7H-pyrano[4,3-b]pyridin-8-ol (50 mg, 29%) as a yellow solid. MS ESI calculated for C₁₀H₁₂BrNO₂ [M+H]⁺, 258.01 260.01 found 258.00 260.00.

### Preparation 40B: (7R,14R)-1-(difluoromethoxy)-11-(8-ethyl-8-hydroxy-7,8-dihydro-5H-pyrano[4,3-b]pyridin-3-yl)-6-(methyl-d3)-6,7-dihydro-7,14-methanobenzo[f]benzo[4,5]imidazo[1,2-a][1,4]diazocin-5(14H)-one

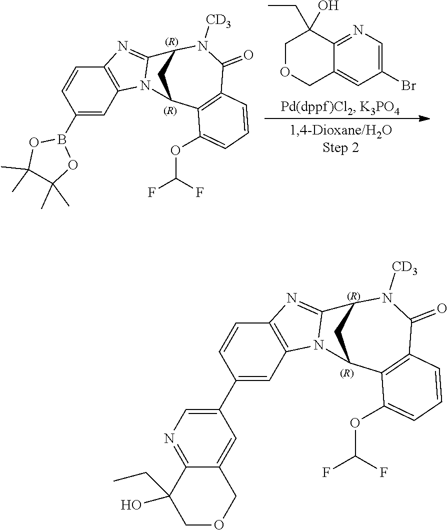

To a solution of (7R,14R)-1-(difluoromethoxy)-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (95 mg, 0.196 mmol) and 3-bromo-8-ethyl-5H,7H-pyrano[4,3-b]pyridin-8-ol (50 mg, 0.196 mmol) in 1,4-dioxane (3 mL) and H₂O (0.5 mL) were added K₃PO₄ (125 mg, 0.588 mmol) and Pd(dppf)Cl₂ (16 mg, 0.020 mmol) at room temperature. After stirring for 2 h at 80° C. under a nitrogen atmosphere, the resulting mixture was concentrated under reduced pressure. The residue was purified by silica gel column chromatography, eluted with DCM/MeOH (0% to 10%) followed by Prep-HPLC with the following conditions: column, C18 silica gel; mobile phase, CH₃CN in water (10 mmol/L NH₄HCO₃), 35% to 70% gradient in 25 min; detector, 254 nm to afford (7R,14R)-1-(difluoromethoxy)-11-(8-ethyl-8-hydroxy-7,8-dihydro-5H-pyrano[4,3-b]pyridin-3-yl)-6-(methyl-d3)-6,7-dihydro-7,14-methanobenzo[f]benzo[4,5]imidazo[1,2-a][1,4]diazocin-5(14H)-one (50 mg, 47%) as a white solid. MS ESI calculated for C₂₉H₂₃D₃F₂N₄O₄ [M+H]⁺, 536.21, found 536.30.

### Example 40 and 41: (7R,14R)-1-(difluoromethoxy)-11-((R)-8-ethyl-8-hydroxy-7,8-dihydro-5H-pyrano[4,3-b]pyridin-3-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((S)-8-ethyl-8-hydroxy-7,8-dihydro-5H-pyrano[4,3-b]pyridin-3-yl)-6-(methyl-d3)-6,7-dihydro-7,14-methanobenzo[f]benzo[4,5]imidazo[1,2-a][1,4]diazocin-5(14H)-one

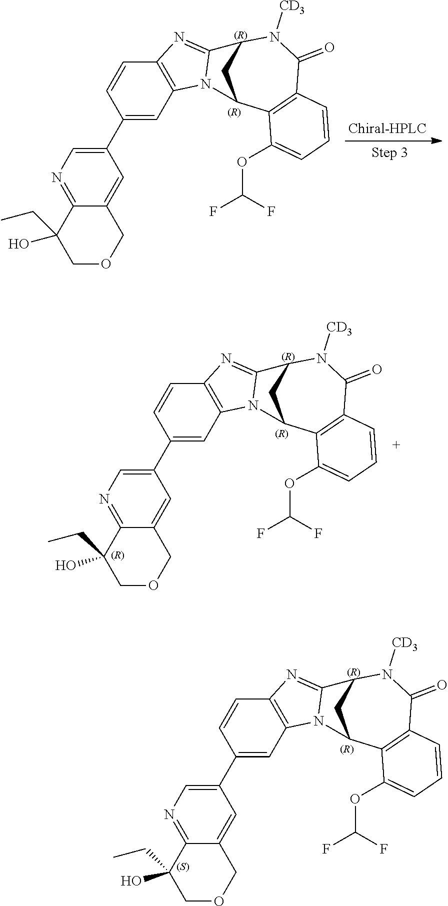

The mixture (50 mg) was purified by Prep-Chiral HPLC with the following conditions (Column: CHIRAL ART Cellulose-SB, 2*25 cm, 5 μm; Mobile Phase A: Hex (10 mM NH₃-MeOH), Mobile Phase B: EtOH; Flow rate: 20 mL/min; Gradient: isocratic 50; Wave Length: 206/232 nm; RT1(min): 11.05; RT2(min): 16.34). The first peak afforded 17 mg (33%) as a white solid. MS ESI calculated for C₂₉H₂₃D₃F₂N₄O₄ [M+H]⁺, 536.21 found 536.25. ¹H NMR (400 MHz, Chloroform-d) δ 8.75 (d, J=2.1 Hz, 1H), 8.53-8.40 (m, 1H), 7.80 (t, J=6.4 Hz, 1H), 7.69 (d, J=1.7 Hz, 1H), 7.62 (s, 1H), 7.46-7.38 (m, 2H), 7.31 (d, J=8.1 Hz, 1H), 6.87 (t, J=72.9 Hz, 1H), 6.29 (d, J=7.2 Hz, 1H), 4.99 (d, J=7.1 Hz, 1H), 4.89 (s, 2H), 4.14 (d, J=11.5 Hz, 1H), 3.79 (d, J=11.6 Hz, 1H), 3.54-3.38 (m, 1H), 2.90 (d, J=13.6 Hz, 1H), 2.13-2.01 (m, 2H), 1.03 (t, J=7.5 Hz, 3H). ¹⁹F NMR (376 MHz, Chloroform-d) δ −80.82. The second peak afforded 15 mg (29%) as a white solid. MS ESI calculated for C₂₉H₂₃D₃F₂N₄O₄ [M+H]⁺, 536.21 found 536.20. ¹H NMR (400 MHz, Chloroform-d) δ 8.78-8.66 (m, 1H), 8.53-8.44 (m, 1H), 7.79 (d, J=8.5 Hz, 1H), 7.68 (s, 1H), 7.54 (d, J=2.2 Hz, 1H), 7.47-7.37 (m, 2H), 7.31 (d, J=8.1 Hz, 1H), 6.86 (t, J=72.9 Hz, 1H), 6.28 (d, J=7.2 Hz, 1H), 4.98 (d, J=7.1 Hz, 1H), 4.88 (s, 2H), 4.12 (d, J=11.5 Hz, 1H), 3.82 (d, J=11.5 Hz, 1H), 3.57-3.43 (m, 1H), 2.89 (d, J=13.6 Hz, 1H), 2.06-1.95 (m, 2H), 1.01 (t, J=7.5 Hz, 3H). ¹⁹F NMR (376 MHz, Chloroform-d) 6-80.78.

### Example 42 and 43: (7R,14R)-1-(difluoromethoxy)-11-((R)-3-hydroxy-3-methyl-2-oxo-2,3-dihydro-1H-pyrrolo[3,2-b]pyridin-6-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((S)-3-hydroxy-3-methyl-2-oxo-2,3-dihydro-1H-pyrrolo[3,2-b]pyridin-6-yl)-6-(methyl-d3)-6,7-dihydro-7,14-methanobenzo[f]benzo[4,5]imidazo[1,2-a][1,4]diazocin-5(14H)-one

### Synthetic Scheme

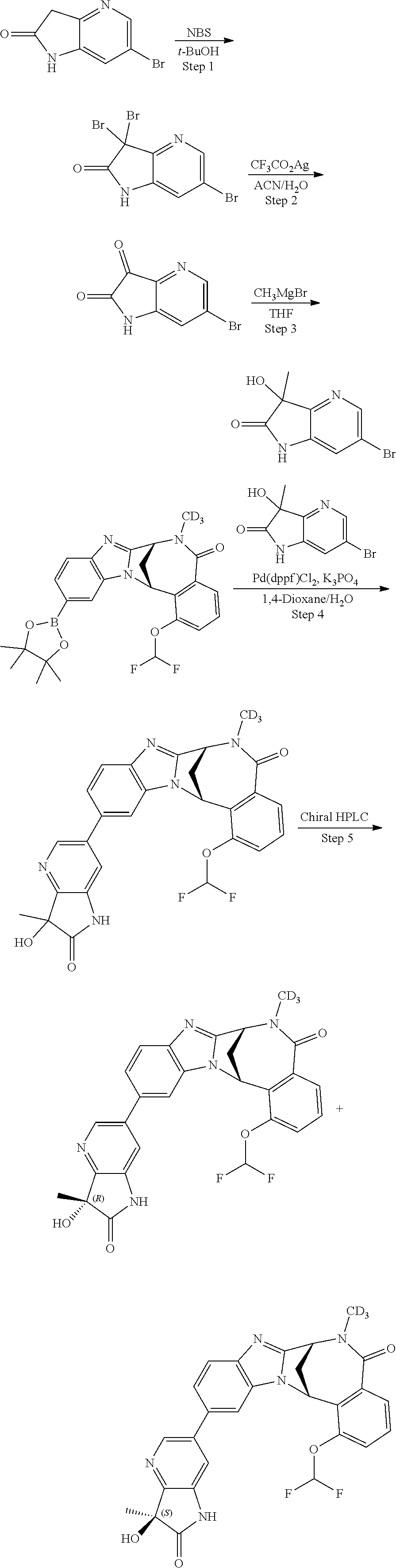

### Preparation 42A: 3,3,6-tribromo-1,3-dihydro-2H-pyrrolo[3,2-b]pyridin-2-one

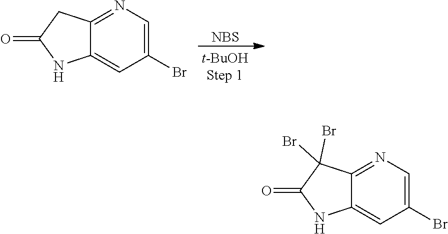 [SMILES: O=C1Cc2ncc(Br)cc2N1 ; "NBS" ; O=C1Nc2cc(Br)cnc2C1(Br)Br]

To a stirred solution of 6-bromo-1,3-dihydro-2H-pyrrolo[3,2-b]pyridin-2-one (5.00 g, 23.470 mmol) in t-BuOH (50 mL) was added NBS (8.35 g, 46.940 mmol) in portions at room temperature. The resulting mixture was stirred for 4 h at room temperature. The resulting mixture was concentrated under reduced pressure. The residue was purified by silica gel column chromatography, eluted with PE/EA (0%˜50%) to afford 3,3,6-tribromo-1,3-dihydro-2H-pyrrolo[3,2-b]pyridin-2-one (4.66 g, 53%) as a brown solid. MS ESI calculated for C₇H₃Br₃N₂O [M+H]⁺, 368.78 370.78 372.78 374.78 found 368.85 370.85 372.85 374.80. H NMR (400 MHz, DMSO-d₆) δ 11.64 (s, 1H), 8.44 (d, J=1.9 Hz, 1H), 7.60 (d, J=1.9 Hz, 1H).

### Preparation 42B: 6-bromo-1H-pyrrolo[3,2-b]pyridine-2,3-dione

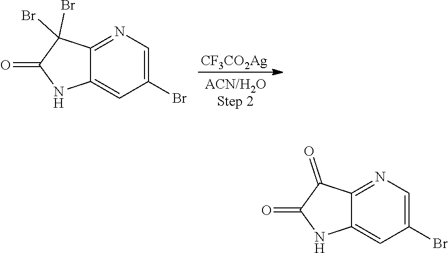

To a stirred solution of 3,3,6-tribromo-1,3-dihydro-2H-pyrrolo[3,2-b]pyridin-2-one (4.56 g, 12.297 mmol) in ACN (182 mL) and H₂O (14 mL) was added CF₃CO₂Ag (5.43 g, 24.594 mmol) at room temperature. The resulting mixture was stirred for 1 h at 80° C. The resulting mixture was concentrated under reduced pressure and purified by silica gel column chromatography, eluted with PE/EA (0%˜50%) to afford 6-bromo-1H-pyrrolo[3,2-b]pyridine-2,3-dione (2.20 g, 68%) as an orange solid. MS ESI calculated for C₇H₃BrN₂O₂ [M+H]⁺, 226.94 228.94 found 227.10 229.10. ¹H NMR (400 MHz, DMSO-d₆) δ 11.21 (s, 1H), 8.43 (d, J=1.9 Hz, 1H), 7.59 (d, J=1.9 Hz, 1H).

### Preparation 42C: 6-bromo-3-hydroxy-3-methyl-1,3-dihydro-2H-pyrrolo[3,2-b]pyridin-2-one

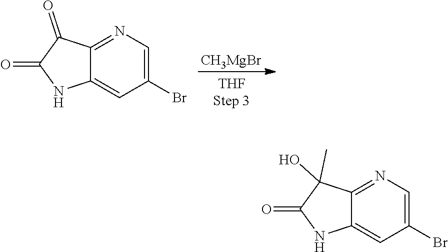

To a stirred solution of 6-bromo-1H-pyrrolo[3,2-b]pyridine-2,3-dione (2.10 g, 9.250 mmol) in THF (100 mL) was added CH₃MgBr (6.17 mL, 18.500 mmol, 3 M in THF) dropwise at 0° C. under nitrogen atmosphere. The resulting mixture was stirred for overnight at room temperature under nitrogen atmosphere. The reaction was quenched by the addition of sat. NH₄Cl (aq.) (50 mL) at 0° C. The resulting mixture was extracted with EtOAc (3×100 mL). The combined organic layers were dried over anhydrous Na₂SO₄. After filtration, the filtrate was concentrated under reduced pressure. The residue was purified by silica gel column chromatography, eluted with CH₂Cl₂/MeOH (0% to 10%) to afford 6-bromo-3-hydroxy-3-methyl-1,3-dihydro-2H-pyrrolo[3,2-b]pyridin-2-one (1.22 g, 54%) as an orange solid. MS ESI calculated for C₈H₇BrN₂O₂ [M+H]⁺, 242.97 244.97 found 243.05 245.05. ¹H NMR (400 MHz, DMSO-d₆) δ 10.58 (s, 1H), 8.23 (d, J=2.0 Hz, 1H), 7.36 (d, J=1.9 Hz, 1H), 6.09 (s, 1H).

### Preparation 42D: (7R,14R)-1-(difluoromethoxy)-11-(3-hydroxy-3-methyl-2-oxo-2,3-dihydro-1H-pyrrolo[3,2-b]pyridin-6-yl)-6-(methyl-d3)-6,7-dihydro-7,14-methanobenzo[f]benzo[4,5]imidazo[1,2-a][1,4]diazocin-5(14H)-one

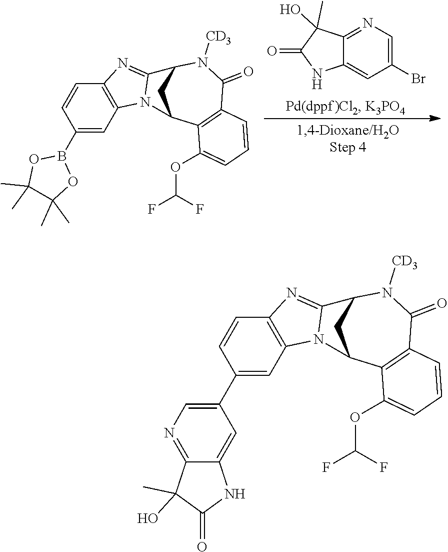

To a stirred solution of (7R,14R)-1-(difluoromethoxy)-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (90 mg, 0.186 mmol) and 6-bromo-3-hydroxy-3-methyl-1,3-dihydro-2H-pyrrolo[3,2-b]pyridin-2-one (54 mg, 0.223 mmol) in 1,4-dioxane (0.8 mL) and H₂O (0.2 mL) were added K₃PO₄ (118 mg, 0.558 mmol) and Pd(dppf)Cl₂ (15 mg, 0.019 mmol) at room temperature. The resulting mixture was stirred for 2 h at 80° C. under nitrogen atmosphere.

The resulting mixture was concentrated under reduced pressure and purified by silica gel column chromatography, eluted with CH₂Cl₂/MeOH (0% to 10%) followed by Prep-HPLC with the following conditions: C18 Column 120 g; Mobile Phase A: Water (10 mmol/L NH₄HCO₃), Mobile Phase B: CH₃CN; Flow rate: 50 mL/min; Gradient: 20 B to 40 B in 40 min; 254/220 nm to afford (7R,14R)-1-(difluoromethoxy)-11-(3-hydroxy-3-methyl-2-oxo-2,3-dihydro-1H-pyrrolo[3,2-b]pyridin-6-yl)-6-(methyl-d3)-6,7-dihydro-7,14-methanobenzo[f]benzo[4,5]imidazo[1,2-a][1,4]diazocin-5(14H)-one (50 mg, 51%) as a white solid. MS ESI calculated for C₂₇H₁₈D₃F₂N₅O₄ [M+H]⁺, 521.17, found 521.25.

### Example 42 and 43: (7R,14R)-1-(difluoromethoxy)-11-((R)-3-hydroxy-3-methyl-2-oxo-2,3-dihydro-1H-pyrrolo[3,2-b]pyridin-6-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((S)-3-hydroxy-3-methyl-2-oxo-2,3-dihydro-1H-pyrrolo[3,2-b]pyridin-6-yl)-6-(methyl-d3)-6,7-dihydro-7,14-methanobenzo[f]benzo[4,5]imidazo[1,2-a][1,4]diazocin-5(14H)-one

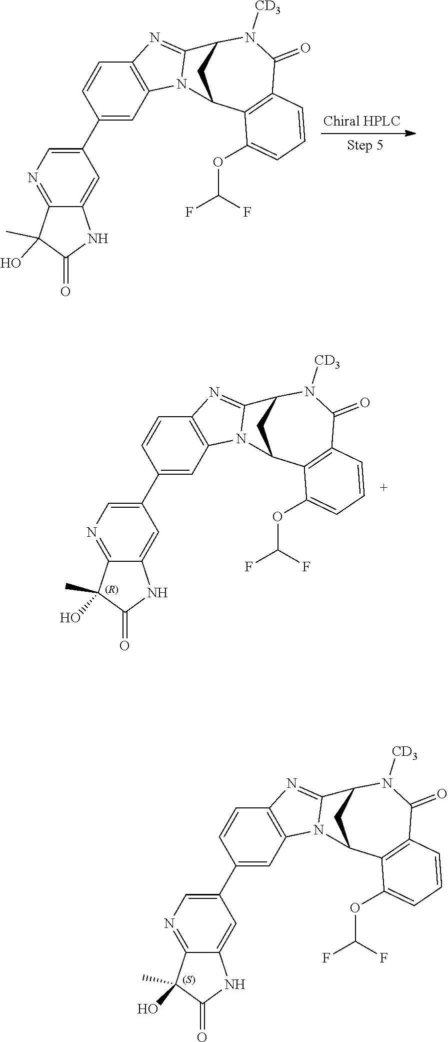

The mixture (50 mg) was purified by Prep-Chiral HPLC with the following conditions (Column: CHIRAL ART Cellulose-SB, 3*25 cm, 5 μm; Mobile Phase A: Hex (10 mM NH₃-MeOH), Mobile Phase B: EtOH; Flow rate: 40 mL/min; Gradient: isocratic 50; Wave Length: 220/230 nm; RT (min): 10.2; RT (min): 17.6). The first peak afforded (14 mg, 27%) as a white solid. MS ESI calculated for C₂₇H₁₈D₃F₂N₅O₄ [M+H]⁺, 521.17, found 521.25. ¹H NMR (400 MHz, DMSO-d₆) δ 10.57 (s, 1H), 8.35 (d, J=1.9 Hz, 1H), 8.29-8.26 (m, 1H), 7.82-7.62 (m, 3H), 7.52-7.43 (m, 3H), 7.31 (d, J=2.2 Hz, 1H), 6.30 (d, J=7.0 Hz, 1H), 6.09-6.00 (m, 1H), 5.25 (d, J=7.2 Hz, 1H), 3.54-3.50 (m, 1H), 2.83 (d, J=13.7 Hz, 1H), 1.42 (s, 3H). ¹⁹F NMR (376 MHz, DMSO-d₆) δ −81.41, −81.86, −81.92, −82.37. The second peak afforded (14 mg, 28%) as a white solid. MS ESI calculated for C₂₇H₁₈D₃F₂N₅O₄ [M+H]⁺, 521.17, found 521.20. ¹H NMR (400 MHz, DMSO-d₆) δ 10.57 (s, 1H), 8.35 (d, J=1.9 Hz, 1H), 8.29-8.26 (m, 1H), 7.84-7.62 (m, 3H), 7.52-7.45 (m, 3H), 7.31 (d, J=1.9 Hz, 1H), 6.30 (d, J=7.1 Hz, 1H), 6.04 (s, 1H), 5.24 (d, J=7.2 Hz, 1H), 3.54-3.50 (m, 1H), 2.83 (d, J=13.7 Hz, 1H), 1.41 (s, 3H). ¹⁹F NMR (377 MHz, DMSO-d₆) δ −81.40, −81.85, −81.91, −82.36.

### Example 44: (7R,14R)-1-(difluoromethoxy)-11-(3-hydroxy-3-methyl-2,3-dihydrofuro[3,2-b]pyridin-6-yl)-6-(methyl-d3)-6,7-dihydro-7,14-methanobenzo[f]benzo[4,5]imidazo[1,2-a][1,4]diazocin-5(14H)-one

### Synthetic Scheme

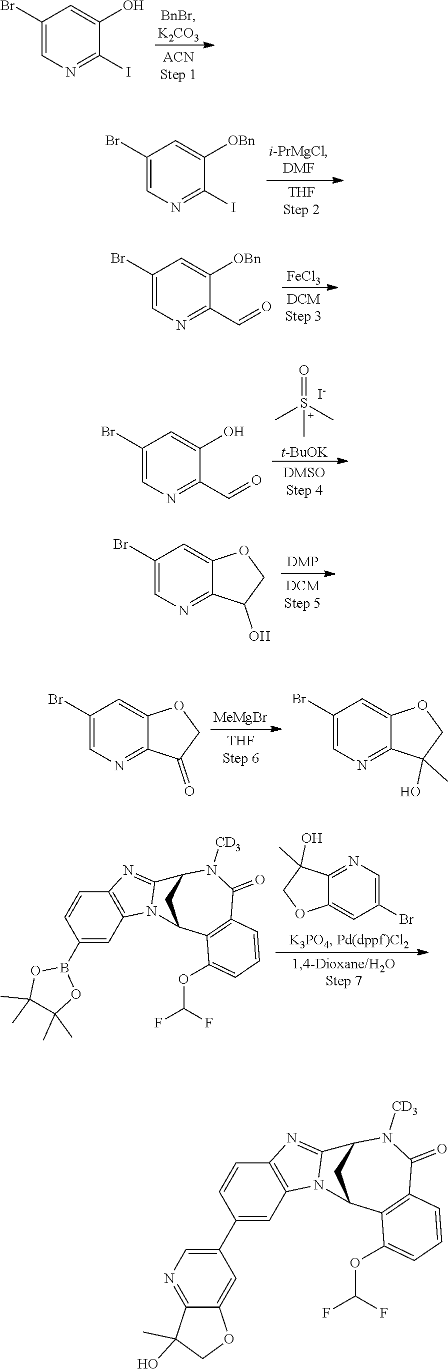

### Preparation 44A: 3-(benzyloxy)-5-bromo-2-iodopyridine

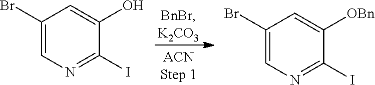 [SMILES: Oc1cc(Br)cnc1I ; Brc1cnc(I)c(OCc2ccccc2)c1]

To a solution of 5-bromo-2-iodopyridin-3-ol (20.00 g, 66.690 mmol) and K₂CO₃ (27.65 g, 200.070 mmol) in ACN (300 mL) was added benzyl bromide (11.98 g, 70.025 mmol) dropwise at 0° C. The resulting mixture was stirred for overnight at room temperature and then cooled down to 0° C., quenched with water at 0° C. The solids were collected by filtration and triturated with 5% EA in PE (500 mL) to afford 3-(benzyloxy)-5-bromo-2-iodopyridine (24.00 g, 92%) as a brown solid. MS ESI calculated for C₁₂H₉BrINO [M+H]⁺, 389.89 391.89 found 389.75 391.75. ¹H NMR (400 MHz, Chloroform-d) δ 8.10 (s, 1H), 7.48-7.34 (m, 5H), 7.15 (s, 1H), 5.15 (s, 2H).

### Preparation 44B: 3-(benzyloxy)-5-bromopyridine-2-carbaldehyde

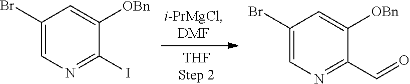 [SMILES: Brc1cnc(I)c(OCc2ccccc2)c1 ; O=Cc1ncc(Br)cc1OCc1ccccc1]

To a solution of 3-(benzyloxy)-5-bromo-2-iodopyridine (24.00 g, 61.536 mmol) in THF (500 mL) cooled to −20° C. was added chloro(propan-2-yl)magnesium; chlorolithium (52.07 mL, 67.690 mmol, 1.3 M in THF) dropwise. The mixture was stirred at −20° C. for 2 h and DMF (6.75 g, 92.304 mmol) was added. The mixture was stirred for 2 h at room temperature, cooled down to −20° C., and quenched with aqueous NH₄Cl solution. The resulting solution was extracted with EA (500 mL) twice. The combined organic layers were washed with brine (500 mL) twice, dried over anhydrous sodium sulfate, concentrated under reduced pressure, and purified by silica gel chromatography (EA/PE, 1/10) to give 3-(benzyloxy)-5-bromopyridine-2-carbaldehyde (10.30 g, 57%) as a brown solid. MS ESI calculated for C₁₃H₁₀BrNO₂ [M+H]⁺, 291.99 293.99, found 292.00 294.00. ¹H NMR (300 MHz, Chloroform-d) δ 10.37 (s, 1H), 8.48 (s, 1H), 7.65 (d, J=1.7 Hz, 1H), 7.52-7.37 (m, 5H), 5.26 (s, 2H).

### Preparation 44C: 5-bromo-3-hydroxypyridine-2-carbaldehyde

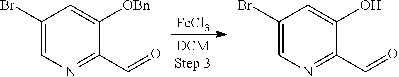

A solution of 3-(benzyloxy)-5-bromopyridine-2-carbaldehyde (10.30 g, 35.258 mmol) and FeCl₃ (11.44 g, 70.516 mmol) in DCM (60 mL) was stirred for overnight at room temperature. The reaction was quenched with water at room temperature. The resulting mixture was filtered, the filter cake was washed with CH₂Cl₂ (3×100 mL). The filtrate was concentrated under reduced pressure. The residue was purified by silica gel column chromatography, eluted with PE/EA (2/1) to afford 5-bromo-3-hydroxypyridine-2-carbaldehyde (4.60 g, 64%) as a yellow solid. MS ESI calculated for C₆H₄BrNO₂ [M+H]⁺, 201.94 203.94, found 201.85 203.85. ¹H NMR (400 MHz, Chloroform-d) δ 10.81 (s, 1H), 10.03 (s, 1H), 8.38 (d, J=1.8 Hz, 1H), 7.58 (d, J=1.5 Hz, 1H).

### Preparation 44D: 6-bromo-2H,3H-furo[3,2-b]pyridin-3-ol

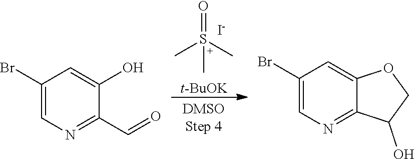

To a solution of 5-bromo-3-hydroxypyridine-2-carbaldehyde (4.00 g, 19.801 mmol) in DMSO (50 mL) were added iodotrimethyl-lambda6-sulfanone (10.89 g, 49.502 mmol) and t-BuOK (5.55 g, 49.502 mmol) in portions for a period of 20 min. The mixture was stirred at room temperature for 1 h, cooled to 0° C., and quenched with saturated NH₄Cl solution (15 mL) at 0° C. The resulting solution was extracted with EA (100 mL) four times. The combined organic layers were washed with brine, dried over anhydrous sodium sulfate, concentrated under reduced pressure, and purified by silica gel chromatography (EA/PE, 1/1) to give 6-bromo-2H,3H-furo[3,2-b]pyridin-3-ol (2.10 g, 49%) as a yellow solid. MS ESI calculated for C₇H₆BrNO₂ [M+H]⁺, 215.96 217.96 found 215.85 217.85. ¹H NMR (400 MHz, Chloroform-d) δ 8.18 (d, J=1.6 Hz, 1H), 7.37 (d, J=1.6 Hz, 1H), 5.44-5.41 (m, 1H), 4.77-4.72 (m, 1H), 4.61-4.56 (m, 1H).

### Preparation 44E: 6-bromo-2H-furo[3,2-b]pyridin-3-one

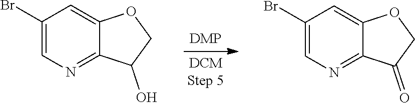 [SMILES: OC1COc2cc(Br)cnc21 ; O=C1COc2cc(Br)cnc21]

A solution of 6-bromo-2H,3H-furo[3,2-b]pyridin-3-ol (500 mg, 2.314 mmol) and DMP (1.96 g, 4.628 mmol) in DCM (15 mL) was stirred for 3 h at room temperature under nitrogen atmosphere. The resulting mixture was concentrated under reduced pressure. The residue was purified by silica gel column chromatography, eluted with PE/EA (3/1) to afford 6-bromo-2H-furo[3,2-b]pyridin-3-one (400 mg, 81%) as a yellow solid. MS ESI calculated for C₇H₄BrNO₂ [M+H]⁺, 213.94 215.94, found 213.85 215.85. ¹H NMR (400 MHz, Chloroform-d) δ 8.59 (d, J=1.7 Hz, 1H), 7.76 (d, J=1.7 Hz, 1H), 4.77 (s, 2H).

### Preparation 44F: 6-bromo-3-methyl-2H-furo[3,2-b]pyridin-3-ol

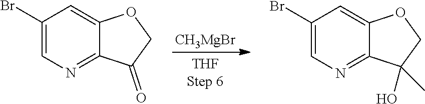

To a stirred solution of 6-bromo-2H-furo[3,2-b]pyridin-3-one (400 mg, 1.869 mmol) in THF (5 mL) was added CH₃MgBr (1.25 mL, 3.738 mmol) dropwise at 0° C. under nitrogen atmosphere. The resulting mixture was stirred for 2 h at room temperature under nitrogen atmosphere. The reaction was quenched with water at 0° C. The resulting mixture was extracted with CH₂Cl₂ (3×20 mL). The combined organic layers were washed with brine (3×30 mL), dried over anhydrous Na₂SO₄. After filtration, the filtrate was concentrated under reduced pressure. The residue was purified by silica gel column chromatography, eluted with CH₂Cl₂/MeOH (10/1) to afford 6-bromo-3-methyl-2H-furo[3,2-b]pyridin-3-ol (100 mg, 23%) as a light yellow liquid. MS ESI calculated for C₇H₄BrNO₂ [M+H]⁺, 229.97 231.97, found 229.85 231.85.

### Example 44: (7R,14R)-1-(difluoromethoxy)-11-(3-hydroxy-3-methyl-2,3-dihydrofuro[3,2-b]pyridin-6-yl)-6-(methyl-d3)-6,7-dihydro-7,14-methanobenzo[f]benzo[4,5]imidazo[1,2-a][1,4]diazocin-5(14H)-one

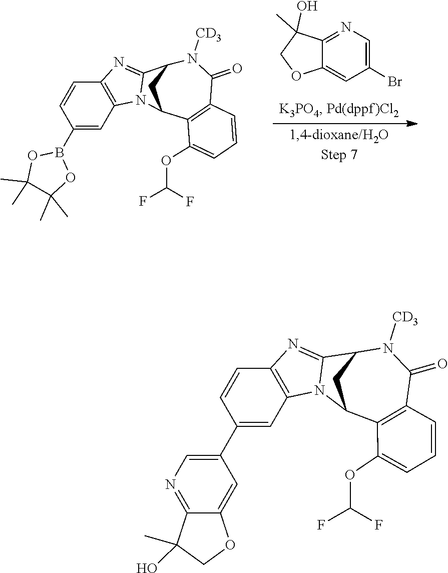 [SMILES: [2H]C([2H])([2H])N1C(=O)c2cccc(OC(F)F)c2[C@H]2C[C@@H]1c1nc3ccc(B4OC(C)(C)C(C)(C)O4)cc3n12 ; CC1(O)COc2cc(Br)cnc21 ; [2H]C([2H])([2H])N1C(=O)c2cccc(OC(F)F)c2[C@H]2C[C@@H]1c1nc3ccc(-c4cnc5c(c4)OCC5(C)O)cc3n12]

To a stirred solution of (7R,14R)-1-(difluoromethoxy)-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (80 mg, 0.165 mmol) and 6-bromo-3-methyl-2H-furo[3,2-b]pyridin-3-ol (57 mg, 0.247 mmol) in 1,4-dioxane (1 mL) and H₂O (0.2 mL) were added Pd(dppf)Cl₂ (13 mg, 0.017 mmol) and K₃PO₄ (105 mg, 0.495 mmol) at room temperature under nitrogen atmosphere. The resulting mixture was stirred for 1 h at 80° C. under nitrogen atmosphere. The mixture was concentrated under reduce pressure and purified by silica gel column chromatography, eluted with CH₂Cl₂/MeOH (10/1) followed by reversed-phase flash chromatography with the following conditions: column, C18 silica gel; mobile phase, CH₃CN in water (10 mmol/L NH₄HCO₃), 30% to 70% gradient in 20 min; detector, 254 nm. This resulted in (7R,14R)-1-(difluoromethoxy)-11-(3-hydroxy-3-methyl-2,3-dihydrofuro[3,2-b]pyridin-6-yl)-6-(methyl-d3)-6,7-dihydro-7,14-methanobenzo[f]benzo[4,5]imidazo[1,2-a][1,4]diazocin-5(14H)-one (32 mg, 38%) as a white solid. MS ESI calculated for C₂₇H₁₉D₃F₂N₄O₄ [M+H]⁺, 508.18 found 508.10. ¹H NMR (400 MHz, Chloroform-d) δ 8.49 (d, J=7.6 Hz, 1H), 8.36-8.30 (m, 1H), 7.79 (d, J=8.5 Hz, 1H), 7.65 (s, 1H), 7.42 (t, J=8.3 Hz, 2H), 7.38-7.34 (m, 1H), 7.32 (d, J=8.2 Hz, 1H), 6.84 (t, J=72.8 Hz, 1H), 6.29 (d, J=7.2 Hz, 1H), 5.00 (d, J=7.1 Hz, 1H), 4.74-4.67 (m, 1H), 4.48 (d, J=10.1 Hz, 1H), 3.55-3.43 (m, 1H), 2.89 (d, J=13.5 Hz, 1H), 1.82 (s, 3H). ¹⁹F NMR (377 MHz, Chloroform-d) δ −80.24, −80.27, −80.68, −80.71, −80.79, −80.80, −81.24, −81.25.

### Example 45: (7R,14R)-11-(8-amino-8-methyl-7,8-dihydro-5H-pyrano[4,3-b]pyridin-3-yl)-1-(difluoromethoxy)-6-(methyl-d3)-6,7-dihydro-7,14-methanobenzo[f]benzo[4,5]imidazo[1,2-a][1,4]diazocin-5(14H)-one

### Synthetic Scheme

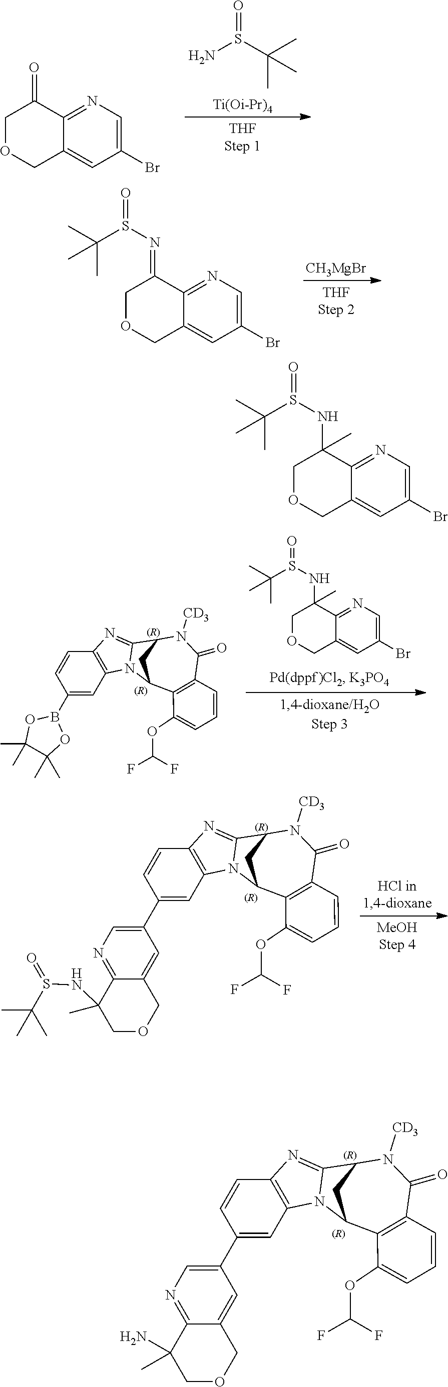

### Preparation 45A: N-[(8Z)-3-bromo-5H,7H-pyrano[4,3-b]pyridin-8-ylidene]-2-methylpropane-2-sulfinamide

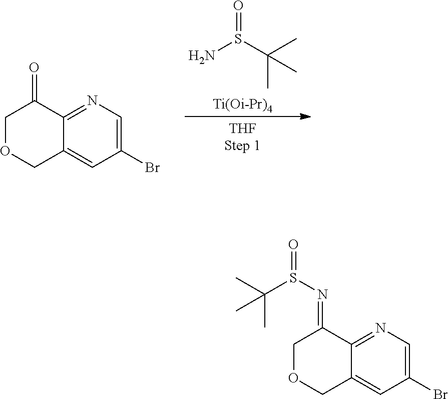

A solution of 3-bromo-5H,7H-pyrano[4,3-b]pyridin-8-one (180 mg, 0.789 mmol), tert-butanesulfinamide (191 mg, 1.579 mmol) and Ti(Oi-Pr)₄ (448 mg, 1.579 mmol) in THF (3 mL) was stirred for overnight at room temperature under nitrogen atmosphere. The reaction was quenched with 20 mL sat. NH₄Cl (aq.) at 25° C. and was extracted with EtOAc (3×20 mL). The combined organic layers were washed with brine (3×40 mL), dried over anhydrous Na₂SO₄. After filtration, the filtrate was concentrated under reduced pressure. The residue was purified by silica gel column chromatography, eluted with PE/EA (2/1) to afford N-[(8Z)-3-bromo-5H,7H-pyrano[4,3-b]pyridin-8-ylidene]-2-methylpropane-2-sulfinamide (120 mg, 45%) as an orange solid. MS ESI calculated for C₁₂H₁₅BrN₂O₂S [M+H]⁺, 331.00 333.00 found 330.80 332.80. ¹H NMR (400 MHz, Chloroform-d) δ 8.75 (d, J=2.0 Hz, 1H), 7.69 (d, J=2.0 Hz, 1H), 5.37 (d, J=17.2 Hz, 1H), 5.11 (d, J=17.2 Hz, 1H), 4.79 (s, 2H), 1.36 (s, 9H).

### Preparation 45B: N-{3-bromo-8-methyl-5H,7H-pyrano[4,3-b]pyridin-8-yl}-2-methylpropane-2-sulfinamide

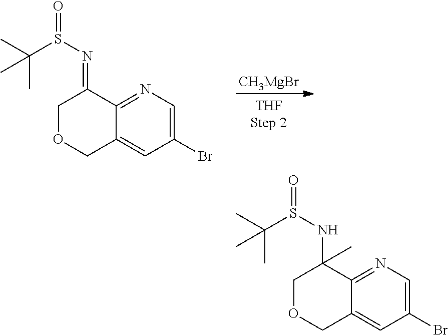 [SMILES: CC(C)(C)S(=O)/N=C1\COCc2cc(Br)cnc21 ; "CH3MgBr" ; CC1(NS(=O)C(C)(C)C)COCc2cc(Br)cnc21]

To a stirred solution of N-[(8Z)-3-bromo-5H,7H-pyrano[4,3-b]pyridin-8-ylidene]-2-methylpropane-2-sulfinamide (120 mg, 0.362 mmol) in THF (5 mL) was added CH₃MgBr (0.24 mL, 0.724 mmol, 3M in THF) dropwise at −10° C. under nitrogen atmosphere. The resulting mixture was stirred for 3 h at room temperature under nitrogen atmosphere. The reaction was quenched with 1 mL sat. NH₄Cl (aq.) at 0° C. and then concentrated under vacuum. The residue was purified by silica gel column chromatography, eluted with PE/EA (1/1) to afford N-{3-bromo-8-methyl-5H,7H-pyrano[4,3-b]pyridin-8-yl}-2-methylpropane-2-sulfinamide (60 mg, 47%) as a yellow solid. MS ESI calculated for C₁₃H₁₉BrN₂O₂S [M+H]⁺, 347.04 349.03 found 347.15 349.15.

### Preparation 45C: N-(3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-8-methyl-7,8-dihydro-5H-pyrano[4,3-b]pyridin-8-yl)-2-methylpropane-2-sulfinamide

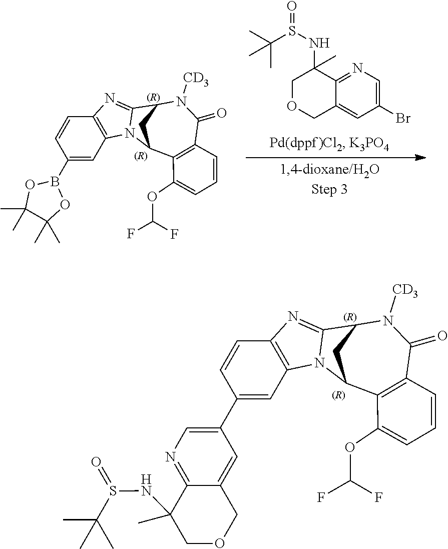

To a solution of (7R,14R)-1-(difluoromethoxy)-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (80 mg, 0.165 mmol) and N-{3-bromo-8-methyl-5H,7H-pyrano[4,3-b]pyridin-8-yl}-2-methylpropane-2-sulfinamide (57 mg, 0.165 mmol) in 1,4-dioxane (2 mL) and H₂O (0.4 mL) were added K₃PO₄ (105 mg, 0.495 mmol) and Pd(dppf)Cl₂ (14 mg, 0.017 mmol) at room temperature. After stirring for overnight at 80° C. under a nitrogen atmosphere, the resulting mixture was concentrated under reduced pressure and purified by silica gel column chromatography, eluted with CH₂Cl₂/MeOH (10/1) to afford N-(3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-8-methyl-7,8-dihydro-5H-pyrano[4,3-b]pyridin-8-yl)-2-methylpropane-2-sulfinamide (90 mg, 87%) as a yellow solid. MS ESI calculated for C₃₂H₃₀D₃F₂N₅O₄S [M+H]⁺, 625.24, found 625.30.

### Example 45: (7R,14R)-11-(8-amino-8-methyl-7,8-dihydro-5H-pyrano[4,3-b]pyridin-3-yl)-1-(difluoromethoxy)-6-(methyl-d3)-6,7-dihydro-7,14-methanobenzo[f]benzo[4,5]imidazo[1,2-a][1,4]diazocin-5(14H)-one

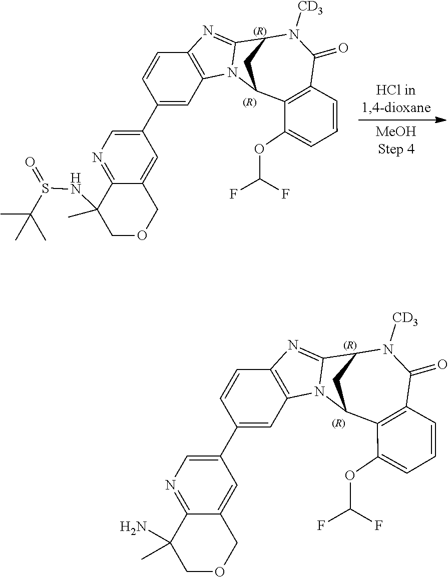

To a stirred solution of N-(3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-8-methyl-7,8-dihydro-5H-pyrano[4,3-b]pyridin-8-yl)-2-methylpropane-2-sulfinamide (90 mg, 0.144 mmol) in MeOH (2 mL) was added HCl(gas) in 1,4-dioxane (1 mL) dropwise at 0° C. under nitrogen atmosphere. The resulting mixture was stirred for 1 h at room temperature. The resulting mixture was concentrated under vacuum and purified by Prep-HPLC with the following conditions (Column: YMC-Actus Triart C18 ExRS 30*150 mm, 5 μm; Mobile Phase A: Water (10 mmol/L NH₄HCO₃), Mobile Phase B: ACN; Flow rate: 60 mL/min mL/min; Gradient: 17% B to 35% B in 10 min; Wave Length: 254 nm/220 nm; RT1(min): 11.22) to afford (7R,14R)-11-(8-amino-8-methyl-7,8-dihydro-5H-pyrano[4,3-b]pyridin-3-yl)-1-(difluoromethoxy)-6-(methyl-d3)-6,7-dihydro-7,14-methanobenzo[f]benzo[4,5]imidazo[1,2-a][1,4]diazocin-5(14H)-one (46 mg, 61%) as a white solid. MS ESI calculated for C₂₈H₂₂D₃F₂N₅O₃ [M+H]⁺, 521.21, found 521.30. ¹H NMR (400 MHz, DMSO-d₆) δ 8.71 (d, J=2.0 Hz, 1H), 8.32-8.24 (m, 1H), 7.73 (d, J=8.4 Hz, 1H), 7.71-7.68 (m, 2H), 7.68 (t, J=72.8 Hz, 1H), 7.55-7.47 (m, 3H), 6.29 (d, J=7.2 Hz, 1H), 5.24 (d, J=7.2 Hz, 1H), 4.81 (s, 2H), 3.80-3.65 (m, 2H), 3.58-3.46 (m, 1H), 2.83 (d, J=13.6 Hz, 1H), 2.19 (s, 2H), 1.36 (s, 3H). ¹⁹F NMR (377 MHz, DMSO-d₆) δ −81.50, −81.52, −81.95, −81.96, −82.27, −82.29, −82.72, −82.74.

### Example 46 and 47: (7R,14R)-1-(difluoromethoxy)-11-((R)-3-hydroxy-3-methyl-2,3-dihydrofuro[3,2-b]pyridin-6-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((S)-3-hydroxy-3-methyl-2,3-dihydrofuro[3,2-b]pyridin-6-yl)-6-(methyl-d3)-6,7-dihydro-7,14-methanobenzo[f]benzo[4,5]imidazo[1,2-a][1,4]diazocin-5(14H)-one

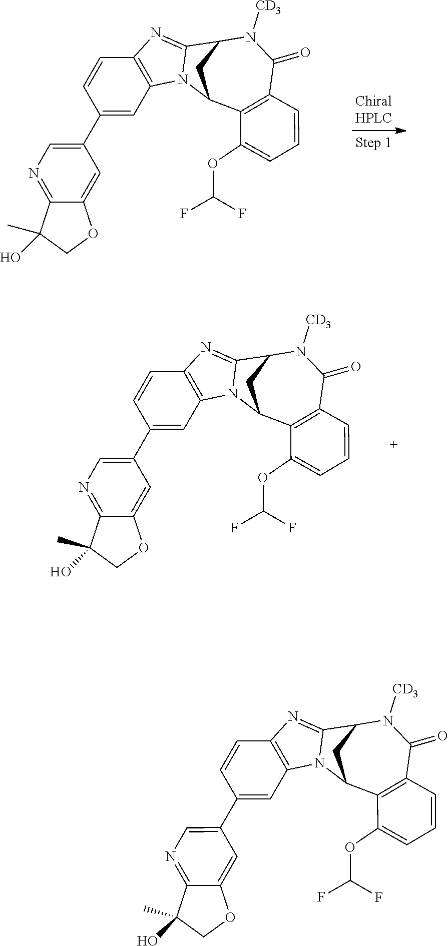 [SMILES: [2H]C([2H])([2H])N1C(=O)c2cccc(OC(F)F)c2[C@H]2C[C@@H]1c1nc3ccc(-c4cnc5c(c4)OCC5(C)O)cc3n12 ; [2H]C([2H])([2H])N1C(=O)c2cccc(OC(F)F)c2[C@H]2C[C@@H]1c1nc3ccc(-c4cnc5c(c4)OC[C@]5(C)O)cc3n12 ; [2H]C([2H])([2H])N1C(=O)c2cccc(OC(F)F)c2[C@H]2C[C@@H]1c1nc3ccc(-c4cnc5c(c4)OC[C@@]5(C)O)cc3n12]

The mixture (50 mg) was resolved by Chiral-HPLC with the following conditions (Column: JW-CHIRAL ART Cellulose-SZ, 3.0*50 mm; 3 um; Mobile Phase A: Hex (0.1% 2M NH₃-MeOH), Mobile Phase B: EtOH; Flow rate: 40 mL/min; Gradient: isocratic 30; Wave Length: 202/306 nm; RT1(min): 14.0; RT2(min): 17.0;). The first peak afforded (10 mg, 19%) as a white solid. MS ESI calculated for C₂₇H₁₉D₃F₂N₄O₄ [M+H]⁺, 508.18, found 508.20. ¹H NMR (400 MHz, Chloroform-d) δ 8.51-8.46 (m, 1H), 8.32 (d, J=1.7 Hz, 1H), 7.76 (d, J=8.5 Hz, 1H), 7.61 (d, J=1.3 Hz, 1H), 7.44-7.36 (m, 2H), 7.30 (d, J=10.7 Hz, 2H), 6.83 (t, J=72.8 Hz, 1H), 6.26 (d, J=7.1 Hz, 1H), 4.97 (d, J=7.0 Hz, 1H), 4.69 (d, J=10.1 Hz, 1H), 4.46 (d, J=10.1 Hz, 1H), 3.53-3.43 (m, 1H), 2.88 (d, J=13.6 Hz, 1H), 1.80 (s, 3H). ¹⁹F NMR (377 MHz, Chloroform-d) δ −80.23, −80.68, −80.81, −81.25. The second peak afforded (10 mg, 20%) as a white solid. MS ESI calculated for C₂₇H₁₉D₃F₂N₄O₄ [M+H]⁺, 508.18, found 508.10. ¹H NMR (400 MHz, Chloroform-d) δ 8.53-8.46 (m, 1H), 8.29 (s, 1H), 7.78 (d, J=8.5 Hz, 1H), 7.65 (s, 1H), 7.46-7.37 (m, 3H), 7.32 (d, J=8.2 Hz, 1H), 6.86 (t, J=72.8 Hz, 1H), 6.31 (d, J=6.6 Hz, 1H), 5.05 (d, J=9.8 Hz, 1H), 4.73 (d, J=10.1 Hz, 1H), 4.50 (d, J=10.1 Hz, 1H), 3.58 -3.48 (m, 1H), 2.91 (d, J=13.6 Hz, 1H), 1.86 (s, 3H). ¹⁹F NMR (377 MHz, Chloroform-d) δ −80.31, −80.76, −80.81, −81.25.

### Example 48: (7R,14R)-1-(difluoromethoxy)-11-(4-hydroxy-3,4-dihydro-2H-pyrano[3,2-b]pyridin-7-yl)-6-(methyl-d3)-6,7-dihydro-7,14-methanobenzo[f]benzo[4,5]imidazo[1,2-a][1,4]diazocin-5(14H)-one

### Synthetic Scheme

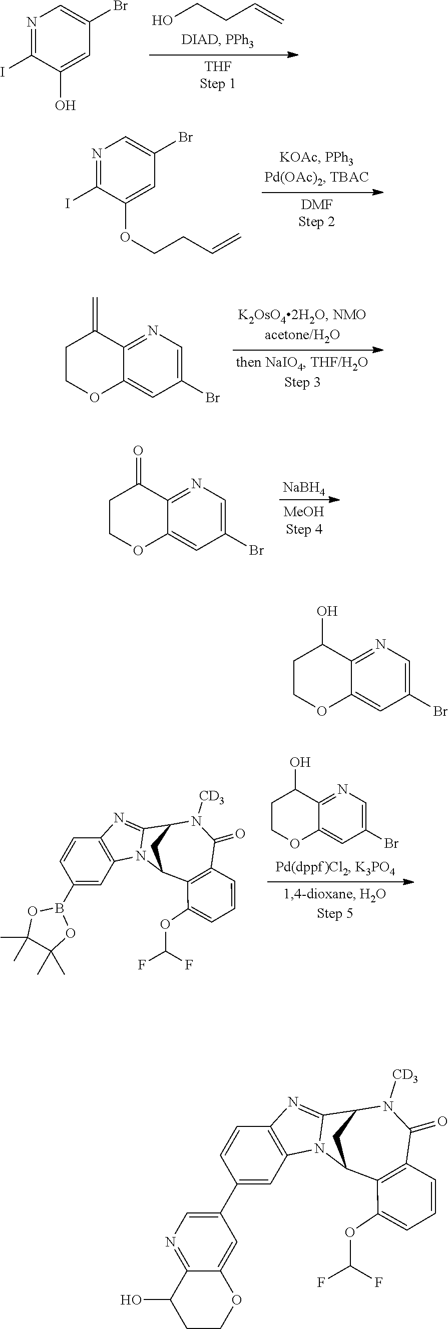

### Preparation 48A: 5-bromo-3-(but-3-en-1-yloxy)-2-iodopyridine

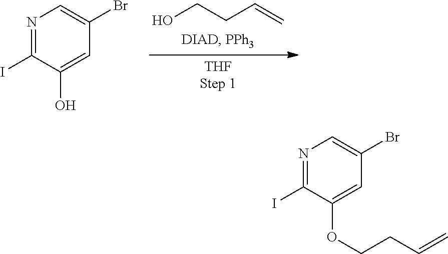

To a stirred solution of 5-bromo-2-iodopyridin-3-ol (24.00 g, 127.643 mmol) and PPh₃ (40.18 g, 153.188 mmol) in THF (300 mL) were added DIAD (28.39 g, 140.399 mmol) and 3-buten-1-ol (9.66 g, 133.968 mmol) at 0° C. under nitrogen atmosphere. The mixture was stirred for 4 h at 70° C. The resulting mixture was concentrated under vacuum and purified by silica gel column chromatography, eluted with CH₂Cl₂/PE (0˜30%) to afford 5-bromo-3-(but-3-en-1-yloxy)-2-iodopyridine (31.01 g, 69%) as a light yellow oil. MS ESI calculated for C₉H₉BrINO [M+H]⁺, 353.89 355.89 found 353.90 355.60. ¹H NMR (300 MHz, Chloroform-d) δ 8.08 (d, J=2.0 Hz, 1H), 7.08 (d, J=2.0 Hz, 1H), 6.03-5.84 (m, 1H), 5.31-5.12 (m, 2H), 4.06 (t, J=6.5 Hz, 2H), 2.69-2.55 (m, 2H).

### Preparation 48B: 7-bromo-4-methylene-3,4-dihydro-2H-pyrano[3,2-b]pyridine

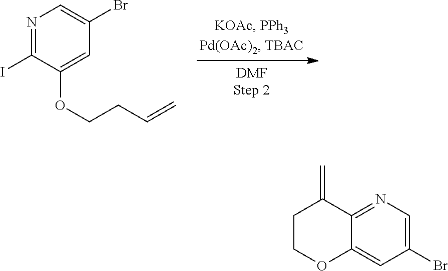

To a stirred solution of 5-bromo-3-(but-3-en-1-yloxy)-2-iodopyridine (16.00 g, 45.200 mmol), tetrabutylazanium chloride (25.12 g, 90.400 mmol) and PPh₃ (3.56 g, 13.560 mmol) in DMF (160 mL) were added KOAc (22.18 g, 226.000 mmol) and Pd(OAc)₂ (1.01 g, 4.520 mmol) at room temperature under nitrogen atmosphere. The mixture was stirred for 3 h at 105° C. The reaction was quenched with water at room temperature. The resulting mixture was extracted with EtOAc (3×800 mL). The combined organic layers were washed with brine (2×500 mL), dried over anhydrous Na₂SO₄. After filtration, the filtrate was concentrated under reduced pressure. The residue was purified by silica gel column chromatography, eluted with CH₂Cl₂/PE (0˜20%) to afford 7-bromo-4-methylene-3,4-dihydro-2H-pyrano[3,2-b]pyridine (6.90 g, 67%) as a light red oil. MS ESI calculated for C₉H₈BrNO [M+H]⁺, 225.98 227.98 found 226.00 228.00. ¹H NMR (400 MHz, Chloroform-d) δ 8.23 (d, J=2.0 Hz, 1H), 7.34 (d, J=2.0 Hz, 1H), 6.23-6.17 (m, 1H), 5.14-5.08 (m, 1H), 4.28-4.21 (m, 2H), 2.85-2.77 (m, 2H).

### Preparation 48C: 7-bromo-2H,3H-pyrano[3,2-b]pyridin-4-one

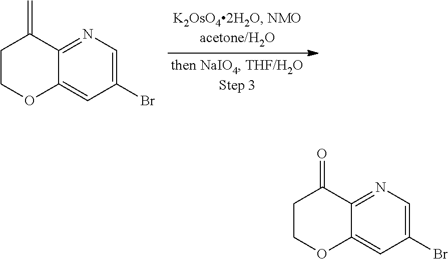

To a stirred solution of 7-bromo-4-methylene-3,4-dihydro-2H-pyrano[3,2-b]pyridine (1.00 g, 4.423 mmol) in acetone (8 mL) were added K₂OsO₄·2H₂O (81 mg, 0.221 mmol) and NMO (1.04 g, 8.846 mmol) in H₂O (2 mL) at room temperature under nitrogen atmosphere. The mixture was stirred for 1 h at room temperature. Na₂S₂O₄ (1.99 g, 9.731 mmol, 85%) was added to above mixture and stirred for 1 h. The resulting mixture was filtered, the filter cake was washed with MeOH (3×5 mL). The filtrate was concentrated under reduced pressure. The resulting mixture was concentrated under vacuum. The residue was dissolved in THF (8 mL) and H₂O (4 mL). Then NaIO₄ (1.89 g, 8.846 mmol) was added and stirred for additional 1 h. The resulting mixture was extracted with EtOAc (3×200 mL). The combined organic layers were washed with brine (1×100 mL), dried over anhydrous Na₂SO₄. After filtration, the filtrate was concentrated under reduced pressure. The residue was purified by silica gel column chromatography, eluted with PE/EA (0˜40%) to afford 7-bromo-2H,3H-pyrano[3,2-b]pyridin-4-one (450 mg, 45%) as a light yellow solid. MS ESI calculated for C₈H₆BrNO₂ [M+H]⁺, 227.96 229.96 found 227.80 229.80. ¹H NMR (400 MHz, Chloroform-d) δ 8.48 (d, J=1.9 Hz, 1H), 7.61 (d, J=1.9 Hz, 1H), 4.63 (t, J=6.5 Hz, 2H), 2.98 (t, J=6.5 Hz, 2H).

### Preparation 48D: 7-bromo-3,4-dihydro-2H-pyrano[3,2-b]pyridin-4-ol

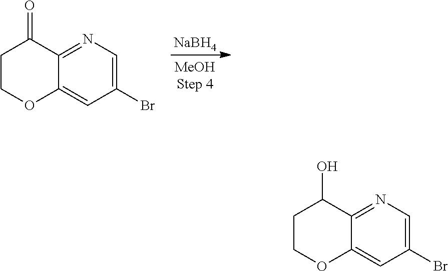 [SMILES: O=C1CCOc2cc(Br)cnc21 ; "NaBH4" ; OC1CCOc2cc(Br)cnc21]

To a stirred solution of 7-bromo-2H,3H-pyrano[3,2-b]pyridin-4-one (200 mg, 0.877 mmol) in MeOH (1 mL) was added NaBH₄ (66 mg, 1.754 mmol) in portions at room temperature. The mixture was stirred for 2 h at room temperature. The reaction was quenched by the addition of sat. NH₄Cl (aq.) (3 mL) at 0° C. The residue was purified by reversed-phase flash chromatography with the following conditions: column, C18 silica gel; mobile phase, MeCN in Water (0.1% FA), 5% to 40% gradient in 25 min; detector, 254 nm. This resulted in 7-bromo-3,4-dihydro-2H-pyrano[3,2-b]pyridin-4-ol (184 mg, 91%) as a light brown liquid. MS ESI calculated for C₈H₈BrNO₂ [M+H]⁺, 229.97 231.97, found 229.90 231.80. ¹H NMR (400 MHz, Chloroform-d) δ 8.25-8.20 (m, 1H), 7.41-7.36 (m, 1H), 4.88-4.80 (m, 1H), 4.40-4.23 (m, 2H), 2.35-2.23 (m, 1H), 2.22-2.10 (m, 1H).

### Example 48: (7R,14R)-1-(difluoromethoxy)-11-(4-hydroxy-3,4-dihydro-2H-pyrano[3,2-b]pyridin-7-yl)-6-(methyl-d3)-6,7-dihydro-7,14-methanobenzo[f]benzo[4,5]imidazo[1,2-a][1,4]diazocin-5(14H)-one

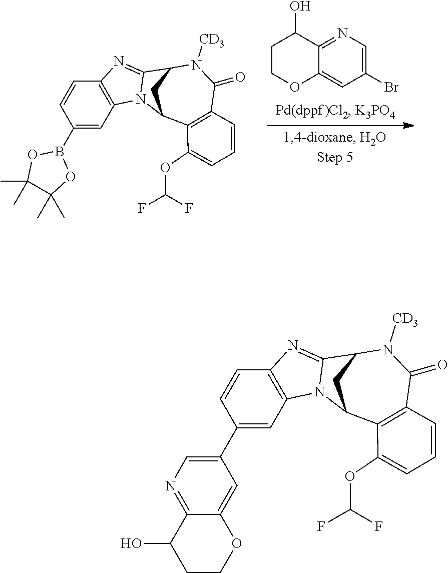

To a stirred mixture of (7R,14R)-1-(difluoromethoxy)-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (50 mg, 0.103 mmol), 7-bromo-3,4-dihydro-2H-pyrano[3,2-b]pyridin-4-ol (29 mg, 0.124 mmol) and K₃PO₄ (44 mg, 0.206 mmol) in 1,4-dioxane (1 mL) and H₂O (0.2 mL) was added Pd(dppf)Cl₂ (8 mg, 0.010 mmol) at room temperature under nitrogen atmosphere. The mixture was stirred for 16 h at 80° C. The resulting mixture was concentrated under vacuum and purified by silica gel column chromatography, eluted with CH₂Cl₂/MeOH (0˜10%) followed by Prep-HPLC with the following conditions (Column: XBridge Prep Phenyl OBD Column 19*250 mm, 5 m; Mobile Phase A: Water (10 mmol/L NH₄HCO₃+0.1% NH₃·H₂O), Mobile Phase B: ACN; Flow rate: 60 mL/min; Gradient: 18% B to 33% B in 10 min; Wave Length: 254 nm; RT1(min): 14.07) to afford (7R,14R)-1-(difluoromethoxy)-11-(4-hydroxy-3,4-dihydro-2H-pyrano[3,2-b]pyridin-7-yl)-6-(methyl-d3)-6,7-dihydro-7,14-methanobenzo[f]benzo[4,5]imidazo[1,2-a][1,4]diazocin-5(14H)-one (32 mg, 61%) as a white solid. MS ESI calculated for C₂₇H₁₉D₃F₂N₄O₄ [M+H]⁺, 508.18 found 508.05. ¹H NMR (400 MHz, Methanol-d₄) δ 8.44-8.32 (m, 2H), 7.81 (d, J=1.6 Hz, 1H), 7.72 (d, J=8.5 Hz, 1H), 7.56-7.51 (m, 1H), 7.49-7.45 (m, 3H), 7.44-7.08 (m, 1H), 6.43 (d, J=7.1 Hz, 1H), 5.22-5.14 (m, 1H), 4.80 (t, J=3.9 Hz, 1H), 4.39-4.30 (m, 2H), 3.64-3.51 (m, 1H), 2.90 (d, J=13.7 Hz, 1H), 2.28-2.06 (m, 2H). ¹⁹F NMR (377 MHz, Methanol-d₄) δ −82.82, −82.86, −83.28, −83.31, −83.80, −83.85, −84.25, −84.31.

### Example 49 and 50: (7R,14R)-1-(difluoromethoxy)-12-fluoro-11-((R)-7-hydroxy-7-methyl-6,7-dihydro-5H-cyclopenta[b]pyridin-3-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-12-fluoro-11-((S)-7-hydroxy-7-methyl-6,7-dihydro-5H-cyclopenta[b]pyridin-3-yl)-6-(methyl-d3)-6,7-dihydro-7,14-methanobenzo[f]benzo[4,5]imidazo[1,2-a][1,4]diazocin-5(14H)-one

### Synthetic Scheme

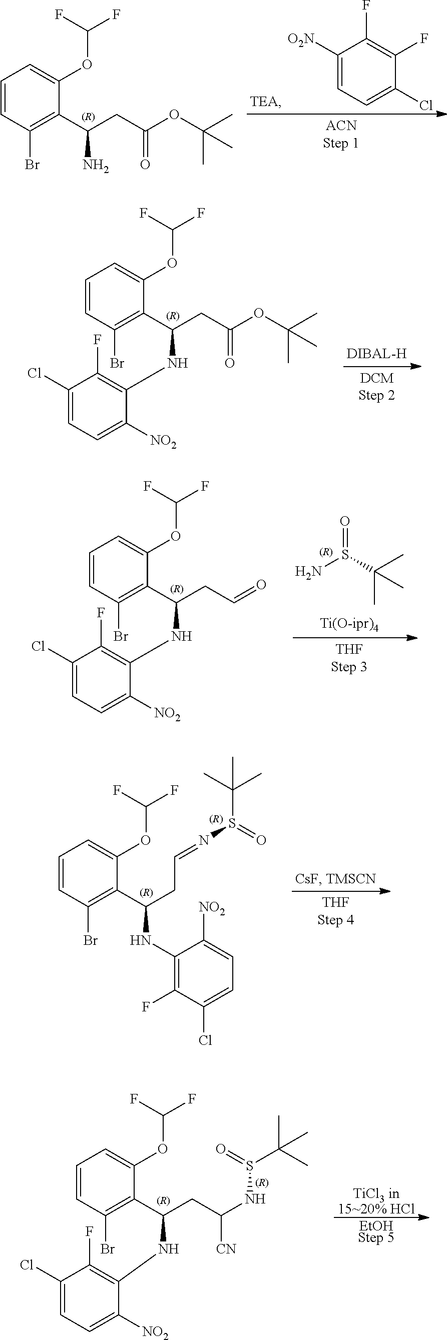

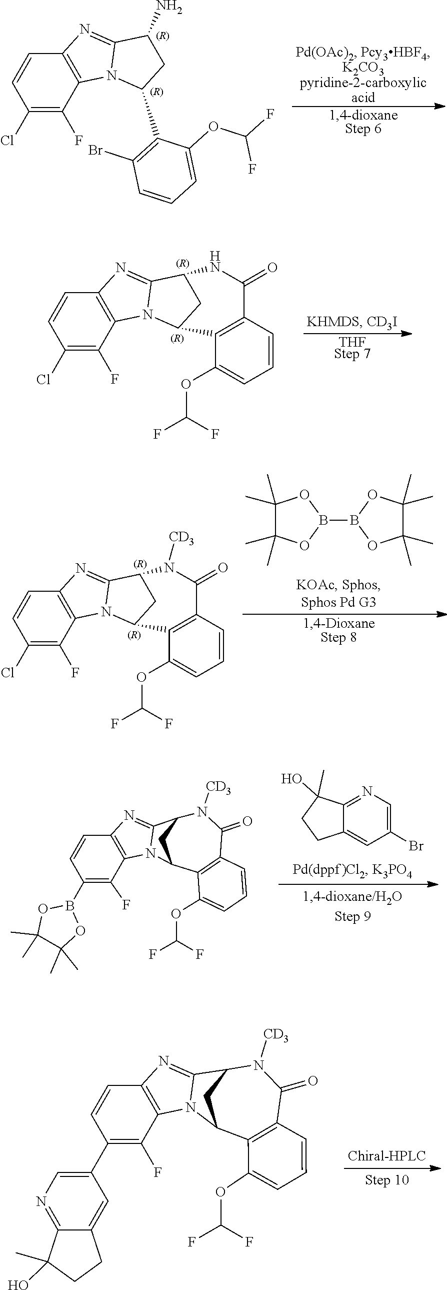

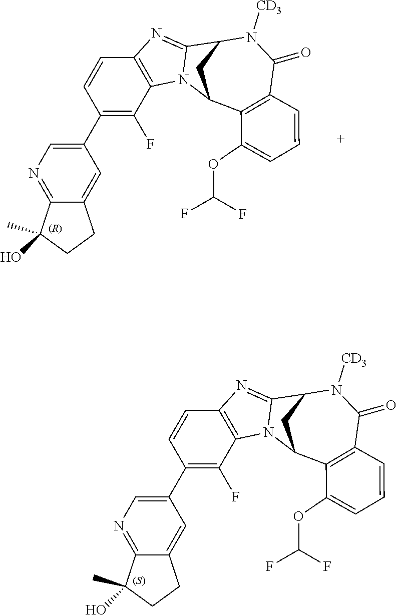

### Preparation 49A: tert-butyl (3R)-3-[2-bromo-6-(difluoromethoxy)phenyl]-3-[(3-chloro-2-fluoro-6-nitrophenyl)amino]propanoate

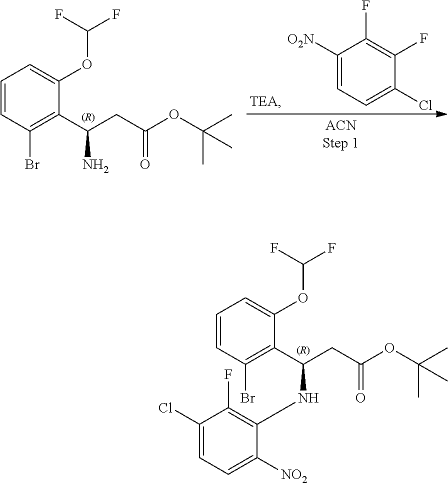

A solution of tert-butyl (3R)-3-amino-3-[2-bromo-6-(difluoromethoxy)phenyl]propanoate (35.00 g, 95.575 mmol), 1-chloro-2,3-difluoro-4-nitrobenzene (22.20 g, 114.690 mmol) and TEA (14.51 g, 143.363 mmol) in ACN (350 mL) was stirred for 3 h at 50° C. The resulting mixture was concentrated under reduced pressure and purified by silica gel column chromatography, eluted with CH₂Cl₂/PE (10/1) to afford tert-butyl (3R)-3-[2-bromo-6-(difluoromethoxy)phenyl]-3-[(3-chloro-2-fluoro-6-nitrophenyl)amino]propanoate (48.20 g, 93%) as a yellow oil. MS ESI calculated for C₂₀H₁₉BrClF₃N₂O₅ [M+H]⁺, 539.01 541.01, found 539.05 541.05. ¹H NMR (400 MHz, Chloroform-d) δ 8.51-8.37 (m, 1H), 7.89 (d, J=9.3 Hz, 1H), 7.44 (d, J=7.0 Hz, 1H), 7.19-7.09 (m, 2H), 6.84-6.45 (m, 2H), 6.17-6.07 (m, 1H), 3.12-2.97 (m, 1H), 2.87-2.76 (m, 1H), 1.34 (s, 9H). ¹⁹F NMR (377 MHz, Chloroform-d) δ −80.14, −80.60, −80.94, −81.38, −123.07.

### Preparation 49B: (3R)-3-[2-bromo-6-(difluoromethoxy)phenyl]-3-[(3-chloro-2-fluoro-6-nitrophenyl)amino]propanal

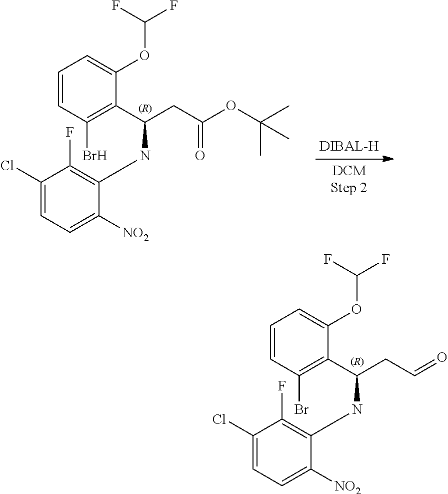

To a stirred solution of tert-butyl (3R)-3-[2-bromo-6-(difluoromethoxy)phenyl]-3-[(3-chloro-2-fluoro-6-nitrophenyl)amino]propanoate (31.00 g, 57.436 mmol) in DCM (465 mL) was added DIBAL-H (63.0 mL, 63.180 mmol) dropwise at −78° C. The resulting mixture was stirred for 3 h at −78° C. under nitrogen atmosphere. The reaction was quenched with HCl (1N) at −78° C. The resulting mixture was extracted with CH₂Cl₂ (3×500 mL). The combined organic layers were washed with brine (3×500 mL), dried over anhydrous Na₂SO₄. After filtration, the filtrate was concentrated under reduced pressure. The residue was purified by silica gel column chromatography, eluted with CH₂Cl₂/PE (1/1) to afford (3R)-3-[2-bromo-6-(difluoromethoxy)phenyl]-3-[(3-chloro-2-fluoro-6-nitrophenyl)amino]propanal (23.80 g, 88%) as a yellow oil. MS ESI calculated for C₁₆H₁₁BrClF₃N₂O₄ [M+H]⁺, 466.95 468.95, found 466.80 468.80. ¹H NMR (400 MHz, Chloroform-d) δ 9.82 (s, 1H), 8.39 (d, J=9.0 Hz, 1H), 7.93-7.84 (m, 1H), 7.46-7.40 (m, 1H), 7.19-7.09 (m, 2H), 6.88-6.47 (m, 2H), 6.34-6.24 (m, 1H), 3.44-3.30 (m, 1H), 3.10-2.98 (m, 1H).

### Preparation 49C: (R)—N-[(3R)-3-[2-bromo-6-(difluoromethoxy)phenyl]-3-[(3-chloro-2-fluoro-6-nitrophenyl)amino]propylidene]-2-methylpropane-2-sulfinamide

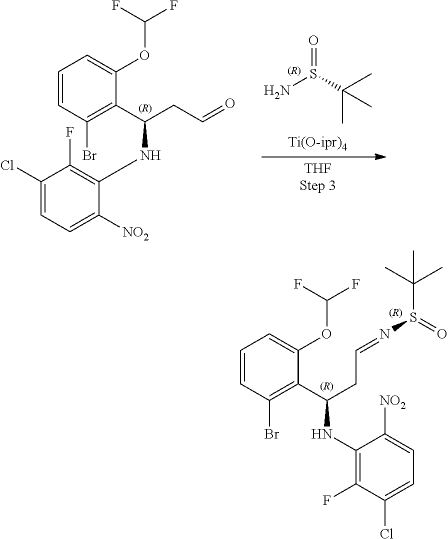

A solution of (3R)-3-[2-bromo-6-(difluoromethoxy)phenyl]-3-[(3-chloro-2-fluoro-6-nitrophenyl)amino]propanal (23.80 g, 50.896 mmol), (R)-2-methylpropane-2-sulfinamide (12.34 g, 101.792 mmol) and Ti(Oi-Pr)₄ (28.93 g, 101.792 mmol) in DCM (240 mL) was stirred for 2 h at room temperature. The resulting mixture was diluted with water (200 mL). The resulting mixture was extracted with CH₂Cl₂ (3×300 mL). The combined organic layers were washed with brine (3×500 mL), dried over anhydrous Na₂SO₄. After filtration, the filtrate was concentrated under reduced pressure. The residue was purified by silica gel column chromatography, eluted with CH₂Cl₂/PE (1/1) to afford (R)—N-[(3R)-3-[2-bromo-6-(difluoromethoxy)phenyl]-3-[(3-chloro-2-fluoro-6-nitrophenyl)amino]propylidene]-2-methylpropane-2-sulfinamide (24.10 g, 82%) as a yellow oil. MS ESI calculated for C₂₀H₂₀BrClF₃N₃O₄S [M−H]⁻, 568.00 570.00, found 567.80 569.80. ¹H NMR (400 MHz, Chloroform-d) δ 8.47 (d, J=8.0 Hz, 1H), 8.12 (t, J=3.9 Hz, 1H), 7.89 (d, J=9.3 Hz, 1H), 7.44 (d, J=7.5 Hz, 1H), 7.21-7.09 (m, 2H), 6.85-6.44 (m, 2H), 6.30-6.15 (m, 1H), 3.54-3.29 (m, 1H), 3.17-3.03 (m, 1H), 1.11 (s, 9H).

### Preparation 49D: (R)—N-[(3R)-3-[2-bromo-6-(difluoromethoxy)phenyl]-3-[(3-chloro-2-fluoro-6-nitrophenyl)amino]-1-cyanopropyl]-2-methylpropane-2-sulfinamide

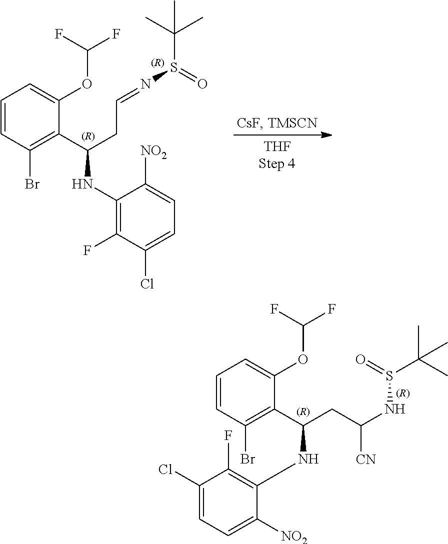

A solution of (R)—N-[(3R)-3-[2-bromo-6-(difluoromethoxy)phenyl]-3-[(3-chloro-2-fluoro-6-nitrophenyl)amino]propylidene]-2-methylpropane-2-sulfinamide (24.00 g, 42.046 mmol), CsF (12.77 g, 84.092 mmol) and TMSCN (8.34 g, 84.092 mmol) in THF (240 mL) was stirred for 16 h at room temperature. The reaction was quenched with sat. NaHCO₃ (aq.) at room temperature. The resulting mixture was extracted with EtOAc (3×300 mL). The combined organic layers were washed with brine (3×500 mL), dried over anhydrous Na₂SO₄. After filtration, the filtrate was concentrated under reduced pressure. The residue was purified by silica gel column chromatography, eluted with PE/EA (1/1) to afford (R)—N-[(3R)-3-[2-bromo-6-(difluoromethoxy)phenyl]-3-[(3-chloro-2-fluoro-6-nitrophenyl)amino]-1-cyanopropyl]-2-methylpropane-2-sulfinamide (21.10 g, 84%) as a yellow solid. MS ESI calculated for C₂₁H₂₁BrClF₃N₄O₄S [M+H]⁺, 597.01 599.01, found 597.05 599.05.

### Preparation 49E: (3R,5R)-3-[2-bromo-6-(difluoromethoxy)phenyl]-11-chloro-12-fluoro-2,7-diazatricyclo[6.4.0.0\hat{ }{2,6}]dodeca-1(8),6,9,11-tetraen-5-amine

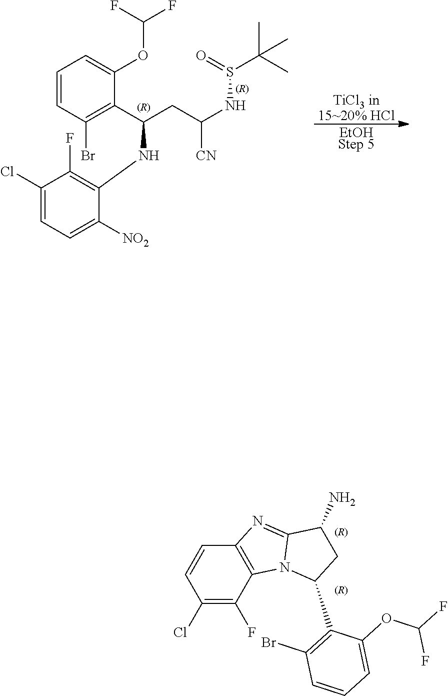

To a stirred solution of TiCl₃ (58.96 g, 66.912 mmol, 17% in HCl) in EtOH (65 mL) was added (R)—N-[(3R)-3-[2-bromo-6-(difluoromethoxy)phenyl]-3-[(3-chloro-2-fluoro-6-nitrophenyl)amino]-1-cyanopropyl]-2-methylpropane-2-sulfinamide (5.00 g, 8.364 mmol) in EtOH (35 mL) dropwise at 75° C. The resulting mixture was stirred for 2 h at 75° C. The resulting mixture was concentrated under vacuum. The residue was basified to pH 9 with Na₂CO₃. The resulting mixture was extracted with EtOAc (3×100 mL). The combined organic layers were washed with brine (3×200 mL), dried over anhydrous Na₂SO₄. After filtration, the filtrate was concentrated under reduced pressure. The residue was purified by silica gel column chromatography, eluted with CH₂Cl₂/MeOH (15/1) to afford (3R,5R)-3-[2-bromo-6-(difluoromethoxy)phenyl]-11-chloro-12-fluoro-2,7-diazatricyclo[6.4.0.0\hat{ }{2,6}]dodeca-1(8),6,9,11-tetraen-5-amine (3.08 g, 82%) as a yellow solid. MS ESI calculated for C₁₇H₁₂BrClF₃N₃O [M+H]⁺, 445.98 447.98, found 446.05 448.05.

### Preparation 49F: (7R,14R)-11-chloro-1-(difluoromethoxy)-12-fluoro-6,7-dihydro-7,14-methanobenzo[f]benzo[4,5]imidazo[1,2-a][1,4]diazocin-5(14H)-one

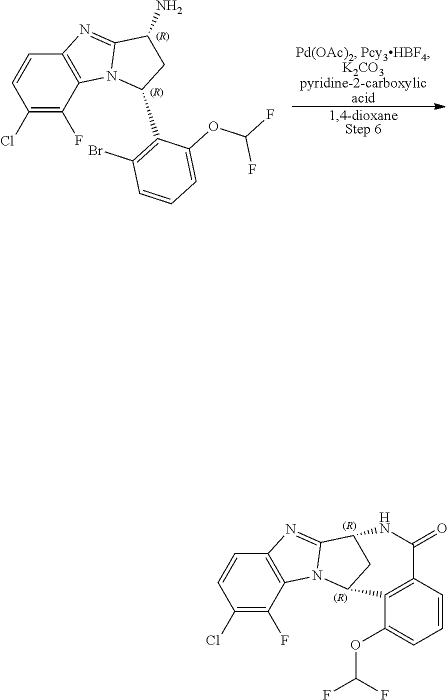

To a solution of (3R,5R)-3-[2-bromo-6-(difluoromethoxy)phenyl]-11-chloro-12-fluoro-2,7-diazatricyclo[6.4.0.0\hat{ }{2,6}]dodeca-1(8),6,9,11-tetraen-5-amine (2.80 g, 6.269 mmol) in 1,4-dioxane (100 mL) was added PCy₃·HBF₄ (461 mg, 1.254 mmol), pyridine-2-carboxylic acid (386 mg, 3.135 mmol), Pd(OAc)₂ (141 mg, 0.627 mmol) and K₂CO₃ (4.33 g, 31.345 mmol) in a pressure tank. The mixture was purged with nitrogen for 2 min and then was pressurized to 10 atm. with carbon monoxide at 110° C. for 16 h. The reaction mixture was cooled to room temperature. The resulting mixture was concentrated under vacuum. The residue was purified by silica gel column chromatography, eluted with CH₂Cl₂/MeOH (10/1) to afford ((7R,14R)-11-chloro-1-(difluoromethoxy)-12-fluoro-6,7-dihydro-7,14-methanobenzo[f]benzo[4,5]imidazo[1,2-a][1,4]diazocin-5(14H)-one (850 mg, 34%) as a yellow solid. MS ESI calculated for C₁₈H₁₁C1F₃N₃O₂ [M+H]⁺, 394.05, found 394.05. ¹H NMR (400 MHz, Chloroform-d) δ 8.47-8.36 (m, 1H), 7.97 (d, J=6.2 Hz, 1H), 7.48-7.36 (m, 3H), 7.23-7.14 (m, 1H), 6.96-6.52 (m, 2H), 4.95 (t, J=6.4 Hz, 1H), 3.57-3.34 (m, 1H), 2.84 (d, J=13.3 Hz, 1H). ¹⁹F NMR (377 MHz, Chloroform-d) δ −78.59, −79.03, −82.55, −82.58, −83.00, −83.02, −134.02.

### Preparation 49G: (7R,14R)-11-chloro-1-(difluoromethoxy)-12-fluoro-6-(methyl-d3)-6,7-dihydro-7,14-methanobenzo[f]benzo[4,5]imidazo[1,2-a][1,4]diazocin-5(14H)-one

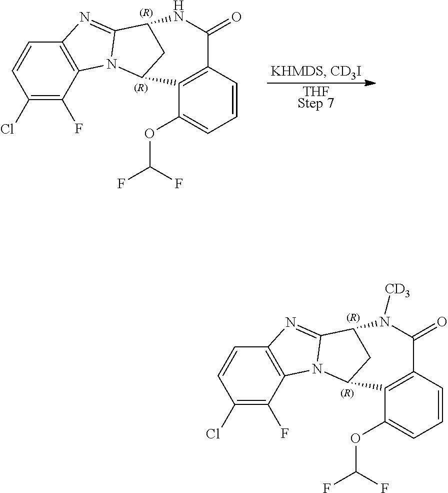

To a stirred solution of (7R,14R)-11-chloro-1-(difluoromethoxy)-12-fluoro-6,7-dihydro-7,14-methanobenzo[f]benzo[4,5]imidazo[1,2-a][1,4]diazocin-5(14H)-one (550 mg, 1.397 mmol) in THF (15 mL) was added KHMDS (2.0 mL, 1.676 mmol, 1 M in THF) dropwise at 0° C. The resulting mixture was stirred for 1 h at 0° C. under nitrogen atmosphere. To the above mixture was added iodomethane-d₃ (263 mg, 1.816 mmol) at room temperature. The resulting mixture was stirred for additional 2 h at room temperature. The reaction was quenched with sat. NH₄Cl (aq.) at room temperature. The resulting mixture was extracted with EtOAc (3×50 mL). The combined organic layers were washed with brine (3×100 mL), dried over anhydrous Na₂SO₄.

After filtration, the filtrate was concentrated under reduced pressure. The residue was purified by silica gel column chromatography, eluted with CH₂Cl₂/MeOH (10/1) to afford (7R,14R)-11-chloro-1-(difluoromethoxy)-12-fluoro-6-(methyl-d3)-6,7-dihydro-7,14-methanobenzo[f]benzo[4,5]imidazo[1,2-a][1,4]diazocin-5(14H)-one (460 mg, 80%) as a light yellow solid. MS ESI calculated for C₁₉H₁₀D₃C1F₃N₃O₂ [M+H]⁺, 411.08, found 411.20. ¹H NMR (400 MHz, Chloroform-d) δ 8.43 (d, J=8.0 Hz, 1H), 7.51-7.34 (m, 3H), 7.23-7.13 (m, 1H), 6.96-6.54 (m, 1H), 6.47 (d, J=7.4 Hz, 1H), 4.92 (d, J=7.0 Hz, 1H), 3.51-3.36 (m, 1H), 2.85 (d, J=13.7 Hz, 1H).

### Preparation 49H: (7R,14R)-1-(difluoromethoxy)-12-fluoro-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one

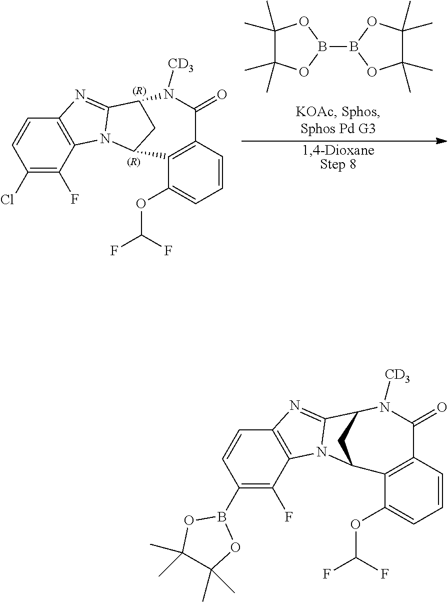

To a stirred solution of (7R,14R)-11-chloro-1-(difluoromethoxy)-12-fluoro-6-(methyl-d3)-6,7-dihydro-7,14-methanobenzo[f]benzo[4,5]imidazo[1,2-a][1,4]diazocin-5(14H)-one (300 mg, 0.730 mmol) and BPD (556 mg, 2.190 mmol) in 1,4-dioxane (10 mL) were added SPhos (60 mg, 0.146 mmol), SPhos Pd Gen.3 (57 mg, 0.073 mmol) and KOAc (215 mg, 2.190 mmol) at room temperature under nitrogen atmosphere. The resulting mixture was stirred for 1 h at 80° C. under nitrogen atmosphere. The resulting mixture was concentrated under reduced pressure and purified by silica gel column chromatography, eluted with PE/EA/EtOH (6/3/1) to afford (7R,14R)-1-(difluoromethoxy)-12-fluoro-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (270 mg, 73%) as a white solid. MS ESI calculated for C₂₅H₂₂D3BF₃N₃O₄ [M+H]⁺, 503.21 found 503.30.

### Preparation 49I: (7R,14R)-1-(difluoromethoxy)-12-fluoro-11-(7-hydroxy-7-methyl-6,7-dihydro-5H-cyclopenta[b]pyridin-3-yl)-6-(methyl-d3)-6,7-dihydro-7,14-methanobenzo[f]benzo[4,5]imidazo[1,2-a][1,4]diazocin-5(14H)-one

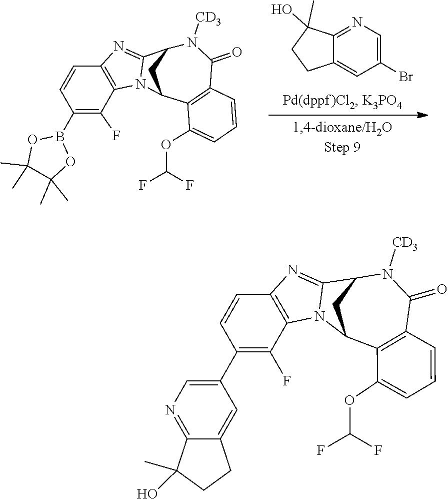 [SMILES: [2H]C([2H])([2H])N1C(=O)c2cccc(OC(F)F)c2[C@H]2C[C@@H]1c1nc3ccc(B4OC(C)(C)C(C)(C)O4)c(F)c3n12 ; CC1(O)CCc2cc(Br)cnc21 ; [2H]C([2H])([2H])N1C(=O)c2cccc(OC(F)F)c2[C@H]2C[C@@H]1c1nc3ccc(-c4cnc5c(c4)CCC5(C)O)c(F)c3n12]

To a stirred solution of (7R,14R)-1-(difluoromethoxy)-12-fluoro-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (90 mg, 0.179 mmol) and 3-bromo-7-methyl-6,7-dihydro-5H-cyclopenta[b]pyridin-7-ol (49 mg, 0.215 mmol) in 1,4-dioxane (0.8 mL) and H₂O (0.2 mL) were added Pd(dppf)Cl₂ (15 mg, 0.018 mmol) and K₃PO₄ (114 mg, 0.537 mmol) at room temperature under nitrogen atmosphere. The resulting mixture was stirred for 2 h at 80° C. under nitrogen atmosphere. The resulting mixture was concentrated under vacuum. The residue was purified by silica gel column chromatography, eluted with CH₂Cl₂/MeOH (10/1) followed by reversed-phase flash chromatography with the following conditions: column, C18 silica gel; mobile phase, CH₃CN in water (10 mmol/L NH₄HCO₃), 30% to 45% gradient in 15 min; detector, 254/220 nm to afford (7R,14R)-1-(difluoromethoxy)-12-fluoro-11-(7-hydroxy-7-methyl-6,7-dihydro-5H-cyclopenta[b]pyridin-3-yl)-6-(methyl-d3)-6,7-dihydro-7,14-methanobenzo[f]benzo[4,5]imidazo[1,2-a][1,4]diazocin-5(14H)-one (45 mg, 48%) as a white solid. MS ESI calculated for C₂₈H₂₀D₃F₃N₄O₃ [M+H]⁺, 524.19, found 524.25.

### Example 49 and 50: (7R,14R)-1-(difluoromethoxy)-12-fluoro-11-((R)-7-hydroxy-7-methyl-6,7-dihydro-5H-cyclopenta[b]pyridin-3-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-12-fluoro-11-((S)-7-hydroxy-7-methyl-6,7-dihydro-5H-cyclopenta[b]pyridin-3-yl)-6-(methyl-d3)-6,7-dihydro-7,14-methanobenzo[f]benzo[4,5]imidazo[1,2-a][1,4]diazocin-5(14H)-one

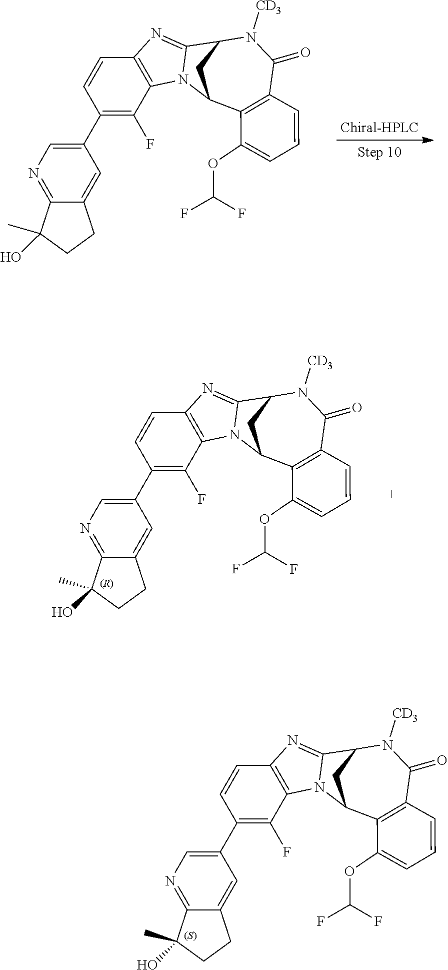

(7R,14R)-1-(difluoromethoxy)-12-fluoro-11-(7-hydroxy-7-methyl-6,7-dihydro-5H-cyclopenta[b]pyridin-3-yl)-6-(methyl-d3)-6,7-dihydro-7,14-methanobenzo[f]benzo[4,5]imidazo[1,2-a][1,4]diazocin-5(14H)-one (45 mg) was separated by Prep-Chiral HPLC with the following conditions (Column: CHIRAL ART Cellulose-SZ, 3*25 cm, 5 μm; Mobile Phase A: Hex (10 mM NH₃-MeOH), Mobile Phase B: EtOH; Flow rate: 40 mL/min; Gradient: isocratic 30; Wave Length: 203/230 nm; RT1(min): 11.177; RT2(min): 13.551). The first peak afforded 13 mg (30%) as a white solid. MS ESI calculated for C₂₈H₂₀D₃F₃N₄O₃ [M+H]⁺, 524.19, found 524.15. ¹H NMR (400 MHz, Chloroform-d) δ 8.57 (s, 1H), 8.50-8.42 (m, 1H), 7.81 (s, 1H), 7.59 (d, J=8.4 Hz, 1H), 7.42 (t, J=8.2 Hz, 1H), 7.36 (t, J=1.5 Hz, 1H), 7.24-7.17 (m, 1H), 6.92-6.48 (m, 2H), 4.96 (d, J=7.0 Hz, 1H), 3.55-3.41 (m, 1H), 3.14-3.04 (m, 1H), 2.99-2.91 (m, 1H), 2.87 (d, J=13.5 Hz, 1H), 2.44-2.29 (m, 2H), 1.72 (s, 3H). ¹⁹F NMR (377 MHz, Chloroform-d) δ −79.03, −79.47, −82.26, −82.28, −82.70, −82.72, −137.25, −137.27. The second peak afforded 12 mg (28%) as a white solid. MS ESI calculated for C₂₈H₂₀D₃F₃N₄O₃ [M+H]⁺, 524.19, found 524.15. ¹H NMR (400 MHz, Chloroform-d) δ 8.57 (s, 1H), 8.49-8.40 (m, 1H), 7.90 (s, 1H), 7.60 (d, J=8.4 Hz, 1H), 7.43 (t, J=8.1 Hz, 1H), 7.39-7.34 (m, 1H), 7.23-7.18 (m, 1H), 6.92-6.53 (m, 1H), 6.51 (d, J=7.4 Hz, 1H), 4.96 (d, J=6.9 Hz, 1H), 3.55-3.43 (m, 1H), 3.19-3.06 (m, 1H), 3.02-2.92 (m, 1H), 2.88 (d, J=13.5 Hz, 1H), 2.47-2.31 (m, 2H), 1.76 (s, 3H). ¹⁹F NMR (377 MHz, Chloroform-d) δ −79.27, −79.71, −82.23, −82.25, −82.67, −82.69, −137.09, −137.11.

### Example 51 and 52: (7R,14R)-1-(difluoromethoxy)-12-fluoro-11-((R)-4-hydroxy-4-methyl-3,4-dihydro-2H-pyrano[3,2-b]pyridin-7-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-12-fluoro-11-((S)-4-hydroxy-4-methyl-3,4-dihydro-2H-pyrano[3,2-b]pyridin-7-yl)-6-(methyl-d3)-6,7-dihydro-7,14-methanobenzo[f]benzo[4,5]imidazo[1,2-a][1,4]diazocin-5(14H)-one

### Synthetic Scheme

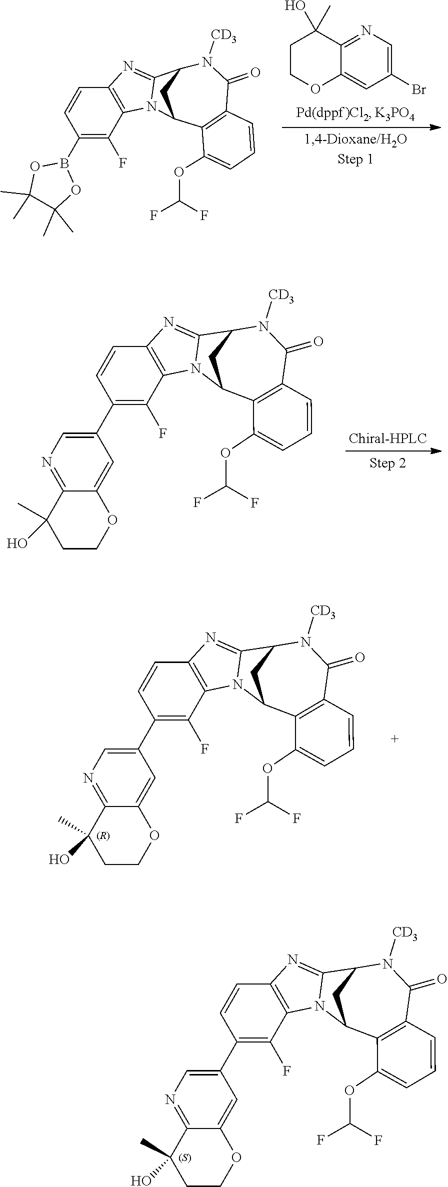

### Preparation 51A: (7R,14R)-1-(difluoromethoxy)-12-fluoro-11-(4-hydroxy-4-methyl-3,4-dihydro-2H-pyrano[3,2-b]pyridin-7-yl)-6-(methyl-d3)-6,7-dihydro-7,14-methanobenzo[f]benzo[4,5]imidazo[1,2-a][1,4]diazocin-5(14H)-one

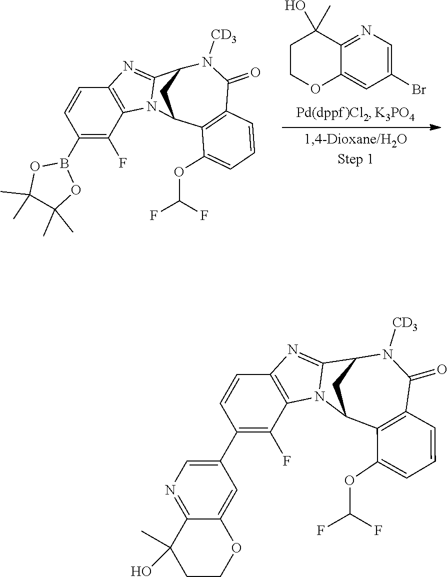

To a stirred solution of (7R,14R)-1-(difluoromethoxy)-12-fluoro-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (90 mg, 0.179 mmol) and 7-bromo-4-methyl-3,4-dihydro-2H-pyrano[3,2-b]pyridin-4-ol (87 mg, 0.358 mmol) in 1,4-dioxane (0.8 mL) and H₂O (0.2 mL) were added Pd(dppf)Cl₂ (15 mg, 0.018 mmol) and K₃PO₄ (114 mg, 0.537 mmol) at room temperature under nitrogen atmosphere. The resulting mixture was stirred for 1 h at 80° C. under nitrogen atmosphere. The resulting mixture was concentrated under vacuum. The residue was purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 30% to 45% gradient in 15 min; detector, 254/220 nm to afford (7R,14R)-1-(difluoromethoxy)-12-fluoro-11-(4-hydroxy-4-methyl-3,4-dihydro-2H-pyrano[3,2-b]pyridin-7-yl)-6-(methyl-d3)-6,7-dihydro-7,14-methanobenzo[f]benzo[4,5]imidazo[1,2-a][1,4]diazocin-5(14H)-one (65 mg, 67%) as a white solid. MS ESI calculated for C₂₈H₂₀D₃F₃N₄O₄ [M+H]⁺, 540.19, found 540.20.

### Example 51 and 52: (7R,14R)-1-(difluoromethoxy)-12-fluoro-11-((R)-4-hydroxy-4-methyl-3,4-dihydro-2H-pyrano[3,2-b]pyridin-7-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-12-fluoro-11-((S)-4-hydroxy-4-methyl-3,4-dihydro-2H-pyrano[3,2-b]pyridin-7-yl)-6-(methyl-d3)-6,7-dihydro-7,14-methanobenzo[f]benzo[4,5]imidazo[1,2-a][1,4]diazocin-5(14H)-one

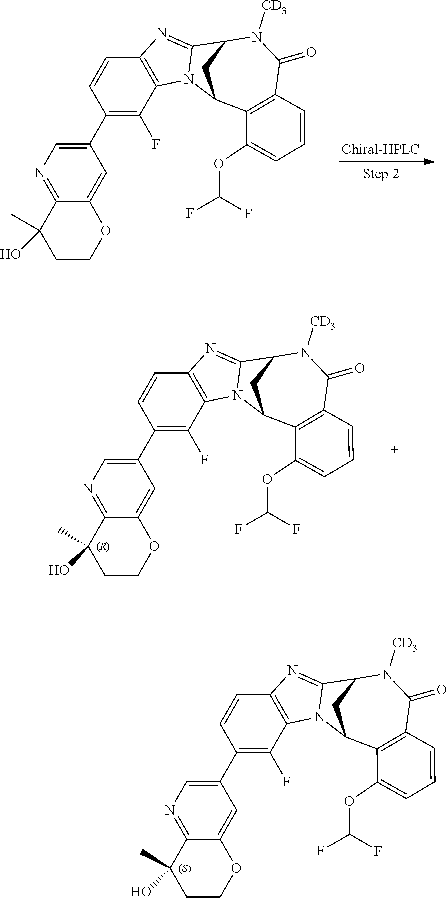

(7R,14R)-1-(difluoromethoxy)-12-fluoro-11-(4-hydroxy-4-methyl-3,4-dihydro-2H-pyrano[3,2-b]pyridin-7-yl)-6-(methyl-d3)-6,7-dihydro-7,14-methanobenzo[f]benzo[4,5]imidazo[1,2-a][1,4]diazocin-5(14H)-one (65 mg) was separated by Prep-Chiral HPLC with the following conditions (Column: CHIRALPAK-IK, 3*25 mm, 5 μm; Mobile Phase A: Hex (10 mM NH₃-MeOH), Mobile Phase B: EtOH; Flow rate: 40 mL/min; Gradient: isocratic 30; Wave Length: 202/220 nm; RT1 (min): 16.8; RT2 (min): 22.1). The first peak afforded 22 mg (34%) as a white solid. MS ESI calculated for C₂₈H₂₀D₃F₃N₄O₄ [M+H]⁺, 540.19, found 540.25. ¹H NMR (400 MHz, Chloroform-d) δ 8.49-8.41 (m, 1H), 8.35 (t, J=1.8 Hz, 1H), 7.58 (d, J=8.5 Hz, 1H), 7.47-7.39 (m, 2H), 7.39-7.33 (m, 1H), 7.24-7.17 (m, 1H), 6.94-6.53 (m, 1H), 6.51 (d, J=7.3 Hz, 1H), 4.95 (d, J=7.0 Hz, 1H), 4.49-4.40 (m, 1H), 4.36-4.26 (m, 1H), 3.81 (s, 1H), 3.55-3.41 (m, 1H), 2.87 (d, J=13.5 Hz, 1H), 2.42-2.30 (m, 1H), 2.25-2.15 (m, 1H), 1.78 (s, 3H). ¹⁹F NMR (377 MHz, Chloroform-d) δ −78.92, −79.36, −82.31, −82.33, −82.75, −82.77, −136.85, −136.87. The second peak afforded 22 mg (34%) as a white solid. MS ESI calculated for C₂₈H₂₀D₃F₃N₄O₄ [M+H]⁺, 540.19, found 540.25. ¹H NMR (400 MHz, Chloroform-d) δ 8.49-8.41 (m, 1H), 8.35 (t, J=1.7 Hz, 1H), 7.58 (d, J=8.4 Hz, 1H), 7.46-7.40 (m, 2H), 7.38-7.34 (m, 1H), 7.24-7.19 (m, 1H), 6.91-6.53 (m, 1H), 6.51 (d, J=7.4 Hz, 1H), 4.95 (d, J=7.0 Hz, 1H), 4.49-4.39 (m, 1H), 4.37-4.26 (m, 1H), 3.86 (s, 1H), 3.55-3.43 (m, 1H), 2.87 (d, J=13.5 Hz, 1H), 2.40-2.30 (m, 1H), 2.23-2.14 (m, 1H), 1.79 (s, 3H). ¹⁹F NMR (377 MHz, Chloroform-d) δ −79.03, −79.47, −82.26, −82.28, −82.70, −82.72, −136.80, −136.82.

### Example 53 and 54: (7R,14R)-1-(difluoromethoxy)-12-fluoro-11-((S)-3-hydroxy-3-methyl-2,3-dihydrofuro[3,2-b]pyridin-6-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-12-fluoro-11-((R)-3-hydroxy-3-methyl-2,3-dihydrofuro[3,2-b]pyridin-6-yl)-6-(methyl-d3)-6,7-dihydro-7,14-methanobenzo[f]benzo[4,5]imidazo[1,2-a][1,4]diazocin-5(14H)-one

### Synthetic Scheme

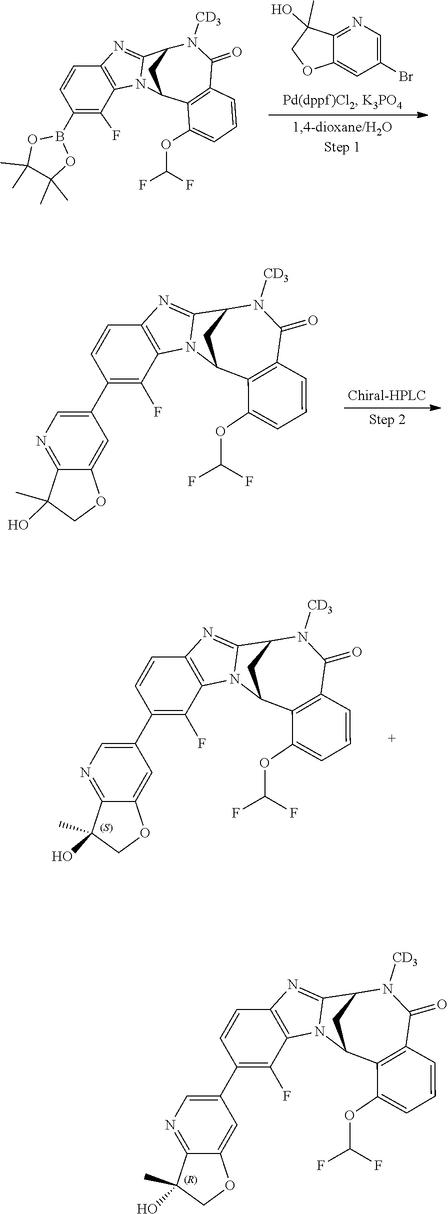

### Preparation 53A: (7R,14R)-1-(difluoromethoxy)-12-fluoro-11-(3-hydroxy-3-methyl-2,3-dihydrofuro[3,2-b]pyridin-6-yl)-6-(methyl-d3)-6,7-dihydro-7,14-methanobenzo[f]benzo[4,5]imidazo[1,2-a][1,4]diazocin-5(14H)-one

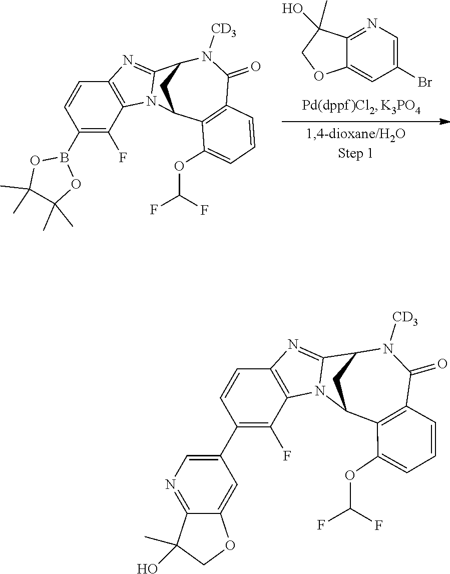 [SMILES: [2H]C([2H])([2H])N1C(=O)c2cccc(OC(F)F)c2[C@H]2C[C@@H]1c1nc3ccc(B4OC(C)(C)C(C)(C)O4)c(F)c3n12 ; CC1(O)COc2cc(Br)cnc21 ; [2H]C([2H])([2H])N1C(=O)c2cccc(OC(F)F)c2[C@H]2C[C@@H]1c1nc3ccc(-c4cnc5c(c4)OCC5(C)O)c(F)c3n12]

To a stirred solution of (7R,14R)-1-(difluoromethoxy)-12-fluoro-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (80 mg, 0.159 mmol) and 6-bromo-3-methyl-2H-furo[3,2-b]pyridin-3-ol (55 mg, 0.238 mmol) in H₂O (0.2 mL) and 1,4-dioxane (1 mL) were added Pd(dppf)Cl₂ (13 mg, 0.016 mmol) and K₃PO₄ (101 mg, 0.477 mmol) at room temperature. The resulting mixture was stirred for 2 h at 80° C. under nitrogen atmosphere. The resulting mixture was concentrated under reduced pressure and purified by silica gel column chromatography, eluted with CH₂Cl₂/MeOH (10/1) to afford (7R,14R)-1-(difluoromethoxy)-12-fluoro-11-(3-hydroxy-3-methyl-2,3-dihydrofuro[3,2-b]pyridin-6-yl)-6-(methyl-d3)-6,7-dihydro-7,14-methanobenzo[f]benzo[4,5]imidazo[1,2-a][1,4]diazocin-5(14H)-one (40 mg, 48%) as a white solid. MS ESI calculated for C₂₇H₁₈D₃F₃N₄O₄ [M+H]⁺, 526.17, found 526.30.

### Example 53 and 54: (7R,14R)-1-(difluoromethoxy)-12-fluoro-11-((S)-3-hydroxy-3-methyl-2,3-dihydrofuro[3,2-b]pyridin-6-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-12-fluoro-11-((R)-3-hydroxy-3-methyl-2,3-dihydrofuro[3,2-b]pyridin-6-yl)-6-(methyl-d3)-6,7-dihydro-7,14-methanobenzo[f]benzo[4,5]imidazo[1,2-a][1,4]diazocin-5(14H)-one

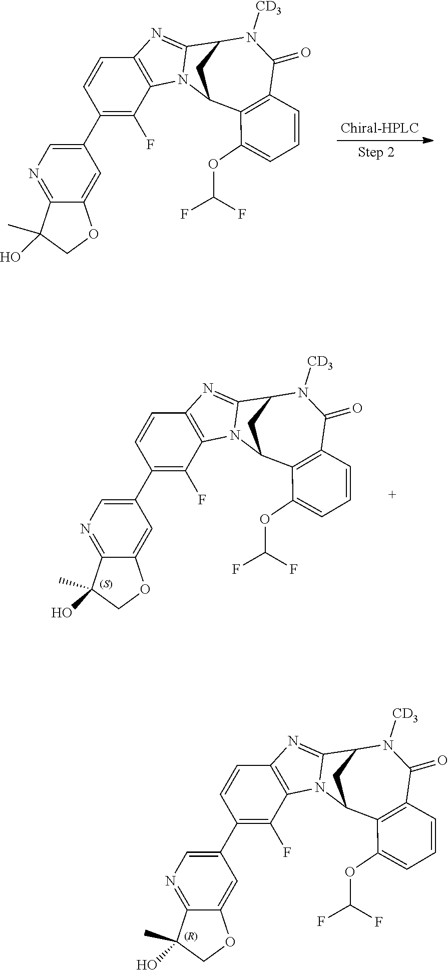

The mixture (40 mg) was resolved by Prep-Chiral HPLC with the following conditions (Column: CHIRALPAK-IK, 3*25 mm, 5 μm; Mobile Phase A: Hex (10 mM NH₃-MeOH), Mobile Phase B: EtOH; Flow rate: 40 mL/min; Gradient: isocratic 30; Wave Length: 202/220 nm; RT1(min): 17.9; RT2(min): 21.7). The first peak afforded (3 mg, 7%) as a white solid. MS ESI calculated for C₂₇H₁₈D₃F₃N₄O₄ [M+H]⁺, 526.17, found 526.15. ¹H NMR (400 MHz, Chloroform-d) δ 8.46-8.40 (m, 1H), 8.22 (t, J=1.6 Hz, 1H), 7.58-7.51 (m, 1H), 7.45-7.37 (m, 1H), 7.37-7.32 (m, 1H), 7.31-7.28 (m, 1H), 7.22-7.15 (m, 1H), 6.89-6.51 (m, 1H), 6.48 (d, J=7.4 Hz, 1H), 4.95 (d, J=7.0 Hz, 1H), 4.69 (d, J=10.1 Hz, 1H), 4.47 (d, J=10.1 Hz, 1H), 3.54-3.42 (m, 1H), 2.86 (d, J=13.6 Hz, 1H), 1.81 (s, 3H). ¹⁹F NMR (377 MHz, Chloroform-d) δ −78.88, −79.31, −82.25, −82.70, −136.94. The second peak afforded (2 mg, 5%) as a white solid. MS ESI calculated for C₂₇H₁₈D₃F₃N₄O₄ [M+H]⁺, 526.17, found 526.15. ¹H NMR (400 MHz, Chloroform-d) δ 8.43 (d, J=8.1 Hz, 1H), 8.23 (s, 1H), 7.55 (d, J=8.4 Hz, 1H), 7.41 (t, J=8.1 Hz, 1H), 7.34 (d, J=8.0 Hz, 1H), 7.28 (s, 1H), 7.22-7.15 (m, 1H), 6.88-6.51 (m, 1H), 6.49 (d, J=7.3 Hz, 1H), 4.95 (d, J=6.9 Hz, 1H), 4.69 (d, J=10.1 Hz, 1H), 4.46 (d, J=10.1 Hz, 1H), 3.53-3.42 (m, 1H), 2.86 (d, J=13.6 Hz, 1H), 1.80 (s, 3H). ¹⁹F NMR (377 MHz, Chloroform-d) δ −78.86, −79.30, −82.22, −82.66, −136.95.

### Example 55 and 56: (7R,14R)-11-((R)-3-amino-3-methyl-2,3-dihydrofuro[3,2-b]pyridin-6-yl)-1-(difluoromethoxy)-6-(methyl-d3)-6,7-dihydro-7,14-methanobenzo[f]benzo[4,5]imidazo[1,2-a][1,4]diazocin-5(14H)-one and (7R,14R)-11-((S)-3-amino-3-methyl-2,3-dihydrofuro[3,2-b]pyridin-6-yl)-1-(difluoromethoxy)-6-(methyl-d3)-6,7-dihydro-7,14-methanobenzo[f]benzo[4,5]imidazo[1,2-a][1,4]diazocin-5(14H)-one

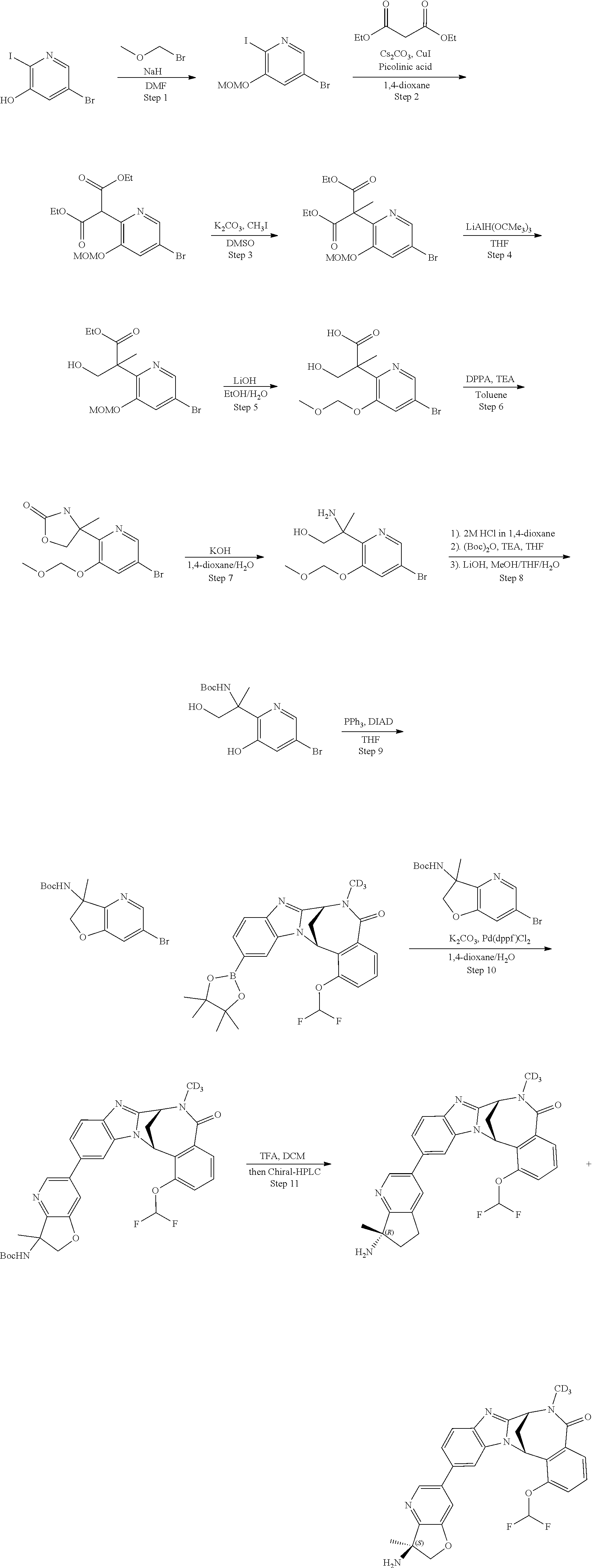

### Preparation 55A: 5-bromo-2-iodo-3-(methoxymethoxy)pyridine

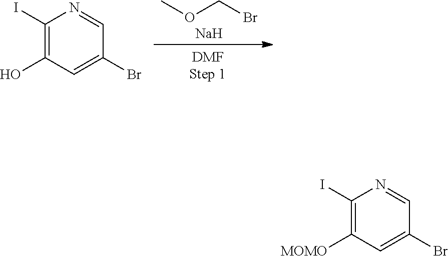

To a stirred solution of 5-bromo-2-iodopyridin-3-ol (25.00 g, 83.363 mmol) in DMF (200 mL) was added NaH (5.00 g, 125.044 mmol, 60%) at 0° C. The resulting mixture was stirred for 30 min at room temperature. To the above mixture was added bromo(methoxy)methane (12.50 g, 100.036 mmol) at room temperature. The resulting mixture was stirred for additional 16 h at room temperature. The reaction was quenched by the addition of ice water (300 mL) at 0° C. The resulting mixture was extracted with EtOAc (3×300 mL). The combined organic layers were washed with brine (3×500 mL), dried over anhydrous Na₂SO₄. After filtration, the filtrate was concentrated under reduced pressure. The residue was purified by silica gel column chromatography, eluted with PE/EA (5/1) to afford 5-bromo-2-iodo-3-(methoxymethoxy)pyridine (23.00 g, 80%) as a white solid. MS ESI calculated for C₇H₇BrINO₂ [M+H]⁺, 343.87 345.87 found 343.90 345.90. ¹H NMR (400 MHz, Chloroform-d) δ 8.14 (d, J=2.4 Hz, 1H), 7.42 (d, J=2.4 Hz, 1H), 5.26 (s, 2H), 3.52 (s, 3H).

### Preparation 55B: 1,3-diethyl 2-[5-bromo-3-(methoxymethoxy)pyridin-2-yl]propanedioate

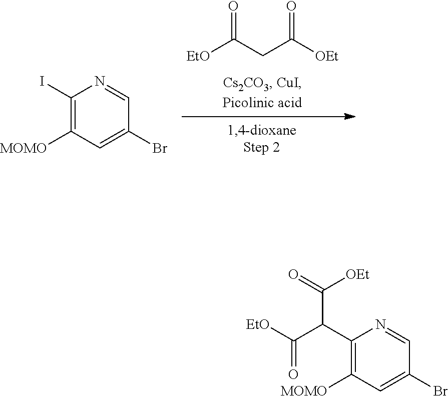

To a solution of 5-bromo-2-iodo-3-(methoxymethoxy)pyridine (23.00 g, 66.871 mmol) in 1,4-dioxane (230 mL) were added diethyl malonate (21.42 g, 133.742 mmol), Cs₂CO₃ (65.36 g, 200.613 mmol), CuI (1.27 g, 6.687 mmol) and pyridine-2-carboxylic acid (1.65 g, 13.374 mmol). The resulting mixture was heated to 70° C. and stirred overnight. water (300 mL) and EtOAc (300 mL) were added and the aqueous layer extracted with EtOAc (3×300 mL). The combined organic layers were washed brine (3×500 mL), dried Na₂SO₄ and concentrated. The residue was purified by silica gel column chromatography, eluted with PE/EA (2/1) to afford 1,3-diethyl 2-[5-bromo-3-(methoxymethoxy)pyridin-2-yl]propanedioate (20.30 g, 80%) as a light yellow solid. MS ESI calculated for C₁₄H₁₈BrNO₆ [M+H]⁺, 376.03 378.03 found 375.95 377.95. ¹H NMR (400 MHz, Chloroform-d) δ 8.29 (d, J=1.9 Hz, 1H), 7.63 (d, J=1.9 Hz, 1H), 5.19 (s, 2H), 5.04 (s, 1H), 4.31-4.23 (m, 4H), 3.46 (s, 3H), 1.30-1.25 (m, 6H).

### Preparation 55C: 1,3-diethyl 2-[5-bromo-3-(methoxymethoxy)pyridin-2-yl]-2-methylpropanedioate

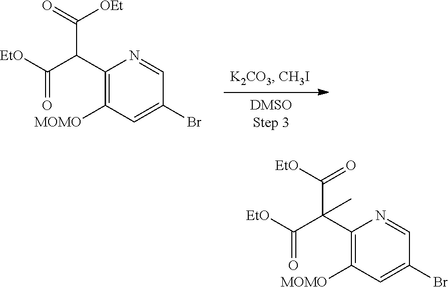

To a stirred solution of 1,3-diethyl 2-[5-bromo-3-(methoxymethoxy)pyridin-2-yl]propanedioate (4.20 g, 11.164 mmol) and K₂CO₃ (1.85 g, 13.397 mmol) in DMSO (12 mL) was added CH₃I (1.90 g, 13.397 mmol) dropwise at room temperature under nitrogen atmosphere. The resulting mixture was stirred for additional 3 h at room temperature. The resulting mixture was diluted with water (50 mL) and EtOAc (40 mL). The mixture was extracted with EtOAc (3×40 mL). The combined organic layers were washed with brine (3×100 mL), dried over anhydrous Na₂SO₄. After filtration, the filtrate was concentrated under reduced pressure to afford 1,3-diethyl 2-[5-bromo-3-(methoxymethoxy)pyridin-2-yl]-2-methylpropanedioate (4.00 g, 91%) as an orange oil. MS ESI calculated for C₁₅H₂₀BrNO₆ [M+H]⁺, 390.05 392.05 found 390.05 392.05. ¹H NMR (400 MHz, Chloroform-d) δ 8.23 (d, J=1.9 Hz, 1H), 7.65 (d, J=1.9 Hz, 1H), 5.14 (s, 2H), 4.27-4.19 (m, 4H), 3.47 (s, 3H), 1.86 (s, 3H), 1.25 (t, J=7.1 Hz, 6H).

### Preparation 55D: ethyl 2-[5-bromo-3-(methoxymethoxy)pyridin-2-yl]-3-hydroxy-2-methylpropanoate

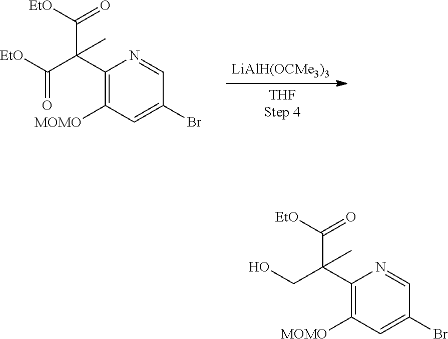

To a solution of 1,3-diethyl 2-[5-bromo-3-(methoxymethoxy)pyridin-2-yl]-2-methylpropanedioate (3.50 g, 8.969 mmol) in THF (20 mL) was added Lithium tri-tert-butoxyaluminum hydride (22.42 mL, 22.422 mmol, 1.0 M solution in THF) dropwise at room temperature. The reaction mixture was heated to reflux for 30 min, after which it was quenched with 10% (w/v) aq. KHSO₄ (80 mL) and extracted with CH₂Cl₂. The aqueous layer was isolated and extracted again with CH₂Cl₂. The combined CH₂Cl₂ layers were washed with brine, dried over Na₂SO₄, filtered, and concentrated to dryness. The residue was purified by silica gel column chromatography, eluted with PE/EA (1/1) to afford ethyl 2-[5-bromo-3-(methoxymethoxy)pyridin-2-yl]-3-hydroxy-2-methylpropanoate (2.50 g, 80%) as a yellow oil. MS ESI calculated for C₁₃H₁₈BrNO₅ [M+H]⁺, 348.04 350.04 found 348.05 350.05. ¹H NMR (400 MHz, Chloroform-d) δ 8.24 (d, J=2.0 Hz, 1H), 7.65 (d, J=2.0 Hz, 1H), 5.19-5.10 (m, 2H), 4.19-4.06 (m, 3H), 3.95 (d, J=11.1 Hz, 1H), 3.47 (s, 3H), 1.56 (s, 3H), 1.20 (t, J=7.1 Hz, 3H).

### Preparation 55E: 2-[5-bromo-3-(methoxymethoxy)pyridin-2-yl]-3-hydroxy-2-methylpropanoic acid

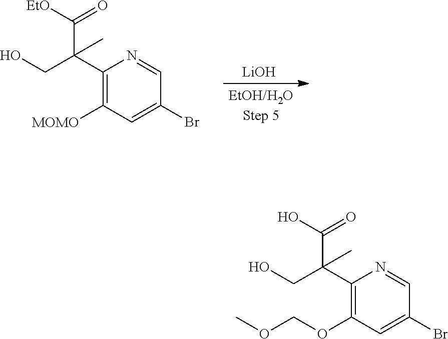

To a stirred solution of ethyl 2-[5-bromo-3-(methoxymethoxy)pyridin-2-yl]-3-hydroxy-2-methylpropanoate (3.8 g, 10.913 mmol) in EtOH (20 mL) and H₂O (20 mL) was added LiOH·H₂O (2.06 g, 49.094 mmol) at room temperature. The resulting mixture was stirred for 3 h at 80° C. The mixture was acidified to pH 6 with 1M HCl (aq.). The resulting mixture was extracted with EtOAc (4×100 mL). The combined organic layers were dried over anhydrous Na₂SO₄. After filtration, the filtrate was concentrated under reduced pressure. The residue was purified by silica gel column chromatography, eluted with PE/EA (1/1) to afford 2-[5-bromo-3-(methoxymethoxy)pyridin-2-yl]-3-hydroxy-2-methylpropanoic acid (1.20 g, 34%) as a white solid. MS ESI calculated for C₁₁H₁₄BrNO₅ [M+H]⁺, 320.01 322.01 found 320.00 322.00. ¹H NMR (400 MHz, Chloroform-d) δ 8.23 (d, J=1.9 Hz, 1H), 7.66 (d, J=1.9 Hz, 1H), 5.21-5.13 (m, 2H), 4.17-4.01 (m, 2H), 3.46 (s, 3H), 1.53 (s, 3H).

### Preparation 55F: 4-[5-bromo-3-(methoxymethoxy)pyridin-2-yl]-4-methyl-1,3-oxazolidin-2-one

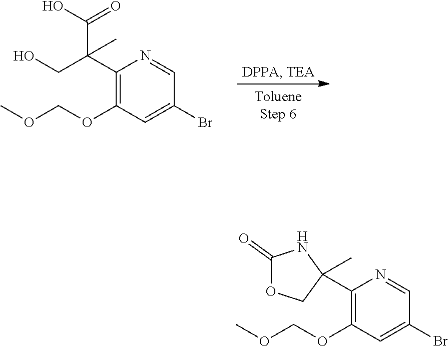 [SMILES: COCOc1cc(Br)cnc1C(C)(CO)C(=O)O ; "DPPA, TEA" ; COCOc1cc(Br)cnc1C1(C)COC(=O)N1]

To a stirred solution of 2-[5-bromo-3-(methoxymethoxy)pyridin-2-yl]-3-hydroxy-2-methylpropanoic acid (1.20 g, 3.748 mmol) and TEA (0.6 mL, 4.498 mmol) in Toluene (20 mL) was added DPPA (1.24 g, 4.498 mmol) dropwise at room temperature under nitrogen atmosphere. The resulting mixture was stirred for 3 h at 80° C. The resulting mixture was concentrated under vacuum. The residue was purified by silica gel column chromatography, eluted with PE/EA (1/2) to afford 4-[5-bromo-3-(methoxymethoxy)pyridin-2-yl]-4-methyl-1,3-oxazolidin-2-one (900 mg, 75%) as a white solid. MS ESI calculated for C₁₁H₁₃BrN₂O₄ [M+H]⁺, 317.01 319.01 found 317.10 319.10. ¹H NMR (400 MHz, Chloroform-d) δ 8.26 (d, J=1.8 Hz, 1H), 7.69 (d, J=1.8 Hz, 1H), 6.25 (s, 1H), 5.29 (s, 2H), 4.92 (d, J=9.0 Hz, 1H), 4.44 (d, J=8.9 Hz, 1H), 3.52 (s, 3H), 1.63 (s, 3H).

### Preparation 55G: 2-amino-2-[5-bromo-3-(methoxymethoxy)pyridin-2-yl]propan-1-ol

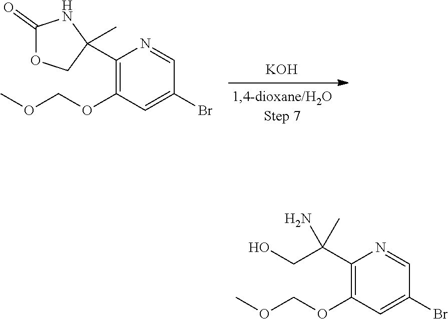 [SMILES: COCOc1cc(Br)cnc1C1(C)COC(=O)N1 ; "KOH" ; COCOc1cc(Br)cnc1C(C)(N)CO]

A solution of 4-[5-bromo-3-(methoxymethoxy)pyridin-2-yl]-4-methyl-1,3-oxazolidin-2-one (900 mg, 2.838 mmol) and KOH (1.59 g, 28.380 mmol) in EtOH (20 mL) and H₂O (10 mL) was stirred for overnight at 80° C. under nitrogen atmosphere. The resulting mixture was extracted with EtOAc (3×15 mL). The combined organic layers were washed with brine (3×20 mL), dried over anhydrous Na₂SO₄. After filtration, the filtrate was concentrated under reduced pressure. The residue was purified by silica gel column chromatography, eluted with CH₂Cl₂/MeOH (5/1) to afford 2-amino-2-[5-bromo-3-(methoxymethoxy)pyridin-2-yl]propan-1-ol (610 mg, 73%) as a yellow solid. MS ESI calculated for C₁₀H₁₅BrN₂O₃ [M+H]⁺, 291.03 293.03 found 290.85 292.85. ¹H NMR (400 MHz, DMSO-d₆) δ 8.25 (d, J=1.9 Hz, 1H), 7.65 (d, J=1.9 Hz, 1H), 5.36 (s, 2H), 4.50 (brs, 1H), 3.78 (d, J=10.3 Hz, 1H), 3.57 (d, J=10.4 Hz, 1H), 3.42 (s, 3H), 3.00 (s, 2H), 1.34 (s, 3H).

### Preparation 55H: tert-butyl N-[2-(5-bromo-3-hydroxypyridin-2-yl)-1-hydroxypropan-2-yl]carbamate

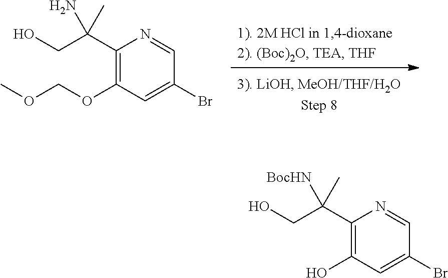 [SMILES: COCOc1cc(Br)cnc1C(C)(N)CO ; CC(C)(C)OC(=O)NC(C)(CO)c1ncc(Br)cc1O]

A solution of 2-amino-2-[5-bromo-3-(methoxymethoxy)pyridin-2-yl]propan-1-ol (610 mg, 2.095 mmol) in 2 M HCl(gas) in 1,4-dioxane (4 mL) was stirred for 1 h at room temperature. The resulting mixture was concentrated under vacuum. To a stirred solution of the above residue and TEA (2.4 mL, 16.760 mmol) in THF (5 mL) were added Boc₂O (1.10 g, 5.028 mmol) and DMAP (13 mg, 0.105 mmol) at room temperature. The resulting mixture was stirred for overnight at room temperature. The resulting mixture was concentrated under vacuum. To a stirred solution of the above residue and THF (5 mL) in H₂O (5 mL) and MeOH (5 mL) was added LiOH·H₂O (703 mg, 16.760 mmol) at room temperature. The resulting mixture was stirred for overnight at room temperature. The resulting mixture was diluted with EtOAc (50 mL) and H₂O (50 mL) and extracted with EtOAc (2×50 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EA (1/1) to afford tert-butyl N-[2-(5-bromo-3-hydroxypyridin-2-yl)-1-hydroxypropan-2-yl]carbamate (320 mg, 43%) as a white solid. MS ESI calculated for C₁₃H₁₉BrN₂O₄ [M+H]⁺, 347.05 349.05 found 347.10 349.10. ¹H NMR (400 MHz, Chloroform-d) δ 10.60 (s, 1H), 8.07 (d, J=2.0 Hz, 1H), 7.49 (d, J=1.8 Hz, 1H), 6.22 (s, 1H), 4.27 (d, J=11.4 Hz, 1H), 3.67 (d, J=11.5 Hz, 1H), 1.68 (s, 3H), 1.43 (s, 9H).

### Preparation 55I: tert-butyl N-{6-bromo-3-methyl-2H-furo[3,2-b]pyridin-3-yl}carbamate

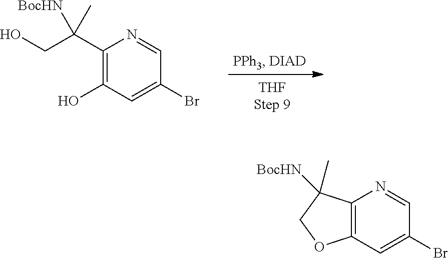 [SMILES: CC(C)(C)OC(=O)NC(C)(CO)c1ncc(Br)cc1O ; CC(C)(C)OC(=O)NC1(C)COc2cc(Br)cnc21]

To a stirred solution of tert-butyl N-[2-(5-bromo-3-hydroxypyridin-2-yl)-1-hydroxypropan-2-yl]carbamate (320 mg, 0.922 mmol) and PPh₃ (362 mg, 1.383 mmol) in THF (6 mL) was added DIAD (279 mg, 1.383 mmol) dropwise at 0° C. under nitrogen atmosphere. The resulting mixture was stirred for 2 h at room temperature. The resulting mixture was concentrated under vacuum. The residue was purified by silica gel column chromatography, eluted with PE/EA (5/1) to afford tert-butyl N-{6-bromo-3-methyl-2H-furo[3,2-b]pyridin-3-yl}carbamate (260 mg, 85%) as a white solid. MS ESI calculated for C₁₃H₁₇BrN₂O₃ [M+H]⁺, 329.04 331.04 found 329.05 331.05. ¹H NMR (400 MHz, Chloroform-d) δ 8.15 (d, J=1.8 Hz, 1H), 7.27 (d, J=1.8 Hz, 1H), 4.90 (d, J=9.7 Hz, 1H), 4.60 (d, J=9.7 Hz, 1H), 1.64 (s, 3H), 1.40 (s, 9H).

### Preparation 55J: tert-butyl (6-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-3-methyl-2,3-dihydrofuro[3,2-b]pyridin-3-yl)carbamate

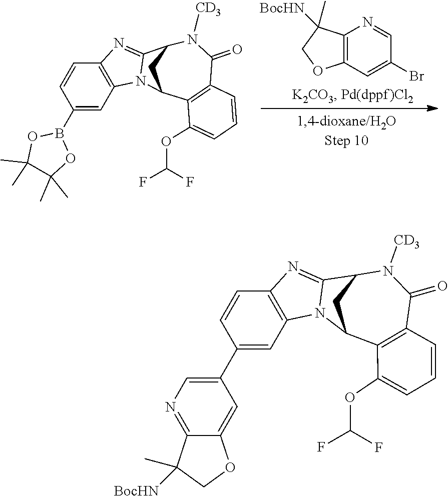 [SMILES: [2H]C([2H])([2H])N1C(=O)c2cccc(OC(F)F)c2[C@H]2C[C@@H]1c1nc3ccc(B4OC(C)(C)C(C)(C)O4)cc3n12 ; CC(C)(C)OC(=O)NC1(C)COc2cc(Br)cnc21 ; [2H]C([2H])([2H])N1C(=O)c2cccc(OC(F)F)c2[C@H]2C[C@@H]1c1nc3ccc(-c4cnc5c(c4)OCC5(C)NC(=O)OC(C)(C)C)cc3n12]

To a solution of (7R,14R)-1-(difluoromethoxy)-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (60 mg, 0.124 mmol) and tert-butyl N-{6-bromo-3-methyl-2H-furo[3,2-b]pyridin-3-yl}carbamate (40 mg, 0.124 mmol) in 1,4-dioxane (1 mL) and H₂O (0.2 mL) were added K₂CO₃ (34.24 mg, 0.248 mmol) and Pd(dppf)Cl₂ (10 mg, 0.012 mmol). After stirring for 2 h at 80° C. under a nitrogen atmosphere, the resulting mixture was concentrated under reduced pressure. The residue was purified by silica gel column chromatography, eluted with CH₂Cl₂/MeOH (12/1) to afford tert-butyl (6-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-3-methyl-2,3-dihydrofuro[3,2-b]pyridin-3-yl)carbamate (110 mg, crude) as a light yellow solid. MS ESI calculated for C₃₂H₂₈D₃F₂N₅O₅ [M+H]⁺, 607.25, found 607.25.

### Example 55 and 56: (7R,14R)-11-((R)-3-amino-3-methyl-2,3-dihydrofuro[3,2-b]pyridin-6-yl)-1-(difluoromethoxy)-6-(methyl-d3)-6,7-dihydro-7,14-methanobenzo[f]benzo[4,5]imidazo[1,2-a][1,4]diazocin-5(14H)-one and (7R,14R)-11-((S)-3-amino-3-methyl-2,3-dihydrofuro[3,2-b]pyridin-6-yl)-1-(difluoromethoxy)-6-(methyl-d3)-6,7-dihydro-7,14-methanobenzo[f]benzo[4,5]imidazo[1,2-a][1,4]diazocin-5(14H)-one

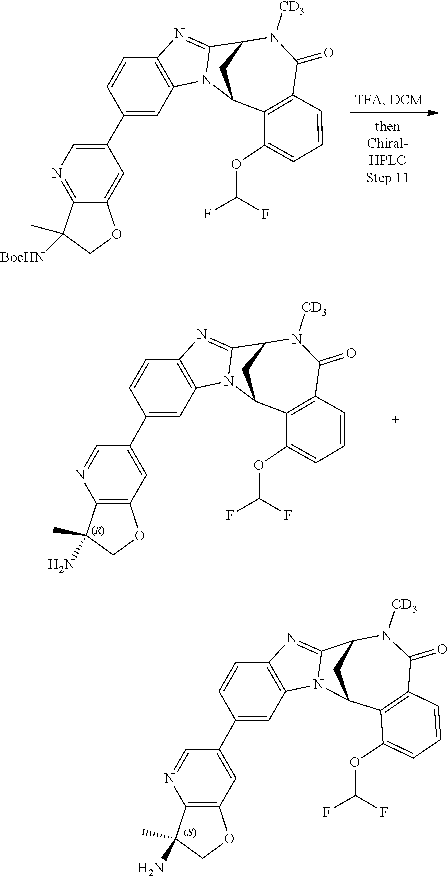

To a stirred solution of tert-butyl (6-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-3-methyl-2,3-dihydrofuro[3,2-b]pyridin-3-yl)carbamate (110 mg, 0.181 mmol) in DCM (3 mL) was added TFA (1 mL) dropwise at room temperature. The resulting mixture was stirred for 1 h at room temperature. The resulting mixture was concentrated under vacuum. The residue was purified by reversed-phase flash chromatography with the following conditions: column, C18 silica gel; mobile phase, CH₃CN in Water (10 mmol/L NH₄HCO₃), 5% to 95% gradient in 25 min; detector, 254 nm followed by Chiral-HPLC with the following conditions (Column: CHIRAL ART Cellulose-SZ, 3*25 cm, 5 μm; Mobile Phase A: Hex (10 mM NH₃-MeOH), Mobile Phase B: EtOH; Flow rate: 40 mL/min; Gradient: isocratic 50; Wave Length: 209/220 nm; RT1(min): 10.38; RT2(min): 16.353.). The first peak afforded 17 mg (18%) as a white solid. MS ESI calculated for C₂₇H₂₀D₃F₂N₅O₃ [M+H]⁺, 507.20, found 507.20. ¹H NMR (400 MHz, Chloroform-d) δ 8.54-8.44 (m, 1H), 8.34 (d, J=1.8 Hz, 1H), 7.79-7.72 (m, 1H), 7.55 (s, 1H), 7.45-7.35 (m, 2H), 7.33-7.28 (m, 1H), 7.23 (d, J=2.4 Hz, 1H), 6.84 (t, J=72.8 Hz, 1H), 6.25 (d, J=7.2 Hz, 1H), 4.97 (d, J=7.0 Hz, 1H), 4.60 (d, J=9.8 Hz, 1H), 4.51 (d, J=9.8 Hz, 1H), 3.59-3.43 (m, 1H), 2.88 (d, J=13.5 Hz, 1H), 1.74 (s, 3H). ¹⁹F NMR (377 MHz, Chloroform-d) δ −80.24, −80.69, −80.82, −81.27. The second peak afforded 18 mg (19%) as a white solid. MS ESI calculated for C₂₇H₂₀D₃F₂N₅O₃ [M+H]⁺, 507.20, found 507.20. ¹H NMR (400 MHz, Chloroform-d) δ 8.52-8.45 (m, 1H), 8.31 (d, J=1.7 Hz, 1H), 7.81-7.74 (m, 1H), 7.61 (s, 1H), 7.45-7.35 (m, 2H), 7.34-7.29 (m, 1H), 7.28-7.27 (m, 1H), 7.04-6.63 (m, 1H), 6.31-6.24 (m, 1H), 5.00-4.93 (m, 1H), 4.58-4.45 (m, 2H), 3.57-3.41 (m, 1H), 2.88 (d, J=13.6 Hz, 1H), 1.67 (s, 3H). ¹⁹F NMR (377 MHz, Chloroform-d) δ −80.16, −80.61, −80.78, −81.23.

### Example 57 and 58: (7R,14R)-11-((R)-3-amino-3-methyl-2,3-dihydrofuro[3,2-b]pyridin-6-yl)-1-(difluoromethoxy)-6-methyl-6,7-dihydro-7,14-methanobenzo[f]benzo[4,5]imidazo[1,2-a][1,4]diazocin-5(14H)-one and (7R,14R)-11-((S)-3-amino-3-methyl-2,3-dihydrofuro[3,2-b]pyridin-6-yl)-1-(difluoromethoxy)-6-methyl-6,7-dihydro-7,14-methanobenzo[f]benzo[4,5]imidazo[1,2-a][1,4]diazocin-5(14H)-one

### Synthetic Scheme

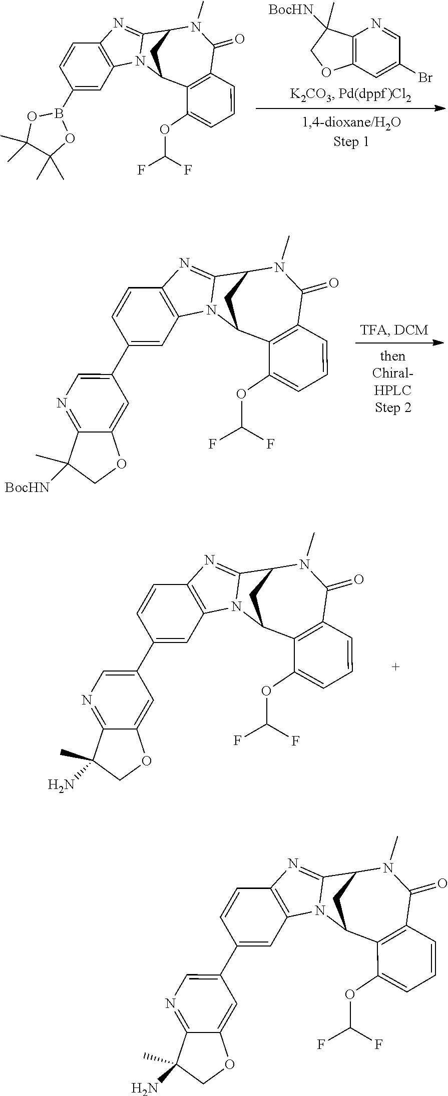 [SMILES: CN1C(=O)c2cccc(OC(F)F)c2[C@H]2C[C@@H]1c1nc3ccc(B4OC(C)(C)C(C)(C)O4)cc3n12 ; CC(C)(C)OC(=O)NC1(C)COc2cc(Br)cnc21 ; CN1C(=O)c2cccc(OC(F)F)c2[C@H]2C[C@@H]1c1nc3ccc(-c4cnc5c(c4)OCC5(C)NC(=O)OC(C)(C)C)cc3n12 ; CN1C(=O)c2cccc(OC(F)F)c2[C@H]2C[C@@H]1c1nc3ccc(-c4cnc5c(c4)OC[C@]5(C)N)cc3n12 ; CN1C(=O)c2cccc(OC(F)F)c2[C@H]2C[C@@H]1c1nc3ccc(-c4cnc5c(c4)OC[C@@]5(C)N)cc3n12]

### Preparation 57A: tert-butyl (6-((7R,14R)-1-(difluoromethoxy)-6-methyl-5-oxo-5,6,7,14-tetrahydro-7,14-methanobenzo[f]benzo[4,5]imidazo[1,2-a][1,4]diazocin-11-yl)-3-methyl-2,3-dihydrofuro[3,2-b]pyridin-3-yl)carbamate

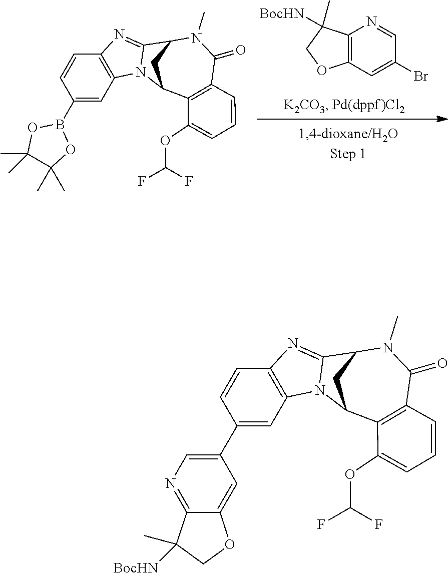 [SMILES: CN1C(=O)c2cccc(OC(F)F)c2[C@H]2C[C@@H]1c1nc3ccc(B4OC(C)(C)C(C)(C)O4)cc3n12 ; CC(C)(C)OC(=O)NC1(C)COc2cc(Br)cnc21 ; CN1C(=O)c2cccc(OC(F)F)c2[C@H]2C[C@@H]1c1nc3ccc(-c4cnc5c(c4)OCC5(C)NC(=O)OC(C)(C)C)cc3n12]

To a solution of (7R,14R)-1-(difluoromethoxy)-6-methyl-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (90 mg, 0.159 mmol) and tert-butyl N-{6-bromo-3-methyl-2H-furo[3,2-b]pyridin-3-yl}carbamate (52 mg, 0.159 mmol) in 1,4-dioxane (1 mL) and H₂O (0.2 mL) were added K₂CO₃ (54 mg, 0.398 mmol) and Pd(dppf)Cl₂ (12 mg, 0.016 mmol). After stirring for 2 h at 80° C. under a nitrogen atmosphere, the resulting mixture was concentrated under reduced pressure and purified by silica gel column chromatography, eluted with CH₂Cl₂/MeOH (12/1) to afford tert-butyl (6-((7R,14R)-1-(difluoromethoxy)-6-methyl-5-oxo-5,6,7,14-tetrahydro-7,14-methanobenzo[f]benzo[4,5]imidazo[1,2-a][1,4]diazocin-11-yl)-3-methyl-2,3-dihydrofuro[3,2-b]pyridin-3-yl)carbamate (115 mg, crude) as a light yellow solid. MS ESI calculated for C₃₂H₃₁F₂N₅O₅ [M+H]⁺, 604.23 found 604.25.

### Example 57 and 58: (7R,14R)-11-((R)-3-amino-3-methyl-2,3-dihydrofuro[3,2-b]pyridin-6-yl)-1-(difluoromethoxy)-6-methyl-6,7-dihydro-7,14-methanobenzo[f]benzo[4,5]imidazo[1,2-a][1,4]diazocin-5(14H)-one and (7R,14R)-11-((S)-3-amino-3-methyl-2,3-dihydrofuro[3,2-b]pyridin-6-yl)-1-(difluoromethoxy)-6-methyl-6,7-dihydro-7,14-methanobenzo[f]benzo[4,5]imidazo[1,2-a][1,4]diazocin-5(14H)-one

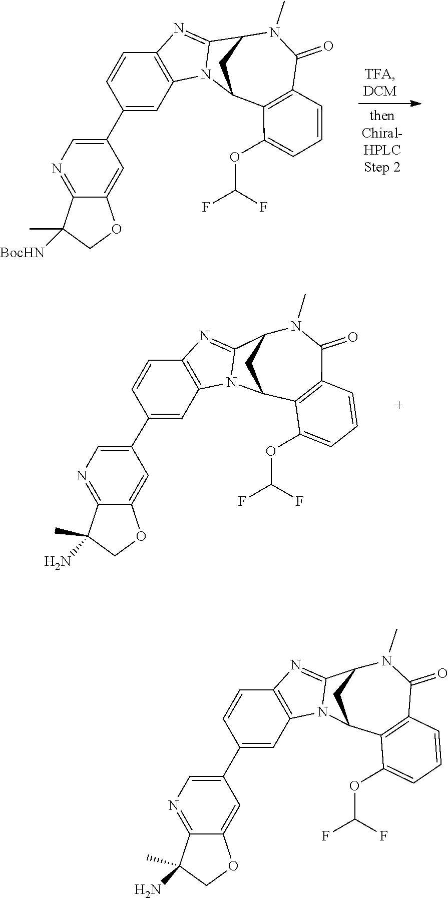 [SMILES: CN1C(=O)c2cccc(OC(F)F)c2[C@H]2C[C@@H]1c1nc3ccc(-c4cnc5c(c4)OCC5(C)NC(=O)OC(C)(C)C)cc3n12 ; CN1C(=O)c2cccc(OC(F)F)c2[C@H]2C[C@@H]1c1nc3ccc(-c4cnc5c(c4)OC[C@]5(C)N)cc3n12 ; CN1C(=O)c2cccc(OC(F)F)c2[C@H]2C[C@@H]1c1nc3ccc(-c4cnc5c(c4)OC[C@@]5(C)N)cc3n12]

To a stirred solution of tert-butyl (6-((7R,14R)-1-(difluoromethoxy)-6-methyl-5-oxo-5,6,7,14-tetrahydro-7,14-methanobenzo[f]benzo[4,5]imidazo[1,2-a][1,4]diazocin-11-yl)-3-methyl-2,3-dihydrofuro[3,2-b]pyridin-3-yl)carbamate (115 mg, 0.191 mmol) in DCM (3 mL) was added TFA (1 mL) dropwise at room temperature under nitrogen atmosphere. The resulting mixture was stirred for 1 h at room temperature. The resulting mixture was concentrated under vacuum and purified by reversed-phase flash chromatography with the following conditions: column, C18 silica gel; mobile phase, CH₃CN in Water (10 mmol/L NH₄HCO₃), 5% to 95% gradient in 25 min; detector, 254 nm to afford the mixture (94 mg). The mixture was purified by Prep-Chiral HPLC with the following conditions (Column: CHIRAL ART Cellulose-SZ, 3*25 cm, 5 μm; Mobile Phase A: Hex (10 mM NH₃-MeOH), Mobile Phase B: ETOH; Flow rate: 40 mL/min; Gradient: isocratic 50; Wave Length: 220/254 nm; RT1(min): 11.44; RT2(min): 20.65). The first peak afforded 24 mg (25%) as a white solid. MS ESI calculated for C₂₇H₂₃F₂N₅O₃ [M+H]⁺, 504.18 found 504.10. ¹H NMR (400 MHz, Chloroform-d) δ 8.53-8.45 (m, 1H), 8.34 (d, J=1.8 Hz, 1H), 7.81-7.74 (m, 1H), 7.63 (d, J=1.7 Hz, 1H), 7.47-7.38 (m, 2H), 7.34-7.27 (m, 1H), 7.25 (d, J=1.8 Hz, 1H), 6.83 (t, J=72.8 Hz, 1H), 6.27 (d, J=7.2 Hz, 1H), 4.97 (d, J=7.0 Hz, 1H), 4.49 (s, 2H), 3.52 (s, 3H), 3.50-3.42 (m, 1H), 2.88 (d, J=13.5 Hz, 1H), 1.65 (s, 3H). ¹⁹F NMR (377 MHz, Chloroform-d) δ −80.17, −80.61, −80.78, −81.23. The second peak afforded 23 mg (29%) as a white solid. MS ESI calculated for C₂₇H₂₃F₂N₅O₃ [M+H]⁺, 504.18 found 504.10. ¹H NMR (400 MHz, Chloroform-d) δ 8.54-8.45 (m, 1H), 8.33 (d, J=1.8 Hz, 1H), 7.78 (d, J=8.5 Hz, 1H), 7.64 (d, J=1.8 Hz, 1H), 7.46-7.37 (m, 2H), 7.31 (d, J=8.1 Hz, 1H), 7.27-7.26 (m, 1H), 6.84 (t, J=72.8 Hz, 1H), 6.28 (d, J=7.2 Hz, 1H), 4.97 (d, J=7.1 Hz, 1H), 4.48 (s, 2H), 3.52 (s, 3H), 3.50-3.42 (m, 1H), 2.88 (d, J=13.5 Hz, 1H), 1.64 (s, 3H). ¹⁹F NMR (377 MHz, Chloroform-d) δ −80.14, −80.59, −80.78, −81.22.

### Example 59 and 60: (7R,14R)-11-((S)-3-amino-3-methyl-2,3-dihydrofuro[3,2-b]pyridin-6-yl)-1-(difluoromethoxy)-6-(methyl-d3)-6,7-dihydro-7,14-methanobenzo[f]pyrido[3′,2′:4,5]imidazo[1,2-a][1,4]diazocin-5(14H)-one and (7R,14R)-11-((R)-3-amino-3-methyl-2,3-dihydrofuro[3,2-b]pyridin-6-yl)-1-(difluoromethoxy)-6-(methyl-d3)-6,7-dihydro-7,14-methanobenzo[f]pyrido[3′,2′:4,5]imidazo[1,2-a][1,4]diazocin-5(14H)-one

### Synthetic Scheme

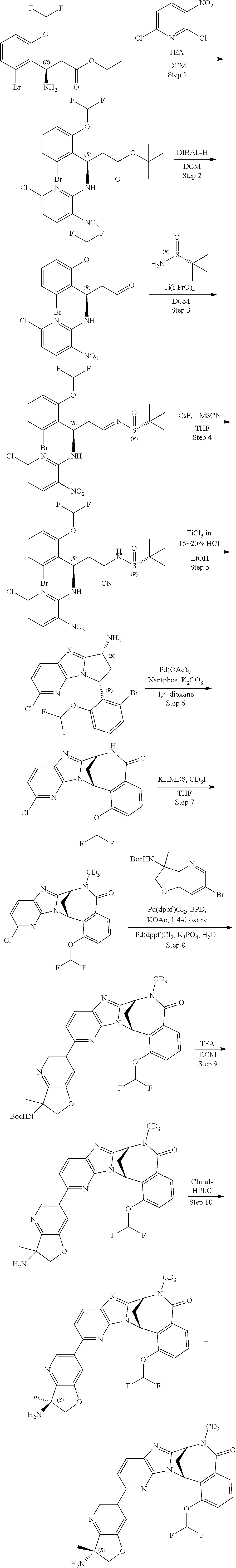

### Preparation 59A: tert-butyl (R)-3-(2-bromo-6-(difluoromethoxy)phenyl)-3-((6-chloro-3-nitropyridin-2-yl)amino)propanoate

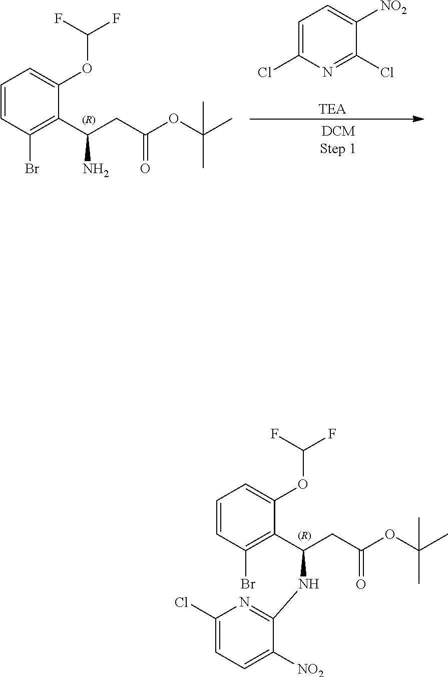

To a stirred mixture of tert-butyl (3R)-3-amino-3-[2-bromo-6-(difluoromethoxy)phenyl]propanoate (5.00 g, 13.654 mmol) and 2,6-dichloro-3-nitropyridine (3.43 g, 17.750 mmol) in DCM (50 mL) was added TEA (2.85 mL, 20.481 mmol) at room temperature. The resulting mixture was stirred for 16 h at room temperature. The resulting mixture was concentrated under reduced pressure and purified by silica gel column chromatography, eluted with PE/DCM (1:1) to afford tert-butyl (R)-3-(2-bromo-6-(difluoromethoxy)phenyl)-3-((6-chloro-3-nitropyridin-2-yl)amino)propanoate (6.50 g, 91%) as a yellow oil. MS ESI calculated for C₁₉H₁₉BrClF₂N₃O₅ [M+H]⁺, 522.02 524.02 found 522.05 524.05. ¹H NMR (400 MHz, Chloroform-d) δ 9.16 (d, J=8.5 Hz, 1H), 8.31 (d, J=8.6 Hz, 1H), 7.46-7.38 (m, 1H), 7.18-7.10 (m, 2H), 6.91-6.68 (m, 1H), 6.61 (d, J=8.6 Hz, 1H), 6.59-6.51 (m, 1H), 3.17-3.05 (m, 1H), 2.90-2.79 (m, 1H), 1.36 (s, 9H).

### Preparation 59B: (R)-3-(2-bromo-6-(difluoromethoxy)phenyl)-3-((6-chloro-3-nitropyridin-2-yl)amino)propanal

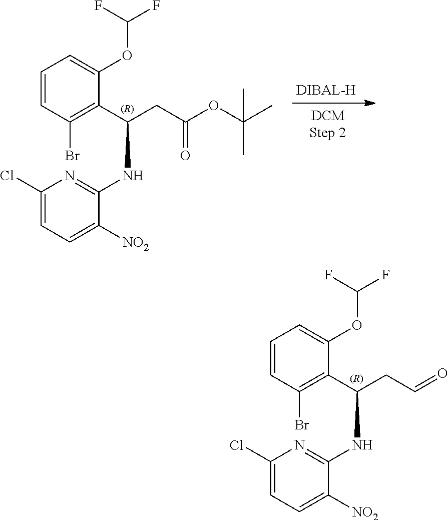

To a stirred mixture of tert-butyl (R)-3-(2-bromo-6-(difluoromethoxy)phenyl)-3-((6-chloro-3-nitropyridin-2-yl)amino)propanoate (6.50 g, 12.435 mmol) in DCM (97 mL) was added 1 M DIBAL-H in DCM (13.68 mL, 13.679 mmol) at −78° C. under nitrogen atmosphere. The resulting mixture was stirred for 3 h at −78° C. under nitrogen atmosphere. The reaction was quenched with water at −78° C. The resulting mixture was filtered, the filter cake was washed with DCM (3×100 mL). The filtrate was extracted with CH₂Cl₂ (3×200 mL). The resulting mixture was concentrated under vacuum. The residue was purified by silica gel column chromatography, eluted with PE/EA (5:1) to afford (R)-3-(2-bromo-6-(difluoromethoxy)phenyl)-3-((6-chloro-3-nitropyridin-2-yl)amino)propanal (5.00 g, 89%) as a yellow oil. MS ESI calculated for C₁₅H₁₁BrClF₂N₃O₄ [M+H]⁺, 449.96 451.96 found 449.90 451.90. ¹H NMR (400 MHz, Chloroform-d) δ 9.82 (s, 1H), 9.17 (d, J=8.7 Hz, 1H), 8.33 (d, J=8.5 Hz, 1H), 7.51-7.43 (m, 1H), 7.26-7.15 (m, 2H), 6.93-6.56 (m, 3H), 3.39-3.31 (m, 1H), 3.09-3.00 (m, 1H).

### Preparation 59C: (R)—N—((R,E)-3-(2-bromo-6-(difluoromethoxy)phenyl)-3-((6-chloro-3-nitropyridin-2-yl)amino)propylidene)-2-methylpropane-2-sulfinamide

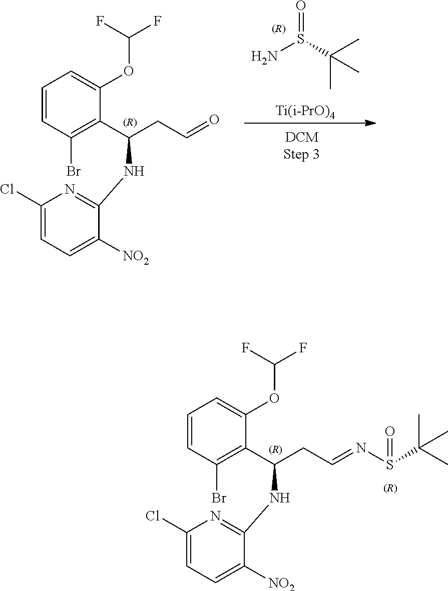

To a stirred mixture of (R)-3-(2-bromo-6-(difluoromethoxy)phenyl)-3-((6-chloro-3-nitropyridin-2-yl)amino)propanal (5.80 g, 12.871 mmol) and (R)-2-methylpropane-2-sulfinamide (1.87 g, 15.445 mmol) in DCM (59 mL) was added Ti(Oi-Pr)₄ (5.49 g, 19.306 mmol) at room temperature. The resulting mixture was stirred for 16 h at room temperature. The reaction was quenched with water at room temperature. The resulting mixture was filtered, the filter cake was washed with DCM (3×50 mL). The filtrate was extracted with CH₂Cl₂ (3×200 mL). The resulting mixture was concentrated under vacuum. The residue was purified by silica gel column chromatography, eluted with PE/EA (5:1) to afford (R)—N—((R,E)-3-(2-bromo-6-(difluoromethoxy)phenyl)-3-((6-chloro-3-nitropyridin-2-yl)amino)propylidene)-2-methylpropane-2-sulfinamide (5.00 g, 70%) as a yellow oil. MS ESI calculated for C₁₉H₂₀BrClF₂N₄O₄S [M+H]⁺, 553.00 555.00 found 552.95 554.95. ¹H NMR (400 MHz, Chloroform-d) δ 9.18 (d, J=8.6 Hz, 1H), 8.33-8.28 (m, 1H), 8.12-8.06 (m, 1H), 7.47-7.41 (m, 1H), 7.19-7.12 (m, 2H), 6.76-6.70 (m, 1H), 6.67 (d, J=5.4 Hz, 1H), 6.65-6.59 (m, 1H), 3.55-3.39 (m, 1H), 3.14-3.05 (m, 1H), 1.12 (s, 9H).

### Preparation 59D: (R)—N-((3R)-3-(2-bromo-6-(difluoromethoxy)phenyl)-3-((6-chloro-3-nitropyridin-2-yl)amino)-1-cyanopropyl)-2-methylpropane-2-sulfinamide

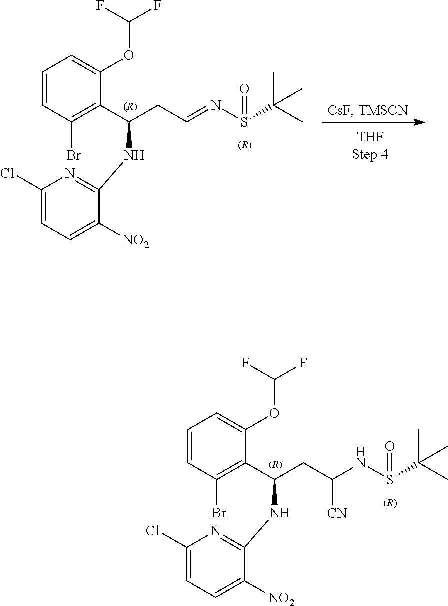

To a stirred mixture of (R)—N—((R,E)-3-(2-bromo-6-(difluoromethoxy)phenyl)-3-((6-chloro-3-nitropyridin-2-yl)amino)propylidene)-2-methylpropane-2-sulfinamide (4.50 g, 8.126 mmol) and CsF (2.47 g, 16.252 mmol) in THF (45 mL) was added TMSCN (1.61 g, 16.252 mmol) at 0° C. under nitrogen atmosphere. The resulting mixture was stirred for 2 h at room temperature under nitrogen atmosphere. The reaction was quenched with water at room temperature. The aqueous layer was extracted with CH₂Cl₂ (3×100 mL). The resulting mixture was concentrated under vacuum. The residue was purified by silica gel column chromatography, eluted with PE/EA (1:1) to afford (R)—N-((3R)-3-(2-bromo-6-(difluoromethoxy)phenyl)-3-((6-chloro-3-nitropyridin-2-yl)amino)-1-cyanopropyl)-2-methylpropane-2-sulfinamide (4.50 g, 95%) as a yellow oil. MS ESI calculated for C₂₀H₂₁BrClF₂N₅O₄S [M+H]⁺, 580.02 582.01 found 580.00 582.00. ¹H NMR (400 MHz, Chloroform-d) δ 9.46-9.05 (m, 1H), 8.40-8.31 (m, 1H), 7.48-7.41 (m, 1H), 7.22-7.11 (m, 2H), 6.83-6.74 (m, 1H), 6.71-6.65 (m, 1H), 6.56-6.44 (m, 1H), 4.35-4.25 (m, 1H), 4.18 (d, J=9.2 Hz, 1H), 2.91-2.72 (m, 1H), 2.48-2.37 (m, 1H), 1.27 (s, 9H).

### Preparation 59E: (6R,8R)-8-(2-bromo-6-(difluoromethoxy)phenyl)-2-chloro-7,8-dihydro-6H-pyrrolo[2′,1′:2,3]imidazo[4,5-b]pyridin-6-amine

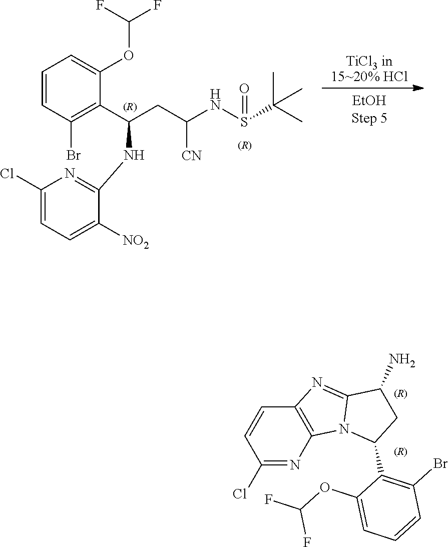

To a stirred mixture of (R)—N-((3R)-3-(2-bromo-6-(difluoromethoxy)phenyl)-3-((6-chloro-3-nitropyridin-2-yl)amino)-1-cyanopropyl)-2-methylpropane-2-sulfinamide (4.50 g, 7.748 mmol) in EtOH (45 mL) was added TiCl₃ in 15˜20% HCl (47.79 g, 6.888 mmol) at room temperature. The resulting mixture was stirred for 16 h at 80° C. The resulting mixture was diluted with EtOAc (100 mL). The residue was basified to pH 7 with saturated NaHCO₃ (aq.).

The resulting mixture was filtered, the filter cake was washed with EtOAc (3×100 mL). The filtrate was washed with 2×100 mL of water. The organic layer was concentrated under vacuum. The residue was purified by silica gel column chromatography, eluted with CH₂Cl₂/MeOH (10:1) to afford (6R,8R)-8-(2-bromo-6-(difluoromethoxy)phenyl)-2-chloro-7,8-dihydro-6H-pyrrolo[2′,1′:2,3]imidazo[4,5-b]pyridin-6-amine (2.00 g, 60%) as a colorless oil. MS ESI calculated for C₁₆H₁₂BrClF₂N₄O [M+H]⁺, 428.99 430.99 found 428.90 430.90. ¹H NMR (400 MHz, Chloroform-d) δ 7.94-7.88 (m, 1H), 7.61-7.49 (m, 1H), 7.26-7.20 (m, 1H), 7.18-7.11 (m, 1H), 7.00-6.74 (m, 1H), 6.29-6.06 (m, 1H), 4.76-4.62 (m, 1H), 3.66-3.52 (m, 1H), 3.02-2.66 (m, 1H), 2.59-2.41 (m, 1H).

### Preparation 59F: (7R,14R)-11-chloro-1-(difluoromethoxy)-6,7-dihydro-7,14-methanobenzo[f]pyrido[3′,2′:4,5]imidazo[1,2-a][1,4]diazocin-5(14H)-one

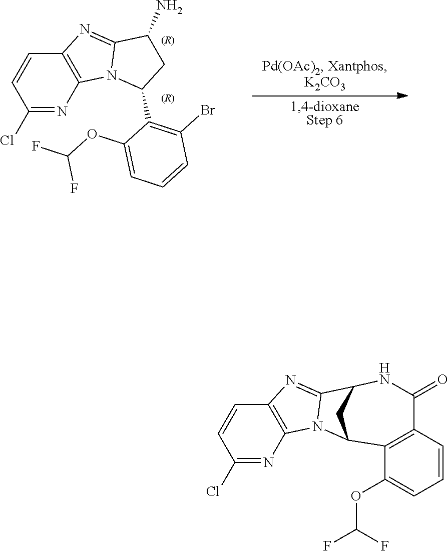

To a stirred mixture of (6R,8R)-8-(2-bromo-6-(difluoromethoxy)phenyl)-2-chloro-7,8-dihydro-6H-pyrrolo[2′,1′:2,3]imidazo[4,5-b]pyridin-6-amine (250 mg, 0.582 mmol) and K₂CO₃ (160 mg, 1.164 mmol) in 1,4-dioxane (10 mL) were added XantPhos (34 mg, 0.058 mmol) and Pd(OAc)₂ (13 mg, 0.058 mmol) at room temperature under carbon monoxide atmosphere. The resulting mixture was stirred for 16 h at 100° C. under carbon monoxide atmosphere. The resulting mixture was concentrated under vacuum. The residue was purified by silica gel column chromatography, eluted with CH₂Cl₂/MeOH (10:1) to afford (7R,14R)-11-chloro-1-(difluoromethoxy)-6,7-dihydro-7,14-methanobenzo[f]pyrido[3′,2′:4,5]imidazo[1,2-a][1,4]diazocin-5(14H)-one (50 mg, 23%) as a yellow solid. MS ESI calculated for C₁₇H₁₁ClF₂N₄O₂ [M+H]⁺, 377.05 found 376.95. ¹H NMR (400 MHz, Chloroform-d) δ 8.48-8.35 (m, 1H), 7.94 (d, J=8.5 Hz, 1H), 7.53-7.39 (m, 2H), 7.30 (d, J=16.2 Hz, 1H), 7.22-6.93 (m, 2H), 6.55 (d, J=7.4 Hz, 1H), 4.94 (t, J=6.6 Hz, 1H), 3.52-3.39 (m, 1H), 2.87 (d, J=13.4 Hz, 1H). ¹⁹F NMR (377 MHz, Chloroform-d) δ −76.51, −76.96, −82.32, −82.76.

### Preparation 59G: (7R,14R)-11-chloro-1-(difluoromethoxy)-6-(methyl-d3)-6,7-dihydro-7,14-methanobenzo[f]pyrido[3′,2′:4,5]imidazo[1,2-a][1,4]diazocin-5(14H)-one

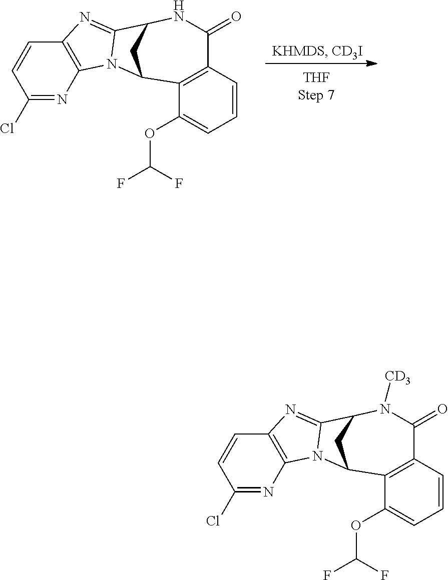

To a stirred mixture of (7R,14R)-11-chloro-1-(difluoromethoxy)-6,7-dihydro-7,14-methanobenzo[f]pyrido[3′,2′:4,5]imidazo[1,2-a][1,4]diazocin-5(14H)-one (500 mg, 1.327 mmol) in THF (2.5 mL) was added 1M KHMDS in THF (1.73 mL, 1.725 mmol, 1 M in THF) at −78° C. under nitrogen atmosphere. The resulting mixture was stirred for 1 h at −78° C. To the above mixture was added CD₃I (385 mg, 2.654 mmol) at −78° C. The resulting mixture was stirred for additional 16 h at room temperature. The resulting mixture was concentrated under vacuum and purified by silica gel column chromatography, eluted with CH₂Cl₂/MeOH (10:1) to afford (7R,14R)-11-chloro-1-(difluoromethoxy)-6-(methyl-d3)-6,7-dihydro-7,14-methanobenzo[f]pyrido[3′,2′:4,5]imidazo[1,2-a][1,4]diazocin-5(14H)-one (400 mg, 77%) as a yellow solid. MS ESI calculated for C₁₈H₁₀D₃ClF₂N₄O₂ [M+H]⁺, 394.09 found 394.10. ¹H NMR (400 MHz, Chloroform-d) δ 8.50-8.45 (m, 1H), 7.96-7.89 (m, 1H), 7.47-7.39 (m, 2H), 7.24-7.20 (m, 1H), 7.15-6.90 (m, 1H), 6.47 (d, J=7.4 Hz, 1H), 5.05-4.97 (m, 1H), 3.53-3.38 (m, 1H), 2.89 (d, J=13.7 Hz, 1H). ¹⁹F NMR (377 MHz, Chloroform-d) δ −76.87, −77.31, −82.27, −82.71.

### Preparation 59H: tert-butyl (6-((7R,14R)-1-(difluoromethoxy)-6-(methyl-d3)-5-oxo-5,6,7,14-tetrahydro-7,14-methanobenzo[f]pyrido[3′,2′:4,5]imidazo[1,2-a][1,4]diazocin-11-yl)-3-methyl-2,3-dihydrofuro[3,2-b]pyridin-3-yl)carbamate

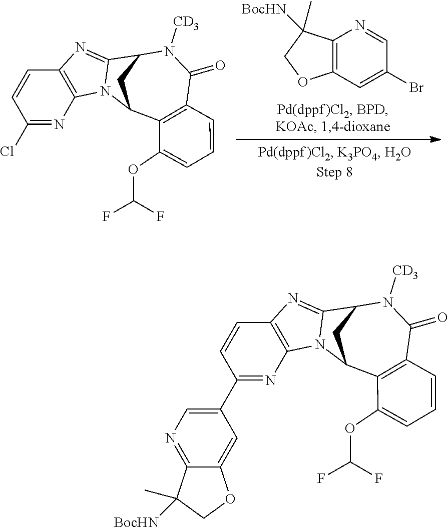 [SMILES: [2H]C([2H])([2H])N1C(=O)c2cccc(OC(F)F)c2[C@H]2C[C@@H]1c1nc3ccc(Cl)nc3n12 ; CC(C)(C)OC(=O)NC1(C)COc2cc(Br)cnc21 ; [2H]C([2H])([2H])N1C(=O)c2cccc(OC(F)F)c2[C@H]2C[C@@H]1c1nc3ccc(-c4cnc5c(c4)OCC5(C)NC(=O)OC(C)(C)C)nc3n12]

To a stirred mixture of tert-butyl N-{6-bromo-3-methyl-2H-furo[3,2-b]pyridin-3-yl}carbamate (71 mg, 0.216 mmol) and BPD (71 mg, 0.281 mmol) in 1,4-dioxane (4 mL) were added KOAc (53 mg, 0.540 mmol) and Pd(dppf)Cl₂·CH₂Cl₂ (18 mg, 0.022 mmol) at room temperature under nitrogen atmosphere. The resulting mixture was stirred for 3 h at 110° C. under nitrogen atmosphere. The mixture was allowed to cool down to room temperature. To the above mixture was added (7R,14R)-11-chloro-1-(difluoromethoxy)-6-(methyl-d3)-6,7-dihydro-7,14-methanobenzo[f]pyrido[3′,2′:4,5]imidazo[1,2-a][1,4]diazocin-5(14H)-one (85 mg, 0.216 mmol), Pd(dppf)Cl₂·CH₂Cl₂ (18 mg, 0.022 mmol), K₃PO₄ (137 mg, 0.648 mmol) and H₂O (0.4 mL) at room temperature. The resulting mixture was stirred for additional 3 h at 80° C. The resulting mixture was concentrated under reduced pressure and purified by silica gel column chromatography, eluted with CH₂Cl₂/MeOH (10:1) to afford tert-butyl (6-((7R,14R)-1-(difluoromethoxy)-6-(methyl-d3)-5-oxo-5,6,7,14-tetrahydro-7,14-methanobenzo[f]pyrido[3′,2′:4,5]imidazo[1,2-a][1,4]diazocin-11-yl)-3-methyl-2,3-dihydrofuro[3,2-b]pyridin-3-yl)carbamate (110 mg, 84%) as a white solid. MS ESI calculated for C₃₁H₂₇D₃F₂N₆O₅ [M+H]⁺, 608.24 found 608.25.

### Preparation 59I: (7R,14R)-11-(3-amino-3-methyl-2,3-dihydrofuro[3,2-b]pyridin-6-yl)-1-(difluoromethoxy)-6-(methyl-d3)-6,7-dihydro-7,14-methanobenzo[f]pyrido[3′,2′:4,5]imidazo[1,2-a][1,4]diazocin-5(14H)-one

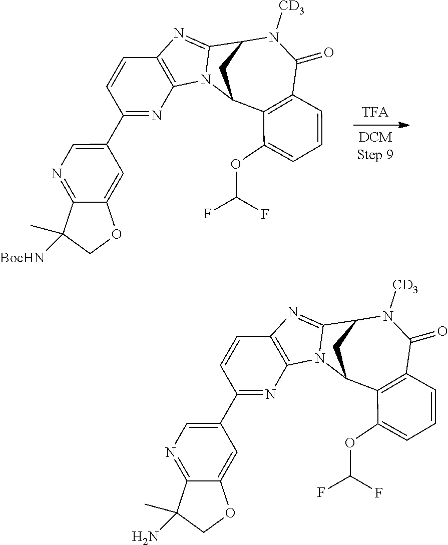 [SMILES: [2H]C([2H])([2H])N1C(=O)c2cccc(OC(F)F)c2[C@H]2C[C@@H]1c1nc3ccc(-c4cnc5c(c4)OCC5(C)NC(=O)OC(C)(C)C)nc3n12 ; [2H]C([2H])([2H])N1C(=O)c2cccc(OC(F)F)c2[C@H]2C[C@@H]1c1nc3ccc(-c4cnc5c(c4)OCC5(C)N)nc3n12]

To a stirred mixture of tert-butyl (6-((7R,14R)-1-(difluoromethoxy)-6-(methyl-d3)-5-oxo-5,6,7,14-tetrahydro-7,14-methanobenzo[f]pyrido[3′,2′:4,5]imidazo[1,2-a][1,4]diazocin-11-yl)-3-methyl-2,3-dihydrofuro[3,2-b]pyridin-3-yl)carbamate (110 mg, 0.181 mmol) in DCM (3 mL) was added TFA (1 mL) at room temperature. The resulting mixture was stirred for 1 h, then concentrated under vacuum. The residue was purified by reversed-phase flash chromatography with the following conditions: column, C18 silica gel; mobile phase, CH₃CN in Water (10 mmol/L NH₄HCO₃), 20% to 55% gradient in 30 min; detector, 254 nm. This resulted in (7R,14R)-11-(3-amino-3-methyl-2,3-dihydrofuro[3,2-b]pyridin-6-yl)-1-(difluoromethoxy)-6-(methyl-d3)-6,7-dihydro-7,14-methanobenzo[f]pyrido[3′,2′:4,5]imidazo[1,2-a][1,4]diazocin-5(14H)-one (60 mg, 65%) as a white solid. MS ESI calculated for C₂₆H₁₉D₃F₂N₆O₃ [M+H]⁺, 508.19, found 508.15.

### Example 59 and 60: (7R,14R)-11-((S)-3-amino-3-methyl-2,3-dihydrofuro[3,2-b]pyridin-6-yl)-1-(difluoromethoxy)-6-(methyl-d3)-6,7-dihydro-7,14-methanobenzo[f]pyrido[3′,2′:4,5]imidazo[1,2-a][1,4]diazocin-5(14H)-one and (7R,14R)-11-((R)-3-amino-3-methyl-2,3-dihydrofuro[3,2-b]pyridin-6-yl)-1-(difluoromethoxy)-6-(methyl-d3)-6,7-dihydro-7,14-methanobenzo[f]pyrido[3′,2′:4,5]imidazo[1,2-a][1,4]diazocin-5(14H)-one

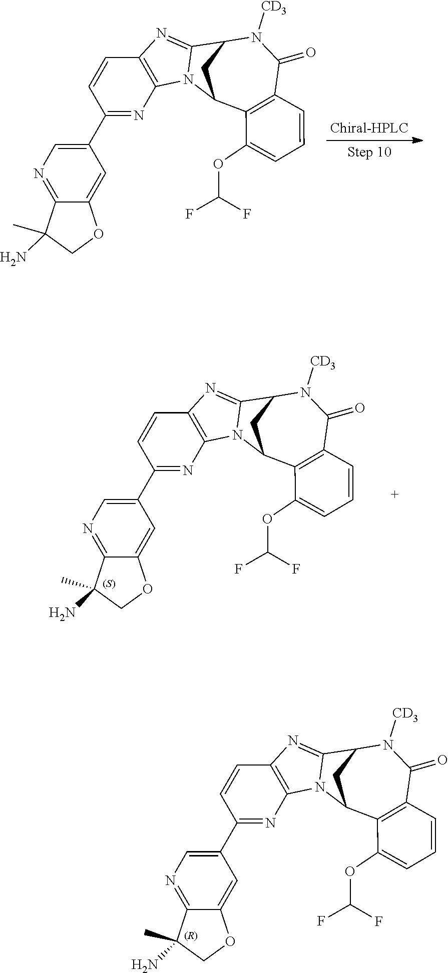

(7R,14R)-11-(3-amino-3-methyl-2,3-dihydrofuro[3,2-b]pyridin-6-yl)-1-(difluoromethoxy)-6-(methyl-d3)-6,7-dihydro-7,14-methanobenzo[f]pyrido[3′,2′:4,5]imidazo[1,2-a][1,4]diazocin-5(14H)-one (60 mg) was resolved by Prep-Chiral HPLC with the following conditions: CHIRAL ART Cellulose-SZ, 3*25 cm, 5 m; Mobile Phase A: Hex (10 mM NH₃-MeOH), Mobile Phase B: EtOH; Flow rate: 40 mL/min; Gradient: isocratic 50; Wave Length: 200/210 nm; RT1(min): 14.145; RT2(min): 22.583. The first peak afforded 14 mg (16%) as a white solid. MS ESI calculated for C₂₆H₁₉D₃F₂N₆O₃ [M+H]⁺, 508.19 found 508.25. ¹H NMR (400 MHz, Chloroform-d) δ 8.65 (d, J=1.8 Hz, 1H), 8.50-8.44 (m, 1H), 8.04 (d, J=8.5 Hz, 1H), 7.66 (d, J=1.8 Hz, 1H), 7.60 (d, J=8.5 Hz, 1H), 7.43-7.38 (m, 2H), 7.31-6.91 (m, 1H), 6.53 (d, J=7.0 Hz, 1H), 4.97 (d, J=7.0 Hz, 1H), 4.54-4.41 (m, 2H), 3.55-3.41 (m, 1H), 2.90 (d, J=13.6 Hz, 1H), 1.62 (s, 3H). ¹⁹F NMR (377 MHz, Chloroform-d) δ −78.03, −78.48, −81.74, −82.18. The second peak afforded 13 mg (14%) as a white solid. MS ESI calculated for C₂₆H₁₉D₃F₂N₆O₃ [M+H]⁺, 508.19 found 508.20. ¹H NMR (400 MHz, Chloroform-d) δ 8.63 (d, J=1.7 Hz, 1H), 8.50-8.44 (m, 1H), 8.03 (d, J=8.4 Hz, 1H), 7.67 (d, J=1.8 Hz, 1H), 7.59 (d, J=8.4 Hz, 1H), 7.44-7.38 (m, 2H), 7.30-6.90 (m, 1H), 6.53 (d, J=7.0 Hz, 1H), 4.97 (d, J=7.0 Hz, 1H), 4.52-4.46 (m, 2H), 3.55-3.43 (m, 1H), 2.89 (d, J=13.6 Hz, 1H), 1.63 (s, 3H). ¹⁹F NMR (377 MHz, Chloroform-d) δ −77.98, −78.43, −81.77, −82.21.

### Example 61 and 62: (7R,14R)-11-((S)-3-amino-3-methyl-2,3-dihydrofuro[3,2-b]pyridin-6-yl)-1-(difluoromethoxy)-10-fluoro-6-(methyl-d3)-6,7-dihydro-7,14-methanobenzo[f]benzo[4,5]imidazo[1,2-a][1,4]diazocin-5(14H)-one and (7R,14R)-11-((R)-3-amino-3-methyl-2,3-dihydrofuro[3,2-b]pyridin-6-yl)-1-(difluoromethoxy)-10-fluoro-6-(methyl-d3)-6,7-dihydro-7,14-methanobenzo[f]benzo[4,5]imidazo[1,2-a][1,4]diazocin-5(14H)-one

### Synthetic Scheme

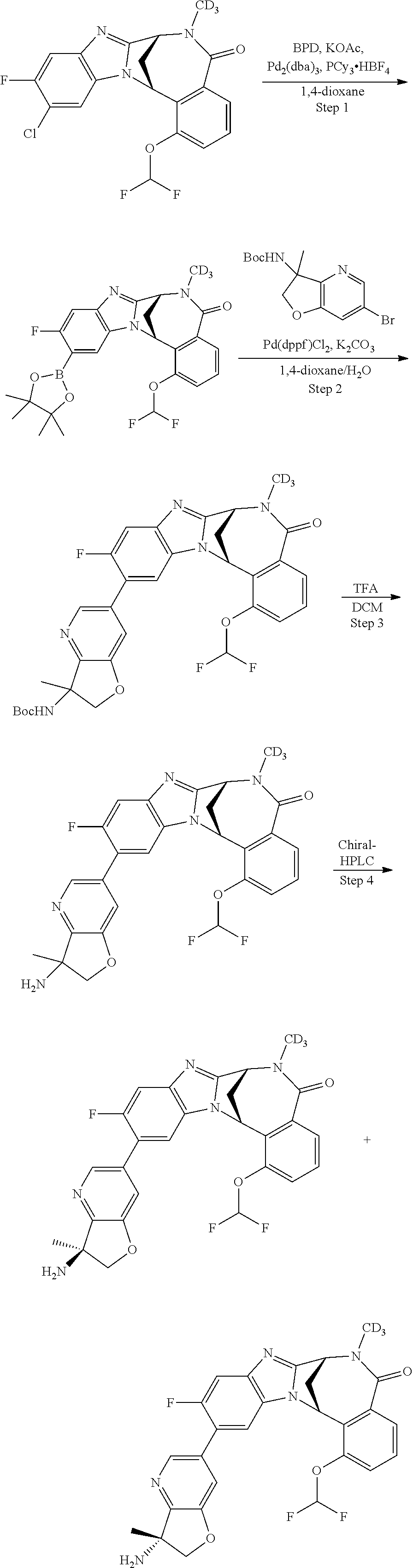

### Preparation 61A: (7R,14R)-1-(difluoromethoxy)-10-fluoro-6-(methyl-d₃)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one

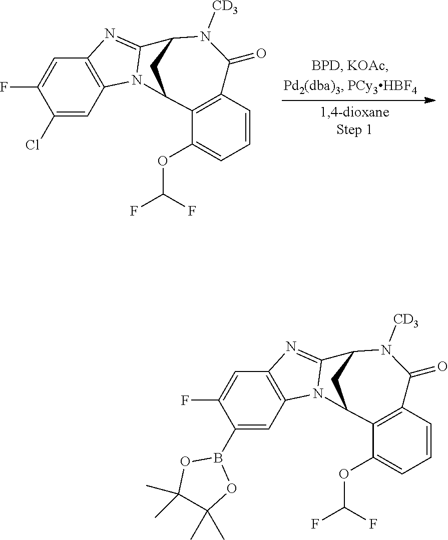 [SMILES: [2H]C([2H])([2H])N1C(=O)c2cccc(OC(F)F)c2[C@H]2C[C@@H]1c1nc3cc(F)c(Cl)cc3n12 ; [2H]C([2H])([2H])N1C(=O)c2cccc(OC(F)F)c2[C@H]2C[C@@H]1c1nc3cc(F)c(B4OC(C)(C)C(C)(C)O4)cc3n12]

To a stirred solution of (7R,14R)-11-chloro-1-(difluoromethoxy)-10-fluoro-6-(methyl-d₃)-6,7-dihydro-7,14-methanobenzo[f]benzo[4,5]imidazo[1,2-a][1,4]diazocin-5(14H)-one (200 mg, 0.487 mmol) and BPD (185 mg, 0.730 mmol) in 1,4-dioxane (5 mL) were added KOAc (143 mg, 1.461 mmol), PCy₃·HBF₄ (27 mg, 0.073 mmol) and Pd₂(dba)₃ (45 mg, 0.049 mmol) at room temperature. The resulting mixture was stirred for 12 h at 140° C. under nitrogen atmosphere. The resulting mixture was concentrated under reduced pressure. The residue was purified by silica gel column chromatography, eluted with CH₂Cl₂/MeOH (8:1) to afford (7R,14R)-1-(difluoromethoxy)-10-fluoro-6-(methyl-d₃)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (210 mg, 86%) as a yellow oil. MS ESI calculated for C₂₅H₂₂D3BF₃N₃O₄ [M+H]⁺, 503.21 found 503.30. ¹H NMR (400 MHz, Chloroform-d) δ 8.50-8.43 (m, 1H), 7.84 (d, J=4.8 Hz, 1H), 7.44-7.38 (m, 1H), 7.37-7.29 (m, 2H), 7.05-6.68 (m, 1H), 6.25 (d, J=7.2 Hz, 1H), 4.96 (d, J=7.2 Hz, 1H), 3.51-3.39 (m, 1H), 2.85 (d, J=13.6 Hz, 1H), 1.28-1.26 (m, 12H).

### Preparation 61B: tert-butyl (6-((7R,14R)-1-(difluoromethoxy)-10-fluoro-6-(methyl-d₃)-5-oxo-5,6,7,14-tetrahydro-7,14-methanobenzo[f]benzo[4,5]imidazo[1,2-a][1,4]diazocin-11-yl)-3-methyl-2,3-dihydrofuro[3,2-b]pyridin-3-yl)carbamate

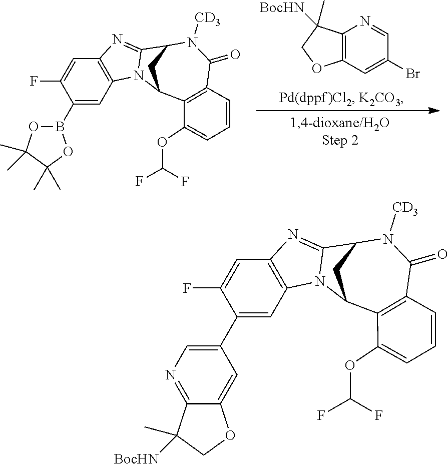 [SMILES: [2H]C([2H])([2H])N1C(=O)c2cccc(OC(F)F)c2[C@H]2C[C@@H]1c1nc3cc(F)c(B4OC(C)(C)C(C)(C)O4)cc3n12 ; CC(C)(C)OC(=O)NC1(C)COc2cc(Br)cnc21 ; [2H]C([2H])([2H])N1C(=O)c2cccc(OC(F)F)c2[C@H]2C[C@@H]1c1nc3cc(F)c(-c4cnc5c(c4)OCC5(C)NC(=O)OC(C)(C)C)cc3n12]

To a solution of (7R,14R)-1-(difluoromethoxy)-10-fluoro-6-(methyl-d₃)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (100 mg, 0.199 mmol) and tert-butyl (6-bromo-3-methyl-2,3-dihydrofuro[3,2-b]pyridin-3-yl)carbamate (56 mg, 0.169 mmol) in 1,4-dioxane (2.5 mL) and H₂O (0.2 mL) were added K₂CO₃ (55 mg, 0.398 mmol) and Pd(dppf)Cl₂·CH₂Cl₂ (16 mg, 0.020 mmol) at room temperature. The resulting mixture was stirred for 2 h at 80° C. under nitrogen atmosphere. The resulting mixture was concentrated under reduced pressure. The residue was purified by silica gel column chromatography, eluted with CH₂Cl₂/MeOH (10:1) to afford tert-butyl (6-((7R,14R)-1-(difluoromethoxy)-10-fluoro-6-(methyl-d₃)-5-oxo-5,6,7,14-tetrahydro-7,14-methanobenzo[f]benzo[4,5]imidazo[1,2-a][1,4]diazocin-11-yl)-3-methyl-2,3-dihydrofuro[3,2-b]pyridin-3-yl)carbamate (98 mg, 79%) as a brown solid. MS ESI calculated for C₃₂H₂₇D₃F₃N₅O₅ [M+H]⁺, 625.24 found 625.40. ¹H NMR (400 MHz, Chloroform-d) δ 8.50 (d, J=8.2 Hz, 1H), 8.22 (d, J=5.6 Hz, 1H), 7.57-7.28 (m, 5H), 7.01-6.59 (m, 1H), 6.26 (d, J=6.7 Hz, 1H), 5.08-4.98 (m, 1H), 4.97-4.57 (m, 2H), 3.56-3.40 (m, 1H), 2.99-2.79 (m, 1H), 1.75 (s, 3H), 1.42 (s, 9H).

### Preparation 61C: (7R,14R)-11-(3-amino-3-methyl-2,3-dihydrofuro[3,2-b]pyridin-6-yl)-1-(difluoromethoxy)-10-fluoro-6-(methyl-d₃)-6,7-dihydro-7,14-methanobenzo[f]benzo[4,5]imidazo[1,2-a][1,4]diazocin-5(14H)-one

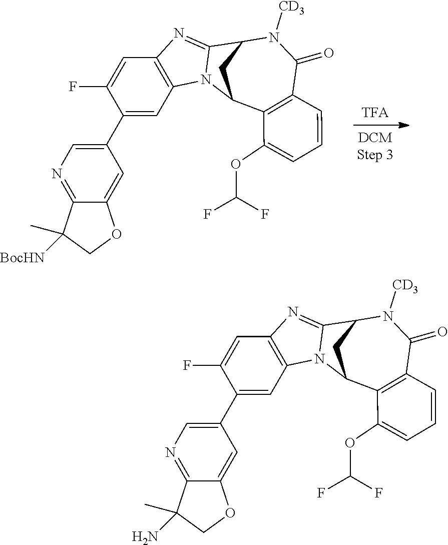 [SMILES: [2H]C([2H])([2H])N1C(=O)c2cccc(OC(F)F)c2[C@H]2C[C@@H]1c1nc3cc(F)c(-c4cnc5c(c4)OCC5(C)NC(=O)OC(C)(C)C)cc3n12 ; [2H]C([2H])([2H])N1C(=O)c2cccc(OC(F)F)c2[C@H]2C[C@@H]1c1nc3cc(F)c(-c4cnc5c(c4)OCC5(C)N)cc3n12]

To a stirred solution of tert-butyl (6-((7R,14R)-1-(difluoromethoxy)-10-fluoro-6-(methyl-d3)-5-oxo-5,6,7,14-tetrahydro-7,14-methanobenzo[f]benzo[4,5]imidazo[1,2-a][1,4]diazocin-11-yl)-3-methyl-2,3-dihydrofuro[3,2-b]pyridin-3-yl)carbamate (95 mg, 0.152 mmol) in DCM (2.5 mL) was added TFA (0.5 mL) dropwise at room temperature. The resulting mixture was stirred for 1 h at room temperature. The resulting mixture was basified and concentrated under reduced pressure. The residue was purified by Prep-TLC (CH₂Cl₂/MeOH 10/1) to afford (7R,14R)-11-(3-amino-3-methyl-2,3-dihydrofuro[3,2-b]pyridin-6-yl)-1-(difluoromethoxy)-10-fluoro-6-(methyl-d₃)-6,7-dihydro-7,14-methanobenzo[f]benzo[4,5]imidazo[1,2-a][1,4]diazocin-5(14H)-one (50 mg, 63%) as a white solid. MS ESI calculated for C₂₇H₁₉D₃F₃N₅O₃ [M+H]⁺, 525.19, found 525.25.

### Example 61 and 62: (7R,14R)-11-((S)-3-amino-3-methyl-2,3-dihydrofuro[3,2-b]pyridin-6-yl)-1-(difluoromethoxy)-10-fluoro-6-(methyl-d3)-6,7-dihydro-7,14-methanobenzo[f]benzo[4,5]imidazo[1,2-a][1,4]diazocin-5(14H)-one and (7R,14R)-11-((R)-3-amino-3-methyl-2,3-dihydrofuro[3,2-b]pyridin-6-yl)-1-(difluoromethoxy)-10-fluoro-6-(methyl-d3)-6,7-dihydro-7,14-methanobenzo[f]benzo[4,5]imidazo[1,2-a][1,4]diazocin-5(14H)-one

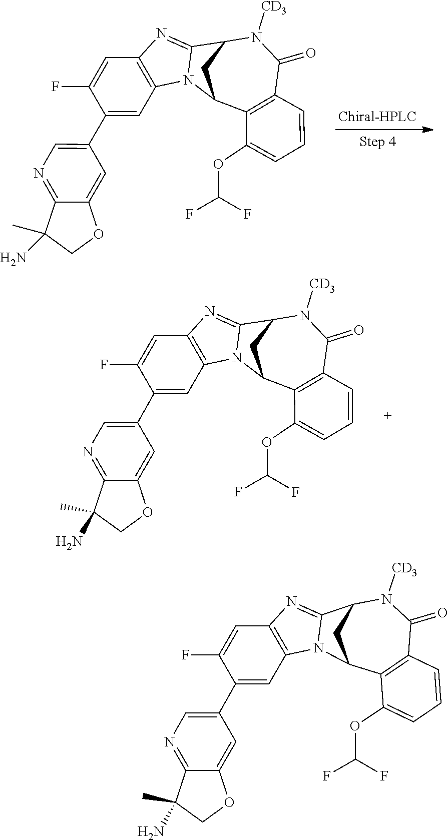 [SMILES: [2H]C([2H])([2H])N1C(=O)c2cccc(OC(F)F)c2[C@H]2C[C@@H]1c1nc3cc(F)c(-c4cnc5c(c4)OCC5(C)N)cc3n12 ; [2H]C([2H])([2H])N1C(=O)c2cccc(OC(F)F)c2[C@H]2C[C@@H]1c1nc3cc(F)c(-c4cnc5c(c4)OC[C@@]5(C)N)cc3n12 ; [2H]C([2H])([2H])N1C(=O)c2cccc(OC(F)F)c2[C@H]2C[C@@H]1c1nc3cc(F)c(-c4cnc5c(c4)OC[C@]5(C)N)cc3n12]

(7R,14R)-11-(3-amino-3-methyl-2,3-dihydrofuro[3,2-b]pyridin-6-yl)-1-(difluoromethoxy)-10-fluoro-6-(methyl-d₃)-6,7-dihydro-7,14-methanobenzo[f]benzo[4,5]imidazo[1,2-a][1,4]diazocin-5(14H)-one (50 mg) was purified by Prep-Chiral HPLC with the following conditions (Column: CHIRALPAK-IK, 3*25 mm, 5 μm; Mobile Phase A: Hex (10 mM NH₃-MeOH), Mobile Phase B: EtOH; Flow rate: 40 mL/min; Gradient: isocratic 50; Wave Length: 200/210 nm; RT1(min): 8.144; RT2(min): 11.079). The first peak afforded 15 mg (36%) as a white solid. MS ESI calculated for C₂₇H₁₉D₃F₃N₅O₃ [M+H]⁺, 525.19 found 525.15. ¹H NMR (400 MHz, Chloroform-d) δ 8.49 (d, J=8.3 Hz, 1H), 8.24 (s, 1H), 7.48 (d, J=11.0 Hz, 1H), 7.45-7.39 (m, 2H), 7.31 (d, J=8.1 Hz, 1H), 7.24 (s, 1H), 6.82 (t, J=72.7 Hz, 1H), 6.23 (d, J=7.2 Hz, 1H), 4.96 (d, J=7.1 Hz, 1H), 4.61 (d, J=9.9 Hz, 1H), 4.51 (d, J=9.8 Hz, 1H), 3.56-3.37 (m, 1H), 2.87 (d, J=13.6 Hz, 1H), 1.72 (s, 3H). ¹⁹F NMR (377 MHz, 8) δ −80.36, −80.81, −80.98, −81.42, −122.70. The second peak afforded 13 mg (31%) as a white solid. MS ESI calculated for C₂₇H₁₉D₃F₃N₅O₃ [M+H]⁺, 525.19 found 525.10. ¹H NMR (400 MHz, Chloroform-d) δ 8.53-8.47 (m, 1H), 8.20 (s, 1H), 7.49 (d, J=11.0 Hz, 1H), 7.43 (t, J=8.2 Hz, 2H), 7.34-7.29 (m, 1H), 7.27-7.26 (m, 1H), 6.81 (t, J=72.7 Hz, 1H), 6.24 (d, J=7.2 Hz, 1H), 4.97 (d, J=7.1 Hz, 1H), 4.58-4.46 (m, 2H), 3.55-3.44 (m, 1H), 2.88 (d, J=13.6 Hz, 1H), 1.68 (s, 3H). ¹⁹F NMR (377 MHz, Chloroform-d) δ −80.28, −80.73, −80.87, −81.31, −122.72.

### Example 63 and 64: (7R,14R)-1-(difluoromethoxy)-11-((S)-3-hydroxy-3-methyl-2,3-dihydrofuro[3,2-b]pyridin-6-yl)-6-(methyl-d3)-6,7-dihydro-7,14-methanobenzo[f]pyrido[3′,2′:4,5]imidazo[1,2-a][1,4]diazocin-5(14H)-one and (7R,14R)-1-(difluoromethoxy)-11-((R)-3-hydroxy-3-methyl-2,3-dihydrofuro[3,2-b]pyridin-6-yl)-6-(methyl-d3)-6,7-dihydro-7,14-methanobenzo[f]pyrido[3′,2′:4,5]imidazo[1,2-a][1,4]diazocin-5(14H)-one

### Synthetic Scheme

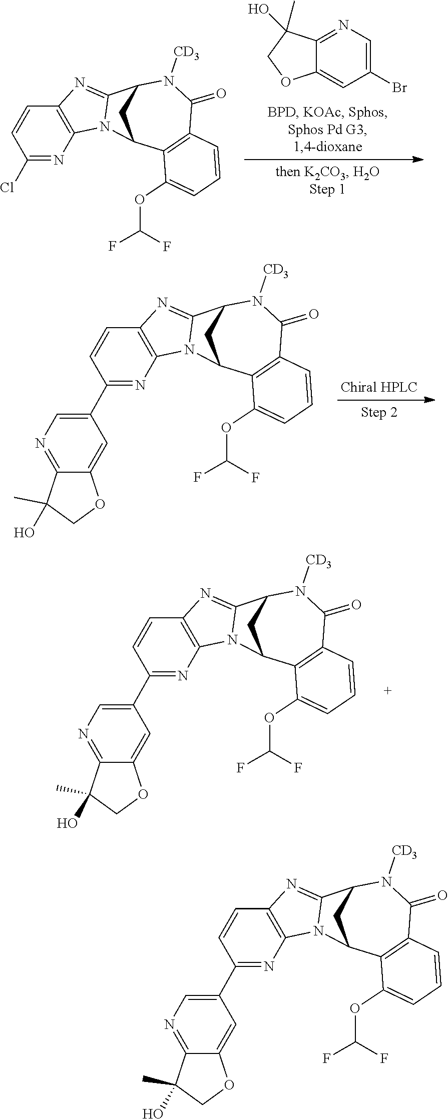 [SMILES: [2H]C([2H])([2H])N1C(=O)c2cccc(OC(F)F)c2[C@H]2C[C@@H]1c1nc3ccc(Cl)nc3n12 ; CC1(O)COc2cc(Br)cnc21 ; [2H]C([2H])([2H])N1C(=O)c2cccc(OC(F)F)c2[C@H]2C[C@@H]1c1nc3ccc(-c4cnc5c(c4)OCC5(C)O)nc3n12 ; [2H]C([2H])([2H])N1C(=O)c2cccc(OC(F)F)c2[C@H]2C[C@@H]1c1nc3ccc(-c4cnc5c(c4)OC[C@@]5(C)O)nc3n12 ; [2H]C([2H])([2H])N1C(=O)c2cccc(OC(F)F)c2[C@H]2C[C@@H]1c1nc3ccc(-c4cnc5c(c4)OC[C@]5(C)O)nc3n12]

### Preparation 63A: (7R,14R)-1-(difluoromethoxy)-11-(3-hydroxy-3-methyl-2,3-dihydrofuro[3,2-b]pyridin-6-yl)-6-(methyl-d3)-6,7-dihydro-7,14-methanobenzo[f]pyrido[3′,2′:4,5]imidazo[1,2-a][1,4]diazocin-5(14H)-one

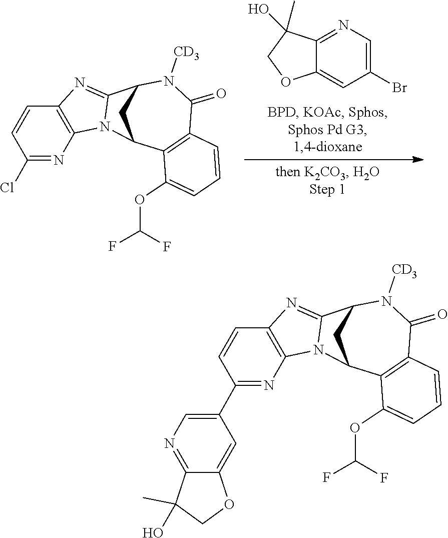 [SMILES: [2H]C([2H])([2H])N1C(=O)c2cccc(OC(F)F)c2[C@H]2C[C@@H]1c1nc3ccc(Cl)nc3n12 ; CC1(O)COc2cc(Br)cnc21 ; [2H]C([2H])([2H])N1C(=O)c2cccc(OC(F)F)c2[C@H]2C[C@@H]1c1nc3ccc(-c4cnc5c(c4)OCC5(C)O)nc3n12]

A mixture of 6-bromo-3-methyl-2,3-dihydrofuro[3,2-b]pyridin-3-ol (70 mg, 0.305 mmol), BPD (97 mg, 0.381 mmol), KOAc (75 mg, 0.762 mmol), SPhos (21 mg, 0.051 mmol) and SPhos Pd Gen.3 (20 mg, 0.025 mmol) in 1,4-dioxane (1 mL) at room temperature. The resulting mixture was stirred for 2 h at 100° C. under nitrogen atmosphere. The mixture was allowed to cool down to room temperature. To the above mixture was added (7R,14R)-11-chloro-1-(difluoromethoxy)-6-(methyl-d3)-6,7-dihydro-7,14-methanobenzo[f]pyrido[3′,2′:4,5]imidazo[1,2-a][1,4]diazocin-5(14H)-one (100 mg, 0.254 mmol), K₂CO₃ (70 mg, 0.508 mmol) and H₂O (0.2 mL) at room temperature under nitrogen atmosphere. The resulting mixture was stirred for additional overnight at 80° C. The resulting mixture was concentrated under vacuum and purified by silica gel column chromatography, eluted with CH₂Cl₂/MeOH (10/1) followed by reversed-phase flash chromatography with the following conditions: column, C18 silica gel; mobile phase, CH₃CN in water (10 mmol/L NH₄HCO₃), 30% to 50% gradient in 20 min; detector, 254/220 nm to afford (7R,14R)-1-(difluoromethoxy)-11-(3-hydroxy-3-methyl-2,3-dihydrofuro[3,2-b]pyridin-6-yl)-6-(methyl-d3)-6,7-dihydro-7,14-methanobenzo[f]pyrido[3′,2′:4,5]imidazo[1,2-a][1,4]diazocin-5(14H)-one (100 mg, 77%) as a white solid. MS ESI calculated for C₂₆H₁₈D₃F₂N₅O₄ [M+H]⁺, 509.17, found 509.20.

### Example 63 and 64: (7R,14R)-1-(difluoromethoxy)-11-((S)-3-hydroxy-3-methyl-2,3-dihydrofuro[3,2-b]pyridin-6-yl)-6-(methyl-d3)-6,7-dihydro-7,14-methanobenzo[f]pyrido[3′,2′:4,5]imidazo[1,2-a][1,4]diazocin-5(14H)-one and (7R,14R)-1-(difluoromethoxy)-11-((R)-3-hydroxy-3-methyl-2,3-dihydrofuro[3,2-b]pyridin-6-yl)-6-(methyl-d3)-6,7-dihydro-7,14-methanobenzo[f]pyrido[3′,2′:4,5]imidazo[1,2-a][1,4]diazocin-5(14H)-one

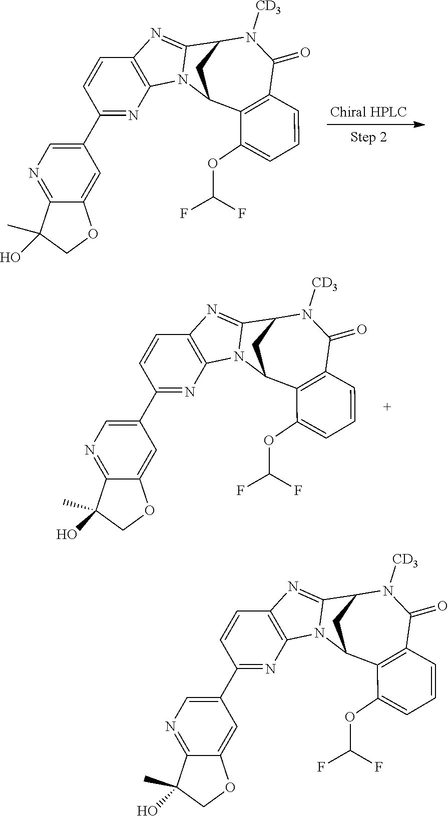 [SMILES: [2H]C([2H])([2H])N1C(=O)c2cccc(OC(F)F)c2[C@H]2C[C@@H]1c1nc3ccc(-c4cnc5c(c4)OCC5(C)O)nc3n12 ; [2H]C([2H])([2H])N1C(=O)c2cccc(OC(F)F)c2[C@H]2C[C@@H]1c1nc3ccc(-c4cnc5c(c4)OC[C@@]5(C)O)nc3n12 ; [2H]C([2H])([2H])N1C(=O)c2cccc(OC(F)F)c2[C@H]2C[C@@H]1c1nc3ccc(-c4cnc5c(c4)OC[C@]5(C)O)nc3n12]

(7R,14R)-1-(difluoromethoxy)-11-(3-hydroxy-3-methyl-2,3-dihydrofuro[3,2-b]pyridin-6-yl)-6-(methyl-d3)-6,7-dihydro-7,14-methanobenzo[f]pyrido[3′,2′:4,5]imidazo[1,2-a][1,4]diazocin-5(14H)-one (100 mg) was separated by Prep-Chiral HPLC with the following conditions (Column: CHIRALPAK-IK, 3*25 mm, 5 μm; Mobile Phase A: Hex (10 mM NH₃-MeOH), Mobile Phase B: EtOH; Flow rate: 40 mL/min; Gradient: isocratic 50; Wave Length: 209/215 nm; RT1(min): 15.4; RT2(min): 21.4). The first peak afforded 29 mg (29%) as a white solid. MS ESI calculated for C₂₆H₁₈D₃F₂N₅O₄ [M+H]⁺, 509.17, found 509.00. ¹H NMR (400 MHz, Chloroform-d) δ 8.66 (d, J=1.7 Hz, 1H), 8.51-8.44 (m, 1H), 8.02 (d, J=8.4 Hz, 1H), 7.75-7.72 (m, 1H), 7.58 (d, J=8.5 Hz, 1H), 7.46-7.36 (m, 2H), 7.24-6.87 (m, 1H), 6.54 (d, J=7.3 Hz, 1H), 4.97 (d, J=7.0 Hz, 1H), 4.70 (d, J=10.2 Hz, 1H), 4.48 (d, J=10.1 Hz, 1H), 3.56-3.42 (m, 1H), 2.90 (d, J=13.6 Hz, 1H), 1.82 (s, 3H). ¹⁹F NMR (377 MHz, Chloroform-d) δ −78.06, −78.51, −81.63, −82.08. The second peak afforded 40 mg (40%) as a white solid. MS ESI calculated for C₂₆H₁₈D₃F₂N₅O₄ [M+H]⁺, 509.17, found 509.15. ¹H NMR (400 MHz, Chloroform-d) δ 8.69 (d, J=1.7 Hz, 1H), 8.49-8.43 (m, 1H), 8.05 (d, J=8.4 Hz, 1H), 7.87-7.82 (m, 1H), 7.61 (d, J=8.5 Hz, 1H), 7.44-7.36 (m, 2H), 7.25-6.85 (m, 1H), 6.53 (d, J=7.4 Hz, 1H), 4.99 (d, J=7.1 Hz, 1H), 4.73 (d, J=10.1 Hz, 1H), 4.51 (d, J=10.1 Hz, 1H), 3.55-3.42 (m, 1H), 2.90 (d, J=13.6 Hz, 1H), 1.86 (s, 3H). ¹⁹F NMR (377 MHz, Chloroform-d) δ −78.26, −78.71, −81.64, −82.09, −220.44.

### Example 65 and 66: (7R,14R)-11-((S)-3-amino-3-methyl-2,3-dihydrofuro[3,2-b]pyridin-6-yl)-1-(difluoromethoxy)-12-fluoro-6-(methyl-d3)-6,7-dihydro-7,14-methanobenzo[f]benzo[4,5]imidazo[1,2-a][1,4]diazocin-5(14H)-one and (7R,14R)-11-((R)-3-amino-3-methyl-2,3-dihydrofuro[3,2-b]pyridin-6-yl)-1-(difluoromethoxy)-12-fluoro-6-(methyl-d3)-6,7-dihydro-7,14-methanobenzo[f]benzo[4,5]imidazo[1,2-a][1,4]diazocin-5(14H)-one

### Synthetic Scheme

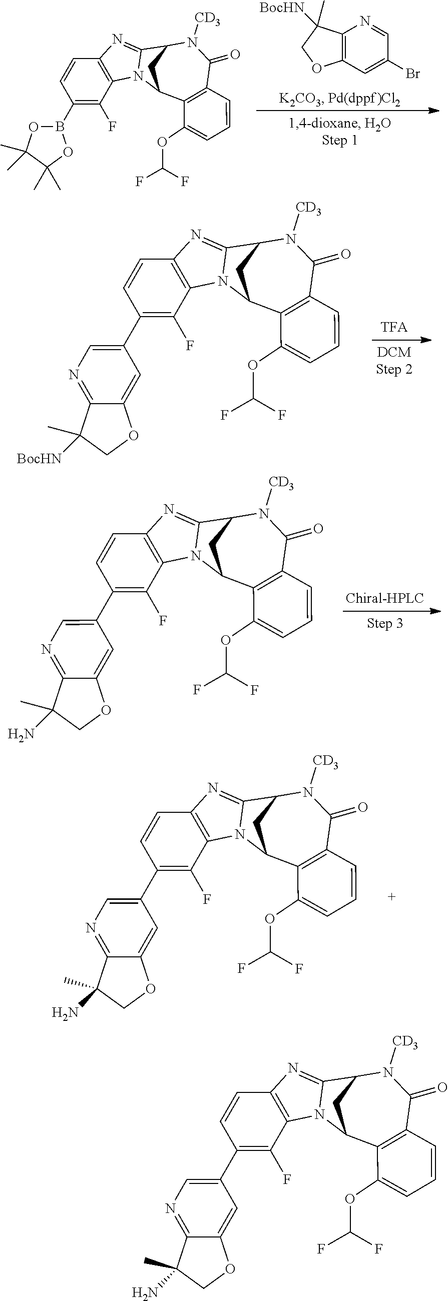

### Preparation 65A: tert-butyl (6-((7R,14R)-1-(difluoromethoxy)-12-fluoro-6-(methyl-d₃)-5-oxo-5,6,7,14-tetrahydro-7,14-methanobenzo[f]benzo[4,5]imidazo[1,2-a][1,4]diazocin-11-yl)-3-methyl-2,3-dihydrofuro[3,2-b]pyridin-3-yl)carbamate

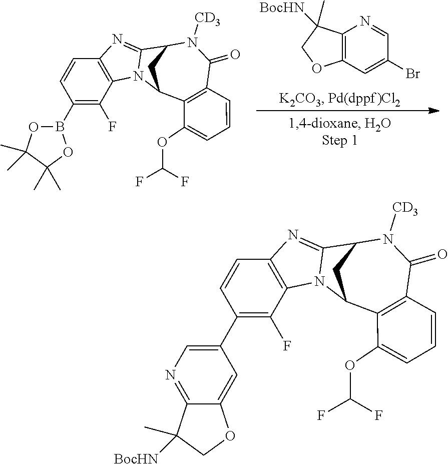 [SMILES: [2H]C([2H])([2H])N1C(=O)c2cccc(OC(F)F)c2[C@H]2C[C@@H]1c1nc3ccc(B4OC(C)(C)C(C)(C)O4)c(F)c3n12 ; CC(C)(C)OC(=O)NC1(C)COc2cc(Br)cnc21 ; [2H]C([2H])([2H])N1C(=O)c2cccc(OC(F)F)c2[C@H]2C[C@@H]1c1nc3ccc(-c4cnc5c(c4)OCC5(C)NC(=O)OC(C)(C)C)c(F)c3n12]

To a solution of (7R,14R)-1-(difluoromethoxy)-12-fluoro-6-(methyl-d₃)-11-4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (100 mg, 0.199 mmol) and tert-butyl (6-bromo-3-methyl-2,3-dihydrofuro[3,2-b]pyridin-3-yl)carbamate (66 mg, 0.199 mmol) in 1,4-dioxane (2.5 mL) and H₂O (0.5 mL) were added K₂CO₃ (55 mg, 0.398 mmol) and Pd(dppf)Cl₂ (16 mg, 0.020 mmol) at room temperature. The resulting mixture was stirred for 2 h at 80° C. under nitrogen atmosphere. The resulting mixture was concentrated under reduced pressure. The residue was purified by silica gel column chromatography, eluted with CH₂Cl₂/MeOH (10:1) to afford tert-butyl (6-((7R,14R)-1-(difluoromethoxy)-12-fluoro-6-(methyl-d₃)-5-oxo-5,6,7,14-tetrahydro-7,14-methanobenzo[f]benzo[4,5]imidazo[1,2-a][1,4]diazocin-11-yl)-3-methyl-2,3-dihydrofuro[3,2-b]pyridin-3-yl)carbamate (98 mg, 79%) as a brown oil. MS ESI calculated for C₃₂H₂₇D₃F₃N₅O₅ [M+H]⁺, 625.24 found 625.30. ¹H NMR (400 MHz, Chloroform-d) δ 8.47-8.42 (m, 1H), 8.24 (s, 1H), 7.61 (d, J=8.4 Hz, 1H), 7.47-7.33 (m, 3H), 7.25-7.20 (m, 1H), 7.00-6.70 (m, 1H), 6.57-6.49 (m, 1H), 5.79-5.54 (m, 1H), 5.07-4.91 (m, 2H), 3.57-3.42 (m, 1H), 2.89 (d, J=13.2 Hz, 1H), 1.81 (s, 3H), 1.47-1.36 (m, 9H).

### Preparation 65B: (7R,14R)-11-(3-amino-3-methyl-2,3-dihydrofuro[3,2-b]pyridin-6-yl)-1-(difluoromethoxy)-12-fluoro-6-(methyl-d₃)-6,7-dihydro-7,14-methanobenzo[f]benzo[4,5]imidazo[1,2-a][1,4]diazocin-5(14H)-one

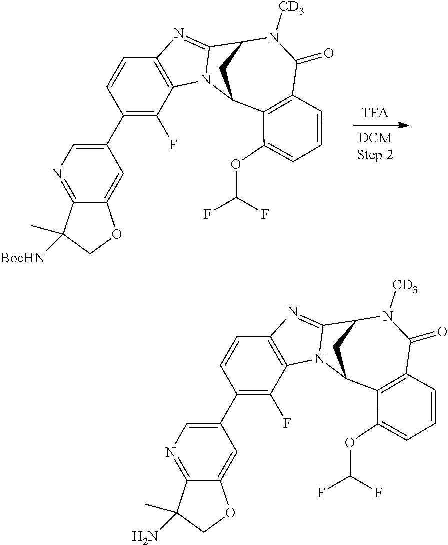 [SMILES: [2H]C([2H])([2H])N1C(=O)c2cccc(OC(F)F)c2[C@H]2C[C@@H]1c1nc3ccc(-c4cnc5c(c4)OCC5(C)NC(=O)OC(C)(C)C)c(F)c3n12 ; [2H]C([2H])([2H])N1C(=O)c2cccc(OC(F)F)c2[C@H]2C[C@@H]1c1nc3ccc(-c4cnc5c(c4)OCC5(C)N)c(F)c3n12]

To a stirred solution of tert-butyl (6-((7R,14R)-1-(difluoromethoxy)-12-fluoro-6-(methyl-d₃)-5-oxo-5,6,7,14-tetrahydro-7,14-methanobenzo[f]benzo[4,5]imidazo[1,2-a][1,4]diazocin-11-yl)-3-methyl-2,3-dihydrofuro[3,2-b]pyridin-3-yl)carbamate (98 mg, 0.157 mmol) in DCM (2.5 mL) was added TFA (0.5 mL) dropwise at room temperature. The resulting mixture was stirred for 1 h at room temperature. The resulting mixture was concentrated under reduced pressure. The residue was purified by Prep-TLC (CH₂Cl₂/MeOH 10:1) to afford (7R,14R)-11-(3-amino-3-methyl-2,3-dihydrofuro[3,2-b]pyridin-6-yl)-1-(difluoromethoxy)-12-fluoro-6-(methyl-d₃)-6,7-dihydro-7,14-methanobenzo[f]benzo[4,5]imidazo[1,2-a][1,4]diazocin-5(14H)-one (50 mg, 62%) as a white solid. MS ESI calculated for C₂₇H₁₉D₃F₃N₅O₃ [M+H]⁺, 525.19, found 525.20.

### Example 65 and 66: (7R,14R)-11-((S)-3-amino-3-methyl-2,3-dihydrofuro[3,2-b]pyridin-6-yl)-1-(difluoromethoxy)-12-fluoro-6-(methyl-d3)-6,7-dihydro-7,14-methanobenzo[f]benzo[4,5]imidazo[1,2-a][1,4]diazocin-5(14H)-one and (7R,14R)-11-((R)-3-amino-3-methyl-2,3-dihydrofuro[3,2-b]pyridin-6-yl)-1-(difluoromethoxy)-12-fluoro-6-(methyl-d3)-6,7-dihydro-7,14-methanobenzo[f]benzo[4,5]imidazo[1,2-a][1,4]diazocin-5(14H)-one

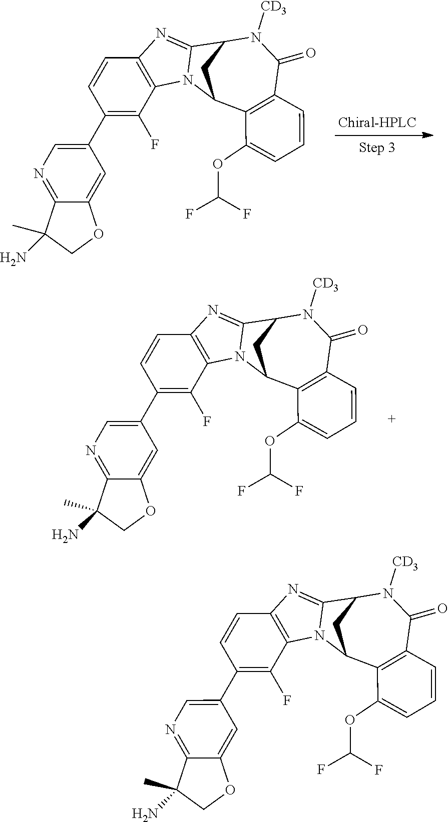 [SMILES: [2H]C([2H])([2H])N1C(=O)c2cccc(OC(F)F)c2[C@H]2C[C@@H]1c1nc3ccc(-c4cnc5c(c4)OCC5(C)N)c(F)c3n12 ; [2H]C([2H])([2H])N1C(=O)c2cccc(OC(F)F)c2[C@H]2C[C@@H]1c1nc3ccc(-c4cnc5c(c4)OC[C@@]5(C)N)c(F)c3n12 ; [2H]C([2H])([2H])N1C(=O)c2cccc(OC(F)F)c2[C@H]2C[C@@H]1c1nc3ccc(-c4cnc5c(c4)OC[C@]5(C)N)c(F)c3n12]

(7R,14R)-11-(3-amino-3-methyl-2,3-dihydrofuro[3,2-b]pyridin-6-yl)-1-(difluoromethoxy)-12-fluoro-6-(methyl-d₃)-6,7-dihydro-7,14-methanobenzo[f]benzo[4,5]imidazo[1,2-a][1,4]diazocin-5(14H)-one (50 mg) was purified by Prep-Chiral HPLC with the following conditions (Column: CHIRAL ART Cellulose-SZ, 3*25 cm, 5 μm; Mobile Phase A: Hex (10 mM NH₃-MeOH), Mobile Phase B: ETOH; Flow rate: 40 mL/min; Gradient: isocratic 50; Wave Length: 216/304 nm; RT1(min): 12.8; RT2(min): 18.5). The first peak afforded 10 mg (23%) as a white solid. MS ESI calculated for C₂₇H₁₉D₃F₃N₅O₃ [M+H]⁺, 525.19, found 525.40. ¹H NMR (400 MHz, Chloroform-d)) δ 8.50-8.39 (m, 1H), 8.24 (s, 1H), 7.60-7.54 (m, 1H), 7.42 (t, J=8.2 Hz, 1H), 7.38-7.32 (m, 1H), 7.25-7.16 (m, 2H), 6.89-6.51 (m, 1H), 6.49 (d, J=7.3 Hz, 1H), 4.94 (d, J=6.9 Hz, 1H), 4.55-4.44 (m, 2H), 3.54-3.41 (m, 1H), 2.86 (d, J=13.5 Hz, 1H), 1.66 (s, 3H). ¹⁹F NMR (377 MHz, Chloroform-d)) δ −78.74, −79.17, −79.18, −82.23, −82.25, −82.67, −82.70, −137.11, −137.12, −137.13, −137.14. The second peak afforded 11 mg (27%) as a white solid. MS ESI calculated for C₂₇H₁₉D₃F₃N₅O₃ [M+H]⁺, 525.19, found 525.15. ¹H NMR (400 MHz, Chloroform-d) δ 8.51-8.40 (m, 1H), 8.23 (s, 1H), 7.55 (d, J=8.4 Hz, 1H), 7.41 (t, J=8.2 Hz, 1H), 7.36-7.32 (m, 1H), 7.25-7.18 (m, 2H), 6.90-6.51 (m, 1H), 6.50 (d, J=7.3 Hz, 1H), 4.94 (d, J=7.0 Hz, 1H), 4.55-4.43 (m, 2H), 3.55-3.40 (m, 1H), 2.86 (d, J=13.5 Hz, 1H), 1.65 (s, 3H). ¹⁹F NMR (377 MHz, Chloroform-d) δ −78.72, −78.73, −79.16, −79.17, −82.21, −82.23, −82.65, −82.67, 137.10, −137.11, −137.12, −137.13.

### Example 67 and 68: (7R,14R)-1-(difluoromethoxy)-11-((R)-7-hydroxy-7-methyl-6,7-dihydro-5H-cyclopenta[b]pyridin-3-yl)-6-(methyl-d3)-6,7-dihydro-7,14-methanobenzo[f]pyrido[3′,2′:4,5]imidazo[1,2-a][1,4]diazocin-5(14H)-one and (7R,14R)-1-(difluoromethoxy)-11-((S)-7-hydroxy-7-methyl-6,7-dihydro-5H-cyclopenta[b]pyridin-3-yl)-6-(methyl-d3)-6,7-dihydro-7,14-methanobenzo[f]pyrido[3′,2′:4,5]imidazo[1,2-a][1,4]diazocin-5(14H)-one

### Synthetic Scheme

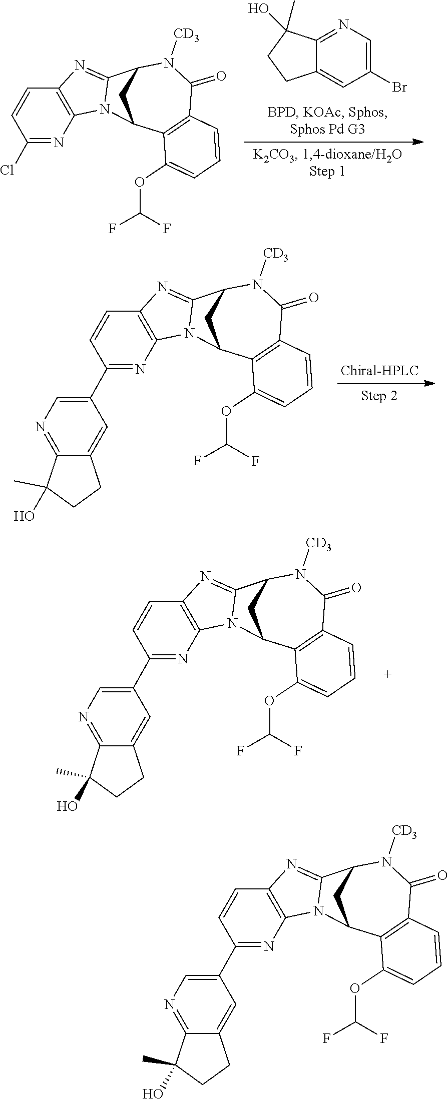 [SMILES: [2H]C([2H])([2H])N1C(=O)c2cccc(OC(F)F)c2[C@H]2C[C@@H]1c1nc3ccc(Cl)nc3n12 ; CC1(O)CCc2cc(Br)cnc21 ; [2H]C([2H])([2H])N1C(=O)c2cccc(OC(F)F)c2[C@H]2C[C@@H]1c1nc3ccc(-c4cnc5c(c4)CCC5(C)O)nc3n12 ; [2H]C([2H])([2H])N1C(=O)c2cccc(OC(F)F)c2[C@H]2C[C@@H]1c1nc3ccc(-c4cnc5c(c4)CC[C@@]5(C)O)nc3n12 ; [2H]C([2H])([2H])N1C(=O)c2cccc(OC(F)F)c2[C@H]2C[C@@H]1c1nc3ccc(-c4cnc5c(c4)CC[C@]5(C)O)nc3n12]

### Preparation 67A: (7R,14R)-1-(difluoromethoxy)-11-(7-hydroxy-7-methyl-6,7-dihydro-5H-cyclopenta[b]pyridin-3-yl)-6-(methyl-d3)-6,7-dihydro-7,14-methanobenzo[f]pyrido[3′,2′:4,5]imidazo[1,2-a][1,4]diazocin-5(14H)-one

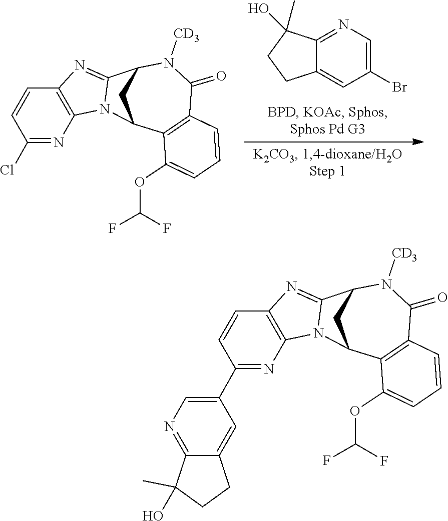 [SMILES: [2H]C([2H])([2H])N1C(=O)c2cccc(OC(F)F)c2[C@H]2C[C@@H]1c1nc3ccc(Cl)nc3n12 ; CC1(O)CCc2cc(Br)cnc21 ; [2H]C([2H])([2H])N1C(=O)c2cccc(OC(F)F)c2[C@H]2C[C@@H]1c1nc3ccc(-c4cnc5c(c4)CCC5(C)O)nc3n12]

A mixture of 3-bromo-7-methyl-5H,6H-cyclopenta[b]pyridin-7-ol (50 mg, 0.219 mmol), BPD (69 mg, 0.274 mmol), SPhos (12 mg, 0.031 mmol), SPhos Pd Gen.3 (11 mg, 0.015 mmol) and KOAc (53 mg, 0.547 mmol) in 1,4-dioxane (2 mL) at room temperature under nitrogen atmosphere. The resulting mixture was stirred for 2 h at 100° C. under nitrogen atmosphere. The mixture was allowed to cool down to room temperature. To the above mixture was added (7R,14R)-11-chloro-1-(difluoromethoxy)-6-(methyl-d3)-6,7-dihydro-7,14-methanobenzo[f]pyrido[3′,2′:4,5]imidazo[1,2-a][1,4]diazocin-5(14H)-one (71 mg, 0.182 mmol), K₂CO₃ (42 mg, 0.305 mmol) and H₂O (0.4 mL) at room temperature under nitrogen atmosphere. The resulting mixture was stirred for additional overnight at 80° C. The reaction was quenched with water at room temperature. The resulting mixture was extracted with EtOAc (3×30 mL). The combined organic layers were dried over anhydrous Na₂SO₄. After filtration, the filtrate was concentrated under reduced pressure. The residue was purified by silica gel column chromatography, eluted with CH₂Cl₂/MeOH (10:1) to afford (7R,14R)-1-(difluoromethoxy)-11-(7-hydroxy-7-methyl-6,7-dihydro-5H-cyclopenta[b]pyridin-3-yl)-6-(methyl-d3)-6,7-dihydro-7,14-methanobenzo[f]pyrido[3′,2′:4,5]imidazo[1,2-a][1,4]diazocin-5(14H)-one (80 mg, 86%) as a colorless oil. MS ESI calculated for C₂₇H₂₀D₃F₂N₅O₃ [M+H]⁺, 507.20 found 507.10.

### Example 67 and 68: (7R,14R)-1-(difluoromethoxy)-11-((R)-7-hydroxy-7-methyl-6,7-dihydro-5H-cyclopenta[b]pyridin-3-yl)-6-(methyl-d3)-6,7-dihydro-7,14-methanobenzo[f]pyrido[3′,2′:4,5]imidazo[1,2-a][1,4]diazocin-5(14H)-one and (7R,14R)-1-(difluoromethoxy)-11-((S)-7-hydroxy-7-methyl-6,7-dihydro-5H-cyclopenta[b]pyridin-3-yl)-6-(methyl-d3)-6,7-dihydro-7,14-methanobenzo[f]pyrido[3′,2′:4,5]imidazo[1,2-a][1,4]diazocin-5(14H)-one

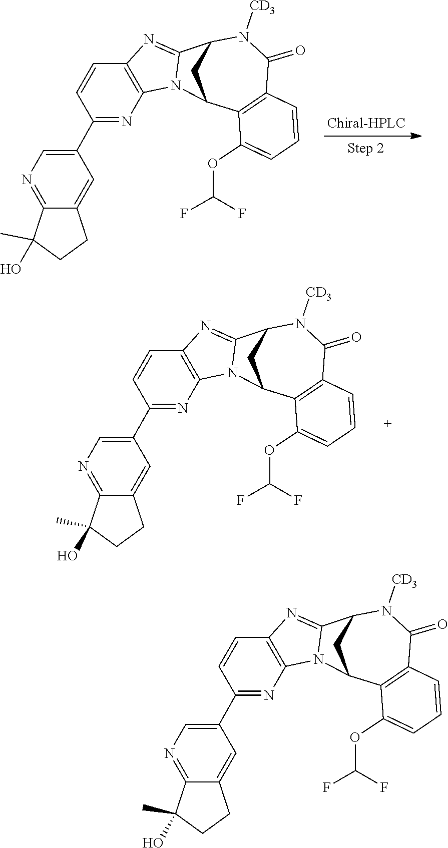 [SMILES: [2H]C([2H])([2H])N1C(=O)c2cccc(OC(F)F)c2[C@H]2C[C@@H]1c1nc3ccc(-c4cnc5c(c4)CCC5(C)O)nc3n12 ; [2H]C([2H])([2H])N1C(=O)c2cccc(OC(F)F)c2[C@H]2C[C@@H]1c1nc3ccc(-c4cnc5c(c4)CC[C@@]5(C)O)nc3n12 ; [2H]C([2H])([2H])N1C(=O)c2cccc(OC(F)F)c2[C@H]2C[C@@H]1c1nc3ccc(-c4cnc5c(c4)CC[C@]5(C)O)nc3n12]

(7R,14R)-1-(difluoromethoxy)-11-(7-hydroxy-7-methyl-6,7-dihydro-5H-cyclopenta[b]pyridin-3-yl)-6-(methyl-d3)-6,7-dihydro-7,14-methanobenzo[f]pyrido[3′,2′:4,5]imidazo[1,2-a][1,4]diazocin-5(14H)-one (80 mg) was purified by Prep-Chial-HPLC with the following conditions (Column: CHIRAL ART Cellulose-SZ, 3*25 cm, 5 μm; Mobile Phase A: Hex (10 mM NH₃-MeOH), Mobile Phase B: EtOH; Flow rate: 40 mL/min; Gradient: isocratic 50; Wave Length: 223/312 nm; RT1(min): 8.095; RT2(min): 11.802). The first peak afforded 28 mg (35%) as a white solid. MS ESI calculated for C₂₇H₂₀D₃F₂N₅O₃ [M+H]⁺, 507.20 found 507.25. ¹H NMR (400 MHz, Chloroform-d) δ 8.96 (d, J=2.0 Hz, 1H), 8.53-8.41 (m, 1H), 8.22-8.14 (m, 1H), 8.06 (d, J=8.4 Hz, 1H), 7.64 (d, J=8.4 Hz, 1H), 7.44-7.36 (m, 2H), 7.20-6.91 (m, 1H), 6.53 (d, J=7.3 Hz, 1H), 4.98 (d, J=6.8 Hz, 1H), 3.54-3.42 (m, 1H), 3.14-3.04 (m, 1H), 2.96-2.87 (m, 2H), 2.41-2.26 (m, 2H), 1.65 (s, 3H). ¹⁹F NMR (377 MHz, Chloroform-d) δ −77.96, −78.40, −81.82, −82.26. The second peak afforded 26 mg (32%) as a white solid. MS ESI calculated for C₂₇H₂₀D₃F₂N₅O₃ [M+H]⁺, 507.20 found 507.25. ¹H NMR (400 MHz, Chloroform-d) δ 8.95 (d, J=2.1 Hz, 1H), 8.49-8.45 (m, 1H), 8.19 (d, J=1.7 Hz, 1H), 8.06 (d, J=8.4 Hz, 1H), 7.64 (d, J=8.5 Hz, 1H), 7.41-7.36 (m, 2H), 7.14-6.92 (m, 1H), 6.53 (d, J=7.3 Hz, 1H), 4.98 (d, J=7.0 Hz, 1H), 3.56-3.42 (m, 1H), 3.16-3.04 (m, 1H), 2.98-2.85 (m, 2H), 2.41-2.28 (m, 2H), 1.65 (s, 3H). ¹⁹F NMR (377 MHz, Chloroform-d) δ −77.89, −78.34, −81.83, −82.27.

### Example 69 and 70: (7R,14R)-1-(difluoromethoxy)-11-((R)-4-hydroxy-4-methyl-3,4-dihydro-2H-pyrano[3,2-b]pyridin-7-yl)-6-(methyl-d3)-6,7-dihydro-7,14-methanobenzo[f]pyrido[3′,2′:4,5]imidazo[1,2-a][1,4]diazocin-5(14H)-one and (7R,14R)-1-(difluoromethoxy)-11-((S)-4-hydroxy-4-methyl-3,4-dihydro-2H-pyrano[3,2-b]pyridin-7-yl)-6-(methyl-d3)-6,7-dihydro-7,14-methanobenzo[f]pyrido[3′,2′:4,5]imidazo[1,2-a][1,4]diazocin-5(14H)-one

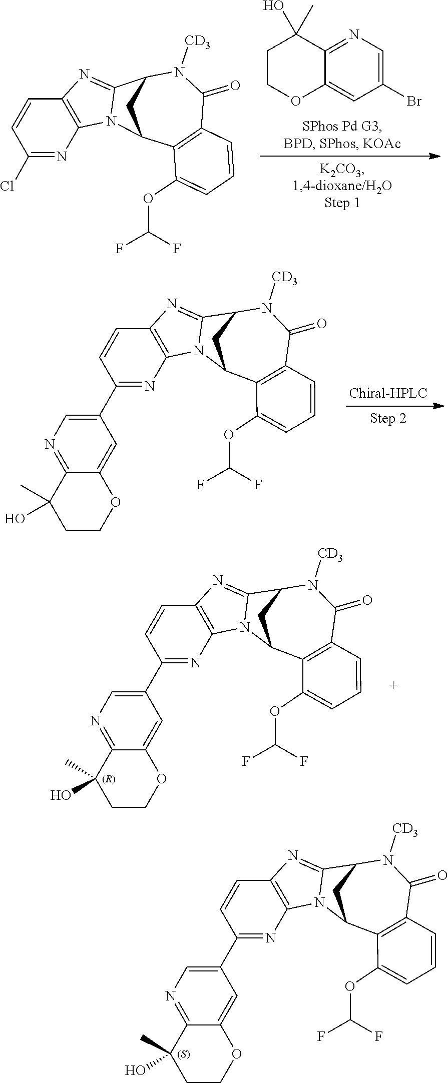

### Preparation 69A: (7R,14R)-1-(difluoromethoxy)-11-(4-hydroxy-4-methyl-3,4-dihydro-2H-pyrano[3,2-b]pyridin-7-yl)-6-(methyl-d3)-6,7-dihydro-7,14-methanobenzo[f]pyrido[3′,2′:4,5]imidazo[1,2-a][1,4]diazocin-5(14H)-one

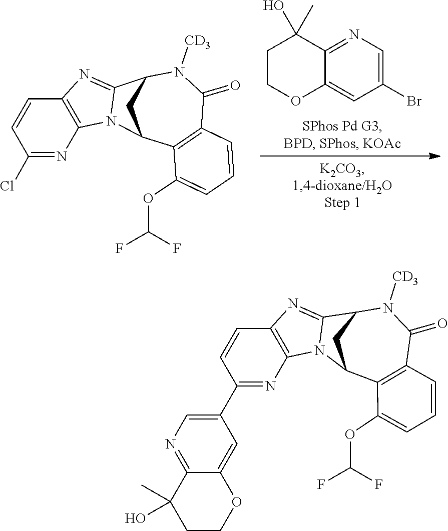

To a stirred solution of 7-bromo-4-methyl-3,4-dihydro-2H-pyrano[3,2-b]pyridin-4-ol (60 mg, 0.246 mmol), BPD (94 mg, 0.369 mmol) and KOAc (72 mg, 0.738 mmol) in 1,4-dioxane (1.2 mL) were added SPhos Pd G3 (19 mg, 0.025 mmol) and SPhos (20 mg, 0.049 mmol) at room temperature. The resulting mixture was stirred for 2 h at 100° C. under nitrogen atmosphere. The mixture was allowed to cool down to room temperature. To the above mixture were added (7R,14R)-11-chloro-1-(difluoromethoxy)-6-(methyl-d3)-6,7-dihydro-7,14-methanobenzo[f]pyrido[3′,2′:4,5]imidazo[1,2-a][1,4]diazocin-5(14H)-one (97 mg, 0.246 mmol), K₂CO₃ (68 mg, 0.492 mmol) and H₂O (0.2 mL). The resulting mixture was stirred for additional overnight at 80° C. under nitrogen atmosphere. The reaction was quenched with water at room temperature. The mixture was extracted with EtOAc (3×10 mL). The combined organic layers were washed with brine (3×10 mL), dried over anhydrous Na₂SO₄. After filtration, the filtrate was concentrated under reduced pressure. The residue was purified by reversed-phase flash chromatography with the following conditions: column, C18 silica gel; mobile phase, CH₃CN in Water (0.1% FA), 10% to 50% gradient in 10 min; detector, 254 nm. This resulted in (7R,14R)-1-(difluoromethoxy)-11-(4-hydroxy-4-methyl-3,4-dihydro-2H-pyrano[3,2-b]pyridin-7-yl)-6-(methyl-d3)-6,7-dihydro-7,14-methanobenzo[f]pyrido[3′,2′:4,5]imidazo[1,2-a][1,4]diazocin-5(14H)-one (80 mg, 62%) as a light yellow solid. MS ESI calculated for C₂₇H₂₀D₃F₂N₅O₄ [M+H]⁺, 523.20 found 523.25.

### Example 69 and 70: (7R,14R)-1-(difluoromethoxy)-11-((R)-4-hydroxy-4-methyl-3,4-dihydro-2H-pyrano[3,2-b]pyridin-7-yl)-6-(methyl-d3)-6,7-dihydro-7,14-methanobenzo[f]pyrido[3′,2′:4,5]imidazo[1,2-a][1,4]diazocin-5(14H)-one and (7R,14R)-1-(difluoromethoxy)-11-((S)-4-hydroxy-4-methyl-3,4-dihydro-2H-pyrano[3,2-b]pyridin-7-yl)-6-(methyl-d3)-6,7-dihydro-7,14-methanobenzo[f]pyrido[3′,2′:4,5]imidazo[1,2-a][1,4]diazocin-5(14H)-one

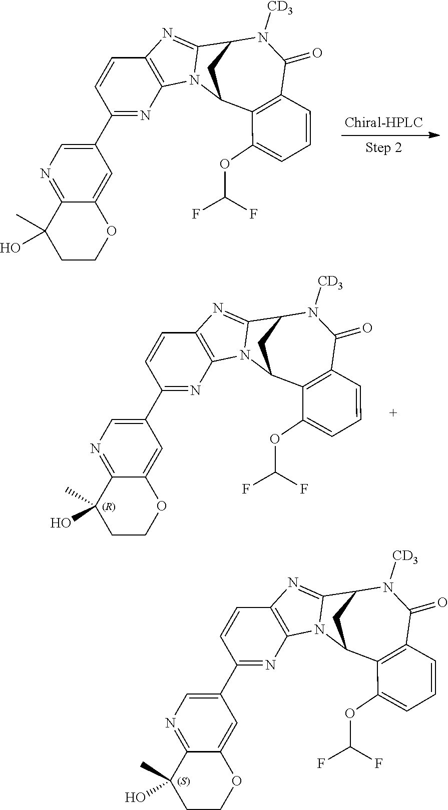

(7R,14R)-1-(difluoromethoxy)-11-(4-hydroxy-4-methyl-3,4-dihydro-2H-pyrano[3,2-b]pyridin-7-yl)-6-(methyl-d3)-6,7-dihydro-7,14-methanobenzo[f]pyrido[3′,2′:4,5]imidazo[1,2-a][1,4]diazocin-5(14H)-one (80 mg) was purified by Prep-Chiral HPLC with the following conditions (Column: CHIRALPAK-IK, 3*25 mm, 5 μm; Mobile Phase A: Hex (10 mM NH₃-MeOH), Mobile Phase B: EtOH; Flow rate: 40 mL/min; Gradient: isocratic 50; Wave Length: 317/200 nm; RT1(min): 11.324; RT2(min): 17.151). The first peak afforded 31 mg (39%) as a white solid. MS ESI calculated for C₂₇H₂₀D₃F₂N₅O₄ [M+H]⁺, 523.20, found 523.25. ¹H NMR (300 MHz, Chloroform-d) δ 8.72 (d, J=1.9 Hz, 1H), 8.53-8.41 (m, 1H), 8.05 (d, J=8.4 Hz, 1H), 7.73 (s, 1H), 7.60 (d, J=8.4 Hz, 1H), 7.46-7.33 (m, 2H), 7.36-6.83 (m, 1H), 6.53 (d, J=7.3 Hz, 1H), 4.97 (d, J=6.9 Hz, 1H), 4.49-4.35 (m, 1H), 4.37-4.23 (m, 1H), 3.55-3.40 (m, 1H), 2.89 (d, J=13.7 Hz, 1H), 2.39-2.11 (m, 2H), 1.72 (s, 3H). ¹⁹F NMR (282 MHz, Chloroform-d) δ −77.89, −78.48, −81.69, −82.28. The second peak afforded 31 mg (39%) as a white solid. MS ESI calculated for C₂₇H₂₀D₃F₂N₅O₄ [M+H]⁺, 523.20 found 523.25. ¹H NMR (300 MHz, Chloroform-d) δ 8.68 (d, J=1.8 Hz, 1H), 8.52-8.40 (m, 1H), 8.02 (d, J=8.5 Hz, 1H), 7.69 (d, J=1.8 Hz, 1H), 7.58 (d, J=8.4 Hz, 1H), 7.43-7.32 (m, 2H), 7.36-6.83 (m, 1H), 6.52 (d, J=7.5 Hz, 1H), 4.97 (d, J=7.0 Hz, 1H), 4.48-4.34 (m, 1H), 4.35-4.22 (m, 1H), 3.55-3.42 (m, 1H), 2.88 (d, J=13.4 Hz, 1H), 2.38-2.10 (m, 2H), 1.69 (s, 3H). ¹⁹F NMR (282 MHz, Chloroform-d) δ −77.83, −78.43, −81.74, −82.33.

### I. Biological Evaluation

### TNF-α Induced HEK Blue Cellular Assay

Test articles were diluted in DMSO and serially diluted into 384 well assay plate (Corning 3765), at final concentrations ranging from 30 mM to 0.5 nM. HEK Blue™ TNFα reporter cells were added at a final density of 10,000 cell per well in assay media [DMEM (Gibco, cat #21063-029), 10% fetal bovine serum (ExcelBio, cat #FND500), 1% Penicillin-Streptomycin (Solarbio, cat #P1400-100]. TNF-α (R&D 210-TA-020/CF) was then added to the assay plate at a final concentration of 100 μg/ml. This plate was then incubated for 24 hrs at 37° C. and 5% CO₂. Secreted alkaline phosphatase expression was then measured using QUANTI-Blue™(Invivogen), according to manufacturer instructions and read on an Envision microplate reader at 620 nm.

Inhibition data for test compound over a range of concentration was plotted as percentage inhibition of the test compound (100%=maximum inhibition). IC₅₀ values were determined after correcting for background [(sample read-mean of low control)/(mean of high control-mean of low control)] whereby the low control is DMSO without stimulation and high control is DMSO with stimulation. The IC₅₀ is defined as the concentration of test compound which produces 5000 inhibition and was quantified using the 4 parameter logistic equation to fit the data.

Representative data for exemplary compounds is presented in Table 3.

TABLE 3

| Ex. No | IC₅₀ value |
|---|---|
| 1 | A |
| 2 | A |
| 3 | A |
| 4 | A |
| 5 | A |
| 6 | A |
| 7 | A |
| 8 | A |
| 9 | A |
| 10 | A |
| 11 | A |
| 12 | A |
| 13 | A |
| 14 | A |
| 15 | A |
| 16 | A |
| 17 | A |
| 18 | B |
| 19 | A |
| 20 | A |
| 21 | A |
| 22 | A |
| 23 | A |
| 24 | A |
| 25 | A |
| 26 | A |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | D |
| 39 | D |
| 40 | D |
| 41 | D |
| 42 | A |
| 43 | A |
| 44 | A |
| 45 | B |
| 46 | A |
| 47 | A |
| 48 | A |
| 49 | B |
| 50 | A |
| 51 | A |
| 52 | A |
| 53 | A |
| 54 | A |
| 55 | A |
| 56 | A |
| 57 | A |
| 58 | A |
| 59 | D |
| 60 | B |
| 61 | A |
| 62 | A |
| 63 | B |
| 64 | B |
| 65 | A |
| 66 | A |
| 67 | D |
| 68 | D |
| 69 | D |
| 70 | B |

Note:
IC₅₀ data are designated within the following ranges:
A: ≤0.1 μM
B: >0.1 μM to ≤1.0 μM
C: >1 μM to ≤10 μM
D: >10 μM to ≤30 μM

### III. Preparation of Pharmaceutical Dosage Forms

### Example 1: Oral Capsule

The active ingredient is a compound of Table 1, or a pharmaceutically acceptable salt or solvate thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### Example 2: Solution for Injection

The active ingredient is a compound of Table 1, or a pharmaceutically acceptable salt or solvate thereof, and is formulated as a solution in sesame oil at a concentration of 50 mg-eq/mL.

The examples and embodiments described herein are for illustrative purposes only and various modifications or changes suggested to persons skilled in the art are to be included within the spirit and purview of this application and scope of the appended claims.

We claim:

1. A compound of Formula (I), or pharmaceutically acceptable salt, solvate, or N-oxide thereof: 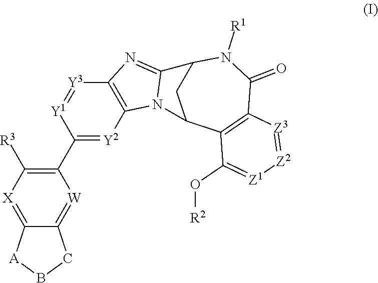
wherein,
W is N or C—R⁴;
X is N, C—F, or C—H;
Y¹ is N, C—F, or C—H;
Y² is N, C—F, or C—H;
Y³ is N, C—F, or C—H;
Z¹ is N or C—H;
Z² is N or C—H;
Z³ is N or C—H;
A is CR⁵R⁶, or N—R⁹;
B is CR⁷R⁸, or N—R¹⁰;
C is —CH₂—, —CH₂CH₂—, —NH—, —O—, —OCH₂—, or —CH₂O—;
R¹ is selected from hydrogen, optionally substituted C1-C6 alkyl, optionally substituted C3-C6 cycloalkyl, or optionally substituted C4-C7 cycloalkylalkyl;
R² is hydrogen, or optionally substituted C1-C4 alkyl;
R³ is hydrogen, methyl, or halogen;
R⁴ is hydrogen, halogen, or optionally substituted C1 alkyl;
R⁵ is —OH, —NH₂, or —NH(optionally substituted C1-C6 alkyl); or optionally, R⁵ and R⁶ are both —OH and join to form an oxo group;
R⁶ is hydrogen, —OH, or optionally substituted C1-C6 alkyl; or optionally, R⁶ and R⁸ are both optionally substituted C1-C6 alkyl and join to form a ring;
R⁷ is hydrogen, —OH, or optionally substituted C1-C6 alkyl; or optionally, R⁷ and R⁸ are both —OH and join to form an oxo group;
R⁸ is hydrogen, —OH, or optionally substituted C1-C6 alkyl;
R⁹ is hydrogen, or optionally substituted C1-C6 alkyl; and
R¹⁰ is hydrogen, or optionally substituted C1-C6 alkyl.

2. A compound of Formula (Ia), or pharmaceutically acceptable salt, solvate, or N-oxide thereof: 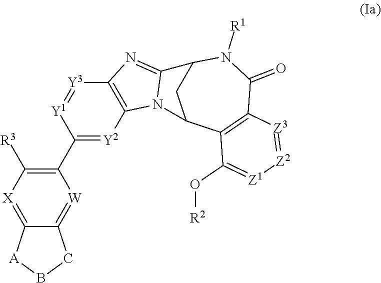
wherein,
W is N or C—R⁴;
X is N, C—F, or C—H;
Y¹ is N, C—F, or C—H;
Y² is N, C—F, or C—H;
Y³ is N, C—F, or C—H;
Z¹ is N or C—H;
Z² is N or C—H;
Z³ is N or C—H;
A is CR⁵R⁶, or N—R⁹;
B is CR⁷R⁸, or N—R¹⁰;
C is —CH₂—, —CH₂CH₂—, —NH—, —O— or —CH₂O—;
R¹ is selected from hydrogen, optionally substituted C1-C6 alkyl, optionally substituted C3-C6 cycloalkyl, or optionally substituted C4-C7 cycloalkylalkyl;
R² is hydrogen, or optionally substituted C1-C4 alkyl;
R³ is hydrogen or halogen;
R⁴ is hydrogen, halogen, or optionally substituted C1 alkyl;
R⁵ is —OH, —NH₂, or —NH(optionally substituted C1-C6 alkyl); or optionally, R⁵ and R⁶ are both —OH and join to form an oxo group;
R⁶ is hydrogen, —OH, or optionally substituted C1-C6 alkyl; or optionally, R⁶ and R⁸ are both optionally substituted C1-C6 alkyl and join to form a ring;
R⁷ is hydrogen, —OH, or optionally substituted C1-C6 alkyl; or optionally, R⁷ and R⁸ are both —OH and join to form an oxo group;
R⁸ is hydrogen, —OH, or optionally substituted C1-C6 alkyl;
R⁹ is hydrogen, or optionally substituted C₁-C₆ alkyl; and
R¹⁰ is hydrogen, or optionally substituted C₁-C₆ alkyl.

3. The compound of claim 1 or 2, or pharmaceutically acceptable salt, solvate, or N-oxide thereof, wherein X is N.

4. The compound of claim 1 or 2, or pharmaceutically acceptable salt, solvate, or N-oxide thereof, wherein X is C—F, or C—H.

5. The compound of claim 1, 2, 3 or 4, or pharmaceutically acceptable salt, solvate, or N-oxide thereof, wherein Z¹ is N.

6. The compound of claim 1, 2, 3 or 4, or pharmaceutically acceptable salt, solvate, or N-oxide thereof, wherein Z¹ is C—H.

7. The compound of any one of claims 1-6, or pharmaceutically acceptable salt, solvate, or N-oxide thereof, wherein Z² is N.

8. The compound of any one of claims 1-6, or pharmaceutically acceptable salt, solvate, or N-oxide thereof, wherein Z² is C—H.

9. The compound of any one of claims 1-8, or pharmaceutically acceptable salt, solvate, or N-oxide thereof, wherein Z³ is N.

10. The compound of any one of claims 1-8, or pharmaceutically acceptable salt, solvate, or N-oxide thereof, wherein Z³ is C—H.

11. The compound of any one of claims 1-10, or pharmaceutically acceptable salt, solvate, or N-oxide thereof, wherein Y¹ is N.

12. The compound of any one of claims 1-10, or pharmaceutically acceptable salt, solvate, or N-oxide thereof, wherein Y¹ is C—F or C—H.

13. The compound of any one of claims 1-12, or pharmaceutically acceptable salt, solvate, or N-oxide thereof, wherein Y² is N.

14. The compound of any one of claims 1-12, or pharmaceutically acceptable salt, solvate, or N-oxide thereof, wherein Y² is C—F or C—H.

15. The compound of any one of claims 1-14, or pharmaceutically acceptable salt, solvate, or N-oxide thereof, wherein Y³ is N.

16. The compound of any one of claims 1-14, or pharmaceutically acceptable salt, solvate, or N-oxide thereof, wherein Y³ is C—F or C—H.

17. The compound of any one of claims 1-16, or pharmaceutically acceptable salt, solvate, or N-oxide thereof, wherein W is N.

18. The compound of any one of claims 1-16, or pharmaceutically acceptable salt, solvate, or N-oxide thereof, wherein W is C—R⁴.

19. The compound of any one of claims 1-18, or pharmaceutically acceptable salt, solvate, or N-oxide thereof, wherein A is CR⁵R⁶; B is CR⁷R⁸; and C is —CH₂— or —CH₂CH₂—.

20. The compound of any one of claims 1-18, or pharmaceutically acceptable salt, solvate, or N-oxide thereof, wherein A is CR⁵R⁶; B is CR⁷R⁸; and C is —O—, —OCH₂—, or —CH₂O—.

21. The compound of any one of claims 1-18, or pharmaceutically acceptable salt, solvate, or N-oxide thereof, wherein A is CR⁵R⁶; B is CR⁷R⁸; and C is —O—, —OCH₂—, or —CH₂O—.

22. The compound of any one of claims 1-18, or pharmaceutically acceptable salt, solvate, or N-oxide thereof, wherein A is CR⁵R⁶, and R⁵ and R⁶ are both —OH joined to form an oxo group; and B is N—R¹⁰.

23. The compound of any one of claims 1-18, or pharmaceutically acceptable salt, solvate, or N-oxide thereof, wherein A is N—R⁹; and B is CR⁷R⁸ and R⁷ and R⁸ are both —OH joined to form an oxo group.

24. The compound of claim 19 or 20, or pharmaceutically acceptable salt, solvate, or N-oxide thereof, wherein A is CR⁵R⁶, and R⁵ and R⁶ are both —OH joined to form an oxo group.

25. The compound of claim 19 or 20, or pharmaceutically acceptable salt, solvate, or N-oxide thereof, wherein R⁶ and R⁸ are both optionally substituted C1-C6 alkyl and join to form a ring.

26. The compound of any one of claims 1-25, or pharmaceutically acceptable salt, solvate, or N-oxide thereof, wherein R¹ is selected from hydrogen or optionally substituted C1-C6 alkyl.

27. The compound of any one of claims 1-25, or pharmaceutically acceptable salt, solvate, or N-oxide thereof, wherein R¹ is selected from CH₃ or CD₃.

28. The compound of any one of claims 1-27, or pharmaceutically acceptable salt, solvate, or N-oxide thereof, wherein R² is selected from optionally substituted C1-C6 alkyl.

29. The compound of any one of claims 1-27, or pharmaceutically acceptable salt, solvate, or N-oxide thereof, wherein R² is selected from optionally substituted C1 alkyl.

30. The compound of any one of claims 1-27, or pharmaceutically acceptable salt, solvate, or N-oxide thereof, wherein R² is —CHF₂.

31. The compound of any one of claims 1-30, or pharmaceutically acceptable salt, solvate, or N-oxide thereof, wherein R³ is hydrogen.

32. The compound of any one of claims 1-30, or pharmaceutically acceptable salt, solvate, or N-oxide thereof, wherein R⁴ is hydrogen.

33. The compound of any one of claims 1-21, 23, or 25-32, or pharmaceutically acceptable salt, solvate, or N-oxide thereof, wherein R⁵ is —OH.

34. The compound of any one of claims 1-21, 23, or 25-32, or pharmaceutically acceptable salt, solvate, or N-oxide thereof, wherein R⁵ is —NH₂.

35. The compound of claim 33 or 34, or pharmaceutically acceptable salt, solvate, or N-oxide thereof, wherein R⁶ is hydrogen.

36. The compound of claim 33 or 34, or pharmaceutically acceptable salt, solvate, or N-oxide thereof, wherein R⁶ is optionally substituted C1-C6 alkyl.

37. The compound of claim 33 or 34, or pharmaceutically acceptable salt, solvate, or N-oxide thereof, wherein R⁶ is —CH₃.

38. A compound described in Table 1, or pharmaceutically acceptable salt, solvate, or N-oxide thereof.

39. A compound described in Table 2, or pharmaceutically acceptable salt, solvate, or N-oxide thereof.

40. A pharmaceutical composition comprising the compound, or pharmaceutically acceptable salt or solvate or N-oxide thereof, of any one of claims 1-39 and a pharmaceutically acceptable excipient or carrier.

41. A method of preparing a pharmaceutical composition comprising mixing the compound, or pharmaceutically acceptable salt or solvate or N-oxide thereof, of any one of claims 1-39, and a pharmaceutically acceptable excipient or carrier.

42. A compound, or pharmaceutically acceptable salt or solvate or N-oxide thereof, of any one of claims 1-39, or the pharmaceutical composition of claim 40, for use in a method of treatment of the human or animal body.

43. A compound, or pharmaceutically acceptable salt or solvate or N-oxide thereof, of any one of claims 1-39, or the pharmaceutical composition of claim 40, for use in a method of treatment of inflammatory or autoimmune disease or disorder.

44. Use of a compound, or pharmaceutically acceptable salt or solvate or N-oxide thereof, of any one of claims 1-39, or the pharmaceutical composition of claim 40, in the manufacture of a medicament for the treatment of an inflammatory or autoimmune disease or disorder.

45. A pharmaceutical composition comprising the compound, or pharmaceutically acceptable salt or solvate or N-oxide thereof, of any one of claims 1-39, for use in an inflammatory or autoimmune disease or disorder in a patient in need thereof.

46. A method of treating an inflammatory or autoimmune disease or disorder in a patient in need thereof, comprising administering to the patient a therapeutically effectively amount of the compound, or pharmaceutically acceptable salt or solvate or N-oxide thereof, of any one of claims 1-39, or the pharmaceutical composition of claim 40.

47. A method of inhibiting TNF-a activity comprising contacting the TNF-a protein with the compound, or pharmaceutically acceptable salt or solvate or N-oxide thereof, of any one of claims 1-39, or the pharmaceutical composition of claim 40, wherein the TNF-α protein is contacted in an in vitro setting.

48. A method of inhibiting TNF-α activity comprising contacting the TNF-α protein with the compound, or pharmaceutically acceptable salt or solvate or N-oxide thereof, of any one of claims 1-39, or the pharmaceutical composition of claim 40, wherein the TNF-α protein is contacted in an in vivo setting.

49. A method of treating or preventing a condition conducive to treatment or prevention by inhibition of TNF-α in a patient comprising administering to the patient a therapeutically effective amount of the compound, or pharmaceutically acceptable salt or solvate or N-oxide thereof, of any one of claims 1-39, or the pharmaceutical composition of claim 40.

50. A pharmaceutical composition comprising the compound, or pharmaceutically acceptable salt or solvate or N-oxide thereof, of any one of claims 1-39, for use in treating or preventing a condition conducive to treatment or prevention by inhibition of TNF-α in a patient.

51. The compound, or pharmaceutically acceptable salt or solvate or N-oxide thereof, of any one of claims 1-39 or the pharmaceutical composition of claim 40, for use in treating or preventing a condition conducive to treatment or prevention by inhibition of TNF-α in a patient.

52. Use of the compound, or pharmaceutically acceptable salt or solvate or N-oxide thereof, of any one of claims 1-39, or the pharmaceutical composition of claim 40, in the preparation of a medicament for treating or preventing a condition conducive to treatment or prevention by inhibition of TNF-α in a patient.
